SEQUENCE LISTING (302 sequences)

SEQ ID NO: 1 - skipped

SEQ ID NO: 2 - protein, 847 aa, Artificial Sequence
  STAT6 Protein Sequence
MSLWGLVSKMPPEKVQRLYVDFPQHLRHLLGDWLESQPWEFLVGSDAFCCNLASALLSDTVQHLQASVGEQGEGSTILQHISTLESIYQRDPLKLVATFRQILQGEKKAVMEQFRHLPMPFHWKQEELKFKTGLRRLQHRVGEIHLLREALQKGAEAGQVSLHSLIETPANGTGPSEALAMLLQETTGELEAAKALVLKRIQIWKRQQQLAGNGAPFEESLAPLQERCESLVDIYSQLQQEVGAAGGELEPKTRASLTGRLDEVLRTLVTSCFLVEKQPPQVLKTQTKFQAGVRFLLGLRFLGAPAKPPLVRADMVTEKQARELSVPQGPGAGAESTGEIINNTVPLENSIPGNCCSALFKNLLLKKIKRCERKGTESVTEEKCAVLFSASFTLGPGKLPIQLQALSLPLVVIVHGNQDNNAKATILWDNAFSEMDRVPFVVAERVPWEKMCETLNLKFMAEVGTNRGLLPEHFLFLAQKIFNDNSLSMEAFQHRSVSWSQFNKEILLGRGFTFWQWFDGVLDLTKRCLRSYWSDRLIIGFISKQYVTSLLLNEPDGTFLLRFSDSEIGGITIAHVIRGQDGSPQIENIQPFSAKDLSIRSLGDRIRDLAQLKNLYPKKPKDEAFRSHYKPEQMGKDGRGYVPATIKMTVERDQPLPTPELQMPTMVPSYDLGMAPDSSMSMQLGPDMVPQVYPPHSHSIPPYQGLSPEESVNVLSAFQEPHLQMPPSLGQMSLPFDQPHPQGLLPCQPQEHAVSSPDPLLCSDVTMVEDSCLSQPVTAFPQGTWIGEDIFPPLLPPTEQDLTKLLLEGQGESGGGSLGAQPLLQPSHYGQSGISMSHMDLRANPSW

SEQ ID NO: 3 - DNA, 3963 bp, Artificial Sequence
  STAT6 mRNA Sequence
ggggcagccactgcttacactgaagagggaggacgggagaggagtgtgtgtgtgtgtgtgtgtgtgtgtgtgtatgtatgtgtgtgctttatcttatttttctttttggtggtggtggtggaaggggggaggtgctagcagggccagccttgaactcgctggacagagctacagacctatggggcctggaagtgcccgctgagaaagggagaagacagcagaggggttgccgaggcaacctccaagtcccagatcatgtctctgtggggtctggtctccaagatgcccccagaaaaagtgcagcggctctatgtcgactttccccaacacctgcggcatcttctgggtgactggctggagagccagccctgggagttcctggtcggctccgacgccttctgctgcaacttggctagtgccctactttcagacactgtccagcaccttcaggcctcggtgggagagcagggggaggggagcaccatcttgcaacacatcagcacccttgagagcatatatcagagggaccccctgaagctggtggccactttcagacaaatacttcaaggagagaaaaaagctgttatggaacagttccgccacttgccaatgcctttccactggaagcaggaagaactcaagtttaagacaggcttgcggaggctgcagcaccgagtaggggagatccaccttctccgagaagccctgcagaagggggctgaggctggccaagtgtctctgcacagcttgatagaaactcctgctaatgggactgggccaagtgaggccctggccatgctactgcaggagaccactggagagctagaggcagccaaagccctagtgctgaagaggatccagatttggaaacggcagcagcagctggcagggaatggcgcaccgtttgaggagagcctggccccactccaggagaggtgtgaaagcctggtggacatttattcccagctacagcaggaggtaggggcggctggtggggagcttgagcccaagacccgggcatcgctgactggccggctggatgaagtcctgagaaccctcgtcaccagttgcttcctggtggagaagcagcccccccaggtactgaagactcagaccaagttccaggctggagttcgattcctgttgggcttgaggttcctgggggccccagccaagcctccgctggtcagggccgacatggtgacagagaagcaggcgcgggagctgagtgtgcctcagggtcctggggctggagcagaaagcactggagaaatcatcaacaacactgtgcccttggagaacagcattcctgggaactgctgctctgccctgttcaagaacctgcttctcaagaagatcaagcggtgtgagcggaagggcactgagtctgtcacagaggagaagtgcgctgtgctcttctctgccagcttcacacttggccccggcaaactccccatccagctccaggccctgtctctgcccctggtggtcatcgtccatggcaaccaagacaacaatgccaaagccactatcctgtgggacaatgccttctctgagatggaccgcgtgccctttgtggtggctgagcgggtgccctgggagaagatgtgtgaaactctgaacctgaagttcatggctgaggtggggaccaaccgggggctgctcccagagcacttcctcttcctggcccagaagatcttcaatgacaacagcctcagtatggaggccttccagcaccgttctgtgtcctggtcgcagttcaacaaggagatcctgctgggccgtggcttcaccttttggcagtggtttgatggtgtcctggacctcaccaaacgctgtctccggagctactggtctgaccggctgatcattggcttcatcagcaaacagtacgttactagccttcttctcaatgagcccgacggaacctttctcctccgcttcagcgactcagagattgggggcatcaccattgcccatgtcatccggggccaggatggctctccacagatagagaacatccagccattctctgccaaagacctgtccattcgctcactgggggaccgaatccgggatcttgctcagctcaaaaatctctatcccaagaagcccaaggatgaggctttccggagccactacaagcctgaacagatgggtaaggatggcaggggttatgtcccagctaccatcaagatgaccgtggaaagggaccaaccacttcctaccccagagctccagatgcctaccatggtgccttcttatgaccttggaatggcccctgattcctccatgagcatgcagcttggcccagatatggtgccccaggtgtacccaccacactctcactccatccccccgtatcaaggcctctccccagaagaatcagtcaacgtgttgtcagccttccaggagcctcacctgcagatgccccccagcctgggccagatgagcctgccctttgaccagcctcacccccagggcctgctgccgtgccagcctcaggagcatgctgtgtccagccctgaccccctgctctgctcagatgtgaccatggtggaagacagctgcctgagccagccagtgacagcgtttcctcagggcacttggattggtgaagacatattccctcctctgctgcctcccactgaacaggacctcactaagcttctcctggaggggcaaggggagtcggggggagggtccttgggggcacagcccctcctgcagccctcccactatgggcaatctgggatctcaatgtcccacatggacctaagggccaaccccagttggtgatcccagctggagggagaacccaaagagacagctcttctactacccccacagacctgctctggacacttgctcatgccctgccaagcagcagatggggagggtgccctcctatccccacctactcctgggtcaggaggaaaagactaacaggagaatgcacagtgggtggagccaatccactccttcctttctatcattcccctgcccacctccttccagcactgactggaagggaagttcaggctctgagacacaccccaacatgcctgcacctgcagcgcgcacacgcacgcacacacacatacagagctctctgagggtgatggggctgagcaggaggggggctgggtaagagcacaggttagggcatggaaggcttctccgcccattctgacccagggcctaggacggataggcaggaacatacagacacatttacactagaggccagggatagaggatattgggtctcagccctaggggaatgggaagcagctcaagggaccctgggtgggagcataggaggggtctggacatgtggttactagtacaggttttgccctgattaaaaaatctcccaaagccccaaattcctgttagccaggtggaggcttctgatacgtgtatgagactatgcaaaagtacaagggctgagattcttcgtgtatagctgtgtgaacgtgtatgtacctaggatatgttaaatgtatagctggcaccttagttgcatgaccacatagaacatgtgtctatctgcttttgcctacgtgacaacacaaatttgggagggtgagacactgcacagaagacagcagcaagtgtgctggcctctctgacatatgctaacccccaaatactctgaatttggagtctgactgtgcccaagtgggtccaagtggctgtgacatctacgtatggctccacacctccaatgctgcctgggagccagggtgagagtctgggtccaggcctggccatgtggccctccagtgtatgagagggccctgcctgctgcatcttttctgttgccccatccaccgccagcttcccttcactcccctatcccattctccctctcaaggcaggggtcatagatcctaagccataaaataaattttattccaaaataacaaaataaataatctactgtacacaatctgaaaa

SEQ ID NO: 4-90 - skipped (87 entries)

SEQ ID NO: 91 - DNA, 15 bp, Artificial Sequence
  Synthetic sequence
ctggtgacgagggtt

SEQ ID NO: 92 - DNA, 15 bp, Artificial Sequence
  Synthetic sequence
cgctcacaccgcttg

SEQ ID NO: 93 - DNA, 15 bp, Artificial Sequence
  Synthetic sequence
aggctagtaacgtac

SEQ ID NO: 94 - DNA, 15 bp, Artificial Sequence
  Synthetic sequence
gaaggctagtaacgt

SEQ ID NO: 95 - DNA, 15 bp, Artificial Sequence
  Synthetic sequence
ggttccgtcgggctc

SEQ ID NO: 96 - DNA, 15 bp, Artificial Sequence
  Synthetic sequence
aggttccgtcgggct

SEQ ID NO: 97 - DNA, 15 bp, Artificial Sequence
  Synthetic sequence
aaggttccgtcgggc

SEQ ID NO: 98 - DNA, 15 bp, Artificial Sequence
  Synthetic sequence
aaaggttccgtcggg

SEQ ID NO: 99 - DNA, 15 bp, Artificial Sequence
  Synthetic sequence
gaaaggttccgtcgg

SEQ ID NO: 100 - DNA, 15 bp, Artificial Sequence
  Synthetic sequence
agaaaggttccgtcg

SEQ ID NO: 101 - DNA, 15 bp, Artificial Sequence
  Synthetic sequence
agtcgctgaagcgga

SEQ ID NO: 102 - DNA, 15 bp, Artificial Sequence
  Synthetic sequence
gagtcgctgaagcgg

SEQ ID NO: 103 - DNA, 15 bp, Artificial Sequence
  Synthetic sequence
cggattcggtccccc

SEQ ID NO: 104 - DNA, 15 bp, Artificial Sequence
  Synthetic sequence
ccggattcggtcccc

SEQ ID NO: 105 - DNA, 15 bp, Artificial Sequence
  Synthetic sequence
cccggattcggtccc

SEQ ID NO: 106 - DNA, 15 bp, Artificial Sequence
  Synthetic sequence
tcccggattcggtcc

SEQ ID NO: 107 - DNA, 15 bp, Artificial Sequence
  Synthetic sequence
gatcccggattcggt

SEQ ID NO: 108 - DNA, 15 bp, Artificial Sequence
  Synthetic sequence
agatcccggattcgg

SEQ ID NO: 109 - DNA, 15 bp, Artificial Sequence
  Synthetic sequence
aagatcccggattcg

SEQ ID NO: 110 - DNA, 15 bp, Artificial Sequence
  Synthetic sequence
tgatacggggggatg

SEQ ID NO: 111 - DNA, 15 bp, Artificial Sequence
  Synthetic sequence
cgtgtgcgcgctgca

SEQ ID NO: 112 - DNA, 16 bp, Artificial Sequence
  Synthetic sequence
actggtgacgagggtt

SEQ ID NO: 113 - DNA, 16 bp, Artificial Sequence
  Synthetic sequence
aactggtgacgagggt

SEQ ID NO: 114 - DNA, 16 bp, Artificial Sequence
  Synthetic sequence
ctcacaccgcttgatc

SEQ ID NO: 115 - DNA, 16 bp, Artificial Sequence
  Synthetic sequence
cggtcagaccagtagc

SEQ ID NO: 116 - DNA, 16 bp, Artificial Sequence
  Synthetic sequence
ctagtaacgtactgtt

SEQ ID NO: 117 - DNA, 16 bp, Artificial Sequence
  Synthetic sequence
gctagtaacgtactgt

SEQ ID NO: 118 - DNA, 16 bp, Artificial Sequence
  Synthetic sequence
ggctagtaacgtactg

SEQ ID NO: 119 - DNA, 16 bp, Artificial Sequence
  Synthetic sequence
aggctagtaacgtact

SEQ ID NO: 120 - DNA, 16 bp, Artificial Sequence
  Synthetic sequence
aaggctagtaacgtac

SEQ ID NO: 121 - DNA, 16 bp, Artificial Sequence
  Synthetic sequence
aagaaggctagtaacg

SEQ ID NO: 122 - DNA, 16 bp, Artificial Sequence
  Synthetic sequence
aaaggttccgtcgggc

SEQ ID NO: 123 - DNA, 16 bp, Artificial Sequence
  Synthetic sequence
agaaaggttccgtcgg

SEQ ID NO: 124 - DNA, 16 bp, Artificial Sequence
  Synthetic sequence
ttcggtcccccagtga

SEQ ID NO: 125 - DNA, 16 bp, Artificial Sequence
  Synthetic sequence
cggattcggtccccca

SEQ ID NO: 126 - DNA, 16 bp, Artificial Sequence
  Synthetic sequence
agatcccggattcggt

SEQ ID NO: 127 - DNA, 16 bp, Artificial Sequence
  Synthetic sequence
agcaagatcccggatt

SEQ ID NO: 128 - DNA, 16 bp, Artificial Sequence
  Synthetic sequence
tgatacggggggatgg

SEQ ID NO: 129 - DNA, 16 bp, Artificial Sequence
  Synthetic sequence
ttgatacggggggatg

SEQ ID NO: 130 - DNA, 16 bp, Artificial Sequence
  Synthetic sequence
agaggccttgatacgg

SEQ ID NO: 131 - DNA, 16 bp, Artificial Sequence
  Synthetic sequence
gggttagcatatgtca

SEQ ID NO: 132 - DNA, 17 bp, Artificial Sequence
  Synthetic sequence
tggatctcccctactcg

SEQ ID NO: 133 - DNA, 17 bp, Artificial Sequence
  Synthetic sequence
actggtgacgagggttc

SEQ ID NO: 134 - DNA, 17 bp, Artificial Sequence
  Synthetic sequence
gctcacaccgcttgatc

SEQ ID NO: 135 - DNA, 17 bp, Artificial Sequence
  Synthetic sequence
cgctcacaccgcttgat

SEQ ID NO: 136 - DNA, 17 bp, Artificial Sequence
  Synthetic sequence
tccgctcacaccgcttg

SEQ ID NO: 137 - DNA, 17 bp, Artificial Sequence
  Synthetic sequence
ttccgctcacaccgctt

SEQ ID NO: 138 - DNA, 17 bp, Artificial Sequence
  Synthetic sequence
accagtagctccggaga

SEQ ID NO: 139 - DNA, 17 bp, Artificial Sequence
  Synthetic sequence
gccggtcagaccagtag

SEQ ID NO: 140 - DNA, 17 bp, Artificial Sequence
  Synthetic sequence
agccggtcagaccagta

SEQ ID NO: 141 - DNA, 17 bp, Artificial Sequence
  Synthetic sequence
agaaggctagtaacgta

SEQ ID NO: 142 - DNA, 17 bp, Artificial Sequence
  Synthetic sequence
aaggttccgtcgggctc

SEQ ID NO: 143 - DNA, 17 bp, Artificial Sequence
  Synthetic sequence
aaaggttccgtcgggct

SEQ ID NO: 144 - DNA, 17 bp, Artificial Sequence
  Synthetic sequence
gaaaggttccgtcgggc

SEQ ID NO: 145 - DNA, 17 bp, Artificial Sequence
  Synthetic sequence
ctgagtcgctgaagcgg

SEQ ID NO: 146 - DNA, 17 bp, Artificial Sequence
  Synthetic sequence
tctgagtcgctgaagcg

SEQ ID NO: 147 - DNA, 17 bp, Artificial Sequence
  Synthetic sequence
atcccggattcggtccc

SEQ ID NO: 148 - DNA, 17 bp, Artificial Sequence
  Synthetic sequence
gatcccggattcggtcc

SEQ ID NO: 149 - DNA, 17 bp, Artificial Sequence
  Synthetic sequence
agatcccggattcggtc

SEQ ID NO: 150 - DNA, 17 bp, Artificial Sequence
  Synthetic sequence
aagatcccggattcggt

SEQ ID NO: 151 - DNA, 17 bp, Artificial Sequence
  Synthetic sequence
gcaagatcccggattcg

SEQ ID NO: 152 - DNA, 17 bp, Artificial Sequence
  Synthetic sequence
cggtcatcttgatggta

SEQ ID NO: 153 - DNA, 17 bp, Artificial Sequence
  Synthetic sequence
tgatacggggggatgga

SEQ ID NO: 154 - DNA, 17 bp, Artificial Sequence
  Synthetic sequence
ttgatacggggggatgg

SEQ ID NO: 155 - DNA, 17 bp, Artificial Sequence
  Synthetic sequence
cttgatacggggggatg

SEQ ID NO: 156 - DNA, 17 bp, Artificial Sequence
  Synthetic sequence
gaggccttgatacgggg

SEQ ID NO: 157 - DNA, 20 bp, Artificial Sequence
  Synthetic sequence
gggggtccctctgatatatg

SEQ ID NO: 158 - DNA, 20 bp, Artificial Sequence
  Synthetic sequence
gtggatctcccctactcggt

SEQ ID NO: 159 - DNA, 20 bp, Artificial Sequence
  Synthetic sequence
agcccaacaggaatcgaact

SEQ ID NO: 160 - DNA, 20 bp, Artificial Sequence
  Synthetic sequence
cgctcacaccgcttgatctt

SEQ ID NO: 161 - DNA, 20 bp, Artificial Sequence
  Synthetic sequence
ccgctcacaccgcttgatct

SEQ ID NO: 162 - DNA, 20 bp, Artificial Sequence
  Synthetic sequence
tccgctcacaccgcttgatc

SEQ ID NO: 163 - DNA, 20 bp, Artificial Sequence
  Synthetic sequence
agtttgccggggccaagtgt

SEQ ID NO: 164 - DNA, 20 bp, Artificial Sequence
  Synthetic sequence
aagggcacgcggtccatctc

SEQ ID NO: 165 - DNA, 20 bp, Artificial Sequence
  Synthetic sequence
aaagggcacgcggtccatct

SEQ ID NO: 166 - DNA, 20 bp, Artificial Sequence
  Synthetic sequence
caaagggcacgcggtccatc

SEQ ID NO: 167 - DNA, 20 bp, Artificial Sequence
  Synthetic sequence
acaaagggcacgcggtccat

SEQ ID NO: 168 - DNA, 20 bp, Artificial Sequence
  Synthetic sequence
ccggagacagcgtttggtga

SEQ ID NO: 169 - DNA, 20 bp, Artificial Sequence
  Synthetic sequence
tccggagacagcgtttggtg

SEQ ID NO: 170 - DNA, 20 bp, Artificial Sequence
  Synthetic sequence
agtagctccggagacagcgt

SEQ ID NO: 171 - DNA, 20 bp, Artificial Sequence
  Synthetic sequence
cggtcagaccagtagctccg

SEQ ID NO: 172 - DNA, 20 bp, Artificial Sequence
  Synthetic sequence
agccggtcagaccagtagct

SEQ ID NO: 173 - DNA, 20 bp, Artificial Sequence
  Synthetic sequence
cagccggtcagaccagtagc

SEQ ID NO: 174 - DNA, 20 bp, Artificial Sequence
  Synthetic sequence
gctagtaacgtactgtttgc

SEQ ID NO: 175 - DNA, 20 bp, Artificial Sequence
  Synthetic sequence
aagaaggctagtaacgtact

SEQ ID NO: 176 - DNA, 20 bp, Artificial Sequence
  Synthetic sequence
gaagaaggctagtaacgtac

SEQ ID NO: 177 - DNA, 20 bp, Artificial Sequence
  Synthetic sequence
agaagaaggctagtaacgta

SEQ ID NO: 178 - DNA, 20 bp, Artificial Sequence
  Synthetic sequence
gagaaaggttccgtcgggct

SEQ ID NO: 179 - DNA, 20 bp, Artificial Sequence
  Synthetic sequence
ggagaaaggttccgtcgggc

SEQ ID NO: 180 - DNA, 20 bp, Artificial Sequence
  Synthetic sequence
cggattcggtcccccagtga

SEQ ID NO: 181 - DNA, 20 bp, Artificial Sequence
  Synthetic sequence
ccggattcggtcccccagtg

SEQ ID NO: 182 - DNA, 20 bp, Artificial Sequence
  Synthetic sequence
atcccggattcggtccccca

SEQ ID NO: 183 - DNA, 20 bp, Artificial Sequence
  Synthetic sequence
agatcccggattcggtcccc

SEQ ID NO: 184 - DNA, 20 bp, Artificial Sequence
  Synthetic sequence
aagatcccggattcggtccc

SEQ ID NO: 185 - DNA, 20 bp, Artificial Sequence
  Synthetic sequence
gcaagatcccggattcggtc

SEQ ID NO: 186 - DNA, 20 bp, Artificial Sequence
  Synthetic sequence
tgagcaagatcccggattcg

SEQ ID NO: 187 - DNA, 20 bp, Artificial Sequence
  Synthetic sequence
tgatacggggggatggagtg

SEQ ID NO: 188 - DNA, 20 bp, Artificial Sequence
  Synthetic sequence
ttgatacggggggatggagt

SEQ ID NO: 189 - DNA, 20 bp, Artificial Sequence
  Synthetic sequence
gggagaggccttgatacggg

SEQ ID NO: 190 - DNA, 20 bp, Artificial Sequence
  Synthetic sequence
gatcaccaactggggttggc

SEQ ID NO: 191 - DNA, 20 bp, Artificial Sequence
  Synthetic sequence
tgcgtgtgcgcgctgcaggt

SEQ ID NO: 192 - DNA, 20 bp, Artificial Sequence
  Synthetic sequence
gtgcgtgtgcgcgctgcagg

SEQ ID NO: 193 - DNA, 20 bp, Artificial Sequence
  Synthetic sequence
gcccttgtacttttgcatag

SEQ ID NO: 194-300 - skipped (107 entries)

SEQ ID NO: 301 - protein, 879 aa, Artificial Sequence
  The PTGFRN Protein
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 302 - protein, 192 aa, Artificial Sequence
  The PTGFRN protein Fragment
GPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEM

(19) United States
(12) Patent Application Publication
(10) Pub. No.: US 2023/0364127 A1
(43) Pub. Date: Nov. 16, 2023

(54) EXTRACELLULAR VESICLE-ASO CONSTRUCTS TARGETING STAT6

(71) Applicant: CODIAK BIOSCIENCES, INC., Cambridge, MA (US)

(72) Inventors: Tim SOOS, Boston, MA (US); Sushrut KAMERKAR, Cambridge, MA (US); Su Chul JANG, Cambridge, MA (US); Kyriakos ECONOMIDES, Grafton, MA (US); Dalia BURZYN, Newton, MA (US)

(73) Assignee: CODIAK BIOSCIENCES, INC., (omitted)

(21) Appl. No.: 18/248,036

(22) PCT Filed: Oct. 6, 2021
(86) PCT No.: PCT/US2021/053825

§ 371 Date: Apr. 5, 2023

Related U.S. Application Data
(60) Provisional application No. 63/244,626, filed on Sep. 15, 2021.
(60) Provisional application No. 63/225,852, filed on Jul. 26, 2021.
(60) Provisional application No. 63/169,753, filed on Apr. 1, 2021.
(60) Provisional application No. 63/160,596, filed on Mar. 12, 2021.
(60) Provisional application No. 63/141,829, filed on Jan. 26, 2021.
(60) Provisional application No. 63/088,312, filed on Oct. 6, 2020.

(51) Int. Cl. A61K 31/713; A61K 39/395; A61P 35/00; A61K 47/69

(52) U.S. Cl. CPC A61K 31/713; A61K 39/3955; A61P 35/00; A61K 47/6911; A61K 2039/505

(57) ABSTRACT

The present disclosure relates to extracellular vesicles, e.g., exosomes, comprising an antisense oligonucleotide (ASO), wherein the ASO comprises a contiguous nucleotide sequence of 10 to 30 nucleotides in length that is complementary to a nucleic acid sequence within a STAT6 transcript. Also provided herein are methods for producing the exosomes and methods for using the exosomes to treat and/or prevent diseases or disorders.

### CROSS-REFERENCE TO RELATED APPLICATIONS

This application claims priority benefit of U.S. Provisional Application Nos. 63/088,312, filed Oct. 6, 2020; 63/141,829 filed Jan. 26, 2021; 63/160,596 filed Mar. 12, 2021; 63/169,753 filed Apr. 1, 2021; 63/225,852 filed Jul. 26, 2021; and 63/244,626 filed Sep. 15, 2021, each of which is herein incorporated by reference in its entirety.

### REFERENCE TO SEQUENCE LISTING SUBMITTED ELECTRONICALLY VIA EFS-WEB

The content of the electronically submitted sequence listing (Name: 4000_12PC06_Seqlisting_ST25.txt, Size: 48,580 bytes; and Date of Creation: Oct. 6, 2021) submitted in this application is incorporated herein by reference in its entirety.

### FIELD OF DISCLOSURE

The present disclosure relates to methods of treating a disease or condition in a subject in need thereof, comprising administering (i) an extracellular vesicle (EV), e.g., exosome, comprising an antisense oligonucleotide (ASO), and (ii) a programmed death-1 (PD-1) antagonist, e.g., an anti-PD-1 antibody; wherein the ASO comprises a contiguous nucleotide sequence of 10 to 30 nucleotides in length that is complementary to a nucleic acid sequence within a STAT6 transcript. In certain aspects of the disclosure, the extracellular vesicle further comprises a scaffold protein.

### BACKGROUND

Exosomes are small extracellular vesicles that are naturally produced by every eukaryotic cell. Exosomes comprise a membrane that encloses an internal space (i.e., lumen). As drug delivery vehicles, EVs, e.g., exosomes, offer many advantages over traditional drug delivery methods as a new treatment modality in many therapeutic areas. In particular, exosomes have intrinsically low immunogenicity, even when administered to a different species.

Antisense oligonucleotides have emerged as a powerful means of regulating target gene expression in vitro or in vivo. However, there remains a need to improve the stability and targeting of ASOs in vivo. Accordingly, new and more effective engineered-EVs (e.g., exosomes), particularly those that can be used to deliver therapeutic agents that can reduce the expression of a gene associated with a disease (e.g., N for cancer), are necessary to better enable therapeutic use and other applications of EV-based technologies.

### SUMMARY OF DISCLOSURE

Certain aspects of the present disclosure are directed to a method of preventing or treating a disease or condition in a subject in need thereof, comprising administering to the subject (i) an extracellular vesicle comprising an antisense oligonucleotide (ASO) and (ii) a programmed death-1 (PD-1) antagonist; wherein the ASO comprises a contiguous nucleotide sequence of 10 to 30 nucleotides in length that is complementary to a nucleic acid sequence within a STAT6 transcript (SEQ ID NO: 1 or SEQ ID NO: 3).

Certain aspects of the present disclosure are directed to a method of increasing or enhancing an immune response in a subject in need thereof, comprising administering to the subject (i) an extracellular vesicle comprising an antisense oligonucleotide (ASO) and (ii) a programmed death-1 (PD-1) antagonist; wherein the ASO comprises a contiguous nucleotide sequence of 10 to 30 nucleotides in length that is complementary to a nucleic acid sequence within a STAT6 transcript (SEQ ID NO: 1 or SEQ ID NO: 3).

In some aspects, the contiguous nucleotide sequence is complementary to a nucleic acid sequence within nucleotides 1 to 2056 of a STAT6 transcript corresponding to a nucleotide sequence as set forth in SEQ ID NO: 3 or nucleotides 2059 to 3963 of a STAT6 transcript corresponding to a nucleotide sequence as set forth in SEQ ID NO: 3.

In some aspects, the ASO is a gapmer, a mixmer, or a totalmer. In some aspects, the ASO comprises one or more nucleoside analogs. In some aspects, one or more of the nucleoside analogs comprises a 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-ANA; or bicyclic nucleoside analog.

In some aspects, one or more of the nucleoside analogs is a sugar modified nucleoside. In some aspects, the sugar modified nucleoside is an affinity enhancing 2′ sugar modified nucleoside. In some aspects, one or more of the nucleoside analogs comprises a nucleoside comprising a bicyclic sugar. In some aspects, one or more of the nucleoside analogs comprises an LNA. In some aspects, one or more of the nucleotide analogs is selected from the group consisting of constrained ethyl nucleoside (cEt), 2′,4′-constrained 2′-O-methoxyethyl (cMOE), α-L-LNA, β-D-LNA, 2′-O,4′-C-ethylene-bridged nucleic acids (ENA), amino-LNA, oxy-LNA, thio-LNA, and any combination thereof. In some aspects, the ASO comprises one or more 5′-methyl-cytosine nucleobases. In some aspects, the contiguous nucleotide sequence is complementary to a nucleic acid sequence within (i) a 5′ untranslated region (UTR); (ii) a coding region; or (iii) a 3′ UTR of the target transcript.

In some aspects, the contiguous nucleotide sequence is complementary to a nucleic acid sequence comprising (i) nucleotides 1-700 of SEQ ID NO: 3; (ii) nucleotides 1000-1500 of SEQ ID NO: 3; (iii) nucleotides 1500-2000 of SEQ ID NO: 3; (iv) nucleotides 2000-2500 of SEQ ID NO: 3; (v) 2500-3000 of SEQ ID NO: 3; (vi) 3000-3700 of SEQ ID NO: 3, (vii) nucleotides 413-803 of SEQ ID NO: 3; (viii) nucleotides 952-1688 of SEQ ID NO: 3; (ix) nucleotides 1726-2489 of SEQ ID NO: 3; (x) nucleotides 2682-2912 of SEQ ID NO: 3; (xi) 2970-3203 of SEQ ID NO: 3; (xii) 3331-3561 of SEQ ID NO: 3; (xiii) nucleotides 463-753 of SEQ ID NO: 3; (xiv) nucleotides 1002-1638 of SEQ ID NO: 3; (xv) nucleotides 1776-2439 of SEQ ID NO: 3; (xvi) nucleotides 2682-2862 of SEQ ID NO: 3; (xvii) 3020-3153 of SEQ ID NO: 3; (xviii) 3381-3511 of SEQ ID NO: 3; (xix) nucleotides 503-713 of SEQ ID NO: 3; (xx) nucleotides 1042-1598 of SEQ ID NO: 3; (xxi) nucleotides 1816-2399 of SEQ ID NO: 3; (xxii) nucleotides 2722-2822 of SEQ ID NO: 3; (xxiii) 3060-3113 of SEQ ID NO: 3; or (xxiv) 3421-3471 of SEQ ID NO: 3. In some aspects, the contiguous nucleotide sequence is complementary to a nucleic acid sequence within (i) nucleotides 513-703 of SEQ ID NO: 3; (ii) nucleotides 1052-1588 of SEQ ID NO: 3; (iii) nucleotides 1826-2389 of SEQ ID NO: 3; (iv) nucleotides 2732-2812 of SEQ ID NO: 3; (v) 3070-3103 of SEQ ID NO: 3; or (vi) 3431-3461 of SEQ ID NO: 3. In some aspects, the ASO comprises a nucleic acid sequence selected from GAAAGGTTCCGTCGGGC (SEQ ID NO: 144), CTGAGTCGCTGAAGCGG (SEQ ID NO: 145), GCCCTTGTACTTTTGCATAG (SEQ ID NO: 193), GCAAGATCCCGGATTCGGTC (SEQ ID NO: 185), and any combination thereof.

In some aspects, the contiguous nucleotide sequence comprises a nucleotide sequence complementary to a sequence selected from the sequences in FIG. 1. In some aspects, the continuous nucleotide sequence is fully complementary to a nucleotide sequence within the target transcript.

In some aspects, the ASO comprises a nucleotide sequence selected from SEQ ID NOs: 91-193, with one or two mismatches. In some aspects, the ASO has a design selected from the group consisting of the designs in FIG. 1, wherein the upper letter is a sugar modified nucleoside and the lower case letter is DNA. In some aspects, the ASO is from 14 to 20 nucleotides in length.

In some aspects, the contiguous nucleotide sequence comprises one or more modified internucleoside linkages. In some aspects, the one or more modified internucleoside linkages is a phosphorothioate linkage. In some aspects, at least 75%, at least 80%, at least 85%, at least 90%, at least 95%, or 100% of internucleoside linkages are modified. In some aspects, each of the internucleoside linkages in the ASO is a phosphorothioate linkage.

In some aspects, the ASO is linked to an anchoring moiety. In some aspects, the anchoring moiety comprises a sterol, GM1, a lipid, a vitamin, a small molecule, a peptide, or a combination thereof. In some aspects, the anchoring moiety comprises cholesterol. In some aspects, the anchoring moiety comprises a phospholipid, a lysophospholipid, a fatty acid, a vitamin (e.g., vitamin D and/or vitamin E), or any combination thereof. In some aspects, the anchoring moiety is associated with the extracellular vesicle. In some aspects, the extracellular vesicle comprises a lipid bilayer, wherein the anchoring moiety is associated with the lipid bilayer of the extracellular vesicle. In some aspects, the ASO is linked to the anchoring moiety on the exterior surface of the EV. In some aspects, the ASO is linked to the anchoring moiety on the luminal surface of the EV. In some aspects, the ASO is linked to the anchoring moiety. In some aspects, the anchoring moiety comprises a scaffold moiety.

In some aspects, the ASO is linked to the EV by a linker. In some aspects, the linker is a polypeptide. In some aspects, the linker is a non-polypeptide moiety. In some aspects, the linker comprise ethylene glycol. In some aspects, the linker comprises HEG, TEG, PEG, or any combination thereof. In some aspects, the linker comprises acrylic phosphoramidite (e.g., Acrydite™), adenylation, azide (NHS Ester), digoxigenin (NHS Ester), cholesterol-TEG, I-LINKER™, an amino modifier (e.g., amino modifier C6, amino modifier C12, amino modifier C6 dT, or Uni-Link™ amino modifier), alkyne, 5′ Hexynyl, 5-Octadiynyl dU, biotinylation (e.g., biotin, biotin (Azide), biotin dT, biotin-TEG, dual biotin, PC biotin, or desthiobiotin), thiol modification (thiol modifier C3 S—S, dithiol or thiol modifier C6 S—S), or any combination thereof. In some aspects, the linker is a cleavable linker. In some aspects, the linker comprises valine-alanine-p-aminobenzylcarbamate or valine-citrulline-p-aminobenzylcarbamate. In some aspects, the linker comprises (i) a maleimide moiety and (ii) valine-alanine-p-aminobenzylcarbamate or valine-citrulline-p-aminobenzylcarbamate.

In some aspects, the extracellular vesicle further comprises an exogenous targeting moiety. In some aspects, the exogenous targeting moiety comprises a peptide, an antibody or an antigen-binding fragment thereof, a chemical compound, an RNA aptamer, or any combination thereof. In some aspects, the exogenous targeting moiety comprises a peptide. In some aspects, the exogenous targeting moiety comprises a microprotein, a designed ankyrin repeat protein (darpin), an anticalin, an adnectin, an aptamer, a peptide mimetic molecule, a natural ligand for a receptor, a camelid nanobody, or any combination thereof. In some aspects, the exogenous targeting moiety comprises a full-length antibody, a single domain antibody, a heavy chain only antibody (VHH), a single chain antibody, a shark heavy chain only antibody (VNAR), an scFv, a Fv, a Fab, a Fab′, a F(ab′)2, or any combination thereof. In some aspects, the antibody is a single chain antibody.

In some aspects, the exogenous targeting moiety targets the exosome to the liver, heart, lungs, brain, kidneys, central nervous system, peripheral nervous system, muscle, bone, joint, skin, intestine, bladder, pancreas, lymph nodes, spleen, blood, bone marrow, or any combination thereof. In some aspects, the exogenous targeting moiety targets the extracellular vesicle to a tumor cell, dendritic cell, T cell, B cell, macrophage, neuron, hepatocyte, Kupffer cell, myeloid-lineage cell (e.g., a neutrophils, monocytes, macrophages, hematopoietic stem cell, an MDSC (e.g., a monocytic MDSC or a granulocytic MDSC)), or any combination thereof.

In some aspects, the extracellular vesicle comprises a scaffold moiety linking the exogenous targeting moiety to the extracellular vesicle. In some aspects, the scaffold moiety is a Scaffold X. In some aspects, the scaffold moiety is a Scaffold Y.

In some aspects, the extracellular vesicle is an exosome.

In some aspects, the PD-1 antagonist comprises an antibody or an antigen-binding portion thereof that specifically bind to human PD-1 and blocks or inhibits the interaction between PD-1 and PD-L1 (“an anti-PD-1 antibody”). In some aspects, the anti-PD-1 antibody is selected from the group consisting of nivolumab, pembrolizumab, PDR001, MEDI-0680, cemiplimab, JS001, BGB-A317, INCSHR1210, TSR-042, GLS-010, AM-0001, STI-1110, AGEN2034, MGA012, IBI308, and any combination thereof.

In some aspects, the PD-1 antagonist comprises an antibody or an antigen-binding portion thereof that specifically bind to human PD-L1 and blocks or inhibits the interaction between PD-1 and PD-L1 (“an anti-PD-L1 antibody”). In some aspects, the anti-PD-L1 antibody is selected from the group consisting of atezolizumab, durvalumab, avelumab, STI-1014, CX-072, KN035, LY3300054, CK-301, BMS-936559, and any combination thereof.

In some aspects, the extracellular vesicle and the PD-1 antagonist are administered concurrently. In some aspects, the extracellular vesicle and the PD-1 antagonist are administered sequentially. In some aspects, the extracellular vesicle and the PD-1 antagonist are administered on different days.

In some aspects, the PD-1 antagonist is linked to or associated with the extracellular vesicle.

In some aspects, the extracellular vesicle, the PD-1 antagonist, or both are administered by a route selected from the groups consisting of parenteral administration, topical administration, intravenous administration, oral administration, subcutaneous administration, intra-arterial administration, intradermal administration, transdermal administration, rectal administration, intracranial administration, intraperitoneal administration, intrathecal administration, intranasal administration, intratumoral administration, intramuscular administration, inhalation, and any combination thereof.

In some aspects, the subject is afflicted with a cancer. In some aspects, the cancer is selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell cancer, squamous cell cancer of the head and neck cancer, colorectal cancer, lymphoma, leukemia, liver cancer, glioblastoma, melanoma, myeloma basal cell cancer, adenocarcinoma, sweat gland cancer, sebaceous gland cancer, papillary cancer, papillary adenocarcinomas, cystadenocarcinoma, medullary cancer, bronchogenic cancer, renal cell cancer, hepatoma, bile duct cancer, choriocarcinoma, seminoma, nonseminoma, embryonal cancer, Wilms' tumor, cervical cancer, testicular cancer, lung cancer, small cell lung cancer, bladder cancer, epithelial cancer, glioma, glioblastoma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, follicular lymphoma, Hodgkin's lymphoma, B cell lymphoma, and any combination thereof.

### BRIEF DESCRIPTION OF FIGURES

FIG. 1 is a table listing various ASO sequences that target the STAT6 (FIG. 1) transcript. The table includes the following information (from left to right): (i) description of the ASO, (ii) the ASO sequence without any particular design or chemical structure, (iii) SEQ ID number designated for the ASO sequence only, (iv) the ASO length, (v) the ASO sequence with a chemical structure, and (vi) the target start and end positions on the target transcript sequence (SEQ ID NO: 3). The ASOs are from 5′ to 3′. The symbols in the chemical structures are as follows: Nb means LNA; dN means DNA; 5MdC means 5-Methyl-dC; Nm means MOE; and s means phosphorothioate.

FIG. 2 is a schematic drawing of an exosome overexpressing PTGFRN loaded with ASOs that target STAT6.

FIG. 3A is a schematic of an experimental design for administering exosomes loaded with an ASO alone or in combination with an anti-PD-1 antibody. FIG. 3B is a graphical representation of the percent of scored lesions in a mouse model following administration of exosomes loaded with ASOs targeting STAT6 alone or in combination with an anti-PD-1 antibody, as compared to exosome only controls, exoASO scramble controls, and anti-CSF1R antibody controls.

FIG. 4A is a schematic illustration of a dosing schedule for administering CD4 and/or CD8 depleting antibodies in mice bearing CT26 tumors. FIGS. 4B-4C are graphical representations of tumor volume over time (FIG. 4B) and tumor growth rate (FIG. 4C).

FIG. 5 is a graphical representation of tumor volume following rechallenge of complete responder CT26 mice, treated with exoASO-STAT6 three times per week (TIW) or once every three days (q3d) for three weeks. Naïve mice with no previous tumors were used as control.

FIG. 6A is a Kaplan-Meier survival curve analysis of data from CT26 mice, log-rank Mantel-Cox test. FIG. 6B is a graphical representation of tumor growth volumes of Balb/C mice bearing subcutaneous CT26 tumors, injected intratumorally with PBS, exosomes only, exoASO Scramble (bug) (BIW, 3 wk) or exoASO STAT6-2039 (PTGFRN+) (0.22, 0.67, 2, 6 and 18 ug) (BIW, 3 wk), n=10 mice per group.

FIGS. 7A-7W show that effective reprogramming of TAMs by STAT6 exoASO results in remodeling of TME. FIG. 7A is a schematic of dosing schedule. Balb/C mice bearing subcutaneous CT26 tumors injected with exoASO Scramble (4 ug), exoASO STAT6-2039 (WT) (4 ug) or Free STAT6 ASO-2039 (4 ug). FIGS. 7B-7C show normalized gene expression analysis by RT-qPCR of changes in Stat6 and Arg1 mRNA expression from whole tumors and CD11b-enriched fractions. FIG. 7D shows nanostring gene expression analysis as depicted by a Volcano plot of changes in gene expression of exoASO STAT6 vs exoASO Scramble baseline, from CD11b enriched fractions from FIGS. 7B-7C. FIG. 7E is a heatmap of common differentially expressed (DE) genes from all groups from FIG. 7D. FIGS. 7F-7K are graphical representation of changes in expression levels of Tgfb1, Csf1r, CD206, Ifna1, Nos2 and Arg1, as indicated. FIGS. 7L-7O are graphical representations of flow cytometry analysis of % of total immune cells, MFI of (CD206+) TAMs and % of regulatory T cells (FoxP3+) within CD4+ or CD45+ immune cell population from tumors of Balb/C mice bearing subcutaneous CT26 tumors, injected intratumorally with PBS, exoASO-Scramble (6 ug) (TIW, 1 wk) or exoASO STAT6-2039 (PTGFRN+) (6 ug) (TIW, 1 wk), n=10 mice per group. FIG. 7P is a tSNE plot from scRNA seq of intra tumoral cells of all groups merged to identify individual immune cell populations. Balb/C mice bearing subcutaneous CT26 tumors were injected intratumorally with exoASO Scramble (6 ug) or exoASO STAT6-2039 (PTGFRN+) (6 ug) (TIW, 1 week). FIGS. 7Q-7S are tSNE plots from data from FIG. 7P, showing global changes in expression and quantification of Stat6 in immune cell populations. FIGS. 7T-7W are tSNE plots from data from FIG. 7P, showing global changes in expression of Nos2 and Retnla (Fizz-1) in immune cell populations. Data are mean±s.e.m. *P<0.05, **P<0.01, ***P<0.001, ****P<0.000. One-way ANOVA with Turkey's multiple comparisons test (B, E, F, H). FIGS. 7X-7AA are graphical representations of the expression level of Gzmb (FIG. 7X), Id2 (FIG. 7Y), Lag3 (FIG. 7Z), and IL7r (FIG. 7AA) in tumor CD8⁺ T cells from exoASO scramble (1) or exoASO-STAT6 (2) treated groups.

FIGS. 8A-8UU and 8WW-8HHH show that systemic administration of exoASO STAT6 results in potent monotherapy anti-tumor response. FIG. 8A is a schematic of a dosing schedule. FIGS. 8B-8H show antitumoral efficacy as represented by liver weight (LW) vs body weight (BW) ratio of C57Bl/6 mice bearing orthotopic Hepa1-6 tumors in the liver, injected intravenously with exosomes only, exoASO Scramble (12 ug) (TIW, 2 wk) or exoASO STAT6-2039 (PTGFRN+) (12 ug) (TIW, 2 wk), or intraperitoneally with anti CSF1R (10 mg/kg) (DIW) or anti-PD1 (10 mg/kg) (DIW). Representative gross images of whole Hepa1-6 tumor livers of the listed groups at study end point are shown. FIGS. 8I-8L show the percentage (%) of tumor cells as calculated by Halo Imaging Analysis Platform from H&E stained sections of livers. FIG. 8M is a graphical representation of the percent tumor area following administration of exoASO scramble, exoASO-STAT6, an anti-PD-1 antibody, and a combination of exoASO-STAT6 and an anti-PD-1 antibody, as indicated. FIGS. 8N-8Q are images of livers from Hepa1-6 tumor-bearing mice corresponding to the treatment groups of FIG. 8M. FIGS. 8R-8U are histological images of representative tumor samples corresponding to the treatment groups of FIG. 8M. FIG. 8V is a graphical representation of the tumor inflammation score following administration of exoASO scramble, exoASO-STAT6, an anti-PD-1 antibody, and a combination of exoASO-STAT6 and an anti-PD-1 antibody, as indicated. FIG. 8W shows normalized gene expression analysis by RT-qPCR of modulation of Stat6 mRNA expression in whole Hepa1-6 tumor livers. FIG. 8X shows Nanostring gene expression analysis as depicted by a Volcano plot of changes in gene expression of exoASO STAT6 vs exoASO Scramble baseline from whole Hepa1-6 tumor livers. FIGS. 8Y-8CC are graphical representation of changes in expression levels of Ccl17, CD206, Myc, H2-K1, and Icos, as indicated. FIGS. 8DD-8II show pathway score analysis of data in FIG. 8X as calculated by nSolver Analysis Software, of cell proliferation, toxicity and TGF Beta signaling. Cell type score profiling of data in FIG. 8X as calculated by nSolver Analysis Software, of CD8 T cells, dendritic cells, and cytotoxic cells. FIGS. 8JJ-8QQ show representative images and quantification of F4/80 (macrophage) and Nos2 expression, performed by immunofluorescence and IHC respectively, in Hepa1-6 tumor sections, quantification was performed using Halo Imaging Analysis Platform. FIGS. 8RR-8HHH show representative images and quantification of Stat6 expression (overlaid with DAPI (FIGS. 8RR-8TT) or with IBA1 (FIGS. 8EEE-8GGG)), cytotoxic T-cells (CD8+; FIGS. 8UU, 8WW, and 8XX) and regulatory T-cells (FoxP3+; FIGS. 8YY-8AAA) performed by immunofluorescence in Hepa1-6 tumor sections, quantification was performed using Halo Imaging Analysis Platform. Data are mean±s.e.m. *P<0.05, **P<0.01, ***P<0.001, ****P<0.000. One-way ANOVA with Turkey's multiple comparisons test (B, C, D, F, G, I), Sidak's multiple comparison test.

FIG. 9A is a graphical representation of the STAT6 signature mean expression (log 2) for various cancer types. FIGS. 9B-9G are Kaplan-Meier curves of overall survival (OS) probability in HCC (FIG. 9B), RCC (FIG. 9C), bladder cancer (FIG. 9D), ovarian cancer (FIG. 9E), stomach cancer (FIG. 9F), and sarcoma (FIG. 9G), based on analysis of patients with high or low STAT6 macrophage gene signature. Results were generated using Kaplan-Meier Plotter, log-rank Mantel-Cox test.

FIG. 10 is a schematic representation of a process for purifying exosomes loaded with an ASO.

FIGS. 11A-11C of ASO concentration (FIG. 11A), free ASO concentration (FIG. 11B), and IC50 (FIG. 11C) for sample stored up to 15 days at room temperature (RT) or at 5° C.

FIGS. 12A-12D are graphical representations of NTA (FIG. 12A), UV (FIG. 12B), AEC-UHPLC (FIG. 12C), and DLS (FIG. 12D) for CC700 filtered intermediates held at room temperature for 15 days.

FIGS. 13A-13D are graphical representations of NTA (FIG. 13A), UV (FIG. 13B), AEC-UHPLC (FIG. 13C), and DLS (FIG. 13D) for CC700 filtered intermediates held at 5° C. for 15 days.

FIGS. 14A-14B are schematic representations of a process for evaluating loading temperature and NaCl/Sucrose in CC700 Column filtration.

FIGS. 15A-15B are graphical representations of ASO-Cy5 signal observed in immune v. non-immune compartment (FIG. 15A) and within a subset of immune cells (FIG. 15B) following IT administration of Cy5-labelled exoASO in mice. FIGS. 15C-15G are bar graphs illustrating localization of Cy5-labelled exoASO and free ASO in the blood (FIG. 15C), spleen (FIG. 15D), bone marrow (FIG. 15E), liver (FIG. 15F), and tumor (FIG. 15G), following IV administration. FIG. 15H is a bar graph showing the Cy5 maximum fluorescence intensity in various cells types following IV administration of Cy5-labelled exoASO. FIG. 15I is a graphical representation of localization of Zr89 labeled PTGFRN exosomes in the liver, spleen, and femur following IV administration. FIGS. 15J and 15K are graphical representations of Stat6 mRNA levels in the liver (FIG. 15J) and spleen (FIG. 15K) following administration of PBS negative control and one or three doses of exoASO-STAT6 and free STAT6 ASO, as indicated. FIGS. 15L-15M are scatter plots illustrating pHrodo dye-labelled exoASO-STAT6 uptake by M1 and M2 macrophages (FIG. 15L) and by M2 macrophages alone (1) or in the presence of the phagocytosis inhibitor cytochalasin D (2), the class A scavenger receptor inhibitor Fucoidan (3), or the non-selective scavenger receptor inhibitor poly(I) (4), as indicated (FIG. 15M).

### DETAILED DESCRIPTION OF DISCLOSURE

Certain aspects of the present disclosure are directed to methods of treating a disease or condition in a subject in need thereof, comprising administering to the subject (i) an extracellular vesicle (EV), e.g., an exosome, comprising an antisense oligonucleotide (ASO), wherein the ASO comprises a contiguous nucleotide sequence of 10 to 30 nucleotides in length that is complementary to a nucleic acid sequence within a STAT6, and (ii) a PD-1 antagonist, e.g., an anti-PD-1 antibody.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). For example, if it is stated that “the ASO reduces expression of STAT6 protein in a cell following administration of the ASO by at least about 60%,” it is implied that the STAT6 levels are reduced by a range of 50% to 70%.

The term “antisense oligonucleotide” (ASO) refers to an oligomer or polymer of nucleosides, such as naturally-occurring nucleosides or modified forms thereof, that are covalently linked to each other through internucleotide linkages. The ASO useful for the disclosure includes at least one non-naturally occurring nucleoside. An ASO is at least partially complementary to a target nucleic acid, such that the ASO hybridizes to the target nucleic acid sequence.

The term “nucleic acids” or “nucleotides” is intended to encompass plural nucleic acids. In some aspects, the term “nucleic acids” or “nucleotides” refers to a target sequence, e.g., pre-mRNAs, mRNAs, or DNAs in vivo or in vitro. When the term refers to the nucleic acids or nucleotides in a target sequence, the nucleic acids or nucleotides can be naturally occurring sequences within a cell. In other aspects, “nucleic acids” or “nucleotides” refer to a sequence in the ASOs of the disclosure. When the term refers to a sequence in the ASOs, the nucleic acids or nucleotides can be non-naturally occurring, i.e., chemically synthesized, enzymatically produced, recombinantly produced, or any combination thereof. In some aspects, the nucleic acids or nucleotides in the ASOs are produced synthetically or recombinantly, but are not a naturally occurring sequence or a fragment thereof. In some aspects, the nucleic acids or nucleotides in the ASOs are not naturally occurring because they contain at least one nucleoside analog that is not naturally occurring in nature.

The term “nucleotide” as used herein, refers to a glycoside comprising a sugar moiety, a base moiety and a covalently linked group (linkage group), such as a phosphate or phosphorothioate internucleotide linkage group, and covers both naturally occurring nucleotides, such as DNA or RNA, and non-naturally occurring nucleotides comprising modified sugar and/or base moieties, which are also referred to as “nucleotide analogs” herein. Herein, a single nucleotide can be referred to as a monomer or unit. In certain aspects, the term “nucleotide analogs” refers to nucleotides having modified sugar moieties. Non-limiting examples of the nucleotides having modified sugar moieties (e.g., LNA) are disclosed elsewhere herein. In other aspects, the term “nucleotide analogs” refers to nucleotides having modified nucleobase moieties. The nucleotides having modified nucleobase moieties include, but are not limited to, 5-methyl-cytosine, isocytosine, pseudoisocytosine, 5-bromouracil, 5-propynyluracil, 6-aminopurine, 2-aminopurine, inosine, diaminopurine, and 2-chloro-6-aminopurine. In some aspects, the terms “nucleotide”, “unit” and “monomer” are used interchangeably. It will be recognized that when referring to a sequence of nucleotides or monomers, what is referred to is the sequence of bases, such as A, T, G, C or U, and analogs thereof.

The term “nucleoside” as used herein is used to refer to a glycoside comprising a sugar moiety and a base moiety, and can therefore be used when referring to the nucleotide units, which are covalently linked by the internucleotide linkages between the nucleotides of the ASO. In the field of biotechnology, the term “nucleotide” is often used to refer to a nucleic acid monomer or unit. In the context of an ASO, the term “nucleotide” can refer to the base alone, i.e., a nucleobase sequence comprising cytosine (DNA and RNA), guanine (DNA and RNA), adenine (DNA and RNA), thymine (DNA) and uracil (RNA), in which the presence of the sugar backbone and internucleotide linkages are implicit. Likewise, particularly in the case of oligonucleotides where one or more of the internucleotide linkage groups are modified, the term “nucleotide” can refer to a “nucleoside.” For example the term “nucleotide” can be used, even when specifying the presence or nature of the linkages between the nucleosides.

The term “nucleotide length” as used herein means the total number of the nucleotides (monomers) in a given sequence. For example, the sequence of ASO-STAT6-1053 (SEQ ID NO: 91) has 15 nucleotides; thus the nucleotide length of the sequence is 15. The term “nucleotide length” is therefore used herein interchangeably with “nucleotide number.”

As one of ordinary skill in the art would recognize, the 5′ terminal nucleotide of an oligonucleotide does not comprise a 5′ internucleotide linkage group, although it can comprise a 5′ terminal group.

The compounds described herein can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates. In some aspects, the asymmetric center can be an asymmetric carbon atom. The term “asymmetric carbon atom” means a carbon atom with four different substituents. According to the Cahn-Ingold-Prelog Convention an asymmetric carbon atom can be of the “R” or “S” configuration.

As used herein, the term “bicyclic sugar” refers to a modified sugar moiety comprising a 4 to 7 membered ring comprising a bridge connecting two atoms of the 4 to 7 membered ring to form a second ring, resulting in a bicyclic structure. In some aspects, the bridge connects the C2′ and C4′ of the ribose sugar ring of a nucleoside (i.e., 2′-4′ bridge), as observed in LNA nucleosides.

The term “region” when used in the context of a nucleotide sequence refers to a section of that sequence. For example, the phrase “region within a nucleotide sequence” or “region within the complement of a nucleotide sequence” refers to a sequence shorter than the nucleotide sequence, but longer than at least 10 nucleotides located within the particular nucleotide sequence or the complement of the nucleotides sequence, respectively. The term “sub-sequence” or “subsequence” can also refer to a region of a nucleotide sequence.

The term “transcript” as used herein can refer to a primary transcript that is synthesized by transcription of DNA and becomes a messenger RNA (mRNA) after processing, i.e., a precursor messenger RNA (pre-mRNA), and the processed mRNA itself. The term “transcript” can be interchangeably used with “pre-mRNA” and “mRNA.” After DNA strands are transcribed to primary transcripts, the newly synthesized primary transcripts are modified in several ways to be converted to their mature, functional forms to produce different proteins and RNAs, such as mRNA, tRNA, rRNA, lncRNA, miRNA and others. Thus, the term “transcript” can include exons, introns, 5′ UTRs, and 3′ UTRs.

The term “expression” as used herein refers to a process by which a polynucleotide produces a gene product, for example, a RNA or a polypeptide. It includes, without limitation, transcription of the polynucleotide into messenger RNA (mRNA) and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

The terms “identical” or percent “identity” in the context of two or more nucleic acids refer to two or more sequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

One such non-limiting example of a sequence alignment algorithm is the algorithm described in Karlin et al., 1990, Proc. Natl. Acad. Sci., 87:2264-2268, as modified in Karlin et al., 1993, Proc. Natl. Acad. Sci., 90:5873-5877, and incorporated into the NBLAST and XBLAST programs (Altschul et al., 1991, Nucleic Acids Res., 25:3389-3402). In certain aspects, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, California) or Megalign (DNASTAR) are additional publicly available software programs that can be used to align sequences. In certain aspects, the percent identity between two nucleotide sequences is determined using the GAP program in the GCG software package (e.g., using a NWSgapdna.CMP matrix and a gap weight of 40, 50, 60, 70, or 90 and a length weight of 1, 2, 3, 4, 5, or 6). In certain alternative aspects, the GAP program in the GCG software package, which incorporates the algorithm of Needleman and Wunsch (J. Mol. Biol. (48):444-453 (1970)) can be used to determine the percent identity between two amino acid sequences (e.g., using either a BLOSUM 62 matrix or a PAM250 matrix, and a gap weight of 16, 14, 12, 10, 8, 6, or 4 and a length weight of 1, 2, 3, 4, 5). Alternatively, in certain aspects, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. One skilled in the art can determine appropriate parameters for maximal alignment by particular alignment software. In certain aspects, the default parameters of the alignment software are used.

In certain aspects, the percentage identity “X” of a first nucleotide sequence to a second nucleotide sequence is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

Different regions within a single polynucleotide target sequence that align with a polynucleotide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

As used herein, the terms “homologous” and “homology” are interchangeable with the terms “identity” and “identical.”

The term “naturally occurring variant thereof” refers to variants of the STAT6 polypeptide sequence or STAT6 nucleic acid sequence (e.g., transcript) which exist naturally within the defined taxonomic group, such as mammalian, such as mouse, monkey, and human. Typically, when referring to “naturally occurring variants” of a polynucleotide the term also can encompass any allelic variant of the STAT6-encoding genomic DNA (which is found at Chromosomal position 1q44 at 247,416,156-247,449,108 (i.e., nucleotides 247,416,156-247,449,108 of GenBank Accession No. NC_000001.11)) by chromosomal translocation or duplication, and the RNA, such as mRNA derived therefrom. “Naturally occurring variants” can also include variants derived from alternative splicing of the STAT6 mRNA. When referenced to a specific polypeptide sequence, e.g., the term also includes naturally occurring forms of the protein, which can therefore be processed, e.g., by co- or post-translational modifications, such as signal peptide cleavage, proteolytic cleavage, glycosylation, etc.

In determining the degree of “complementarity” between the ASOs of the disclosure (or regions thereof) and the target region of the nucleic acid which encodes mammalian STAT6 (e.g., the STAT6 gene), such as those disclosed herein, the degree of “complementarity” (also, “homology” or “identity”) is expressed as the percentage identity (or percentage homology) between the sequence of the ASO (or region thereof) and the sequence of the target region (or the reverse complement of the target region) that best aligns therewith. The percentage is calculated by counting the number of aligned bases that are identical between the two sequences, dividing by the total number of contiguous monomers in the ASO, and multiplying by 100. In such a comparison, if gaps exist, it is preferable that such gaps are merely mismatches rather than areas where the number of monomers within the gap differs between the ASO of the disclosure and the target region.

The term “complement” as used herein indicates a sequence that is complementary to a reference sequence. It is well known that complementarity is the base principle of DNA replication and transcription as it is a property shared between two DNA or RNA sequences, such that when they are aligned antiparallel to each other, the nucleotide bases at each position in the sequences will be complementary, much like looking in the mirror and seeing the reverse of things. Therefore, for example, the complement of a sequence of 5′“ATGC”3′ can be written as 3′“TACG”5′ or 5′“GCAT”3′. The terms “reverse complement”, “reverse complementary”, and “reverse complementarity” as used herein are interchangeable with the terms “complement”, “complementary”, and “complementarity.” In some aspects, the term “complementary” refers to 100% match or complementarity (i.e., fully complementary) to a contiguous nucleic acid sequence within a STAT6 transcript. In some aspects, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a contiguous nucleic acid sequence within a STAT6 transcript.

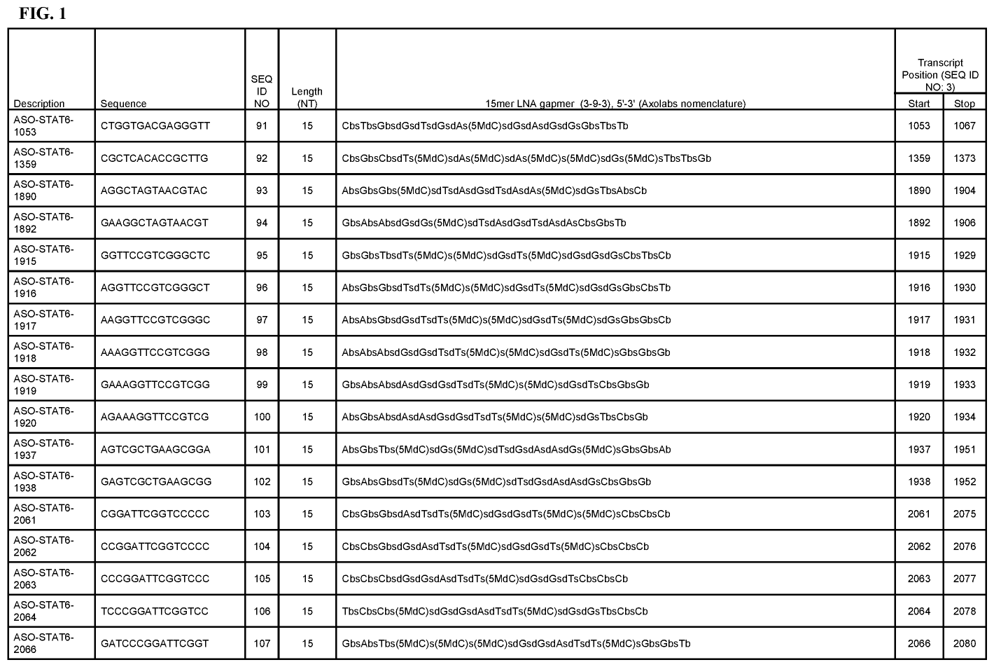

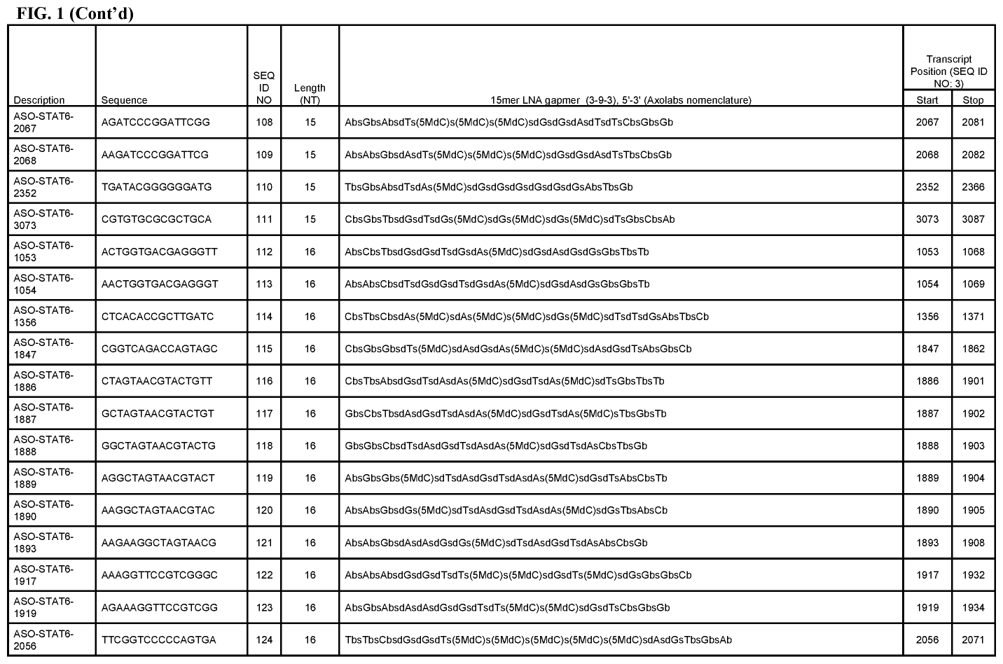

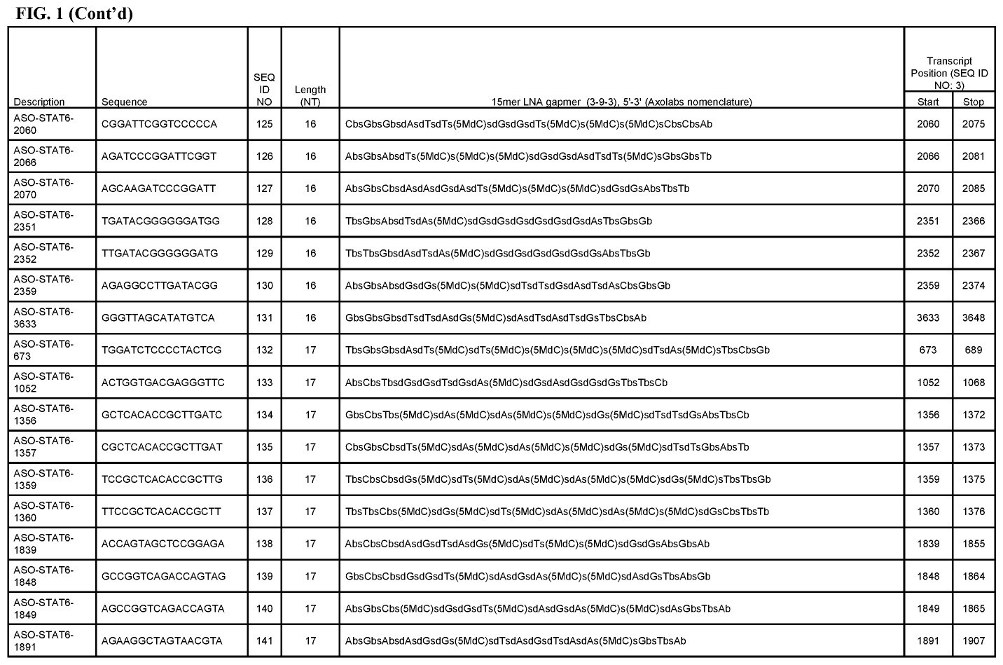

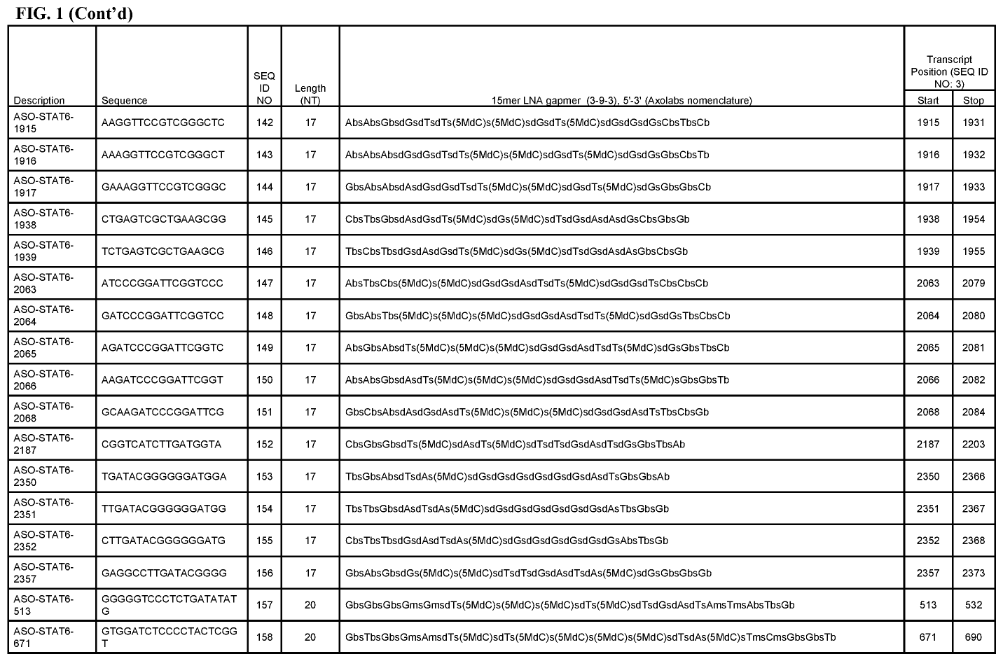

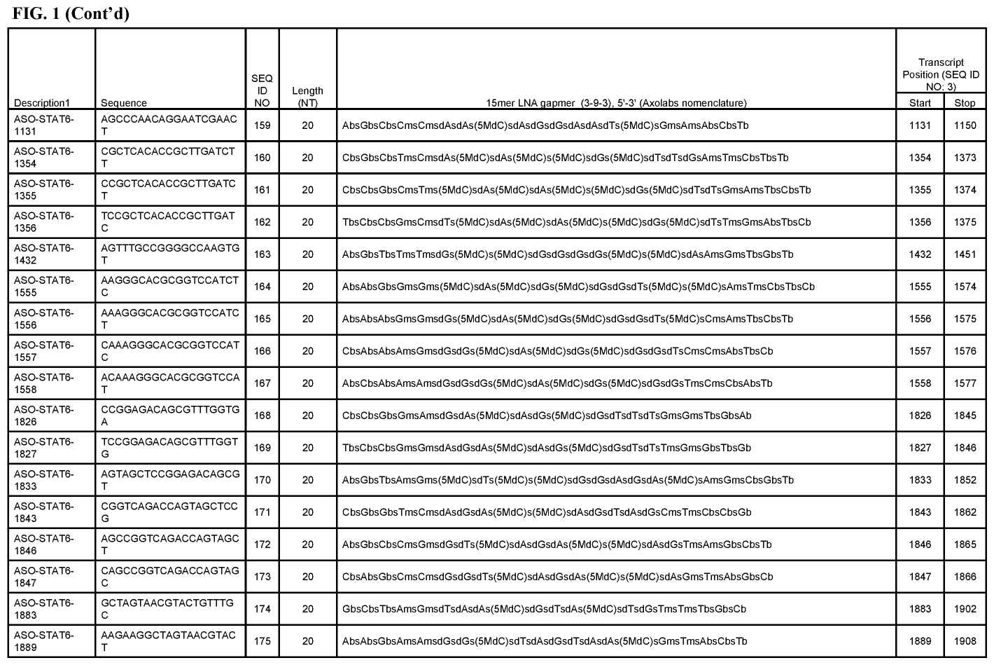

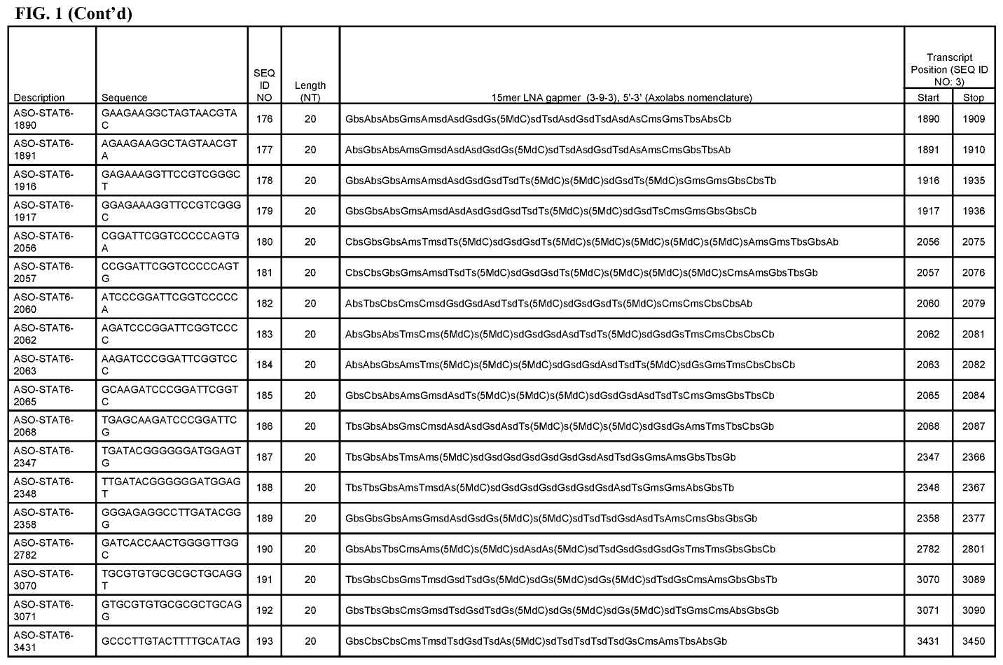

The terms “corresponding to” and “corresponds to,” when referencing two separate nucleic acid or nucleotide sequences can be used to clarify regions of the sequences that correspond or are similar to each other based on homology and/or functionality, although the nucleotides of the specific sequences can be numbered differently. For example, different isoforms of a gene transcript can have similar or conserved portions of nucleotide sequences whose numbering can differ in the respective isoforms based on alternative splicing and/or other modifications. In addition, it is recognized that different numbering systems can be employed when characterizing a nucleic acid or nucleotide sequence (e.g., a gene transcript and whether to begin numbering the sequence from the translation start codon or to include the 5′UTR). Further, it is recognized that the nucleic acid or nucleotide sequence of different variants of a gene or gene transcript can vary. As used herein, however, the regions of the variants that share nucleic acid or nucleotide sequence homology and/or functionality are deemed to “correspond” to one another. For example, a nucleotide sequence of a STAT6 transcript corresponding to nucleotides X to Y of SEQ ID NO: 1 (“reference sequence”) refers to an STAT6 transcript sequence (e.g., STAT6 pre-mRNA or mRNA) that has an identical sequence or a similar sequence to nucleotides X to Y of SEQ ID NO: 1, wherein X is the start site and Y is the end site (as shown in FIG. 1). A person of ordinary skill in the art can identify the corresponding X and Y residues in the STAT6 transcript sequence by aligning the STAT6 transcript sequence with SEQ ID NO: 1.

The terms “corresponding nucleotide analog” and “corresponding nucleotide” are intended to indicate that the nucleobase in the nucleotide analog and the naturally occurring nucleotide have the same pairing, or hybridizing, ability. For example, when the 2-deoxyribose unit of the nucleotide is linked to an adenine, the “corresponding nucleotide analog” contains a pentose unit (different from 2-deoxyribose) linked to an adenine.

The annotation of ASO chemistry is as follows Beta-D-oxy LNA nucleotides are designated by OxyB where B designates a nucleotide base such as thymine (T), uridine (U), cytosine (C), 5-methylcytosine (MC), adenine (A) or guanine (G), and thus include OxyA, OxyT, OxyMC, OxyC and OxyG. DNA nucleotides are designated by DNAb, where the lower case b designates a nucleotide base such as thymine (T), uridine (U), cytosine (C), 5-methylcytosine (Mc), adenine (A) or guanine (G), and thus include DNAa, DNAt, DNA and DNAg. The letter M before C or c indicates 5-methylcytosine. The letter “s” indicates a phosphorothioate internucleotide linkage.

The term “ASO Number” or “ASO No.” as used herein refers to a unique number given to a nucleotide sequence having the detailed chemical structure of the components, e.g., nucleosides (e.g., DNA), nucleoside analogs (e.g., beta-D-oxy-LNA), nucleobase (e.g., A, T, G, C, U, or MC), and backbone structure (e.g., phosphorothioate or phosphorodiester). For example, ASO-STAT6-1053 can refer to STAT6-1053 (SEQ ID NO: 91).

“Potency” is normally expressed as an IC₅₀ or EC₅₀ value, in μM, nM or pM unless otherwise stated. Potency can also be expressed in terms of percent inhibition. IC₅₀ is the median inhibitory concentration of a therapeutic molecule. EC₅₀ is the median effective concentration of a therapeutic molecule relative to a vehicle or control (e.g., saline). In functional assays, IC₅₀ is the concentration of a therapeutic molecule that reduces a biological response, e.g., transcription of mRNA or protein expression, by 50% of the biological response that is achieved by the therapeutic molecule. In functional assays, EC₅₀ is the concentration of a therapeutic molecule that produces 50% of the biological response, e.g., transcription of mRNA or protein expression. IC₅₀ or EC₅₀ can be calculated by any number of means known in the art.

As used herein, the term “inhibiting,” e.g., the expression of STAT6 gene transcript and/or STAT6 protein refers to the ASO reducing the expression of the STAT6 gene transcript and/or STAT6 protein in a cell or a tissue. In some aspects, the term “inhibiting” refers to complete inhibition (100% inhibition or non-detectable level) of STAT6 gene transcript or STAT6 protein. In other aspects, the term “inhibiting” refers to at least 5%, at least 10%, at least 15%, at least 20%, at least 25%, at least 30%, at least 35%, at least 40%, at least 45%, at least 50%, at least 60%, at least 70%, at least 80%, at least 90%, at least 95% or at least 99% inhibition of STAT6 gene transcript and/or STAT6 protein expression in a cell or a tissue.

As used herein, the term “extracellular vesicle” or “EV” refers to a cell-derived vesicle comprising a membrane that encloses an internal space. Extracellular vesicles comprise all membrane-bound vesicles (e.g., exosomes, nanovesicles) that have a smaller diameter than the cell from which they are derived. In some aspects, extracellular vesicles range in diameter from 20 nm to 1000 nm, and can comprise various macromolecular payload either within the internal space (i.e., lumen), displayed on the external surface of the extracellular vesicle, and/or spanning the membrane. In some aspects, the payload can comprise nucleic acids, proteins, carbohydrates, lipids, small molecules, and/or combinations thereof. In certain aspects, an extracellular vehicle comprises a scaffold moiety. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, the extracellular vesicles are produced by cells that express one or more transgene products.

As used herein, the term “exosome” refers to an extracellular vesicle with a diameter between 20-300 nm (e.g., between 40-200 nm). Exosomes comprise a membrane that encloses an internal space (i.e., lumen), and, in some aspects, can be generated from a cell (e.g., producer cell) by direct plasma membrane budding or by fusion of the late endosome with the plasma membrane. In certain aspects, an exosome comprises a scaffold moiety. As described infra, exosome can be derived from a producer cell, and isolated from the producer cell based on its size, density, biochemical parameters, or a combination thereof. In some aspects, the EVs, e.g., exosomes, of the present disclosure are produced by cells that express one or more transgene products.

As used herein, the term “nanovesicle” refers to an extracellular vesicle with a diameter between 20-250 nm (e.g., between 30-150 nm) and is generated from a cell (e.g., producer cell) by direct or indirect manipulation such that the nanovesicle would not be produced by the cell without the manipulation. Appropriate manipulations of the cell to produce the nanovesicles include but are not limited to serial extrusion, treatment with alkaline solutions, sonication, or combinations thereof. In some aspects, production of nanovesicles can result in the destruction of the producer cell. In some aspects, population of nanovesicles described herein are substantially free of vesicles that are derived from cells by way of direct budding from the plasma membrane or fusion of the late endosome with the plasma membrane. In certain aspects, a nanovesicle comprises a scaffold moiety. Nanovesicles, once derived from a producer cell, can be isolated from the producer cell based on its size, density, biochemical parameters, or a combination thereof.

As used herein the term “surface-engineered EVs, e.g., exosomes” (e.g., Scaffold X-engineered EVs, e.g., exosomes) refers to an EV, e.g., exosome, with the membrane or the surface of the EV, e.g., exosome, modified in its composition so that the surface of the engineered EV, e.g., exosome, is different from that of the EV, e.g., exosome, prior to the modification or of the naturally occurring EV, e.g., exosome. The engineering can be on the surface of the EV, e.g., exosome, or in the membrane of the EV, e.g., exosome, so that the surface of the EV, e.g., exosome, is changed. For example, the membrane is modified in its composition of a protein, a lipid, a small molecule, a carbohydrate, etc. The composition can be changed by a chemical, a physical, or a biological method or by being produced from a cell previously or concurrently modified by a chemical, a physical, or a biological method. Specifically, the composition can be changed by a genetic engineering or by being produced from a cell previously modified by genetic engineering. In some aspects, a surface-engineered EV, e.g., exosome, comprises an exogenous protein (i.e., a protein that the EV, e.g., exosome, does not naturally express) or a fragment or variant thereof that can be exposed to the surface of the EV, e.g., exosome, or can be an anchoring point (attachment) for a moiety exposed on the surface of the EV, e.g., exosome. In other aspects, a surface-engineered EV, e.g., exosome, comprises a higher expression (e.g., higher number) of a natural exosome protein (e.g., Scaffold X) or a fragment or variant thereof that can be exposed to the surface of the EV, e.g., exosome, or can be an anchoring point (attachment) for a moiety exposed on the surface of the EV, e.g., exosome.

As used herein the term “lumen-engineered exosome” (e.g., Scaffold Y-engineered exosome) refers to an EV, e.g., exosome, with the membrane or the lumen of the EV, e.g., exosome, modified in its composition so that the lumen of the engineered EV, e.g., exosome, is different from that of the EV, e.g., exosome, prior to the modification or of the naturally occurring EV, e.g., exosome. The engineering can be directly in the lumen or in the membrane of the EV, e.g., exosome so that the lumen of the EV, e.g., exosome is changed. For example, the membrane is modified in its composition of a protein, a lipid, a small molecule, a carbohydrate, etc. so that the lumen of the EV, e.g., exosome is modified. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering. In some aspects, a lumen-engineered exosome comprises an exogenous protein (i.e., a protein that the EV, e.g., exosome does not naturally express) or a fragment or variant thereof that can be exposed in the lumen of the EV, e.g., exosome or can be an anchoring point (attachment) for a moiety exposed on the inner layer of the EV, e.g., exosome. In other aspects, a lumen-engineered EV, e.g., exosome, comprises a higher expression of a natural exosome protein (e.g., Scaffold X or Scaffold Y) or a fragment or variant thereof that can be exposed to the lumen of the exosome or can be an anchoring point (attachment) for a moiety exposed in the lumen of the exosome.

The term “modified,” when used in the context of EVs, e.g., exosomes described herein, refers to an alteration or engineering of an EV, e.g., exosome and/or its producer cell, such that the modified EV, e.g., exosome is different from a naturally-occurring EV, e.g., exosome. In some aspects, a modified EV, e.g., exosome described herein comprises a membrane that differs in composition of a protein, a lipid, a small molecular, a carbohydrate, etc. compared to the membrane of a naturally-occurring EV, e.g., exosome (e.g., membrane comprises higher density or number of natural exosome proteins and/or membrane comprises proteins that are not naturally found in exosomes (e.g., an ASO). In certain aspects, such modifications to the membrane changes the exterior surface of the EV, e.g., exosome (e.g., surface-engineered EVs, e.g., exosomes described herein). In certain aspects, such modifications to the membrane changes the lumen of the EV, e.g., exosome (e.g., lumen-engineered EVs, e.g., exosomes described herein).

As used herein, the term “scaffold moiety” refers to a molecule that can be used to anchor a payload or any other compound of interest (e.g., an ASO) to the EV, e.g., exosome either on the luminal surface or on the exterior surface of the EV, e.g., exosome. In certain aspects, a scaffold moiety comprises a synthetic molecule. In some aspects, a scaffold moiety comprises a non-polypeptide moiety. In other aspects, a scaffold moiety comprises a lipid, carbohydrate, or protein that naturally exists in the EV, e.g., exosome. In some aspects, a scaffold moiety comprises a lipid, carbohydrate, or protein that does not naturally exist in the EV, e.g., exosome. In certain aspects, a scaffold moiety is Scaffold X. In some aspects, a scaffold moiety is Scaffold Y. In further aspects, a scaffold moiety comprises both Scaffold X and Scaffold Y. Non-limiting examples of other scaffold moieties that can be used with the present disclosure include: aminopeptidase N (CD13); Neprilysin, AKA membrane metalloendopeptidase (MME); ectonucleotide pyrophosphatase/phosphodiesterase family member 1 (ENPP1); Neuropilin-1 (NRP1); CD9, CD63, CD81, PDGFR, GPI anchor proteins, lactadherin (MFGE8), LAMP2, and LAMP2B.

As used herein, the term “Scaffold X” refers to exosome proteins that have recently been identified on the surface of exosomes. See, e.g., U.S. Pat. No. 10,195,290, which is incorporated herein by reference in its entirety. Non-limiting examples of Scaffold X proteins include: prostaglandin F2 receptor negative regulator (“the PTGFRN protein”); basigin (“the BSG protein”); immunoglobulin superfamily member 2 (“the IGSF2 protein”); immunoglobulin superfamily member 3 (“the IGSF3 protein”); immunoglobulin superfamily member 8 (“the IGSF8 protein”); integrin beta-1 (“the ITGB1 protein); integrin alpha-4 (“the ITGA4 protein”); 4F2 cell-surface antigen heavy chain (“the SLC3A2 protein”); a class of ATP transporter proteins (“the ATP1A1 protein,” “the ATP1A2 protein,” “the ATP1A3 protein,” “the ATP1A4 protein,” “the ATP1B3 protein,” “the ATP2B1 protein,” “the ATP2B2 protein,” “the ATP2B3 protein,” “the ATP2B protein”); and a functional fragment thereof. In some aspects, a Scaffold X protein can be a whole protein or a fragment thereof (e.g., functional fragment, e.g., the smallest fragment that is capable of anchoring another moiety on the exterior surface or on the luminal surface of the EV, e.g., exosome). In some aspects, a Scaffold X can anchor a moiety (e.g., an ASO) to the external surface or the luminal surface of the exosome.

As used herein, the term “Scaffold Y” refers to exosome proteins that were newly identified within the lumen of exosomes. Non-limiting examples of Scaffold Y proteins that can be used in the compositions and methods disclosed herein include those Scaffold Y proteins disclosed, for example, in International Publication No. WO/2019/099942 or WO 2020/101740, each of which is incorporated herein by reference in its entirety. Further examples of Scaffold Y proteins include: myristoylated alanine rich Protein Kinase C substrate (“the MARCKS protein”); myristoylated alanine rich Protein Kinase C substrate like 1 (“the MARCKSL1 protein”); and brain acid soluble protein 1 (“the BASP1 protein”). In some aspects, a Scaffold Y protein can be a whole protein or a fragment thereof (e.g., functional fragment, e.g., the smallest fragment that is capable of anchoring a moiety to the luminal surface of the exosome). In some aspects, a Scaffold Y can anchor a moiety (e.g., an ASO) to the luminal surface of the EV, e.g., exosome. In some aspects, a Scaffold Y can anchor a moiety (e.g., an ASO) to the exterior surface of the EV, e.g., exosome.

As used herein, the term “fragment” of a protein (e.g., therapeutic protein, Scaffold X, or Scaffold Y) refers to an amino acid sequence of a protein that is shorter than the naturally-occurring sequence, N- and/or C-terminally deleted or any part of the protein deleted in comparison to the naturally occurring protein. As used herein, the term “functional fragment” refers to a protein fragment that retains protein function. Accordingly, in some aspects, a functional fragment of a Scaffold X protein retains the ability to anchor a moiety on the luminal surface or on the exterior surface of the EV, e.g., exosome. Similarly, in certain aspects, a functional fragment of a Scaffold Y protein retains the ability to anchor a moiety on the luminal surface or exterior surface of the EV, e.g., exosome. Whether a fragment is a functional fragment can be assessed by any art known methods to determine the protein content of EVs, e.g., exosomes including Western Blots, FACS analysis and fusions of the fragments with autofluorescent proteins like, e.g., GFP. In certain aspects, a functional fragment of a Scaffold X protein retains at least about 50%, at least about 60%, at least about 70%, at least about 80%, at least about 90% or at least about 100% of the ability, e.g., an ability to anchor a moiety, of the naturally occurring Scaffold X protein. In some aspects, a functional fragment of a Scaffold Y protein retains at least about 50%, at least about 60%, at least about 70%, at least about 80%, at least about 90% or at least about 100% of the ability, e.g., an ability to anchor another molecule, of the naturally occurring Scaffold Y protein.

As used herein, the term “variant” of a molecule (e.g., functional molecule, antigen, Scaffold X and/or Scaffold Y) refers to a molecule that shares certain structural and functional identities with another molecule upon comparison by a method known in the art. For example, a variant of a protein can include a substitution, insertion, deletion, frameshift or rearrangement in another protein.

In some aspects, a variant of a Scaffold X comprises a variant having at least about 70% identity to the full-length, mature PTGFRN, BSG, IGSF2, IGSF3, IGSF8, ITGB1, ITGA4, SLC3A2, or ATP transporter proteins or a fragment (e.g., functional fragment) of the PTGFRN, BSG, IGSF2, IGSF3, IGSF8, ITGB1, ITGA4, SLC3A2, or ATP transporter proteins. In some aspects, variants or variants of fragments of PTGFRN share at least about 70%, at least about 80%, at least about 85%, at least about 90%, at least about 95%, at least about 96%, at least about 97%, at least about 98%, or at least about 99% sequence identity with PTGFRN according to SEQ ID NO: 301 or with a functional fragment thereof. In some aspects, the variant or variant of a fragment of Scaffold X protein disclosed herein retains the ability to be specifically targeted to EVs, e.g., exosomes. In some aspects, the Scaffold X includes one or more mutations, for example, conservative amino acid substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences may be accomplished using readily available software both for online use and for download. Suitable software programs are available from various sources, and for alignment of both protein and nucleotide sequences. One suitable program to determine percent sequence identity is bl2seq, part of the BLAST suite of programs available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity may be curated either automatically or manually.

The polynucleotide variants can contain alterations in the coding regions, non-coding regions, or both. In one aspect, the polynucleotide variants contain alterations which produce silent substitutions, additions, or deletions, but do not alter the properties or activities of the encoded polypeptide. In another aspect, nucleotide variants are produced by silent substitutions due to the degeneracy of the genetic code. In other aspects, variants in which 5-10, 1-5, or 1-2 amino acids are substituted, deleted, or added in any combination. Polynucleotide variants can be produced for a variety of reasons, e.g., to optimize codon expression for a particular host (change codons in the human mRNA to others, e.g., a bacterial host such as E. coli).

Naturally occurring variants are called “allelic variants,” and refer to one of several alternate forms of a gene occupying a given locus on a chromosome of an organism (Genes I I, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-la. They used random mutagenesis to generate over 3,500 individual IL-la mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, polypeptide variants include, e.g., modified polypeptides. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination. In some aspects, Scaffold X and/or Scaffold Y is modified at any convenient location.

As used herein the term “linked to” or “conjugated to” are used interchangeably and refer to a covalent or non-covalent bond formed between a first moiety and a second moiety, e.g., Scaffold X and an ASO, respectively, e.g., a scaffold moiety expressed in or on the extracellular vesicle and an ASO, e.g., Scaffold X (e.g., a PTGFRN protein), respectively, in the luminal surface of or on the external surface of the extracellular vesicle.

The term “encapsulated”, or grammatically different forms of the term (e.g., encapsulation, or encapsulating) refers to a status or process of having a first moiety (e.g., an ASO) inside a second moiety (e.g., an EV, e.g., exosome) without chemically or physically linking the two moieties. In some aspects, the term “encapsulated” can be used interchangeably with “in the lumen of.” Non-limiting examples of encapsulating a first moiety (e.g., an ASO) into a second moiety (e.g., EVs, e.g., exosomes) are disclosed elsewhere herein.

As used herein, the term “producer cell” refers to a cell used for generating an EV, e.g., exosome. A producer cell can be a cell cultured in vitro, or a cell in vivo. A producer cell includes, but not limited to, a cell known to be effective in generating EVs, e.g., exosomes, e.g., HEK293 cells, Chinese hamster ovary (CHO) cells, mesenchymal stem cells (MSCs), BJ human foreskin fibroblast cells, fHDF fibroblast cells, AGE.HN® neuronal precursor cells, CAP® amniocyte cells, adipose mesenchymal stem cells, RPTEC/TERT1 cells. In certain aspects, a producer cell is not an antigen-presenting cell. In some aspects, a producer cell is not a dendritic cell, a B cell, a mast cell, a macrophage, a neutrophil, Kupffer-Browicz cell, cell derived from any of these cells, or any combination thereof. In some aspects, the EVs, e.g., exosomes useful in the present disclosure do not carry an antigen on MHC class I or class II molecule exposed on the surface of the EV, e.g., exosome, but instead can carry an antigen in the lumen of the EV, e.g., exosome or on the surface of the EV, e.g., exosome by attachment to Scaffold X and/or Scaffold Y.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some aspects, isolating or purifying as used herein is the process of removing, partially removing (e.g., a fraction) of the EVs from a sample containing producer cells. In some aspects, an isolated EV composition has no detectable undesired activity or, alternatively, the level or amount of the undesired activity is at or below an acceptable level or amount. In other aspects, an isolated EV composition has an amount and/or concentration of desired EVs at or above an acceptable amount and/or concentration. In other aspects, the isolated EV composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some aspects, isolated EV preparations are substantially free of residual biological products. In some aspects, the isolated EV preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the EV composition contains no detectable producer cells and that only EVs are detectable.

As used herein, the term “payload” refers to an agent that acts on a target (e.g., a target cell) that is contacted with the EV. A non-limiting examples of payload that can be included on the EV, e.g., exosome, is an ASO. Payloads that can be introduced into an EV, e.g., exosome, and/or a producer cell include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, and siRNA), amino acids (e.g., amino acids comprising a detectable moiety or a toxin or that disrupt translation), polypeptides (e.g., enzymes), lipids, carbohydrates, and small molecules (e.g., small molecule drugs and toxins). In certain aspects, a payload comprises an ASO.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. As used herein, the term “antigen” refers to any agent that when introduced into a subject elicits an immune response (cellular or humoral) to itself. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function.

The term “antibody” includes, by way of example, both naturally occurring and non-naturally occurring antibodies; monoclonal and polyclonal antibodies; chimeric and humanized Abs; human or nonhuman Abs; wholly synthetic Abs; and single chain Abs. A nonhuman antibody may be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

As used herein, the term “antagonist,” for example a PD-1 antagonist, refers to any substance that limits, reduces, inhibits, block, or otherwise lowers the activity of a target. For example, a PD-antagonist limits, reduces, inhibits, block, or otherwise lowers the activity of PD-1. Examples of antagonists include, but are not limited to, small molecules, polypeptides (e.g., ligands, receptors, antibodies, and fragments thereof), nucleic acid molecules, and any combination thereof. In certain aspects, the antagonist comprises an antibody or antigen-binding portion thereof.

As used herein, “programmed death-1” or “PD-1” refers to an immunoinhibitory receptor in the CD28 family. PD-1 is expressed predominantly on previously activated T cells in vivo. PD-1 binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863. “PD-1” and “PD-1 receptor” are used interchangeably herein. As used herein, the term “PD-1 activity” or “PD-1 signaling” refers, without limitation, to the interaction between PD-1 and PD-L1 and/or PD-L1. As such, in some aspects, a PD-1 antagonist inhibits or blocks the interaction between PD-1 and PD-L1 (and/or PD-L2). Therefore, in some aspects, a “PD-1 antagonist” comprises an anti-PD-1 antibody, an anti-PD-L1 antibody, an anti-PD-L2 antibody, or any combination thereof.

Any anti-PD-1 antibody or anti-PD-L1 antibody known in the art can be used in the methods disclosed herein. Non-limiting examples of anti-PD-1 antibodies include nivolumab (see U.S. Pat. No. 8,008,449), pembrolizumab, PDR001 (see WO 2015/112900), MEDI-0680 (see WO 2012/145493), cemiplimab (see WO 2015/112800), JS001 (see Si-Yang Liu et al., J. Hematol. Oncol. 10:136 (2017)), BGB-A317 (see WO 2015/35606 and US 2015/0079109), INCSHR1210 (see WO 2015/085847; Si-Yang Liu et al., J. Hematol. Oncol. 10:136 (2017)), TSR-042 (see WO2014/179664), GLS-010 (see Si-Yang Liu et al., J. Hematol. Oncol. 10:136 (2017)), AM-0001, STI-1110 (see WO 2014/194302), AGEN2034 (see WO 2017/040790), MGA012 (see WO 2017/19846), and IBI308 (see WO 2017/024465, WO 2017/025016, WO 2017/132825, and WO 2017/133540) (each of which is incorporated by reference herein in its entirety). Non-limiting examples of anti-PD-L1 antibodies include but are not limited to BMS-936559 (see, e.g., U.S. Pat. No. 7,943,743 and WO 2013/173223), atezolizumab (see U.S. Pat. No. 8,217,149), durvalumab (see WO 2011/066389), avelumab (see WO 2013/079174), STI-1014 (see WO2013/181634), CX-072 (see WO2016/149201), KN035 (see Zhang et al., Cell Discov. 7:3 (March 2017)), LY3300054 (see, e.g., WO 2017/034916), and CK-301 (see Gorelik et al., AACR: Abstract 4606 (April 2016)) (each of which is incorporated by reference herein in its entirety).

The terms “individual,” “subject,” “host,” and “patient,” are used interchangeably herein and refer to any mammalian subject for whom diagnosis, treatment, or therapy is desired, particularly humans. The compositions and methods described herein are applicable to both human therapy and veterinary applications. In some aspects, the subject is a mammal, and in other aspects the subject is a human. As used herein, a “mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

The term “pharmaceutical composition” refers to a preparation which is in such form as to permit the biological activity of the active ingredient to be effective, and which contains no additional components which are unacceptably toxic to a subject to which the composition would be administered. Such composition can be sterile.

As used herein, the term “substantially free” means that the sample comprising EVs, e.g., exosomes, comprise less than 10% of macromolecules by mass/volume (m/v) percentage concentration. Some fractions may contain less than 0.001%, less than 0.01%, less than 0.05%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% (m/v) of macromolecules.

As used herein, the term “macromolecule” means nucleic acids, contaminant proteins, lipids, carbohydrates, metabolites, or a combination thereof.

As used herein, the term “conventional exosome protein” means a protein previously known to be enriched in exosomes, including but is not limited to CD9, CD63, CD81, PDGFR, GPI anchor proteins, lactadherin (MFGE8), LAMP2, and LAMP2B, a fragment thereof, or a peptide that binds thereto.

“Administering,” as used herein, means to give a composition comprising an EV, e.g., exosome, disclosed herein to a subject via a pharmaceutically acceptable route. Routes of administration can be intravenous, e.g., intravenous injection and intravenous infusion. Additional routes of administration include, e.g., subcutaneous, intramuscular, oral, nasal, and pulmonary administration. EVs, e.g., exosomes can be administered as part of a pharmaceutical composition comprising at least one excipient.

An “effective amount” of, e.g., an ASO or an extracellular vesicle as disclosed herein, is an amount sufficient to carry out a specifically stated purpose. An “effective amount” can be determined empirically and in a routine manner, in relation to the stated purpose.

“Treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also includes prophylaxis or prevention of a disease or condition or its symptoms thereof. In one aspect, the “treating” or “treatment” includes inducing hematopoiesis in a subject in need thereof. In some aspects, the disease or condition is associated with a hematopoiesis or a deficiency thereof. In certain aspects, the disease or condition is a cancer. In some aspects, the treating enhances hematopoiesis in a subject having a cancer, wherein the enhanced hematopoiesis comprises increased proliferation and/or differentiation of one or more immune cell in the subject

“Prevent” or “preventing,” as used herein, refers to decreasing or reducing the occurrence or severity of a particular outcome. In some aspects, preventing an outcome is achieved through prophylactic treatment. In some aspects, an EV, e.g., an exosome, comprising an ASO, described herein, is administered to a subject prophylactically. In some aspects, the subject is at risk of developing cancer. In some aspects, the subject is at risk of developing a hematopoietic disorder.

### II. Methods of the Disclosure

Certain aspects of the present disclosure are directed to methods of preventing and/or treating a disease or disorder in a subject in need thereof, comprising administering (i) an extracellular vesicle (EV), e.g., exosome, comprising an antisense oligonucleotide (ASO), and (ii) a PD-1 antagonist. In certain aspects, the ASO comprises a contiguous nucleotide sequence of 10 to 30 nucleotides in length that is complementary to a nucleic acid sequence within a STAT6 transcript. In some aspects, the PD-1 antagonist blocks, inhibits, and/or reduces the interaction between PD-1 and PD-L1. In some aspects, the PD-1 antagonist comprises an antibody or an antigen-binding portion thereof that specifically binds PD-1 (“an anti-PD-1 antibody”). In some aspects, the PD-1 antagonist comprises an antibody or an antigen-binding portion thereof that specifically binds PD-L1 (“an anti-PD-L1 antibody”).

As described herein, ASOs useful for the present disclosure can specifically hybridize to one or more regions of a STAT6 transcript (e.g., pre-mRNA or mRNA), resulting in reduction and/or inhibition of STAT6 protein expression in a cell. Accordingly, EVs (e.g., exosomes) comprising such an ASO (e.g., EVs disclosed herein) can be useful for preventing and/or treating any disease or disorder associated with increased expression of a STAT6 protein.

In some aspects, a disease or disorder that can be treated with the present methods comprises a cancer. In certain aspects, the cancer is associated with increased expression of a STAT6 protein. Non-limiting examples of cancers that can be treated with the present disclosure include a colorectal cancer, lung cancer (e.g., non-small cell lung cancer (NSCLC)), pancreatic cancer (e.g., pancreatic ductal adenocarcinoma (PDAC)), leukemia, uterine cancer, ovarian cancer, bladder cancer, bile duct cancer, gastric cancer, or any combination thereof. In some aspects, the cancer is selected from colon adenocarcinoma, rectum adenocarcinoma, pancreatic adenocarcinoma, pancreatic ductal adenocarcinoma (PDAC), ovarian serous cystadenocarcinoma, acute myelid leukemia, testicular germ cell tumors, lung adenocarcinoma, brain lower grade glioma, glioblastoma multiforme, uveal melanoma, thyroid carcinoma, uterine corpus endometrial carcinoma, uterine carcinosarcoma, pheochromocytoma, paraganglioma, and any combination thereof. In certain aspects, the cancer is a myeloid-rich cancer. In some aspects, the cancer comprises a liver cancer. In some aspects, the cancer comprises hepatocellular cancer (HCC). In some aspects, the cancer comprises pancreatic ductal adenocarcinoma (PDAC), in some aspects, the cancer comprises colorectal carcinoma (CRC). In some aspects, the cancer comprises ovarian cancer. In some aspects, the cancer comprises leptomeningeal cancer. In some aspects, the method disclosed herein increase immune cell, e.g., macrophage, infiltration of a tumor. In certain aspects, the cancer is a myeloid-rich cancer. In certain aspects, the cancer comprises an M2-rich cancer. In some aspects, the cancer comprises a liver cancer. In some aspects, the cancer comprises hepatocellular cancer (HCC). In some aspects, the cancer comprises pancreatic ductal adenocarcinoma (PDAC), in some aspects, the cancer comprises colorectal carcinoma (CRC). In some aspects, the cancer comprises ovarian cancer. In some aspects, the cancer comprises leptomeningeal cancer. In certain aspects, the cancer comprises an intractable tumor of the central nervous system.

In some aspects, the methods disclosed herein treat a tumor of the central nervous system in a subject. In some aspects, the method treats a brain tumor in a subject. In some aspects, the method treats a glioblastoma in a subject. In some aspects, the glioblastoma is a glioblastoma multiforme (GBM). In some aspects, the method treats a leptomeningeal cancer disease in a subject. In some aspects, the EV, e.g., the exosome, comprising the ASO activates macrophages within the central nervous system. In some aspects, the EV, e.g., the exosome, comprising the ASO induces M1 polarization of macrophages within the central nervous system. In some aspects, the EV, e.g., the exosome, comprising the ASO activates meningeal macrophages. In some aspects, the EV, e.g., the exosome, comprising the ASO induces M1 polarization of meningeal macrophages. In some aspects, the EV, e.g., the exosome, comprising the ASO induces tumor infiltration of meningeal macrophages.

When administered to a subject with a cancer, in certain aspects, EVs (e.g., exosome) of the present disclosure can up-regulate an immune response and enhance the tumor targeting of the subject's immune system. In some aspects, the cancer being treated is characterized by infiltration of leukocytes (T-cells, B-cells, macrophages, dendritic cells, monocytes) into the tumor microenvironment, or so-called “hot tumors” or “inflammatory tumors”. In some aspects, the cancer being treated is characterized by low levels or undetectable levels of leukocyte infiltration into the tumor microenvironment, or so-called “cold tumors” or “non-inflammatory tumors”. In some aspects, an EV is administered in an amount and for a time sufficient to convert a “cold tumor” into a “hot tumor”, i.e., said administering results in the infiltration of leukocytes (such as T-cells) into the tumor microenvironment. In certain aspects, cancer comprises bladder cancer, cervical cancer, renal cell cancer, testicular cancer, colorectal cancer, lung cancer, head and neck cancer, and ovarian, lymphoma, liver cancer, glioblastoma, melanoma, myeloma, leukemia, pancreatic cancers, or combinations thereof. In other term, “distal tumor” or “distant tumor” refers to a tumor that has spread from the original (or primary) tumor to distant organs or distant tissues, e.g., lymph nodes. In some aspects, the EVs of the disclosure treats a tumor after the metastatic spread. In some aspects, administration of an EV, e.g., exosome, of the present disclosure induces immunological memory.

In some aspects, the EV, e.g., the exosome, comprising the ASO activates tumor-associated macrophages (TAMs). In some aspects, the EV, e.g., the exosome, comprising the ASO induces M1 polarization of TAMs. In some aspects, the EV, e.g., the exosome, comprising the ASO induces expression of M1 markers in TAMs. In some aspects, the EV, e.g., the exosome, comprising the ASO reduces expression of M2 markers in TAMs. In some aspects, the EV, e.g., the exosome, comprising the ASO modulates the expression of one or more gene listed in Tables 4A, 4B, and/or 5. As such, certain aspects of the present disclosure are directed to methods of inducing M1 polarization of TAMs, comprising administering to a subject an EV, e.g., the exosome, comprising an ASO disclosed herein.

In some aspects, the EV, e.g., the exosome, treats a fibrosis in a subject in need thereof. Excessive M2 macrophage activation leads to the continuous production of TGFβ and growth factors that promote proliferation of myofibroblasts, activation of EMT/EndoMT, and extracellular matrix deposition. M2 macrophages represent a break point between wound healing and exacerbation of pro-fibrotic process. In some aspects, the fibrosis is selected from liver fibrosis (NASH), cirrhosis, pulmonary fibrosis, cystic fibrosis, chronic ulcerative colitis/IBD, bladder fibrosis, kidney fibrosis, CAPS (Muckle-Wells syndrome), atrial fibrosis, endomyocardial fibrosis, old myocardial infarction, glial scar, arterial stiffness, arthrofibrosis, Crohn's disease, Dupuytren's contracture, keloid fibrosis, mediastinal fibrosis, myelofibrosis, Peyronie's disease, nephrogenic systemic fibrosis, progressive massive fibrosis, retroperitoneal fibrosis, scleroderma/systemic sclerosis, adhesive capsulitis, and any combination thereof. In some aspects, the EV, e.g., the exosome, treats liver fibrosis (NASH). In some aspects, the EV, e.g., the exosome, treats CAPS (Muckle-Wells syndrome).

In some aspects, the EV, e.g., the exosome, treats a neurodegenerative disease. In some aspects, the neurodegenerative disease is selected from Alzheimer's disease, Parkinson's disease, prion disease, motor neuron disease, Huntington's disease, spinocerebellar ataxia, spinal muscular atrophy, and any combination thereof.

In some aspects, the EV, e.g., the exosome, treats a metabolic disorder/CVD. In some aspects, the metabolic disorder/CVD is selected from an acid-base imbalance, metabolic brain disease, disorder of calcium metabolism, DNA repair-deficiency disorder, glucose metabolism disorder, hyperlactatemia, iron metabolism disorder, lipid metabolism disorder, malabsorption syndrome, metabolic syndrome X, inborn error of metabolism, mitochondrial disease, phosphorus metabolism disorder, porphyrias, proteostasis deficiency, metabolic skin disease, wasting syndrome, water-electrolyte imbalance, and any combination thereof.

In some aspects, the EV (e.g., exosome) promotes anti-tumor immunity in a subject. In some aspects, administration of the EV (e.g., exosome) induces nitric oxide synthase (NOS2) expression in a subject. In some aspects, administration of the EV (e.g., exosome) promotes remodeling of the tumor microenvironment (TME) in a subject. In some aspects, administration of the EV (e.g., exosome) induces activation of tumor-infiltrating lymphocytes, e.g., tumor-infiltrating CD8 T cells, in a subject. In some aspects, administration of the EV (e.g., exosome) leads to increased expression of effector/activator genes by tumor-infiltrating lymphocytes, e.g., tumor-infiltrating CD8 T cells, in a subject. In some aspects, the expression of effector/activator genes Gzmb and Id2 are increased in tumor-infiltrating lymphocytes, e.g., tumor-infiltrating CD8 T cells, following administration of the EV (e.g., exosome). In some aspects, administration of the EV (e.g., exosome) leads to decreased exhaustion of tumor-infiltrating lymphocytes, e.g., tumor-infiltrating CD8 T cells, in a subject. In some aspects, administration of the EV (e.g., exosome) leads to decreased expression of exhaustion markers in tumor-infiltrating lymphocytes, e.g., tumor-infiltrating CD8 T cells. In some aspects, administration of the EV (e.g., exosome) leads to decreased expression of Lag3 in tumor-infiltrating lymphocytes, e.g., tumor-infiltrating CD8 T cells. In some aspects, administration of the EV (e.g., exosome) leads to an increase in the number of tumor-infiltrating lymphocytes, e.g., tumor-infiltrating CD8 T cells. In some aspects, administration of the EV (e.g., exosome) leads to a decrease in the number of Foxp3+ regulatory T cells, e.g., in the TME.

In some aspects, the EVs (e.g., exosomes) and/or the PD-1 antagonist are administered intravenously to the circulatory system of the subject. In some aspects, the EVs and/or the PD-1 antagonist are infused in suitable liquid and administered into a vein of the subject.

In some aspects, the EVs (e.g., exosomes) and/or the PD-1 antagonist are administered intra-arterially to the circulatory system of the subject. In some aspects, the EVs and/or the PD-1 antagonist are infused in suitable liquid and administered into an artery of the subject.

In some aspects, the EVs (e.g., exosomes) and/or the PD-1 antagonist are administered to the subject by intrathecal administration. In some aspects, the EVs (e.g., exosomes) and/or the PD-1 antagonist are administered by intrathecal administration, followed by application of a mechanical convective force to the torso. See, e.g., Verma et al., Alzheimer's Dement. 12:e12030 (2020); which is incorporated by reference herein in its entirety). In some aspects, the mechanical convective force, e.g., the oscillating vest, facilitates spread of the intrathecally dosed EVs, e.g., exosomes, further down the nerve thus allowing for better EV, e.g., exosome, delivery to nerves.

In some aspects, the intra- and trans-compartmental biodistribution of exosomes can be manipulated by exogenous extracorporeal forces acting upon a subject after compartmental delivery of exosomes. This includes the application of mechanical convection, for example by way of applying percussion, vibration, shaking, or massaging of a body compartment or the entire body. Following intrathecal dosing for example, the application of chest wall vibrations by several means including an oscillating mechanical jacket can spread the biodistribution of exosomes along the neuraxis or along cranial and spinal nerves, which can be helpful in the treatment of nerve disorders by drug carrying exosomes.

In some aspects, the application of external mechanical convective forces via an oscillating jacket or other similar means can be used to remove exosomes and other material from the cerebrospinal fluid of the intrathecal space and out to the peripheral circulation. This aspect can help remove endogenous toxic exosomes and other deleterious macromolecules such as beta-amyloid, tau, alpha-synuclein, TDP43, neurofilament and excessive cerebrospinal fluid from the intrathecal space to the periphery for elimination.

In some aspects, exosomes delivered via the intracebroventricular route can be made to translocate throughout the neuraxis by simultaneously incorporating a lumbar puncture and allowing for ventriculo-lumbar perfusion wherein additional fluid is infused into the ventricles after exosome dosing, while allowing the existing neuraxial column of CSF to exit is the lumbar puncture. Ventriculo-lumbar perfusion can allow ICV dosed exosome to spread along the entire neuraxis and completely cover the subarachoid space in order to treat leptomeningeal cancer and other diseases.

In some aspects, the application of external extracorporeal focused ultrasound, thermal energy (heat) or cold may be used to manipulate the compartmental pharmacokinetics and drug release properties of exosomes engineered to be sensitive to these phenomena.

In some aspects, the intracompartmental behavior and biodistribution of exosomes engineered to contain paramagnetic material can be manipulated by the external application of magnets or a magnetic field.

In some aspects, the EVs are administered via an injection into the spinal canal, or into the subarachnoid space so that it reaches the cerebrospinal fluid (CSF).

In some aspects, the EVs (e.g., exosomes) are administered intratumorally into one or more tumors of the subject.

In some aspects, the EVs (e.g., exosomes) are administered to the subject by intranasal administration. In some aspects, the EVs can be insufflated through the nose in a form of either topical administration or systemic administration. In certain aspects, the EVs are administered as nasal spray.

In some aspects, the EVs (e.g., exosomes) are administered to the subject by intraperitoneal administration. In some aspects, the EVs are infused in suitable liquid and injected into the peritoneum of the subject. In some aspects, the intraperitoneal administration results in distribution of the EVs to the lymphatics. In some aspects, the intraperitoneal administration results in distribution of the EVs to the thymus, spleen, and/or bone marrow. In some aspects, the intraperitoneal administration results in distribution of the EVs to one or more lymph nodes. In some aspects, the intraperitoneal administration results in distribution of the EVs to one or more of the cervical lymph node, the inguinal lymph node, the mediastinal lymph node, or the sternal lymph node. In some aspects, the intraperitoneal administration results in distribution of the EVs to the pancreas.

In some aspects, the EVs, e.g., exosomes, are administered to the subject by periocular administration. In some aspects, the s are injected into the periocular tissues. Periocular drug administration includes the routes of subconjunctival, anterior sub-Tenon's, posterior sub-Tenon's, and retrobulbar administration.

### III. Antisense Oligonucleotides (ASOs)

The present disclosure employs antisense oligonucleotides (ASOs) for use in modulating the function of nucleic acid molecules encoding mammalian STAT6, such as the STAT6 nucleic acid, e.g., STAT6 transcript, including STAT6 pre-mRNA, and STAT6 mRNA, or naturally occurring variants of such nucleic acid molecules encoding mammalian STAT6. The term “ASO” in the context of the present disclosure, refers to a molecule formed by covalent linkage of two or more nucleotides (i.e., an oligonucleotide).

In some aspects, the EV, e.g., the exosome, comprises at least one ASO. In some aspects, the EV, e.g., the exosome, comprises at least two ASOs, e.g., a first ASO comprising a first nucleotide sequence and a second ASO comprising a second nucleotide sequence. In some aspects, the EV, e.g., the exosome, comprises at least three ASOs, at least four ASOs, at least five ASOs, at least six ASOs, or more than six ASOs. In some aspects, each of the first ASO, the second ASO, the third ASO, the fourth ASO, the fifth ASO, the sixth ASO, and/or the ninth ASO is different.

In some aspects, the EV, e.g. the exosome, comprises a first ASO and a second ASO, wherein the first ASO comprises a first nucleotide sequence that is complimentary to a first target sequence in a first transcript, and wherein the second ASO comprises a second nucleotide sequence that is complimentary to a second target sequence in the first transcript. In some aspects, the first target sequence does not overlap with the second target sequence. In some aspects, the first target sequence comprises at least one nucleotide that is within the 5′UTR of the transcript, and the second target sequence does not comprise a nucleotide that is within the 5′UTR. In some aspects, the first target sequence comprises at least one nucleotide that is within the 3′UTR of the transcript, and the second target sequence does not comprise a nucleotide that is within the 3′UTR. In some aspects, the first target sequence comprises at least one nucleotide that is within the 5′UTR of the transcript, and the second target sequence comprises at least one nucleotide that is within the 3′UTR.

In some aspects, the first ASO targets a sequence within an exon-intron junction, and the second ASO targets a sequence within an exon-intron junction. In some aspects, the first ASO targets a sequence within an exon-intron junction, and the second ASO targets a sequence within an exon. In some aspects, the first ASO targets a sequence within an exon-intron junction, and the second ASO targets a sequence within an intron. In some aspects, the first ASO targets a sequence within an exon, and the second ASO targets a sequence within an exon. In some aspects, the first ASO targets a sequence within an intron, and the second ASO targets a sequence within an exon. In some aspects, the first ASO targets a sequence within an intron, and the second ASO targets a sequence within an intron.

In some aspects, the EV, e.g. the exosome, comprises a first ASO and a second ASO, wherein the first ASO comprises a first nucleotide sequence that is complimentary to a first target sequence in a first transcript, and wherein the second ASO comprises a second nucleotide sequence that is complimentary to a second target sequence in a second transcript, wherein the first transcript is not the product of the same gene as the second transcript.

The ASO comprises a contiguous nucleotide sequence of from about 10 to about 30, such as 10-20,14-20, 16-20, or 15-25, nucleotides in length. In certain aspects, the ASO is 20 nucleotides in length. In certain aspects, the ASO is 18 nucleotides in length. In certain aspects, the ASO is 19 nucleotides in length. In certain aspects, the ASO is 17 nucleotides in length. In certain aspects, the ASO is 16 nucleotides in length. In certain aspects, the ASO is 15 nucleotides in length. In certain aspects, the ASO is 14 nucleotides in length. In certain aspects, the ASO is 13 nucleotides in length. In certain aspects, the ASO is 12 nucleotides in length. In certain aspects, the ASO is 11 nucleotides in length. In certain aspects, the ASO is 10 nucleotides in length.

In some aspects, the ASO comprises a contiguous nucleotide sequence of from about 10 to about 50 nucleotides in length, e.g., about 10 to about 45, about 10 to about 40, about 10 or about 35, or about 10 to about 30. In certain aspects, the ASO is 21 nucleotides in length. In certain aspects, the ASO is 22 nucleotides in length. In certain aspects, the ASO is 23 nucleotides in length. In certain aspects, the ASO is 24 nucleotides in length. In certain aspects, the ASO is 25 nucleotides in length. In certain aspects, the ASO is 26 nucleotides in length. In certain aspects, the ASO is 27 nucleotides in length. In certain aspects, the ASO is 28 nucleotides in length. In certain aspects, the ASO is 29 nucleotides in length. In certain aspects, the ASO is 30 nucleotides in length. In certain aspects, the ASO is 31 nucleotides in length. In certain aspects, the ASO is 32 nucleotides in length. In certain aspects, the ASO is 33 nucleotides in length. In certain aspects, the ASO is 34 nucleotides in length. In certain aspects, the ASO is 35 nucleotides in length. In certain aspects, the ASO is 36 nucleotides in length. In certain aspects, the ASO is 37 nucleotides in length. In certain aspects, the ASO is 38 nucleotides in length. In certain aspects, the ASO is 39 nucleotides in length. In certain aspects, the ASO is 40 nucleotides in length. In certain aspects, the ASO is 41 nucleotides in length. In certain aspects, the ASO is 42 nucleotides in length. In certain aspects, the ASO is 43 nucleotides in length. In certain aspects, the ASO is 44 nucleotides in length. In certain aspects, the ASO is 45 nucleotides in length. In certain aspects, the ASO is 46 nucleotides in length. In certain aspects, the ASO is 47 nucleotides in length. In certain aspects, the ASO is 48 nucleotides in length. In certain aspects, the ASO is 49 nucleotides in length. In certain aspects, the ASO is 50 nucleotides in length.

The terms “antisense ASO,” “antisense oligonucleotide,” and “oligomer” as used herein are interchangeable with the term “ASO.”

A reference to a SEQ ID number includes a particular nucleobase sequence, but does not include any design or full chemical structure. Furthermore, the ASOs disclosed in the figures herein show a representative design, but are not limited to the specific design shown in the figures unless otherwise indicated. For example, when a claim (or this specification) refers to SEQ ID NO: 101, it includes the nucleotide sequence of SEQ ID NO: 101 only. The design of any ASO disclosed herein can be written as SEQ ID NO: XX, wherein each of the first nucleotide, the second nucleotide, the third nucleotide, the first nucleotide, the second nucleotide, and the Nth nucleotide from the 5′ end is a modified nucleotide, e.g., LNA, and each of the other nucleotides is a non-modified nucleotide (e.g., DNA).

In various aspects, the ASO of the disclosure does not comprise RNA (units). In some aspects, the ASO comprises one or more DNA units. In one aspect, the ASO according to the disclosure is a linear molecule or is synthesized as a linear molecule. In some aspects, the ASO is a single stranded molecule, and does not comprise short regions of, for example, at least 3, 4 or 5 contiguous nucleotides, which are complementary to equivalent regions within the same ASO (i.e. duplexes)—in this regard, the ASO is not (essentially) double stranded. In some aspects, the ASO is essentially not double stranded. In some aspects, the ASO is not a siRNA. In various aspects, the ASO of the disclosure can consist entirely of the contiguous nucleotide region. Thus, in some aspects the ASO is not substantially self-complementary.

In other aspects, the present disclosure includes fragments of ASOs. For example, the disclosure includes at least one nucleotide, at least two contiguous nucleotides, at least three contiguous nucleotides, at least four contiguous nucleotides, at least five contiguous nucleotides, at least six contiguous nucleotides, at least seven contiguous nucleotides, at least eight contiguous nucleotides, or at least nine contiguous nucleotides of the ASOs disclosed herein. Fragments of any of the sequences disclosed herein are contemplated as part of the disclosure.

In some aspects, the ASOs for the present disclosure include a phosphorodiamidate Morpholino oligomer (PMO) or a peptide-conjugated phosphorodiamidate morpholino oligomer (PPMO).

### III.A. ASOs Targeting STAT6

Suitably the ASO of the disclosure is capable of down-regulating (e.g., reducing or removing) expression of the STAT6 mRNA or STAT6 protein. In this regard, the ASO of the disclosure can promote differentiation of M2 macrophages and/or decrease the differentiation of M1 macrophages. In particular, the present disclosure is directed to ASOs that target one or more regions of the STAT6 pre-mRNA (e.g., intron regions, exon regions, and/or exon-intron junction regions).

Unless indicated otherwise, the term “STAT6,” as used herein, can refer to STAT6 from one or more species (e.g., humans, non-human primates, dogs, cats, guinea pigs, rabbits, rats, mice, horses, cattle, and bears).

STAT6 (STAT6) is also known as signal transducer and activator of transcription 6. Synonyms of STAT6/STAT6 are known and include IL-4 STAT; STAT, Interleukin 4-Induced; Transcription Factor IL-4 STAT; STAT6B; STAT6C; and D1251644. The sequence for the human STAT6 gene can be found under publicly available GenBank Accession Number NC_000012.12:c57111413-57095404. The human STAT6 gene is found at chromosome location 12q13.3 at 57111413-57095404, complement.

The sequence for the human STAT6 pre-mRNA transcript (SEQ ID NO: 1) corresponds to the reverse complement of residues 57111413-57095404, complement, of chromosome 12q13.3. The STAT6 mRNA sequence (GenBank Accession No. NM_001178078.1) is provided in SEQ ID NO: 3 (Table 1), except that the nucleotide “t” in SEQ ID NO: 3 is shown as “u” in the mRNA. The sequence for human STAT6 protein can be found under publicly available Accession Numbers: P42226-1, (canonical sequence, SEQ ID NO: 2; Table 1), P42226-2 (SEQ ID NO: 4), and P42226-3 (SEQ ID NO: 5), each of which is incorporated by reference herein in its entirety.

TABLE 1
STAT6 mRNA and Protein Sequences
STAT6 mRNA Sequence
GGGGCAGCCACTGCTTACACTGAAGAGGGAGGACGGGAGAGGAGTGTGTGTGTGTGTGTGTGTGTGTGTGTGTATG
TATGTGTGTGCTTTATCTTATTTTTCTTTTTGGTGGTGGTGGTGGAAGGGGGGAGGTGCTAGCAGGGCCAGCCTTG
AACTCGCTGGACAGAGCTACAGACCTATGGGGCCTGGAAGTGCCCGCTGAGAAAGGGAGAAGACAGCAGAGGGGTT
GCCGAGGCAACCTCCAAGTCCCAGATCATGTCTCTGTGGGGTCTGGTCTCCAAGATGCCCCCAGAAAAAGTGCAGC
GGCTCTATGTCGACTTTCCCCAACACCTGCGGCATCTTCTGGGTGACTGGCTGGAGAGCCAGCCCTGGGAGTTCCT
GGTCGGCTCCGACGCCTTCTGCTGCAACTTGGCTAGTGCCCTACTTTCAGACACTGTCCAGCACCTTCAGGCCTCG
GTGGGAGAGCAGGGGGAGGGGAGCACCATCTTGCAACACATCAGCACCCTTGAGAGCATATATCAGAGGGACCCCC
TGAAGCTGGTGGCCACTTTCAGACAAATACTTCAAGGAGAGAAAAAAGCTGTTATGGAACAGTTCCGCCACTTGCC
AATGCCTTTCCACTGGAAGCAGGAAGAACTCAAGTTTAAGACAGGCTTGCGGAGGCTGCAGCACCGAGTAGGGGAG
ATCCACCTTCTCCGAGAAGCCCTGCAGAAGGGGGCTGAGGCTGGCCAAGTGTCTCTGCACAGCTTGATAGAAACTC
CTGCTAATGGGACTGGGCCAAGTGAGGCCCTGGCCATGCTACTGCAGGAGACCACTGGAGAGCTAGAGGCAGCCAA
AGCCCTAGTGCTGAAGAGGATCCAGATTTGGAAACGGCAGCAGCAGCTGGCAGGGAATGGCGCACCGTTTGAGGAG
AGCCTGGCCCCACTCCAGGAGAGGTGTGAAAGCCTGGTGGACATTTATTCCCAGCTACAGCAGGAGGTAGGGGCGG
CTGGTGGGGAGCTTGAGCCCAAGACCCGGGCATCGCTGACTGGCCGGCTGGATGAAGTCCTGAGAACCCTCGTCAC
CAGTTGCTTCCTGGTGGAGAAGCAGCCCCCCCAGGTACTGAAGACTCAGACCAAGTTCCAGGCTGGAGTTCGATTC
CTGTTGGGCTTGAGGTTCCTGGGGGCCCCAGCCAAGCCTCCGCTGGTCAGGGCCGACATGGTGACAGAGAAGCAGG
CGCGGGAGCTGAGTGTGCCTCAGGGTCCTGGGGCTGGAGCAGAAAGCACTGGAGAAATCATCAACAACACTGTGCC
CTTGGAGAACAGCATTCCTGGGAACTGCTGCTCTGCCCTGTTCAAGAACCTGCTTCTCAAGAAGATCAAGCGGTGT
GAGCGGAAGGGCACTGAGTCTGTCACAGAGGAGAAGTGCGCTGTGCTCTTCTCTGCCAGCTTCACACTTGGCCCCG
GCAAACTCCCCATCCAGCTCCAGGCCCTGTCTCTGCCCCTGGTGGTCATCGTCCATGGCAACCAAGACAACAATGC
CAAAGCCACTATCCTGTGGGACAATGCCTTCTCTGAGATGGACCGCGTGCCCTTTGTGGTGGCTGAGCGGGTGCCC
TGGGAGAAGATGTGTGAAACTCTGAACCTGAAGTTCATGGCTGAGGTGGGGACCAACCGGGGGCTGCTCCCAGAGC
ACTTCCTCTTCCTGGCCCAGAAGATCTTCAATGACAACAGCCTCAGTATGGAGGCCTTCCAGCACCGTTCTGTGTC
CTGGTCGCAGTTCAACAAGGAGATCCTGCTGGGCCGTGGCTTCACCTTTTGGCAGTGGTTTGATGGTGTCCTGGAC
CTCACCAAACGCTGTCTCCGGAGCTACTGGTCTGACCGGCTGATCATTGGCTTCATCAGCAAACAGTACGTTACTA
GCCTTCTTCTCAATGAGCCCGACGGAACCTTTCTCCTCCGCTTCAGCGACTCAGAGATTGGGGGCATCACCATTGC
CCATGTCATCCGGGGCCAGGATGGCTCTCCACAGATAGAGAACATCCAGCCATTCTCTGCCAAAGACCTGTCCATT
CGCTCACTGGGGGACCGAATCCGGGATCTTGCTCAGCTCAAAAATCTCTATCCCAAGAAGCCCAAGGATGAGGCTT
TCCGGAGCCACTACAAGCCTGAACAGATGGGTAAGGATGGCAGGGGTTATGTCCCAGCTACCATCAAGATGACCGT
GGAAAGGGACCAACCACTTCCTACCCCAGAGCTCCAGATGCCTACCATGGTGCCTTCTTATGACCTTGGAATGGCC
CCTGATTCCTCCATGAGCATGCAGCTTGGCCCAGATATGGTGCCCCAGGTGTACCCACCACACTCTCACTCCATCC
CCCCGTATCAAGGCCTCTCCCCAGAAGAATCAGTCAACGTGTTGTCAGCCTTCCAGGAGCCTCACCTGCAGATGCC
CCCCAGCCTGGGCCAGATGAGCCTGCCCTTTGACCAGCCTCACCCCCAGGGCCTGCTGCCGTGCCAGCCTCAGGAG
CATGCTGTGTCCAGCCCTGACCCCCTGCTCTGCTCAGATGTGACCATGGTGGAAGACAGCTGCCTGAGCCAGCCAG
TGACAGCGTTTCCTCAGGGCACTTGGATTGGTGAAGACATATTCCCTCCTCTGCTGCCTCCCACTGAACAGGACCT
CACTAAGCTTCTCCTGGAGGGGCAAGGGGAGTCGGGGGGAGGGTCCTTGGGGGCACAGCCCCTCCTGCAGCCCTCC
CACTATGGGCAATCTGGGATCTCAATGTCCCACATGGACCTAAGGGCCAACCCCAGTTGGTGATCCCAGCTGGAGG
GAGAACCCAAAGAGACAGCTCTTCTACTACCCCCACAGACCTGCTCTGGACACTTGCTCATGCCCTGCCAAGCAGC
AGATGGGGAGGGTGCCCTCCTATCCCCACCTACTCCTGGGTCAGGAGGAAAAGACTAACAGGAGAATGCACAGTGG
GTGGAGCCAATCCACTCCTTCCTTTCTATCATTCCCCTGCCCACCTCCTTCCAGCACTGACTGGAAGGGAAGTTCA
GGCTCTGAGACACACCCCAACATGCCTGCACCTGCAGCGCGCACACGCACGCACACACACATACAGAGCTCTCTGA
GGGTGATGGGGCTGAGCAGGAGGGGGGCTGGGTAAGAGCACAGGTTAGGGCATGGAAGGCTTCTCCGCCCATTCTG
ACCCAGGGCCTAGGACGGATAGGCAGGAACATACAGACACATTTACACTAGAGGCCAGGGATAGAGGATATTGGGT
CTCAGCCCTAGGGGAATGGGAAGCAGCTCAAGGGACCCTGGGTGGGAGCATAGGAGGGGTCTGGACATGTGGTTAC
TAGTACAGGTTTTGCCCTGATTAAAAAATCTCCCAAAGCCCCAAATTCCTGTTAGCCAGGTGGAGGCTTCTGATAC
GTGTATGAGACTATGCAAAAGTACAAGGGCTGAGATTCTTCGTGTATAGCTGTGTGAACGTGTATGTACCTAGGAT
ATGTTAAATGTATAGCTGGCACCTTAGTTGCATGACCACATAGAACATGTGTCTATCTGCTTTTGCCTACGTGACA
ACACAAATTTGGGAGGGTGAGACACTGCACAGAAGACAGCAGCAAGTGTGCTGGCCTCTCTGACATATGCTAACCC
CCAAATACTCTGAATTTGGAGTCTGACTGTGCCCAAGTGGGTCCAAGTGGCTGTGACATCTACGTATGGCTCCACA
CCTCCAATGCTGCCTGGGAGCCAGGGTGAGAGTCTGGGTCCAGGCCTGGCCATGTGGCCCTCCAGTGTATGAGAGG
GCCCTGCCTGCTGCATCTTTTCTGTTGCCCCATCCACCGCCAGCTTCCCTTCACTCCCCTATCCCATTCTCCCTCT
CAAGGCAGGGGTCATAGATCCTAAGCCATAAAATAAATTTTATTCCAAAATAACAAAATAAATAATCTACTGTACA
CAATCTGAAAA (SEQ ID NO: 3)
STAT6 Protein Sequence
MSLWGLVSKMPPEKVQRLYVDFPQHLRHLLGDWLESQPWEFLVGSDAFCCNLASALLSDTVQHLQASVGEQGEGST
ILQHISTLESIYQRDPLKLVATFRQILQGEKKAVMEQFRHLPMPFHWKQEELKFKTGLRRLQHRVGEIHLLREALQ
KGAEAGQVSLHSLIETPANGTGPSEALAMLLQETTGELEAAKALVLKRIQIWKRQQQLAGNGAPFEESLAPLQERC
ESLVDIYSQLQQEVGAAGGELEPKTRASLTGRLDEVLRTLVTSCFLVEKQPPQVLKTQTKFQAGVRFLLGLRFLGA
PAKPPLVRADMVTEKQARELSVPQGPGAGAESTGEIINNTVPLENSIPGNCCSALFKNLLLKKIKRCERKGTESVT
EEKCAVLFSASFTLGPGKLPIQLQALSLPLVVIVHGNQDNNAKATILWDNAFSEMDRVPFVVAERVPWEKMCETLN
LKFMAEVGTNRGLLPEHFLFLAQKIFNDNSLSMEAFQHRSVSWSQFNKEILLGRGFTFWQWFDGVLDLTKRCLRSY
WSDRLIIGFISKQYVTSLLLNEPDGTFLLRFSDSEIGGITIAHVIRGQDGSPQIENIQPFSAKDLSIRSLGDRIRD
LAQLKNLYPKKPKDEAFRSHYKPEQMGKDGRGYVPATIKMTVERDQPLPTPELQMPTMVPSYDLGMAPDSSMSMQL
GPDMVPQVYPPHSHSIPPYQGLSPEESVNVLSAFQEPHLQMPPSLGQMSLPFDQPHPQGLLPCQPQEHAVSSPDPL
LCSDVTMVEDSCLSQPVTAFPQGTWIGEDIFPPLLPPTEQDLTKLLLEGQGESGGGSLGAQPLLQPSHYGQSGISM
SHMDLRANPSW (SEQ ID NO: 2)

Natural variants of the human STAT6 gene product are known. For example, natural variants of human STAT6 protein can contain one or more amino acid substitutions selected from: M118R, D419N, and any combination thereof. Additional variants of human STAT6 protein resulting from alternative splicing are also known in the art. STAT6 Isoform 2 (identifier: P42226-2 at UniProt) differs from the canonical sequence (SEQ ID NO: 3) as follows: deletion of residues 1-174 and substitution of ₁₇₅PSE₁₇₇ with ₁₇₅MEQ₁₇₇ relative to SEQ ID NO: 3. The sequence of STAT6 Isoform 3 (identifier: P42226-3) differs from the canonical sequence (SEQ ID NO: 3) as follows: deletion of residues 1-110 relative to SEQ ID NO: 3. Therefore, the ASOs of the present disclosure can be designed to reduce or inhibit expression of the natural variants of the STAT6 protein.

An example of a target nucleic acid sequence of the ASOs is STAT6 pre-mRNA. SEQ ID NO: 1 represents a human STAT6 genomic sequence (i.e., reverse complement of nucleotides 57111413-57095404, complement, of chromosome 12q13.3). SEQ ID NO: 1 is identical to a STAT6 pre-mRNA sequence except that nucleotide “t” in SEQ ID NO: 1 is shown as “u” in pre-mRNA. In certain aspects, the “target nucleic acid” comprises an intron of a STAT6 protein-encoding nucleic acids or naturally occurring variants thereof, and RNA nucleic acids derived therefrom, e.g., pre-mRNA. In other aspects, the target nucleic acid comprises an exon region of a STAT6 protein-encoding nucleic acids or naturally occurring variants thereof, and RNA nucleic acids derived therefrom, e.g., pre-mRNA. In yet other aspects, the target nucleic acid comprises an exon-intron junction of a STAT6 protein-encoding nucleic acids or naturally occurring variants thereof, and RNA nucleic acids derived therefrom, e.g., pre-mRNA. In some aspects, for example when used in research or diagnostics the “target nucleic acid” can be a cDNA or a synthetic oligonucleotide derived from the above DNA or RNA nucleic acid targets. The human STAT6 protein sequence encoded by the STAT6 pre-mRNA is shown as SEQ ID NO: 3. In other aspects, the target nucleic acid comprises an untranslated region of a STAT6 protein-encoding nucleic acids or naturally occurring variants thereof, e.g., 5′ UTR, 3′ UTR, or both.

In some aspects, an ASO of the disclosure hybridizes to a region within the introns of a STAT6 transcript, e.g., SEQ ID NO: 1. In certain aspects, an ASO of the disclosure hybridizes to a region within the exons of a STAT6 transcript, e.g., SEQ ID NO: 1. In other aspects, an ASO of the disclosure hybridizes to a region within the exon-intron junction of a STAT6 transcript, e.g., SEQ ID NO: 1. In some aspects, an ASO of the disclosure hybridizes to a region within a STAT6 transcript (e.g., an intron, exon, or exon-intron junction), e.g., SEQ ID NO: 1, wherein the ASO has a design according to formula: 5′ A-B-C 3′ as described elsewhere herein.

In some aspects, the ASO targets a mRNA encoding a particular isoform of STAT6 protein (e.g., Isoform 1). In some aspects, the ASO targets all isoforms of STAT6 protein. In other aspects, the ASO targets two isoforms (e.g., Isoform 1 and Isoform 2, Isoform 1 and Isoform 3, or Isoform 2 and Isoform 3) of STAT6 protein.

In some aspects, the ASO comprises a contiguous nucleotide sequence (e.g., 10 to 30 nucleotides in length, e.g., 20 nucleotides in length) that are complementary to a nucleic acid sequence within a STAT6 transcript, e.g., a region corresponding to SEQ ID NO: 1 or SEQ ID NO: 3. In some aspects, the ASO comprises a contiguous nucleotide sequence that hybridizes to a nucleic acid sequence, or a region within the sequence, of a STAT6 transcript (“target region”), wherein the nucleic acid sequence corresponds (i) nucleotides 1-700 of SEQ ID NO: 3; (ii) nucleotides 1000-1500 of SEQ ID NO: 3; (iii) nucleotides 1500-2000 of SEQ ID NO: 3; (iv) nucleotides 2000-2500 of SEQ ID NO: 3; (v) 2500-3000 of SEQ ID NO: 3; or (vi) 3000-3700 of SEQ ID NO: 3 and wherein, optionally, the ASO has one of the designs described herein or a chemical structure shown elsewhere herein.

In some aspects, the ASO comprises a contiguous nucleotide sequence that hybridizes to a nucleic acid sequence, or a region within the sequence, of a STAT6 transcript (“target region”), wherein the nucleic acid sequence corresponds to (i) nucleotides 413-803 of SEQ ID NO: 3; (ii) nucleotides 952-1688 of SEQ ID NO: 3; (iii) nucleotides 1726-2489 of SEQ ID NO: 3; (iv) nucleotides 2682-2912 of SEQ ID NO: 3; (v) 2970-3203 of SEQ ID NO: 3; or (vi) 3331-3561 of SEQ ID NO: 3 and wherein, optionally, the ASO has one of the designs described herein or a chemical structure shown elsewhere herein.

In some aspects, the ASO comprises a contiguous nucleotide sequence that hybridizes to a nucleic acid sequence, or a region within the sequence, of a STAT6 transcript (“target region”), wherein the nucleic acid sequence corresponds to (i) nucleotides 463-753 of SEQ ID NO: 3; (ii) nucleotides 1002-1638 of SEQ ID NO: 3; (iii) nucleotides 1776-2439 of SEQ ID NO: 3; (iv) nucleotides 2682-2862 of SEQ ID NO: 3; (v) 3020-3153 of SEQ ID NO: 3; or (vi) 3381-3511 of SEQ ID NO: 3 and wherein, optionally, the ASO has one of the designs described herein or a chemical structure shown elsewhere herein.

In some aspects, the ASO comprises a contiguous nucleotide sequence that hybridizes to a nucleic acid sequence, or a region within the sequence, of a STAT6 transcript (“target region”), wherein the nucleic acid sequence corresponds to (i) nucleotides 503-713 of SEQ ID NO: 3; (ii) nucleotides 1042-1598 of SEQ ID NO: 3; (iii) nucleotides 1816-2399 of SEQ ID NO: 3; (iv) nucleotides 2722-2822 of SEQ ID NO: 3; (v) 3060-3113 of SEQ ID NO: 3; or (vi) 3421-3471 of SEQ ID NO: 3 and wherein, optionally, the ASO has one of the designs described herein or a chemical structure shown elsewhere herein.

In some aspects, the target region corresponds to nucleotides 1053-1067 of SEQ ID NO: 3 (e.g., ASO-STAT6-1053; SEQ ID NO: 91). In some aspects, the target region corresponds to nucleotides 1359-1373 of SEQ ID NO: 3 (e.g., ASO-STAT6-1359; SEQ ID NO: 92). In some aspects, the target region corresponds to nucleotides 1890-1904 of SEQ ID NO: 3 (e.g., ASO-STAT6-1890; SEQ ID NO: 93). In some aspects, the target region corresponds to nucleotides 1892-1906 of SEQ ID NO: 3 (e.g., ASO-STAT6-1892; SEQ ID NO: 94). In some aspects, the target region corresponds to nucleotides 1915-1929 of SEQ ID NO: 3 (e.g., ASO-STAT6-1915; SEQ ID NO: 95). In some aspects, the target region corresponds to nucleotides 1916-1930 of SEQ ID NO: 3 (e.g., ASO-STAT6-1916; SEQ ID NO: 96). In some aspects, the target region corresponds to nucleotides 1917-1931 of SEQ ID NO: 3 (e.g., ASO-STAT6-1917; SEQ ID NO: 97). In some aspects, the target region corresponds to nucleotides 1918-1932 of SEQ ID NO: 3 (e.g., ASO-STAT6-1918; SEQ ID NO: 98). In some aspects, the target region corresponds to nucleotides 1919-1933 of SEQ ID NO: 3 (e.g., ASO-STAT6-1919; SEQ ID NO: 99). In some aspects, the target region corresponds to nucleotides 1920-1934 of SEQ ID NO: 3 (e.g., ASO-STAT6-1920; SEQ ID NO: 100). In some aspects, the target region corresponds to nucleotides 1937-1951 of SEQ ID NO: 3 (e.g., ASO-STAT6-1937; SEQ ID NO: 101). In some aspects, the target region corresponds to nucleotides 1938-1952 of SEQ ID NO: 3 (e.g., ASO-STAT6-1938; SEQ ID NO: 102). In some aspects, the target region corresponds to nucleotides 2061-2075 of SEQ ID NO: 3 (e.g., ASO-STAT6-2061; SEQ ID NO: 103). In some aspects, the target region corresponds to nucleotides 2062-2076 of SEQ ID NO: 3 (e.g., ASO-STAT6-2062; SEQ ID NO: 104). In some aspects, the target region corresponds to nucleotides 2063-2077 of SEQ ID NO: 3 (e.g., ASO-STAT6-2063; SEQ ID NO: 105). In some aspects, the target region corresponds to nucleotides 2064-2078 of SEQ ID NO: 3 (e.g., ASO-STAT6-2064; SEQ ID NO: 106). In some aspects, the target region corresponds to nucleotides 2066-2080 of SEQ ID NO: 3 (e.g., ASO-STAT6-2066; SEQ ID NO: 107). In some aspects, the target region corresponds to nucleotides 2067-2081 of SEQ ID NO: 3 (e.g., ASO-STAT6-2067; SEQ ID NO: 108). In some aspects, the target region corresponds to nucleotides 2068-2082 of SEQ ID NO: 3 (e.g., ASO-STAT6-2068; SEQ ID NO: 109). In some aspects, the target region corresponds to nucleotides 2352-2366 of SEQ ID NO: 3 (e.g., ASO-STAT6-2352; SEQ ID NO: 110). In some aspects, the target region corresponds to nucleotides 3073-3087 of SEQ ID NO: 3 (e.g., ASO-STAT6-3073; SEQ ID NO: 111). In some aspects, the target region corresponds to nucleotides 1053-1068 of SEQ ID NO: 3 (e.g., ASO-STAT6-1053; SEQ ID NO: 112). In some aspects, the target region corresponds to nucleotides 1054-1069 of SEQ ID NO: 3 (e.g., ASO-STAT6-1054; SEQ ID NO: 113). In some aspects, the target region corresponds to nucleotides 1356-1371 of SEQ ID NO: 3 (e.g., ASO-STAT6-1356; SEQ ID NO: 114). In some aspects, the target region corresponds to nucleotides 1847-1862 of SEQ ID NO: 3 (e.g., ASO-STAT6-1847; SEQ ID NO: 115). In some aspects, the target region corresponds to nucleotides 1886-1901 of SEQ ID NO: 3 (e.g., ASO-STAT6-1886; SEQ ID NO: 116). In some aspects, the target region corresponds to nucleotides 1887-1902 of SEQ ID NO: 3 (e.g., ASO-STAT6-1887; SEQ ID NO: 117). In some aspects, the target region corresponds to nucleotides 1888-1903 of SEQ ID NO: 3 (e.g., ASO-STAT6-1888; SEQ ID NO: 118). In some aspects, the target region corresponds to nucleotides 1889-1904 of SEQ ID NO: 3 (e.g., ASO-STAT6-1889; SEQ ID NO: 119). In some aspects, the target region corresponds to nucleotides 1890-1905 of SEQ ID NO: 3 (e.g., ASO-STAT6-1890; SEQ ID NO: 120). In some aspects, the target region corresponds to nucleotides 1893-1908 of SEQ ID NO: 3 (e.g., ASO-STAT6-1893; SEQ ID NO: 121). In some aspects, the target region corresponds to nucleotides 1917-1932 of SEQ ID NO: 3 (e.g., ASO-STAT6-1917; SEQ ID NO: 122). In some aspects, the target region corresponds to nucleotides 1919-1934 of SEQ ID NO: 3 (e.g., ASO-STAT6-1919; SEQ ID NO: 123). In some aspects, the target region corresponds to nucleotides 2056-2071 of SEQ ID NO: 3 (e.g., ASO-STAT6-2056; SEQ ID NO: 124). In some aspects, the target region corresponds to nucleotides 2060-2075 of SEQ ID NO: 3 (e.g., ASO-STAT6-2060; SEQ ID NO: 125). In some aspects, the target region corresponds to nucleotides 2066-2081 of SEQ ID NO: 3 (e.g., ASO-STAT6-2066; SEQ ID NO: 126). In some aspects, the target region corresponds to nucleotides 2070-2085 of SEQ ID NO: 3 (e.g., ASO-STAT6-2070; SEQ ID NO: 127). In some aspects, the target region corresponds to nucleotides 2351-2366 of SEQ ID NO: 3 (e.g., ASO-STAT6-2351; SEQ ID NO: 128). In some aspects, the target region corresponds to nucleotides 2352-2367 of SEQ ID NO: 3 (e.g., ASO-STAT6-2352; SEQ ID NO: 129). In some aspects, the target region corresponds to nucleotides 2359-2374 of SEQ ID NO: 3 (e.g., ASO-STAT6-2359; SEQ ID NO: 130). In some aspects, the target region corresponds to nucleotides 3633-3648 of SEQ ID NO: 3 (e.g., ASO-STAT6-3633; SEQ ID NO: 131). In some aspects, the target region corresponds to nucleotides 673-689 of SEQ ID NO: 3 (e.g., ASO-STAT6-673; SEQ ID NO: 132). In some aspects, the target region corresponds to nucleotides 1052-1068 of SEQ ID NO: 3 (e.g., ASO-STAT6-1052; SEQ ID NO: 133). In some aspects, the target region corresponds to nucleotides 1356-1372 of SEQ ID NO: 3 (e.g., ASO-STAT6-1356; SEQ ID NO: 134). In some aspects, the target region corresponds to nucleotides 1357-1373 of SEQ ID NO: 3 (e.g., ASO-STAT6-1357; SEQ ID NO: 135). In some aspects, the target region corresponds to nucleotides 1359-1375 of SEQ ID NO: 3 (e.g., ASO-STAT6-1359; SEQ ID NO: 136). In some aspects, the target region corresponds to nucleotides 1360-1376 of SEQ ID NO: 3 (e.g., ASO-STAT6-1360; SEQ ID NO: 137). In some aspects, the target region corresponds to nucleotides 1839-1855 of SEQ ID NO: 3 (e.g., ASO-STAT6-1839; SEQ ID NO: 138). In some aspects, the target region corresponds to nucleotides 1848-1864 of SEQ ID NO: 3 (e.g., ASO-STAT6-1848; SEQ ID NO: 139). In some aspects, the target region corresponds to nucleotides 1849-1865 of SEQ ID NO: 3 (e.g., ASO-STAT6-1849; SEQ ID NO: 140). In some aspects, the target region corresponds to nucleotides 1891-1907 of SEQ ID NO: 3 (e.g., ASO-STAT6-1891; SEQ ID NO: 141). In some aspects, the target region corresponds to nucleotides 1915-1931 of SEQ ID NO: 3 (e.g., ASO-STAT6-1915; SEQ ID NO: 142). In some aspects, the target region corresponds to nucleotides 1916-1932 of SEQ ID NO: 3 (e.g., ASO-STAT6-1916; SEQ ID NO: 143). In some aspects, the target region corresponds to nucleotides 1917-1933 of SEQ ID NO: 3 (e.g., ASO-STAT6-1917; SEQ ID NO: 144). In some aspects, the target region corresponds to nucleotides 1938-1954 of SEQ ID NO: 3 (e.g., ASO-STAT6-1938; SEQ ID NO: 145). In some aspects, the target region corresponds to nucleotides 1939-1955 of SEQ ID NO: 3 (e.g., ASO-STAT6-1939; SEQ ID NO: 146). In some aspects, the target region corresponds to nucleotides 2063-2079 of SEQ ID NO: 3 (e.g., ASO-STAT6-2063; SEQ ID NO: 147). In some aspects, the target region corresponds to nucleotides 2064-2080 of SEQ ID NO: 3 (e.g., ASO-STAT6-2064; SEQ ID NO: 148). In some aspects, the target region corresponds to nucleotides 2065-2081 of SEQ ID NO: 3 (e.g., ASO-STAT6-2065; SEQ ID NO: 149). In some aspects, the target region corresponds to nucleotides 2066-2082 of SEQ ID NO: 3 (e.g., ASO-STAT6-2066; SEQ ID NO: 150). In some aspects, the target region corresponds to nucleotides 2068-2084 of SEQ ID NO: 3 (e.g., ASO-STAT6-2068; SEQ ID NO: 151). In some aspects, the target region corresponds to nucleotides 2187-2203 of SEQ ID NO: 3 (e.g., ASO-STAT6-2187; SEQ ID NO: 152). In some aspects, the target region corresponds to nucleotides 2350-2366 of SEQ ID NO: 3 (e.g., ASO-STAT6-2350; SEQ ID NO: 153). In some aspects, the target region corresponds to nucleotides 2351-2367 of SEQ ID NO: 3 (e.g., ASO-STAT6-2351; SEQ ID NO: 154). In some aspects, the target region corresponds to nucleotides 2352-2368 of SEQ ID NO: 3 (e.g., ASO-STAT6-2352; SEQ ID NO: 155). In some aspects, the target region corresponds to nucleotides 2357-2373 of SEQ ID NO: 3 (e.g., ASO-STAT6-2357; SEQ ID NO: 156). In some aspects, the target region corresponds to nucleotides 513-532 of SEQ ID NO: 3 (e.g., ASO-STAT6-513; SEQ ID NO: 157). In some aspects, the target region corresponds to nucleotides 671-690 of SEQ ID NO: 3 (e.g., ASO-STAT6-671; SEQ ID NO: 158). In some aspects, the target region corresponds to nucleotides 1131-1150 of SEQ ID NO: 3 (e.g., ASO-STAT6-1131; SEQ ID NO: 159). In some aspects, the target region corresponds to nucleotides 1354-1373 of SEQ ID NO: 3 (e.g., ASO-STAT6-1354; SEQ ID NO: 160). In some aspects, the target region corresponds to nucleotides 1355-1374 of SEQ ID NO: 3 (e.g., ASO-STAT6-1355; SEQ ID NO: 161). In some aspects, the target region corresponds to nucleotides 1356-1375 of SEQ ID NO: 3 (e.g., ASO-STAT6-1356; SEQ ID NO: 162). In some aspects, the target region corresponds to nucleotides 1432-1451 of SEQ ID NO: 3 (e.g., ASO-STAT6-1432; SEQ ID NO: 163). In some aspects, the target region corresponds to nucleotides 1555-1574 of SEQ ID NO: 3 (e.g., ASO-STAT6-1555; SEQ ID NO: 164). In some aspects, the target region corresponds to nucleotides 1556-1575 of SEQ ID NO: 3 (e.g., ASO-STAT6-1556; SEQ ID NO: 165). In some aspects, the target region corresponds to nucleotides 1557-1576 of SEQ ID NO: 3 (e.g., ASO-STAT6-1557; SEQ ID NO: 166). In some aspects, the target region corresponds to nucleotides 1558-1577 of SEQ ID NO: 3 (e.g., ASO-STAT6-1558; SEQ ID NO: 167). In some aspects, the target region corresponds to nucleotides 1826-1845 of SEQ ID NO: 3 (e.g., ASO-STAT6-1826; SEQ ID NO: 168). In some aspects, the target region corresponds to nucleotides 1827-1846 of SEQ ID NO: 3 (e.g., ASO-STAT6-1827; SEQ ID NO: 169). In some aspects, the target region corresponds to nucleotides 1833-1852 of SEQ ID NO: 3 (e.g., ASO-STAT6-1833; SEQ ID NO: 170). In some aspects, the target region corresponds to nucleotides 1843-1862 of SEQ ID NO: 3 (e.g., ASO-STAT6-1843; SEQ ID NO: 171). In some aspects, the target region corresponds to nucleotides 1846-1865 of SEQ ID NO: 3 (e.g., ASO-STAT6-1846; SEQ ID NO: 172). In some aspects, the target region corresponds to nucleotides 1847-1866 of SEQ ID NO: 3 (e.g., ASO-STAT6-1847; SEQ ID NO: 173). In some aspects, the target region corresponds to nucleotides 1883-1902 of SEQ ID NO: 3 (e.g., ASO-STAT6-1883; SEQ ID NO: 174). In some aspects, the target region corresponds to nucleotides 1889-1908 of SEQ ID NO: 3 (e.g., ASO-STAT6-1889; SEQ ID NO: 175). In some aspects, the target region corresponds to nucleotides 1890-1909 of SEQ ID NO: 3 (e.g., ASO-STAT6-1890; SEQ ID NO: 176). In some aspects, the target region corresponds to nucleotides 1891-1910 of SEQ ID NO: 3 (e.g., ASO-STAT6-1891; SEQ ID NO: 177). In some aspects, the target region corresponds to nucleotides 1916-1935 of SEQ ID NO: 3 (e.g., ASO-STAT6-1916; SEQ ID NO: 178). In some aspects, the target region corresponds to nucleotides 1917-1936 of SEQ ID NO: 3 (e.g., ASO-STAT6-1917; SEQ ID NO: 179). In some aspects, the target region corresponds to nucleotides 2056-2075 of SEQ ID NO: 3 (e.g., ASO-STAT6-2056; SEQ ID NO: 180). In some aspects, the target region corresponds to nucleotides 2057-2076 of SEQ ID NO: 3 (e.g., ASO-STAT6-2057; SEQ ID NO: 181). In some aspects, the target region corresponds to nucleotides 2060-2079 of SEQ ID NO: 3 (e.g., ASO-STAT6-2060; SEQ ID NO: 182). In some aspects, the target region corresponds to nucleotides 2062-2081 of SEQ ID NO: 3 (e.g., ASO-STAT6-2062; SEQ ID NO: 183). In some aspects, the target region corresponds to nucleotides 2063-2082 of SEQ ID NO: 3 (e.g., ASO-STAT6-2063; SEQ ID NO: 184). In some aspects, the target region corresponds to nucleotides 2065-2084 of SEQ ID NO: 3 (e.g., ASO-STAT6-2065; SEQ ID NO: 185). In some aspects, the target region corresponds to nucleotides 2068-2087 of SEQ ID NO: 3 (e.g., ASO-STAT6-2068; SEQ ID NO: 186). In some aspects, the target region corresponds to nucleotides 2347-2366 of SEQ ID NO: 3 (e.g., ASO-STAT6-2347; SEQ ID NO: 187). In some aspects, the target region corresponds to nucleotides 2348-2367 of SEQ ID NO: 3 (e.g., ASO-STAT6-2348; SEQ ID NO: 188). In some aspects, the target region corresponds to nucleotides 2358-2377 of SEQ ID NO: 3 (e.g., ASO-STAT6-2358; SEQ ID NO: 189). In some aspects, the target region corresponds to nucleotides 2782-2801 of SEQ ID NO: 3 (e.g., ASO-STAT6-2782; SEQ ID NO: 190). In some aspects, the target region corresponds to nucleotides 3070-3089 of SEQ ID NO: 3 (e.g., ASO-STAT6-3070; SEQ ID NO: 191). In some aspects, the target region corresponds to nucleotides 3071-3090 of SEQ ID NO: 3 (e.g., ASO-STAT6-3071; SEQ ID NO: 192). In some aspects, the target region corresponds to nucleotides 3431-3450 of SEQ ID NO: 3 (e.g., ASO-STAT6-3431; SEQ ID NO: 193).

In some aspects, the target region corresponds to nucleotides 1053-1067 of SEQ ID NO: 3 (e.g., ASO-STAT6-1053; SEQ ID NO: 91)±10, ±20, ±30, ±40, ±50, ±60, ±70, ±80, or ±90 nucleotides at the 3′ end and/or the 5′ end. In some aspects, the target region corresponds to nucleotides 1359-1373 of SEQ ID NO: 3 (e.g., ASO-STAT6-1359; SEQ ID NO: 92)±10, ±20, ±30, ±40, ±50, ±60, ±70, ±80, or ±90 nucleotides at the 3′ end and/or the 5′ end. In some aspects, the target region corresponds to nucleotides 1890-1904 of SEQ ID NO: 3 (e.g., ASO-STAT6-1890; SEQ ID NO: 93)±10, ±20, ±30, ±40, ±50, ±60, ±70, ±80, or ±90 nucleotides at the 3′ end and/or the 5′ end. In some aspects, the target region corresponds to nucleotides 1892-1906 of SEQ ID NO: 3 (e.g., ASO-STAT6-1892; SEQ ID NO: 94)±10, ±20, ±30, ±40, ±50, ±60, ±70, ±80, or ±90 nucleotides at the 3′ end and/or the 5′ end. In some aspects, the target region corresponds to nucleotides 1915-1929 of SEQ ID NO: 3 (e.g., ASO-STAT6-1915; SEQ ID NO: 95)±10, ±20, ±30, ±40, ±50, ±60, ±70, ±80, or ±90 nucleotides at the 3′ end and/or the 5′ end. In some aspects, the target region corresponds to nucleotides 1916-1930 of SEQ ID NO: 3 (e.g., ASO-STAT6-1916; SEQ ID NO: 96)±10, ±20, ±30, ±40, ±50, ±60, ±70, ±80, or ±90 nucleotides at the 3′ end and/or the 5′ end. In some aspects, the target region corresponds to nucleotides 1917-1931 of SEQ ID NO: 3 (e.g., ASO-STAT6-1917; SEQ ID NO: 97)±10, ±20, ±30, ±40, ±50, ±60, ±70, ±80, or ±90 nucleotides at the 3′ end and/or the 5′ end. In some aspects, the target region corresponds to nucleotides 1918-1932 of SEQ ID NO: 3 (e.g., ASO-STAT6-1918; SEQ ID NO: 98)±10, ±20, ±30, ±40, ±50, ±60, ±70, ±80, or ±90 nucleotides at the 3′ end and/or the 5′ end. In some aspects, the target region corresponds to nucleotides 1919-1933 of SEQ ID NO: 3 (e.g., ASO-STAT6-1919; SEQ ID NO: 99)±10, ±20, ±30, ±40, ±50, ±60, ±70, ±80, or ±90 nucleotides at the 3′ end and/or the 5′ end. In some aspects, the target region corresponds to nucleotides 1920-1934 of SEQ ID NO: 3 (e.g., ASO-STAT6-1920; SEQ ID NO: 100)±10, ±20, ±30, ±40, ±50, ±60, ±70, ±80, or ±90 nucleotides at the 3′ end and/or the 5′ end. In some aspects, the target region corresponds to nucleotides 1937-1951 of SEQ ID NO: 3 (e.g., ASO-STAT6-1937; SEQ ID NO: 101)±10, ±20, ±30, ±40, ±50, ±60, ±70, ±80, or ±90 nucleotides at the 3′ end and/or the 5′ end. In some aspects, the target region corresponds to nucleotides 1938-1952 of SEQ ID NO: 3 (e.g., ASO-STAT6-1938; SEQ ID NO: 102)±10, ±20, ±30, ±40, ±50, ±60, ±70, ±80, or ±90 nucleotides at the 3′ end and/or the 5′ end. In some aspects, the target region corresponds to nucleotides 2061-2075 of SEQ ID NO: 3 (e.g., ASO-STAT6-2061; SEQ ID NO: 103)±10, ±20, ±30, ±40, ±50, ±60, ±70, ±80, or ±90 nucleotides at the 3′ end and/or the 5′ end. In some aspects, the target region corresponds to nucleotides 2062-2076 of SEQ ID NO: 3 (e.g., ASO-STAT6-2062; SEQ ID NO: 104)±10, ±20, ±30, ±40, ±50, ±60, ±70, ±80, or ±90 nucleotides at the 3′ end and/or the 5′ end. In some aspects, the target region corresponds to nucleotides 2063-2077 of SEQ ID NO: 3 (e.g., ASO-STAT6-2063; SEQ ID NO: 105)±10, ±20, ±30, ±40, ±50, ±60, ±70, ±80, or ±90 nucleotides at the 3′ end and/or the 5′ end. In some aspects, the target region corresponds to nucleotides 2064-2078 of SEQ ID NO: 3 (e.g., ASO-STAT6-2064; SEQ ID NO: 106)±10, ±20, ±30, ±40, ±50, ±60, ±70, ±80, or ±90 nucleotides at the 3′ end and/or the 5′ end. In some aspects, the target region corresponds to nucleotides 2066-2080 of SEQ ID NO: 3 (e.g., ASO-STAT6-2066; SEQ ID NO: 107)±10, ±20, ±30, ±40, ±50, ±60, ±70, ±80, or ±90 nucleotides at the 3′ end and/or the 5′ end. In some aspects, the target region corresponds to nucleotides 2067-2081 of SEQ ID NO: 3 (e.g., ASO-STAT6-2067; SEQ ID NO: 108)±10, ±20, ±30, ±40, ±50, ±60, ±70, ±80, or ±90 nucleotides at the 3′ end and/or the 5′ end. In some aspects, the target region corresponds to nucleotides 2068-2082 of SEQ ID NO: 3 (e.g., ASO-STAT6-2068; SEQ ID NO: 109)±10, ±20, ±30, ±40, ±50, ±60, ±70, ±80, or ±90 nucleotides at the 3′ end and/or the 5′ end. In some aspects, the target region corresponds to nucleotides 2352-2366 of SEQ ID NO: 3 (e.g., ASO-STAT6-2352; SEQ ID NO: 110)±10, ±20, ±30, ±40, ±50, ±60, ±70, ±80, or ±90 nucleotides at the 3′ end and/or the 5′ end. In some aspects, the target region corresponds to nucleotides 3073-3087 of SEQ ID NO: 3 (e.g., ASO-STAT6-3073; SEQ ID NO: 111)±10, ±20, ±30, ±40, ±50, ±60, ±70, ±80, or ±90 nucleotides at the 3′ end and/or the 5′ end. In some aspects, the target region corresponds to nucleotides 1053-1068 of SEQ ID NO: 3 (e.g., ASO-STAT6-1053; SEQ ID NO: 112)±10, ±20, ±30, ±40, ±50, ±60, ±70, ±80, or ±90 nucleotides at the 3′ end and/or the 5′ end. In some aspects, the target region corresponds to nucleotides 1054-1069 of SEQ ID NO: 3 (e.g., ASO-STAT6-1054; SEQ ID NO: 113)±10, ±20, ±30, ±40, ±50, ±60, ±70, ±80, or ±90 nucleotides at the 3′ end and/or the 5′ end. In some aspects, the target region corresponds to nucleotides 1356-1371 of SEQ ID NO: 3 (e.g., ASO-STAT6-1356; SEQ ID NO: 114)±10, ±20, ±30, ±40, ±50, ±60, ±70, ±80, or ±90 nucleotides at the 3′ end and/or the 5′ end. In some aspects, the target region corresponds to nucleotides 1847-1862 of SEQ ID NO: 3 (e.g., ASO-STAT6-1847; SEQ ID NO: 115)±10, ±20, ±30, ±40, ±50, ±60, ±70, ±80, or ±90 nucleotides at the 3′ end and/or the 5′ end. In some aspects, the target region corresponds to nucleotides 1886-1901 of SEQ ID NO: 3 (e.g., ASO-STAT6-1886; SEQ ID NO: 116)±10, ±20, ±30, ±40, ±50, ±60, ±70, ±80, or ±90 nucleotides at the 3′ end and/or the 5′ end. In some aspects, the target region corresponds to nucleotides 1887-1902 of SEQ ID NO: 3 (e.g., ASO-STAT6-1887; SEQ ID NO: 117)±10, ±20, ±30, ±40, ±50, ±60, ±70, ±80, or ±90 nucleotides at the 3′ end and/or the 5′ end. In some aspects, the target region corresponds to nucleotides 1888-1903 of SEQ ID NO: 3 (e.g., ASO-STAT6-1888; SEQ ID NO: 118)±10, ±20, ±30, ±40, ±50, ±60, ±70, ±80, or ±90 nucleotides at the 3′ end and/or the 5′ end. In some aspects, the target region corresponds to nucleotides 1889-1904 of SEQ ID NO: 3 (e.g., ASO-STAT6-1889; SEQ ID NO: 119)±10, ±20, ±30, ±40, ±50, ±60, ±70, ±80, or ±90 nucleotides at the 3′ end and/or the 5′ end. In some aspects, the target region corresponds to nucleotides 1890-1905 of SEQ ID NO: 3 (e.g., ASO-STAT6-1890; SEQ ID NO: 120)±10, ±20, ±30, ±40, ±50, ±60, ±70, ±80, or ±90 nucleotides at the 3′ end and/or the 5′ end. In some aspects, the target region corresponds to nucleotides 1893-1908 of SEQ ID NO: 3 (e.g., ASO-STAT6-1893; SEQ ID NO: 121)±10, ±20, ±30, ±40, ±50, ±60, ±70, ±80, or ±90 nucleotides at the 3′ end and/or the 5′ end. In some aspects, the target region corresponds to nucleotides 1917-1932 of SEQ ID NO: 3 (e.g., ASO-STAT6-1917; SEQ ID NO: 122)±10, ±20, ±30, ±40, ±50, ±60, ±70, ±80, or ±90 nucleotides at the 3′ end and/or the 5′ end. In some aspects, the target region corresponds to nucleotides 1919-1934 of SEQ ID NO: 3 (e.g., ASO-STAT6-1919; SEQ ID NO: 123)±10, ±20, ±30, ±40, ±50, ±60, ±70, ±80, or ±90 nucleotides at the 3′ end and/or the 5′ end. In some aspects, the target region corresponds to nucleotides 2056-2071 of SEQ ID NO: 3 (e.g., ASO-STAT6-2056; SEQ ID NO: 124)±10, ±20, ±30, ±40, ±50, ±60, ±70, ±80, or ±90 nucleotides at the 3′ end and/or the 5′ end. In some aspects, the target region corresponds to nucleotides 2060-2075 of SEQ ID NO: 3 (e.g., ASO-STAT6-2060; SEQ ID NO: 125)±10, ±20, ±30, ±40, ±50, ±60, ±70, ±80, or ±90 nucleotides at the 3′ end and/or the 5′ end. In some aspects, the target region corresponds to nucleotides 2066-2081 of SEQ ID NO: 3 (e.g., ASO-STAT6-2066; SEQ ID NO: 126)±10, ±20, ±30, ±40, ±50, ±60, ±70, ±80, or ±90 nucleotides at the 3′ end and/or the 5′ end. In some aspects, the target region corresponds to nucleotides 2070-2085 of SEQ ID NO: 3 (e.g., ASO-STAT6-2070; SEQ ID NO: 127)±10, ±20, ±30, ±40, ±50, ±60, ±70, ±80, or ±90 nucleotides at the 3′ end and/or the 5′ end. In some aspects, the target region corresponds to nucleotides 2351-2366 of SEQ ID NO: 3 (e.g., ASO-STAT6-2351; SEQ ID NO: 128)±10, ±20, ±30, ±40, ±50, ±60, ±70, ±80, or ±90 nucleotides at the 3′ end and/or the 5′ end. In some aspects, the target region corresponds to nucleotides 2352-2367 of SEQ ID NO: 3 (e.g., ASO-STAT6-2352; SEQ ID NO: 129)±10, ±20, ±30, ±40, ±50, ±60, ±70, ±80, or ±90 nucleotides at the 3′ end and/or the 5′ end. In some aspects, the target region corresponds to nucleotides 2359-2374 of SEQ ID NO: 3 (e.g., ASO-STAT6-2359; SEQ ID NO: 130)±10, ±20, ±30, ±40, ±50, ±60, ±70, ±80, or ±90 nucleotides at the 3′ end and/or the 5′ end. In some aspects, the target region corresponds to nucleotides 3633-3648 of SEQ ID NO: 3 (e.g., ASO-STAT6-3633; SEQ ID NO: 131)±10, ±20, ±30, ±40, ±50, ±60, ±70, ±80, or ±90 nucleotides at the 3′ end and/or the 5′ end. In some aspects, the target region corresponds to nucleotides 673-689 of SEQ ID NO: 3 (e.g., ASO-STAT6-673; SEQ ID NO: 132)±10, ±20, ±30, ±40, ±50, ±60, ±70, ±80, or ±90 nucleotides at the 3′ end and/or the 5′ end. In some aspects, the target region corresponds to nucleotides 1052-1068 of SEQ ID NO: 3 (e.g., ASO-STAT6-1052; SEQ ID NO: 133)±10, ±20, ±30, ±40, ±50, ±60, ±70, ±80, or ±90 nucleotides at the 3′ end and/or the 5′ end. In some aspects, the target region corresponds to nucleotides 1356-1372 of SEQ ID NO: 3 (e.g., ASO-STAT6-1356; SEQ ID NO: 134)±10, ±20, ±30, ±40, ±50, ±60, ±70, ±80, or ±90 nucleotides at the 3′ end and/or the 5′ end. In some aspects, the target region corresponds to nucleotides 1357-1373 of SEQ ID NO: 3 (e.g., ASO-STAT6-1357; SEQ ID NO: 135)±10, ±20, ±30, ±40, ±50, ±60, ±70, ±80, or ±90 nucleotides at the 3′ end and/or the 5′ end. In some aspects, the target region corresponds to nucleotides 1359-1375 of SEQ ID NO: 3 (e.g., ASO-STAT6-1359; SEQ ID NO: 136)±10, ±20, ±30, ±40, ±50, ±60, ±70, ±80, or ±90 nucleotides at the 3′ end and/or the 5′ end. In some aspects, the target region corresponds to nucleotides 1360-1376 of SEQ ID NO: 3 (e.g., ASO-STAT6-1360; SEQ ID NO: 137)±10, ±20, ±30, ±40, ±50, ±60, ±70, ±80, or ±90 nucleotides at the 3′ end and/or the 5′ end. In some aspects, the target region corresponds to nucleotides 1839-1855 of SEQ ID NO: 3 (e.g., ASO-STAT6-1839; SEQ ID NO: 138)±10, ±20, ±30, ±40, ±50, ±60, ±70, ±80, or ±90 nucleotides at the 3′ end and/or the 5′ end. In some aspects, the target region corresponds to nucleotides 1848-1864 of SEQ ID NO: 3 (e.g., ASO-STAT6-1848; SEQ ID NO: 139)±10, ±20, ±30, ±40, ±50, ±60, ±70, ±80, or ±90 nucleotides at the 3′ end and/or the 5′ end. In some aspects, the target region corresponds to nucleotides 1849-1865 of SEQ ID NO: 3 (e.g., ASO-STAT6-1849; SEQ ID NO: 140)±10, ±20, ±30, ±40, ±50, ±60, ±70, ±80, or ±90 nucleotides at the 3′ end and/or the 5′ end. In some aspects, the target region corresponds to nucleotides 1891-1907 of SEQ ID NO: 3 (e.g., ASO-STAT6-1891; SEQ ID NO: 141)±10, ±20, ±30, ±40, ±50, ±60, ±70, ±80, or ±90 nucleotides at the 3′ end and/or the 5′ end. In some aspects, the target region corresponds to nucleotides 1915-1931 of SEQ ID NO: 3 (e.g., ASO-STAT6-1915; SEQ ID NO: 142)±10, ±20, ±30, ±40, ±50, ±60, ±70, ±80, or ±90 nucleotides at the 3′ end and/or the 5′ end. In some aspects, the target region corresponds to nucleotides 1916-1932 of SEQ ID NO: 3 (e.g., ASO-STAT6-1916; SEQ ID NO: 143)±10, ±20, ±30, ±40, ±50, ±60, ±70, ±80, or ±90 nucleotides at the 3′ end and/or the 5′ end. In some aspects, the target region corresponds to nucleotides 1917-1933 of SEQ ID NO: 3 (e.g., ASO-STAT6-1917; SEQ ID NO: 144)±10, ±20, ±30, ±40, ±50, ±60, ±70, ±80, or ±90 nucleotides at the 3′ end and/or the 5′ end. In some aspects, the target region corresponds to nucleotides 1938-1954 of SEQ ID NO: 3 (e.g., ASO-STAT6-1938; SEQ ID NO: 145)±10, ±20, ±30, ±40, ±50, ±60, ±70, ±80, or ±90 nucleotides at the 3′ end and/or the 5′ end. In some aspects, the target region corresponds to nucleotides 1939-1955 of SEQ ID NO: 3 (e.g., ASO-STAT6-1939; SEQ ID NO: 146)±10, ±20, ±30, ±40, ±50, ±60, ±70, ±80, or ±90 nucleotides at the 3′ end and/or the 5′ end. In some aspects, the target region corresponds to nucleotides 2063-2079 of SEQ ID NO: 3 (e.g., ASO-STAT6-2063; SEQ ID NO: 147)±10, ±20, ±30, ±40, ±50, ±60, ±70, ±80, or ±90 nucleotides at the 3′ end and/or the 5′ end. In some aspects, the target region corresponds to nucleotides 2064-2080 of SEQ ID NO: 3 (e.g., ASO-STAT6-2064; SEQ ID NO: 148)±10, ±20, ±30, ±40, ±50, ±60, ±70, ±80, or ±90 nucleotides at the 3′ end and/or the 5′ end. In some aspects, the target region corresponds to nucleotides 2065-2081 of SEQ ID NO: 3 (e.g., ASO-STAT6-2065; SEQ ID NO: 149)±10, ±20, ±30, ±40, ±50, ±60, ±70, ±80, or ±90 nucleotides at the 3′ end and/or the 5′ end. In some aspects, the target region corresponds to nucleotides 2066-2082 of SEQ ID NO: 3 (e.g., ASO-STAT6-2066; SEQ ID NO: 150)±10, ±20, ±30, ±40, ±50, ±60, ±70, ±80, or ±90 nucleotides at the 3′ end and/or the 5′ end. In some aspects, the target region corresponds to nucleotides 2068-2084 of SEQ ID NO: 3 (e.g., ASO-STAT6-2068; SEQ ID NO: 151)±10, ±20, ±30, ±40, ±50, ±60, ±70, ±80, or ±90 nucleotides at the 3′ end and/or the 5′ end. In some aspects, the target region corresponds to nucleotides 2187-2203 of SEQ ID NO: 3 (e.g., ASO-STAT6-2187; SEQ ID NO: 152)±10, ±20, ±30, ±40, ±50, ±60, ±70, ±80, or ±90 nucleotides at the 3′ end and/or the 5′ end. In some aspects, the target region corresponds to nucleotides 2350-2366 of SEQ ID NO: 3 (e.g., ASO-STAT6-2350; SEQ ID NO: 153)±10, ±20, ±30, ±40, ±50, ±60, ±70, ±80, or ±90 nucleotides at the 3′ end and/or the 5′ end. In some aspects, the target region corresponds to nucleotides 2351-2367 of SEQ ID NO: 3 (e.g., ASO-STAT6-2351; SEQ ID NO: 154)±10, ±20, ±30, ±40, ±50, ±60, ±70, ±80, or ±90 nucleotides at the 3′ end and/or the 5′ end. In some aspects, the target region corresponds to nucleotides 2352-2368 of SEQ ID NO: 3 (e.g., ASO-STAT6-2352; SEQ ID NO: 155)±10, ±20, ±30, ±40, ±50, ±60, ±70, ±80, or ±90 nucleotides at the 3′ end and/or the 5′ end. In some aspects, the target region corresponds to nucleotides 2357-2373 of SEQ ID NO: 3 (e.g., ASO-STAT6-2357; SEQ ID NO: 156)±10, ±20, ±30, ±40, ±50, ±60, ±70, ±80, or ±90 nucleotides at the 3′ end and/or the 5′ end. In some aspects, the target region corresponds to nucleotides 513-532 of SEQ ID NO: 3 (e.g., ASO-STAT6-513; SEQ ID NO: 157)±10, ±20, ±30, ±40, ±50, ±60, ±70, ±80, or ±90 nucleotides at the 3′ end and/or the 5′ end. In some aspects, the target region corresponds to nucleotides 671-690 of SEQ ID NO: 3 (e.g., ASO-STAT6-671; SEQ ID NO: 158)±10, ±20, ±30, ±40, ±50, ±60, ±70, ±80, or ±90 nucleotides at the 3′ end and/or the 5′ end. In some aspects, the target region corresponds to nucleotides 1131-1150 of SEQ ID NO: 3 (e.g., ASO-STAT6-1131; SEQ ID NO: 159)±10, ±20, ±30, ±40, ±50, ±60, ±70, ±80, or ±90 nucleotides at the 3′ end and/or the 5′ end. In some aspects, the target region corresponds to nucleotides 1354-1373 of SEQ ID NO: 3 (e.g., ASO-STAT6-1354; SEQ ID NO: 160)±10, ±20, ±30, ±40, ±50, ±60, ±70, ±80, or ±90 nucleotides at the 3′ end and/or the 5′ end. In some aspects, the target region corresponds to nucleotides 1355-1374 of SEQ ID NO: 3 (e.g., ASO-STAT6-1355; SEQ ID NO: 161)±10, ±20, ±30, ±40, ±50, ±60, ±70, ±80, or ±90 nucleotides at the 3′ end and/or the 5′ end. In some aspects, the target region corresponds to nucleotides 1356-1375 of SEQ ID NO: 3 (e.g., ASO-STAT6-1356; SEQ ID NO: 162)±10, ±20, ±30, ±40, ±50, ±60, ±70, ±80, or ±90 nucleotides at the 3′ end and/or the 5′ end. In some aspects, the target region corresponds to nucleotides 1432-1451 of SEQ ID NO: 3 (e.g., ASO-STAT6-1432; SEQ ID NO: 163)±10, ±20, ±30, ±40, ±50, ±60, ±70, ±80, or ±90 nucleotides at the 3′ end and/or the 5′ end. In some aspects, the target region corresponds to nucleotides 1555-1574 of SEQ ID NO: 3 (e.g., ASO-STAT6-1555; SEQ ID NO: 164)±10, ±20, ±30, ±40, ±50, ±60, ±70, ±80, or ±90 nucleotides at the 3′ end and/or the 5′ end. In some aspects, the target region corresponds to nucleotides 1556-1575 of SEQ ID NO: 3 (e.g., ASO-STAT6-1556; SEQ ID NO: 165)±10, ±20, ±30, ±40, ±50, ±60, ±70, ±80, or ±90 nucleotides at the 3′ end and/or the 5′ end. In some aspects, the target region corresponds to nucleotides 1557-1576 of SEQ ID NO: 3 (e.g., ASO-STAT6-1557; SEQ ID NO: 166)±10, ±20, ±30, ±40, ±50, ±60, ±70, ±80, or ±90 nucleotides at the 3′ end and/or the 5′ end. In some aspects, the target region corresponds to nucleotides 1558-1577 of SEQ ID NO: 3 (e.g., ASO-STAT6-1558; SEQ ID NO: 167)±10, ±20, ±30, ±40, ±50, ±60, ±70, ±80, or ±90 nucleotides at the 3′ end and/or the 5′ end. In some aspects, the target region corresponds to nucleotides 1826-1845 of SEQ ID NO: 3 (e.g., ASO-STAT6-1826; SEQ ID NO: 168)±10, ±20, ±30, ±40, ±50, ±60, ±70, ±80, or ±90 nucleotides at the 3′ end and/or the 5′ end. In some aspects, the target region corresponds to nucleotides 1827-1846 of SEQ ID NO: 3 (e.g., ASO-STAT6-1827; SEQ ID NO: 169)±10, ±20, ±30, ±40, ±50, ±60, ±70, ±80, or ±90 nucleotides at the 3′ end and/or the 5′ end. In some aspects, the target region corresponds to nucleotides 1833-1852 of SEQ ID NO: 3 (e.g., ASO-STAT6-1833; SEQ ID NO: 170)±10, ±20, ±30, ±40, ±50, ±60, ±70, ±80, or ±90 nucleotides at the 3′ end and/or the 5′ end. In some aspects, the target region corresponds to nucleotides 1843-1862 of SEQ ID NO: 3 (e.g., ASO-STAT6-1843; SEQ ID NO: 171)±10, ±20, ±30, ±40, ±50, ±60, ±70, ±80, or ±90 nucleotides at the 3′ end and/or the 5′ end. In some aspects, the target region corresponds to nucleotides 1846-1865 of SEQ ID NO: 3 (e.g., ASO-STAT6-1846; SEQ ID NO: 172)±10, ±20, ±30, ±40, ±50, ±60, ±70, ±80, or ±90 nucleotides at the 3′ end and/or the 5′ end. In some aspects, the target region corresponds to nucleotides 1847-1866 of SEQ ID NO: 3 (e.g., ASO-STAT6-1847; SEQ ID NO: 173)±10, ±20, ±30, ±40, ±50, ±60, ±70, ±80, or ±90 nucleotides at the 3′ end and/or the 5′ end. In some aspects, the target region corresponds to nucleotides 1883-1902 of SEQ ID NO: 3 (e.g., ASO-STAT6-1883; SEQ ID NO: 174)±10, ±20, ±30, ±40, ±50, ±60, ±70, ±80, or ±90 nucleotides at the 3′ end and/or the 5′ end. In some aspects, the target region corresponds to nucleotides 1889-1908 of SEQ ID NO: 3 (e.g., ASO-STAT6-1889; SEQ ID NO: 175)±10, ±20, ±30, ±40, ±50, ±60, ±70, ±80, or ±90 nucleotides at the 3′ end and/or the 5′ end. In some aspects, the target region corresponds to nucleotides 1890-1909 of SEQ ID NO: 3 (e.g., ASO-STAT6-1890; SEQ ID NO: 176)±10, ±20, ±30, ±40, ±50, ±60, ±70, ±80, or ±90 nucleotides at the 3′ end and/or the 5′ end. In some aspects, the target region corresponds to nucleotides 1891-1910 of SEQ ID NO: 3 (e.g., ASO-STAT6-1891; SEQ ID NO: 177)±10, ±20, ±30, ±40, ±50, ±60, ±70, ±80, or ±90 nucleotides at the 3′ end and/or the 5′ end. In some aspects, the target region corresponds to nucleotides 1916-1935 of SEQ ID NO: 3 (e.g., ASO-STAT6-1916; SEQ ID NO: 178)±10, ±20, ±30, ±40, ±50, ±60, ±70, ±80, or ±90 nucleotides at the 3′ end and/or the 5′ end. In some aspects, the target region corresponds to nucleotides 1917-1936 of SEQ ID NO: 3 (e.g., ASO-STAT6-1917; SEQ ID NO: 179)±10, ±20, ±30, ±40, ±50, ±60, ±70, ±80, or ±90 nucleotides at the 3′ end and/or the 5′ end. In some aspects, the target region corresponds to nucleotides 2056-2075 of SEQ ID NO: 3 (e.g., ASO-STAT6-2056; SEQ ID NO: 180)±10, ±20, ±30, ±40, ±50, ±60, ±70, ±80, or ±90 nucleotides at the 3′ end and/or the 5′ end. In some aspects, the target region corresponds to nucleotides 2057-2076 of SEQ ID NO: 3 (e.g., ASO-STAT6-2057; SEQ ID NO: 181)±10, ±20, ±30, ±40, ±50, ±60, ±70, ±80, or ±90 nucleotides at the 3′ end and/or the 5′ end. In some aspects, the target region corresponds to nucleotides 2060-2079 of SEQ ID NO: 3 (e.g., ASO-STAT6-2060; SEQ ID NO: 182)±10, ±20, ±30, ±40, ±50, ±60, ±70, ±80, or ±90 nucleotides at the 3′ end and/or the 5′ end. In some aspects, the target region corresponds to nucleotides 2062-2081 of SEQ ID NO: 3 (e.g., ASO-STAT6-2062; SEQ ID NO: 183)±10, ±20, ±30, ±40, ±50, ±60, ±70, ±80, or ±90 nucleotides at the 3′ end and/or the 5′ end. In some aspects, the target region corresponds to nucleotides 2063-2082 of SEQ ID NO: 3 (e.g., ASO-STAT6-2063; SEQ ID NO: 184)±10, ±20, ±30, ±40, ±50, ±60, ±70, ±80, or ±90 nucleotides at the 3′ end and/or the 5′ end. In some aspects, the target region corresponds to nucleotides 2065-2084 of SEQ ID NO: 3 (e.g., ASO-STAT6-2065; SEQ ID NO: 185)±10, ±20, ±30, ±40, ±50, ±60, ±70, ±80, or ±90 nucleotides at the 3′ end and/or the 5′ end. In some aspects, the target region corresponds to nucleotides 2068-2087 of SEQ ID NO: 3 (e.g., ASO-STAT6-2068; SEQ ID NO: 186)±10, ±20, ±30, ±40, ±50, ±60, ±70, ±80, or ±90 nucleotides at the 3′ end and/or the 5′ end. In some aspects, the target region corresponds to nucleotides 2347-2366 of SEQ ID NO: 3 (e.g., ASO-STAT6-2347; SEQ ID NO: 187)±10, ±20, ±30, ±40, ±50, ±60, ±70, ±80, or ±90 nucleotides at the 3′ end and/or the 5′ end. In some aspects, the target region corresponds to nucleotides 2348-2367 of SEQ ID NO: 3 (e.g., ASO-STAT6-2348; SEQ ID NO: 188)±10, ±20, ±30, ±40, ±50, ±60, ±70, ±80, or ±90 nucleotides at the 3′ end and/or the 5′ end. In some aspects, the target region corresponds to nucleotides 2358-2377 of SEQ ID NO: 3 (e.g., ASO-STAT6-2358; SEQ ID NO: 189)±10, ±20, ±30, ±40, ±50, ±60, ±70, ±80, or ±90 nucleotides at the 3′ end and/or the 5′ end. In some aspects, the target region corresponds to nucleotides 2782-2801 of SEQ ID NO: 3 (e.g., ASO-STAT6-2782; SEQ ID NO: 190)±10, ±20, ±30, ±40, ±50, ±60, ±70, ±80, or ±90 nucleotides at the 3′ end and/or the 5′ end. In some aspects, the target region corresponds to nucleotides 3070-3089 of SEQ ID NO: 3 (e.g., ASO-STAT6-3070; SEQ ID NO: 191)±10, ±20, ±30, ±40, ±50, ±60, ±70, ±80, or ±90 nucleotides at the 3′ end and/or the 5′ end. In some aspects, the target region corresponds to nucleotides 3071-3090 of SEQ ID NO: 3 (e.g., ASO-STAT6-3071; SEQ ID NO: 192)±10, ±20, ±30, ±40, ±50, ±60, ±70, ±80, or ±90 nucleotides at the 3′ end and/or the 5′ end. In some aspects, the target region corresponds to nucleotides 3431-3450 of SEQ ID NO: 3 (e.g., ASO-STAT6-3431; SEQ ID NO: 193)±10, ±20, ±30, ±40, ±50, ±60, ±70, ±80, or ±90 nucleotides at the 3′ end and/or the 5′ end).

In some aspects, the ASO is not TGAGCGAATGGACAGGTCTT (SEQ ID NO: 89). In some aspects, the target region corresponds to a contiguous nucleotide sequence of 10 to 30 nucleotides in length that is complementary to a nucleic acid sequence within nucleotides 1-2056 of SEQ ID NO: 3. In some aspects, the target region corresponds to a contiguous nucleotide sequence of 10 to 30 nucleotides in length that is complementary to a nucleic acid sequence within nucleotides 1-2055 of SEQ ID NO: 3. In some aspects, the target region corresponds to a contiguous nucleotide sequence of 10 to 30 nucleotides in length that is complementary to a nucleic acid sequence within nucleotides 1-2054 of SEQ ID NO: 3. In some aspects, the target region corresponds to a contiguous nucleotide sequence of 10 to 30 nucleotides in length that is complementary to a nucleic acid sequence within nucleotides 1-2053 of SEQ ID NO: 3. In some aspects, the target region corresponds to a contiguous nucleotide sequence of 10 to 30 nucleotides in length that is complementary to a nucleic acid sequence within nucleotides 1-2052 of SEQ ID NO: 3. In some aspects, the target region corresponds to a contiguous nucleotide sequence of 10 to 30 nucleotides in length that is complementary to a nucleic acid sequence within nucleotides 1-2051 of SEQ ID NO: 3. In some aspects, the target region corresponds to a contiguous nucleotide sequence of 10 to 30 nucleotides in length that is complementary to a nucleic acid sequence within nucleotides 1-2050 of SEQ ID NO: 3. In some aspects, the target region corresponds to a contiguous nucleotide sequence of 10 to 30 nucleotides in length that is complementary to a nucleic acid sequence within nucleotides 1-2049 of SEQ ID NO: 3. In some aspects, the target region corresponds to a contiguous nucleotide sequence of 10 to 30 nucleotides in length that is complementary to a nucleic acid sequence within nucleotides 1-2048 of SEQ ID NO: 3. In some aspects, the target region corresponds to a contiguous nucleotide sequence of 10 to 30 nucleotides in length that is complementary to a nucleic acid sequence within nucleotides 1-2047 of SEQ ID NO: 3. In some aspects, the target region corresponds to a contiguous nucleotide sequence of 10 to 30 nucleotides in length that is complementary to a nucleic acid sequence within nucleotides 1-2046 of SEQ ID NO: 3. In some aspects, the target region corresponds to a contiguous nucleotide sequence of 10 to 30 nucleotides in length that is complementary to a nucleic acid sequence within nucleotides 1-2045 of SEQ ID NO: 3. In some aspects, the target region corresponds to a contiguous nucleotide sequence of 10 to 30 nucleotides in length that is complementary to a nucleic acid sequence within nucleotides 1-2044 of SEQ ID NO: 3. In some aspects, the target region corresponds to a contiguous nucleotide sequence of 10 to 30 nucleotides in length that is complementary to a nucleic acid sequence within nucleotides 1-2043 of SEQ ID NO: 3. In some aspects, the target region corresponds to a contiguous nucleotide sequence of 10 to 30 nucleotides in length that is complementary to a nucleic acid sequence within nucleotides 1-2042 of SEQ ID NO: 3. In some aspects, the target region corresponds to a contiguous nucleotide sequence of 10 to 30 nucleotides in length that is complementary to a nucleic acid sequence within nucleotides 1-2041 of SEQ ID NO: 3. In some aspects, the target region corresponds to a contiguous nucleotide sequence of 10 to 30 nucleotides in length that is complementary to a nucleic acid sequence within nucleotides 1-2040 of SEQ ID NO: 3. In some aspects, the target region corresponds to a contiguous nucleotide sequence of 10 to 30 nucleotides in length that is complementary to a nucleic acid sequence within nucleotides 1-2039 of SEQ ID NO: 3. In some aspects, the target region corresponds to a contiguous nucleotide sequence of 10 to 30 nucleotides in length that is complementary to a nucleic acid sequence within nucleotides 1-2038 of SEQ ID NO: 3.

In some aspects, the target region corresponds to a contiguous nucleotide sequence of 10 to 30 nucleotides in length that is complementary to a nucleic acid sequence within nucleotides 2041-3963 of SEQ ID NO: 3. In some aspects, the target region corresponds to a contiguous nucleotide sequence of 10 to 30 nucleotides in length that is complementary to a nucleic acid sequence within nucleotides 2042-3963 of SEQ ID NO: 3. In some aspects, the target region corresponds to a contiguous nucleotide sequence of 10 to 30 nucleotides in length that is complementary to a nucleic acid sequence within nucleotides 2043-3963 of SEQ ID NO: 3. In some aspects, the target region corresponds to a contiguous nucleotide sequence of 10 to 30 nucleotides in length that is complementary to a nucleic acid sequence within nucleotides 2044-3963 of SEQ ID NO: 3. In some aspects, the target region corresponds to a contiguous nucleotide sequence of 10 to 30 nucleotides in length that is complementary to a nucleic acid sequence within nucleotides 2045-3963 of SEQ ID NO: 3. In some aspects, the target region corresponds to a contiguous nucleotide sequence of 10 to 30 nucleotides in length that is complementary to a nucleic acid sequence within nucleotides 2046-3963 of SEQ ID NO: 3. In some aspects, the target region corresponds to a contiguous nucleotide sequence of 10 to 30 nucleotides in length that is complementary to a nucleic acid sequence within nucleotides 2047-3963 of SEQ ID NO: 3. In some aspects, the target region corresponds to a contiguous nucleotide sequence of 10 to 30 nucleotides in length that is complementary to a nucleic acid sequence within nucleotides 2048-3963 of SEQ ID NO: 3. In some aspects, the target region corresponds to a contiguous nucleotide sequence of 10 to 30 nucleotides in length that is complementary to a nucleic acid sequence within nucleotides 2049-3963 of SEQ ID NO: 3. In some aspects, the target region corresponds to a contiguous nucleotide sequence of 10 to 30 nucleotides in length that is complementary to a nucleic acid sequence within nucleotides 2050-3963 of SEQ ID NO: 3. In some aspects, the target region corresponds to a contiguous nucleotide sequence of 10 to 30 nucleotides in length that is complementary to a nucleic acid sequence within nucleotides 2051-3963 of SEQ ID NO: 3. In some aspects, the target region corresponds to a contiguous nucleotide sequence of 10 to 30 nucleotides in length that is complementary to a nucleic acid sequence within nucleotides 2052-3963 of SEQ ID NO: 3. In some aspects, the target region corresponds to a contiguous nucleotide sequence of 10 to 30 nucleotides in length that is complementary to a nucleic acid sequence within nucleotides 2053-3963 of SEQ ID NO: 3. In some aspects, the target region corresponds to a contiguous nucleotide sequence of 10 to 30 nucleotides in length that is complementary to a nucleic acid sequence within nucleotides 2054-3963 of SEQ ID NO: 3. In some aspects, the target region corresponds to a contiguous nucleotide sequence of 10 to 30 nucleotides in length that is complementary to a nucleic acid sequence within nucleotides 2055-3963 of SEQ ID NO: 3. In some aspects, the target region corresponds to a contiguous nucleotide sequence of 10 to 30 nucleotides in length that is complementary to a nucleic acid sequence within nucleotides 2056-3963 of SEQ ID NO: 3. In some aspects, the target region corresponds to a contiguous nucleotide sequence of 10 to 30 nucleotides in length that is complementary to a nucleic acid sequence within nucleotides 2057-3963 of SEQ ID NO: 3. In some aspects, the target region corresponds to a contiguous nucleotide sequence of 10 to 30 nucleotides in length that is complementary to a nucleic acid sequence within nucleotides 2058-3963 of SEQ ID NO: 3. In some aspects, the target region corresponds to a contiguous nucleotide sequence of 10 to 30 nucleotides in length that is complementary to a nucleic acid sequence within nucleotides 2059-3963 of SEQ ID NO: 3.

In some aspects, the ASO of the present disclosure hybridizes to multiple target regions within the STAT6 transcript (e.g., genomic sequence, SEQ ID NO: 1). In some aspects, the ASO hybridizes to two different target regions within the STAT6 transcript. In some aspects, the ASO hybridizes to three different target regions within the STAT6 transcript. The sequences of exemplary ASOs that hybridize to multiple target regions, and the start/end sites of the different target regions are provided in FIG. 1A. In some aspects, the ASOs that hybridizes to multiple regions within the STAT6 transcript (e.g., genomic sequence, SEQ ID NO: 1) are more potent (e.g., having lower EC50) at reducing STAT6 expression compared to ASOs that hybridizes to a single region within the STAT6 transcript (e.g., genomic sequence, SEQ ID NO: 1).

### III.B. ASO STAT6 Sequences

In some aspects, the ASOs of the disclosure comprise a contiguous nucleotide sequence which corresponds to the complement of a region of STAT6 transcript, e.g., a nucleotide sequence corresponding to SEQ ID NO: 1 or SEQ ID NO: 3.

In certain aspects, the disclosure provides an ASO from 10-30, such as 10-15 nucleotides, 10-20 nucleotides, 10-25 nucleotides in length, or about 20 nucleotides in length, wherein the contiguous nucleotide sequence has at least about 80%, at least about 85%, at least about 90%, at least about 95%, at least about 96%, at least about 97%, at least about 98%, at least about 99%, or about 100% sequence identity to a region within the complement of: a STAT6 transcript, such as SEQ ID NO: 1 or SEQ ID NO: 3 or naturally occurring variant thereof. Thus, for example, the ASO hybridizes to a single stranded nucleic acid molecule having the sequence of SEQ ID NO: 1 or SEQ ID NO: 3 or a portion thereof.

The ASO can comprise a contiguous nucleotide sequence which is fully complementary (perfectly complementary) to the equivalent region of a nucleic acid which encodes a mammalian STAT6 protein (e.g., SEQ ID NO: 1 or SEQ ID NO: 3). The ASO can comprise a contiguous nucleotide sequence which is fully complementary (perfectly complementary) to a nucleic acid sequence, or a region within the sequence, corresponding to nucleotides X—Y of SEQ ID NO: 1 or SEQ ID NO: 3, wherein X and Y are the start site and the end site, respectively, as shown in FIG. 1.

The ASO can comprise a contiguous nucleotide sequence which is fully complementary (perfectly complementary) to the equivalent region of a mRNA which encodes a mammalian STAT6 protein (e.g., SEQ ID NO: 3). The ASO can comprise a contiguous nucleotide sequence which is fully complementary (perfectly complementary) to a mRNA sequence, or a region within the sequence, corresponding to nucleotides X—Y of SEQ ID NO: 3, wherein X and Y are the start site and the end site, respectively.

In some aspects, the nucleotide sequence of the ASOs of the disclosure or the contiguous nucleotide sequence has at least about 80% sequence identity to a sequence selected from SEQ ID NOs: 91 to 193 (i.e., the sequences in FIG. 1), such as at least about 80%, at least about 85%, at least about 90%, at least about 91%, at least about 92%, at least about 93%, at least about 94%, at least about 95%, at least about 96% sequence identity, at least about 97% sequence identity, at least about 98% sequence identity, at least about 99% sequence identity, such as about 100% sequence identity (homologous). In some aspects, the ASO has a design described elsewhere herein or a chemical structure shown elsewhere herein (e.g., FIG. 1).

In some aspects the ASO (or contiguous nucleotide portion thereof) is selected from, or comprises, one of the sequences selected from the group consisting of SEQ ID NOs: 91 to 193 or a region of at least 10 contiguous nucleotides thereof, wherein the ASO (or contiguous nucleotide portion thereof) can optionally comprise one, two, three, or four mismatches when compared to the corresponding STAT6 transcript.

In some aspects, the ASO comprises a sequence selected from the group consisting of 91 (e.g., ASO-STAT6-1053), 92 (e.g., ASO-STAT6-1359), 93 (e.g., ASO-STAT6-1890), 94 (e.g., ASO-STAT6-1892), 95 (e.g., ASO-STAT6-1915), 96 (e.g., ASO-STAT6-1916), 97 (e.g., ASO-STAT6-1917), 98 (e.g., ASO-STAT6-1918), 99 (e.g., ASO-STAT6-1919), 100 (e.g., ASO-STAT6-1920), 101 (e.g., ASO-STAT6-1937), 102 (e.g., ASO-STAT6-1938), 103 (e.g., ASO-STAT6-2061), 104 (e.g., ASO-STAT6-2062), 105 (e.g., ASO-STAT6-2063), 106 (e.g., ASO-STAT6-2064), 107 (e.g., ASO-STAT6-2066), 108 (e.g., ASO-STAT6-2067), 109 (e.g., ASO-STAT6-2068), 110 (e.g., ASO-STAT6-2352), 111 (e.g., ASO-STAT6-3073), 112 (e.g., ASO-STAT6-1053), 113 (e.g., ASO-STAT6-1054), 114 (e.g., ASO-STAT6-1356), 115 (e.g., ASO-STAT6-1847), 116 (e.g., ASO-STAT6-1886), 117 (e.g., ASO-STAT6-1887), 118 (e.g., ASO-STAT6-1888), 119 (e.g., ASO-STAT6-1889), 120 (e.g., ASO-STAT6-1890), 121 (e.g., ASO-STAT6-1893), 122 (e.g., ASO-STAT6-1917), 123 (e.g., ASO-STAT6-1919), 124 (e.g., ASO-STAT6-2056), 125 (e.g., ASO-STAT6-2060), 126 (e.g., ASO-STAT6-2066), 127 (e.g., ASO-STAT6-2070), 128 (e.g., ASO-STAT6-2351), 129 (e.g., ASO-STAT6-2352), 130 (e.g., ASO-STAT6-2359), 131 (e.g., ASO-STAT6-3633), 132 (e.g., ASO-STAT6-673), 133 (e.g., ASO-STAT6-1052), 134 (e.g., ASO-STAT6-1356), 135 (e.g., ASO-STAT6-1357), 136 (e.g., ASO-STAT6-1359), 137 (e.g., ASO-STAT6-1360), 138 (e.g., ASO-STAT6-1839), 139 (e.g., ASO-STAT6-1848), 140 (e.g., ASO-STAT6-1849), 141 (e.g., ASO-STAT6-1891), 142 (e.g., ASO-STAT6-1915), 143 (e.g., ASO-STAT6-1916), 144 (e.g., ASO-STAT6-1917), 145 (e.g., ASO-STAT6-1938), 146 (e.g., ASO-STAT6-1939), 147 (e.g., ASO-STAT6-2063), 148 (e.g., ASO-STAT6-2064), 149 (e.g., ASO-STAT6-2065), 150 (e.g., ASO-STAT6-2066), 151 (e.g., ASO-STAT6-2068), 152 (e.g., ASO-STAT6-2187), 153 (e.g., ASO-STAT6-2350), 154 (e.g., ASO-STAT6-2351), 155 (e.g., ASO-STAT6-2352), 156 (e.g., ASO-STAT6-2357), 157 (e.g., ASO-STAT6-513), 158 (e.g., ASO-STAT6-671), 159 (e.g., ASO-STAT6-1131), 160 (e.g., ASO-STAT6-1354), 161 (e.g., ASO-STAT6-1355), 162 (e.g., ASO-STAT6-1356), 163 (e.g., ASO-STAT6-1432), 164 (e.g., ASO-STAT6-1555), 165 (e.g., ASO-STAT6-1556), 166 (e.g., ASO-STAT6-1557), 167 (e.g., ASO-STAT6-1558), 168 (e.g., ASO-STAT6-1826), 169 (e.g., ASO-STAT6-1827), 170 (e.g., ASO-STAT6-1833), 171 (e.g., ASO-STAT6-1843), 172 (e.g., ASO-STAT6-1846), 173 (e.g., ASO-STAT6-1847), 174 (e.g., ASO-STAT6-1883), 175 (e.g., ASO-STAT6-1889), 176 (e.g., ASO-STAT6-1890), 177 (e.g., ASO-STAT6-1891), 178 (e.g., ASO-STAT6-1916), 179 (e.g., ASO-STAT6-1917), 180 (e.g., ASO-STAT6-2056), 181 (e.g., ASO-STAT6-2057), 182 (e.g., ASO-STAT6-2060), 183 (e.g., ASO-STAT6-2062), 184 (e.g., ASO-STAT6-2063), 185 (e.g., ASO-STAT6-2065), 186 (e.g., ASO-STAT6-2068), 187 (e.g., ASO-STAT6-2347), 188 (e.g., ASO-STAT6-2348), 189 (e.g., ASO-STAT6-2358), 190 (e.g., ASO-STAT6-2782), 191 (e.g., ASO-STAT6-3070), 192 (e.g., ASO-STAT6-3071), and 193 (e.g., ASO-STAT6-3431).

In some aspects, the ASO comprises the sequence as set forth in SEQ ID NO: 91 (e.g., ASO-STAT6-1053). In some aspects, the ASO comprises the sequence as set forth in SEQ ID NO: 92 (e.g., ASO-STAT6-1359). In some aspects, the ASO comprises the sequence as set forth in SEQ ID NO: 93 (e.g., ASO-STAT6-1890). In some aspects, the ASO comprises the sequence as set forth in SEQ ID NO: 94 (e.g., ASO-STAT6-1892). In some aspects, the ASO comprises the sequence as set forth in SEQ ID NO: 95 (e.g., ASO-STAT6-1915). In some aspects, the ASO comprises the sequence as set forth in SEQ ID NO: 96 (e.g., ASO-STAT6-1916). In some aspects, the ASO comprises the sequence as set forth in SEQ ID NO: 97 (e.g., ASO-STAT6-1917). In some aspects, the ASO comprises the sequence as set forth in SEQ ID NO: 98 (e.g., ASO-STAT6-1918). In some aspects, the ASO comprises the sequence as set forth in SEQ ID NO: 99 (e.g., ASO-STAT6-1919). In some aspects, the ASO comprises the sequence as set forth in SEQ ID NO: 100 (e.g., ASO-STAT6-1920). In some aspects, the ASO comprises the sequence as set forth in SEQ ID NO: 101 (e.g., ASO-STAT6-1937). In some aspects, the ASO comprises the sequence as set forth in SEQ ID NO: 102 (e.g., ASO-STAT6-1938). In some aspects, the ASO comprises the sequence as set forth in SEQ ID NO: 103 (e.g., ASO-STAT6-2061). In some aspects, the ASO comprises the sequence as set forth in SEQ ID NO: 104 (e.g., ASO-STAT6-2062). In some aspects, the ASO comprises the sequence as set forth in SEQ ID NO: 105 (e.g., ASO-STAT6-2063). In some aspects, the ASO comprises the sequence as set forth in SEQ ID NO: 106 (e.g., ASO-STAT6-2064). In some aspects, the ASO comprises the sequence as set forth in SEQ ID NO: 107 (e.g., ASO-STAT6-2066). In some aspects, the ASO comprises the sequence as set forth in SEQ ID NO: 108 (e.g., ASO-STAT6-2067). In some aspects, the ASO comprises the sequence as set forth in SEQ ID NO: 109 (e.g., ASO-STAT6-2068). In some aspects, the ASO comprises the sequence as set forth in SEQ ID NO: 110 (e.g., ASO-STAT6-2352). In some aspects, the ASO comprises the sequence as set forth in SEQ ID NO: 111 (e.g., ASO-STAT6-3073). In some aspects, the ASO comprises the sequence as set forth in SEQ ID NO: 112 (e.g., ASO-STAT6-1053). In some aspects, the ASO comprises the sequence as set forth in SEQ ID NO: 113 (e.g., ASO-STAT6-1054). In some aspects, the ASO comprises the sequence as set forth in SEQ ID NO: 114 (e.g., ASO-STAT6-1356). In some aspects, the ASO comprises the sequence as set forth in SEQ ID NO: 115 (e.g., ASO-STAT6-1847). In some aspects, the ASO comprises the sequence as set forth in SEQ ID NO: 116 (e.g., ASO-STAT6-1886). In some aspects, the ASO comprises the sequence as set forth in SEQ ID NO: 117 (e.g., ASO-STAT6-1887). In some aspects, the ASO comprises the sequence as set forth in SEQ ID NO: 118 (e.g., ASO-STAT6-1888). In some aspects, the ASO comprises the sequence as set forth in SEQ ID NO: 119 (e.g., ASO-STAT6-1889). In some aspects, the ASO comprises the sequence as set forth in SEQ ID NO: 120 (e.g., ASO-STAT6-1890). In some aspects, the ASO comprises the sequence as set forth in SEQ ID NO: 121 (e.g., ASO-STAT6-1893). In some aspects, the ASO comprises the sequence as set forth in SEQ ID NO: 122 (e.g., ASO-STAT6-1917). In some aspects, the ASO comprises the sequence as set forth in SEQ ID NO: 123 (e.g., ASO-STAT6-1919). In some aspects, the ASO comprises the sequence as set forth in SEQ ID NO: 124 (e.g., ASO-STAT6-2056). In some aspects, the ASO comprises the sequence as set forth in SEQ ID NO: 125 (e.g., ASO-STAT6-2060). In some aspects, the ASO comprises the sequence as set forth in SEQ ID NO: 126 (e.g., ASO-STAT6-2066). In some aspects, the ASO comprises the sequence as set forth in SEQ ID NO: 127 (e.g., ASO-STAT6-2070). In some aspects, the ASO comprises the sequence as set forth in SEQ ID NO: 128 (e.g., ASO-STAT6-2351). In some aspects, the ASO comprises the sequence as set forth in SEQ ID NO: 129 (e.g., ASO-STAT6-2352). In some aspects, the ASO comprises the sequence as set forth in SEQ ID NO: 130 (e.g., ASO-STAT6-2359). In some aspects, the ASO comprises the sequence as set forth in SEQ ID NO: 131 (e.g., ASO-STAT6-3633). In some aspects, the ASO comprises the sequence as set forth in SEQ ID NO: 132 (e.g., ASO-STAT6-673). In some aspects, the ASO comprises the sequence as set forth in SEQ ID NO: 133 (e.g., ASO-STAT6-1052). In some aspects, the ASO comprises the sequence as set forth in SEQ ID NO: 134 (e.g., ASO-STAT6-1356). In some aspects, the ASO comprises the sequence as set forth in SEQ ID NO: 135 (e.g., ASO-STAT6-1357). In some aspects, the ASO comprises the sequence as set forth in SEQ ID NO: 136 (e.g., ASO-STAT6-1359). In some aspects, the ASO comprises the sequence as set forth in SEQ ID NO: 137 (e.g., ASO-STAT6-1360). In some aspects, the ASO comprises the sequence as set forth in SEQ ID NO: 138 (e.g., ASO-STAT6-1839). In some aspects, the ASO comprises the sequence as set forth in SEQ ID NO: 139 (e.g., ASO-STAT6-1848). In some aspects, the ASO comprises the sequence as set forth in SEQ ID NO: 140 (e.g., ASO-STAT6-1849). In some aspects, the ASO comprises the sequence as set forth in SEQ ID NO: 141 (e.g., ASO-STAT6-1891). In some aspects, the ASO comprises the sequence as set forth in SEQ ID NO: 142 (e.g., ASO-STAT6-1915). In some aspects, the ASO comprises the sequence as set forth in SEQ ID NO: 143 (e.g., ASO-STAT6-1916). In some aspects, the ASO comprises the sequence as set forth in SEQ ID NO: 144 (e.g., ASO-STAT6-1917). In some aspects, the ASO comprises the sequence as set forth in SEQ ID NO: 145 (e.g., ASO-STAT6-1938). In some aspects, the ASO comprises the sequence as set forth in SEQ ID NO: 146 (e.g., ASO-STAT6-1939). In some aspects, the ASO comprises the sequence as set forth in SEQ ID NO: 147 (e.g., ASO-STAT6-2063). In some aspects, the ASO comprises the sequence as set forth in SEQ ID NO: 148 (e.g., ASO-STAT6-2064). In some aspects, the ASO comprises the sequence as set forth in SEQ ID NO: 149 (e.g., ASO-STAT6-2065). In some aspects, the ASO comprises the sequence as set forth in SEQ ID NO: 150 (e.g., ASO-STAT6-2066). In some aspects, the ASO comprises the sequence as set forth in SEQ ID NO: 151 (e.g., ASO-STAT6-2068). In some aspects, the ASO comprises the sequence as set forth in SEQ ID NO: 152 (e.g., ASO-STAT6-2187). In some aspects, the ASO comprises the sequence as set forth in SEQ ID NO: 153 (e.g., ASO-STAT6-2350). In some aspects, the ASO comprises the sequence as set forth in SEQ ID NO: 154 (e.g., ASO-STAT6-2351). In some aspects, the ASO comprises the sequence as set forth in SEQ ID NO: 155 (e.g., ASO-STAT6-2352). In some aspects, the ASO comprises the sequence as set forth in SEQ ID NO: 156 (e.g., ASO-STAT6-2357). In some aspects, the ASO comprises the sequence as set forth in SEQ ID NO: 157 (e.g., ASO-STAT6-513). In some aspects, the ASO comprises the sequence as set forth in SEQ ID NO: 158 (e.g., ASO-STAT6-671). In some aspects, the ASO comprises the sequence as set forth in SEQ ID NO: 159 (e.g., ASO-STAT6-1131). In some aspects, the ASO comprises the sequence as set forth in SEQ ID NO: 160 (e.g., ASO-STAT6-1354). In some aspects, the ASO comprises the sequence as set forth in SEQ ID NO: 161 (e.g., ASO-STAT6-1355). In some aspects, the ASO comprises the sequence as set forth in SEQ ID NO: 162 (e.g., ASO-STAT6-1356). In some aspects, the ASO comprises the sequence as set forth in SEQ ID NO: 163 (e.g., ASO-STAT6-1432). In some aspects, the ASO comprises the sequence as set forth in SEQ ID NO: 164 (e.g., ASO-STAT6-1555). In some aspects, the ASO comprises the sequence as set forth in SEQ ID NO: 165 (e.g., ASO-STAT6-1556). In some aspects, the ASO comprises the sequence as set forth in SEQ ID NO: 166 (e.g., ASO-STAT6-1557). In some aspects, the ASO comprises the sequence as set forth in SEQ ID NO: 167 (e.g., ASO-STAT6-1558). In some aspects, the ASO comprises the sequence as set forth in SEQ ID NO: 168 (e.g., ASO-STAT6-1826). In some aspects, the ASO comprises the sequence as set forth in SEQ ID NO: 169 (e.g., ASO-STAT6-1827). In some aspects, the ASO comprises the sequence as set forth in SEQ ID NO: 170 (e.g., ASO-STAT6-1833). In some aspects, the ASO comprises the sequence as set forth in SEQ ID NO: 171 (e.g., ASO-STAT6-1843). In some aspects, the ASO comprises the sequence as set forth in SEQ ID NO: 172 (e.g., ASO-STAT6-1846). In some aspects, the ASO comprises the sequence as set forth in SEQ ID NO: 173 (e.g., ASO-STAT6-1847). In some aspects, the ASO comprises the sequence as set forth in SEQ ID NO: 174 (e.g., ASO-STAT6-1883). In some aspects, the ASO comprises the sequence as set forth in SEQ ID NO: 175 (e.g., ASO-STAT6-1889). In some aspects, the ASO comprises the sequence as set forth in SEQ ID NO: 176 (e.g., ASO-STAT6-1890). In some aspects, the ASO comprises the sequence as set forth in SEQ ID NO: 177 (e.g., ASO-STAT6-1891). In some aspects, the ASO comprises the sequence as set forth in SEQ ID NO: 178 (e.g., ASO-STAT6-1916). In some aspects, the ASO comprises the sequence as set forth in SEQ ID NO: 179 (e.g., ASO-STAT6-1917). In some aspects, the ASO comprises the sequence as set forth in SEQ ID NO: 180 (e.g., ASO-STAT6-2056). In some aspects, the ASO comprises the sequence as set forth in SEQ ID NO: 181 (e.g., ASO-STAT6-2057). In some aspects, the ASO comprises the sequence as set forth in SEQ ID NO: 182 (e.g., ASO-STAT6-2060). In some aspects, the ASO comprises the sequence as set forth in SEQ ID NO: 183 (e.g., ASO-STAT6-2062). In some aspects, the ASO comprises the sequence as set forth in SEQ ID NO: 184 (e.g., ASO-STAT6-2063). In some aspects, the ASO comprises the sequence as set forth in SEQ ID NO: 185 (e.g., ASO-STAT6-2065). In some aspects, the ASO comprises the sequence as set forth in SEQ ID NO: 186 (e.g., ASO-STAT6-2068). In some aspects, the ASO comprises the sequence as set forth in SEQ ID NO: 187 (e.g., ASO-STAT6-2347). In some aspects, the ASO comprises the sequence as set forth in SEQ ID NO: 188 (e.g., ASO-STAT6-2348). In some aspects, the ASO comprises the sequence as set forth in SEQ ID NO: 189 (e.g., ASO-STAT6-2358). In some aspects, the ASO comprises the sequence as set forth in SEQ ID NO: 190 (e.g., ASO-STAT6-2782). In some aspects, the ASO comprises the sequence as set forth in SEQ ID NO: 191 (e.g., ASO-STAT6-3070). In some aspects, the ASO comprises the sequence as set forth in SEQ ID NO: 192 (e.g., ASO-STAT6-3071). In some aspects, the ASO comprises the sequence as set forth in SEQ ID NO: 193 (e.g., ASO-STAT6-3431).

In some aspects, the ASOs of the disclosure bind to the target nucleic acid sequence (e.g., STAT6 transcript) and are capable of inhibiting or reducing expression of the STAT6 transcript by at least 10% or 20% compared to the normal (i.e., control) expression level in the cell, e.g., at least about 30%, at least about 40%, at least about 50%, at least about 60%, at least about 70%, at least about 80%, at least about 90%, at least about 95%, at least about 96%, at least about 97%, at least about 98%, at least about 99%, or about 100% compared to the normal expression level (e.g., expression level in cells that have not been exposed to the ASO).

In some aspects, the ASOs of the disclosure are capable of reducing expression of STAT6 mRNA in vitro by at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, or about 100% in target cells when the cells are in contact with the ASO compared to cells that are not in contact with the ASO (e.g., contact with saline).

### III.C. ASO Length

The ASOs can comprise a contiguous nucleotide sequence of a total of 10, 11, 12, 13, 14, 15, 16, 17, 18, 19, 20, 21, 22, 23, 24, 25, 26, 27, 28, 29, or 30 contiguous nucleotides in length. It should be understood that when a range is given for an ASO, or contiguous nucleotide sequence length, the range includes the lower and upper lengths provided in the range, for example from (or between) 10-30, includes both 10 and 30.

In some aspects, the ASOs comprise a contiguous nucleotide sequence of a total of about 14-20, 14, 15, 16, 17, 18, 19, or 20 contiguous nucleotides in length. In certain aspects, the ASOs comprise a contiguous nucleotide sequence of a total of about 20 contiguous nucleotides in length. In certain aspects, ASOs of the present disclosure are 14 nucleotides in length. In certain aspects, ASOs of the present disclosure are 15 nucleotides in length. In certain aspects, ASOs of the present disclosure are 16 nucleotides in length. In certain aspects, ASOs of the present disclosure are 17 nucleotides in length. In certain aspects, ASOs of the present disclosure are 18 nucleotides in length. In certain aspects, ASOs of the present disclosure are 19 nucleotides in length.

### III.D. Nucleosides and Nucleoside Analogs

In one aspect of the disclosure, the ASOs comprise one or more non-naturally occurring nucleoside analogs. “Nucleoside analogs” as used herein are variants of natural nucleosides, such as DNA or RNA nucleosides, by virtue of modifications in the sugar and/or base moieties. Analogs could in principle be merely “silent” or “equivalent” to the natural nucleosides in the context of the oligonucleotide, i.e. have no functional effect on the way the oligonucleotide works to inhibit target gene expression. Such “equivalent” analogs can nevertheless be useful if, for example, they are easier or cheaper to manufacture, or are more stable to storage or manufacturing conditions, or represent a tag or label. In some aspects, however, the analogs will have a functional effect on the way in which the ASO works to inhibit expression; for example by producing increased binding affinity to the target and/or increased resistance to intracellular nucleases and/or increased ease of transport into the cell. Specific examples of nucleoside analogs are described by e.g. Freier & Altmann; Nucl. Acid Res., 1997, 25, 4429-4443 and Uhlmann; Curr. Opinion in Drug Development, 2000, 3(2), 293-213, and in Scheme 1. The ASOs of the present disclosure can contain more than one, more than two, more than three, more than four, more than five, more than six, more than seven, more than eight, more than nine, more than 10, more than 11, more than 12, more than 13, more than 14, more than 15, more than 16, more than 18, more than 19, or more than 20 nucleoside analogs. In some aspects, the nucleoside analogs in the ASOs are the same. In other aspects, the nucleoside analogs in the ASOs are different. The nucleotide analogs in the ASOs can be any one of or combination of the following nucleoside analogs.

In some aspects, the nucleoside analog comprises a 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-ANA; bicyclic nucleoside analog; or any combination thereof. In some aspects, the nucleoside analog comprises a sugar modified nucleoside. In some aspects, the nucleoside analog comprises a nucleoside comprising a bicyclic sugar. In some aspects, the nucleoside analog comprises an LNA.

In some aspects, the nucleoside analog is selected from the group consisting of constrained ethyl nucleoside (cEt), 2′,4′-constrained 2′-O-methoxyethyl (cMOE), α-L-LNA, β-D-LNA, 2′-O,4′-C-ethylene-bridged nucleic acids (ENA), amino-LNA, oxy-LNA, thio-LNA, and any combination thereof. In some aspects, the ASO comprises one or more 5′-methyl-cytosine nucleobases.

III.D.1. Nucleobase

The term nucleobase includes the purine (e.g., adenine and guanine) and pyrimidine (e.g., uracil, thymine and cytosine) moiety present in nucleosides and nucleotides which form hydrogen bonds in nucleic acid hybridization. In the context of the present disclosure, the term nucleobase also encompasses modified nucleobases which may differ from naturally occurring nucleobases, but are functional during nucleic acid hybridization. In some aspects, the nucleobase moiety is modified by modifying or replacing the nucleobase. In this context, “nucleobase” refers to both naturally occurring nucleobases such as adenine, guanine, cytosine, thymidine, uracil, xanthine and hypoxanthine, as well as non-naturally occurring variants. Such variants are for example described in Hirao et al., (2012) Accounts of Chemical Research vol 45 page 2055 and Bergstrom (2009) Current Protocols in Nucleic Acid Chemistry Suppl. 37 1.4.1.

In a some aspects, the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl-cytosine, 5-thiazolo-cytosine, 5-propynyl-cytosine, 5-propynyl-uracil, 5-bromouracil, 5-thiazolo-uracil, 2-thio-uracil, 2′thio-thymine, inosine, diaminopurine, 6-aminopurine, 2-aminopurine, 2,6-diaminopurine, and 2-chloro-6-aminopurine.

The nucleobase moieties may be indicated by the letter code for each corresponding nucleobase, e.g., A, T, G, C, or U, wherein each letter may optionally include modified nucleobases of equivalent function. For example, in the exemplified oligonucleotides, the nucleobase moieties are selected from A, T, G, C, and 5-methyl-cytosine. Optionally, for LNA gapmers, 5-methyl-cytosine LNA nucleosides may be used.

III.D.2. Sugar Modification

The ASO of the disclosure can comprise one or more nucleosides which have a modified sugar moiety, i.e. a modification of the sugar moiety when compared to the ribose sugar moiety found in DNA and RNA. Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradical bridge between the C2′ and C4′ carbons on the ribose ring (LNA), or an unlinked ribose ring which typically lacks a bond between the C2′ and C3′ carbons (e.g., UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in RNA nucleosides. Substituents may, for example be introduced at the 2′, 3′, 4′, or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity.

D. 2.a 2′ Modified Nucleosides

A 2′ sugar modified nucleoside is a nucleoside which has a substituent other than H or —OH at the 2′ position (2′ substituted nucleoside) or comprises a 2′ linked biradical, and includes 2′ substituted nucleosides and LNA (2′-4′ biradical bridged) nucleosides. For example, the 2′ modified sugar may provide enhanced binding affinity (e.g., affinity enhancing 2′ sugar modified nucleoside) and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, 2′-Fluro-DNA, arabino nucleic acids (ANA), and 2′-Fluoro-ANA nucleoside. For further examples, please see, e.g., Freier & Altmann; Nucl. Acid Res., 1997, 25, 4429-4443; Uhlmann, Curr. Opinion in Drug Development, 2000, 3(2), 293-213; and Deleavey and Damha, Chemistry and Biology 2012, 19, 937. Below are illustrations of some 2′ substituted modified nucleosides.

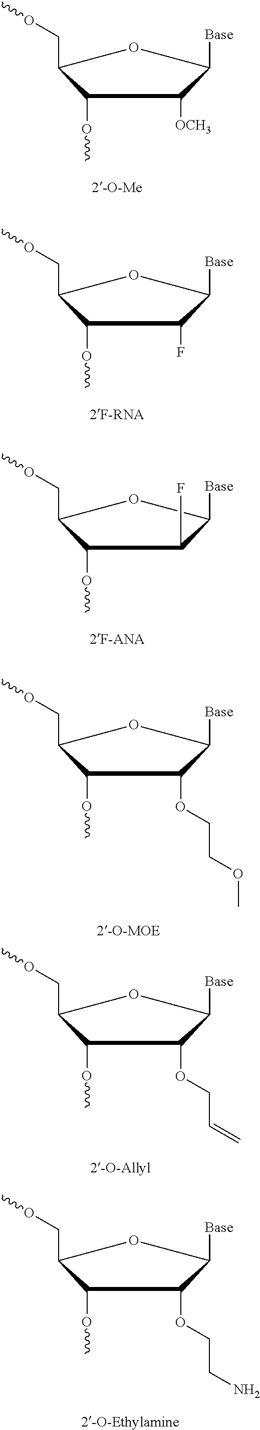

III.D.2.b Locked Nucleic Acid Nucleosides (LNA).

LNA nucleosides are modified nucleosides which comprise a linker group (referred to as a biradical or a bridge) between C2′ and C4′ of the ribose sugar ring of a nucleoside (i.e., 2′-4′ bridge), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al., J. Org. Chem. 2010, Vol 75(5) pp. 1569-81, and Mitsuoka et al., Nucleic Acids Research 2009, 37(4), 1225-1238.

In some aspects, the modified nucleoside or the LNA nucleosides of the ASO of the disclosure has a general structure of the formula I or II:

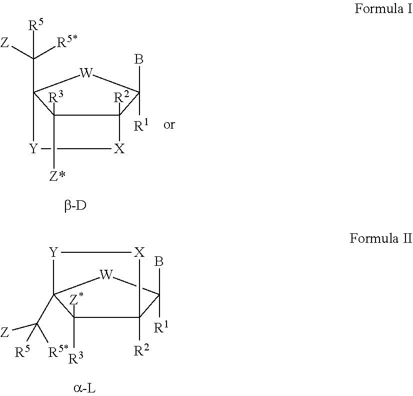

wherein
W is selected from —O—, —S—, —N(R^{a})—, —C(R^{a}R^{b})—, in particular —O—;
B is a nucleobase or a modified nucleobase moiety;
Z is an internucleoside linkage to an adjacent nucleoside or a 5′-terminal group;
Z* is an internucleoside linkage to an adjacent nucleoside or a 3′-terminal group;
R¹, R², R³, R⁵ and R^{5*} are independently selected from hydrogen, halogen, alkyl, alkenyl, alkynyl, hydroxy, alkoxy, alkoxyalkyl, alkenyloxy, carboxyl, alkoxycarbonyl, alkylcarbonyl, formyl, azide, heterocycle and aryl; and
X, Y, R^{a} and R^{b} are as defined herein.

In some aspects, —X—Y—, R^{a} is hydrogen or alkyl, in particular hydrogen or methyl. In some aspects of —X—Y—, R^{b} is hydrogen or alkyl, in particular hydrogen or methyl. In other aspects of —X—Y—, one or both of R^{a} and R^{b} are hydrogen. In further aspects of —X—Y—, only one of R^{a} and R^{b} is hydrogen. In some aspects of —X—Y—, one of R^{a} and R^{b} is methyl and the other one is hydrogen. In certain aspects of —X—Y—, R^{a} and R^{b} are both methyl at the same time.

In some aspects, —X—, R^{a} is hydrogen or alkyl, in particular hydrogen or methyl. In some aspects of —X—, R^{b} is hydrogen or alkyl, in particular hydrogen or methyl. In other aspects of —X—, one or both of R^{a} and R^{b} are hydrogen. In certain aspects of —X—, only one of R^{a} and R^{b} is hydrogen. In certain aspects of —X—, one of R^{a} and R^{b} is methyl and the other one is hydrogen. In other aspects of —X—, R^{a} and R^{b} are both methyl at the same time.

In some aspects, —Y—, R^{a} is hydrogen or alkyl, in particular hydrogen or methyl. In certain aspects of —Y—, R^{b} is hydrogen or alkyl, in particular hydrogen or methyl. In other aspects of —Y—, one or both of R^{a} and R^{b} are hydrogen. In some aspects of —Y—, only one of R^{a} and R^{b} is hydrogen. In other aspects of —Y—, one of R^{a} and R^{b} is methyl and the other one is hydrogen. In some aspects of —Y—, R^{a} and R^{b} are both methyl at the same time.

In some aspects, R¹, R², R³, R⁵ and R^{5*} are independently selected from hydrogen and alkyl, in particular hydrogen and methyl.

In some aspects, R¹, R², R³, R⁵ and R^{5*} are all hydrogen at the same time.

In some aspects, R¹, R², R³, are all hydrogen at the same time, one of R⁵ and R^{5*} is hydrogen and the other one is as defined above, in particular alkyl, more particularly methyl.

In some aspects, R¹, R², R³, are all hydrogen at the same time, one of R⁵ and R^{5*} is hydrogen and the other one is azide.

In some aspects, —X—Y— is —O—CH₂—, W is oxygen and R¹, R², R³, R⁵ and R^{5*} are all hydrogen at the same time. Such LNA nucleosides are disclosed in WO 99/014226, WO 00/66604, WO 98/039352 and WO 2004/046160, which are all hereby incorporated by reference, and include what are commonly known in the art as beta-D-oxy LNA and alpha-L-oxy LNA nucleosides.

In some aspects, —X—Y— is —S—CH₂—, W is oxygen and R¹, R², R³, R⁵ and R^{5*} are all hydrogen at the same time. Such thio LNA nucleosides are disclosed in WO 99/014226 and WO 2004/046160 which are hereby incorporated by reference.

In some aspects, —X—Y— is —NH—CH₂—, W is oxygen and R¹, R², R³, R⁵ and R^{5*} are all hydrogen at the same time. Such amino LNA nucleosides are disclosed in WO 99/014226 and WO 2004/046160, which are hereby incorporated by reference.

In some aspects, —X—Y— is —O—CH₂CH₂— or —OCH₂CH₂CH₂—, W is oxygen, and R¹, R², R³, R⁵ and R^{5*} are all hydrogen at the same time. Such LNA nucleosides are disclosed in WO 00/047599 and Morita et al., Bioorganic & Med. Chem. Lett. 12, 73-76, which are hereby incorporated by reference, and include what are commonly known in the art as 2′-O-4′C-ethylene bridged nucleic acids (ENA).

In some aspects, —X—Y— is —O—CH₂—, W is oxygen, R¹, R², R³ are all hydrogen at the same time, one of R⁵ and R^{5*} is hydrogen and the other one is not hydrogen, such as alkyl, for example methyl. Such 5′ substituted LNA nucleosides are disclosed in WO 2007/134181, which is hereby incorporated by reference.

In some aspects, —X—Y— is —O—CR^{a}R^{b}—, wherein one or both of R^{a} and R^{b} are not hydrogen, in particular alkyl such as methyl, W is oxygen, R¹, R², R³ are all hydrogen at the same time, one of R⁵ and R^{5*} is hydrogen and the other one is not hydrogen, in particular alkyl, for example methyl. Such bis modified LNA nucleosides are disclosed in WO 2010/077578, which is hereby incorporated by reference.

In some aspects, —X—Y— is —O—CH(CH₂—O—CH₃)— (“2′ O-methoxyethyl bicyclic nucleic acid”, Seth et al., J. Org. Chem. 2010, Vol 75(5) pp. 1569-81).

In some aspects, —X—Y— is —O—CHR^{a}—, W is oxygen and R¹, R², R³, R⁵ and R^{5*} are all hydrogen at the same time. Such 6′-substituted LNA nucleosides are disclosed in WO 2010/036698 and WO 2007/090071, which are both hereby incorporated by reference. In such 6′-substituted LNA nucleosides, R^{a} is in particular C1-C6 alkyl, such as methyl.

In some aspects, —X—Y— is —O—CH(CH₂—O—CH₃)—, W is oxygen and R¹, R², R³, R⁵ and R^{5*} are all hydrogen at the same time. Such LNA nucleosides are also known in the art as cyclic MOEs (cMOE) and are disclosed in WO 2007/090071.

In some aspects, —X—Y— is —O—CH(CH₃)—.

In some aspects, —X—Y— is —O—CH₂—O—CH₂— (Seth et al., J. Org. Chem 2010 op. cit.)

In some aspects, —X—Y— is —O—CH(CH₃)—, W is oxygen and R¹, R², R³, R⁵ and R^{5*} are all hydrogen at the same time. Such 6′-methyl LNA nucleosides are also known in the art as cET nucleosides, and may be either (S)-cET or (R)-cET diastereoisomers, as disclosed in WO 2007/090071 (beta-D) and WO 2010/036698 (alpha-L) which are both hereby incorporated by reference.

In some aspects, —X—Y— is —O—CR^{a}R^{b}—, wherein neither R^{a} nor R^{b} is hydrogen, W is oxygen, and R¹, R², R³, R⁵ and R^{5*} are all hydrogen at the same time. In certain aspects, R^{a} and R^{b} are both alkyl at the same time, in particular both methyl at the same time. Such 6′-di-substituted LNA nucleosides are disclosed in WO 2009/006478 which is hereby incorporated by reference.

In some aspects, —X—Y— is —S—CHR^{a}—, W is oxygen, and R¹, R², R³, R⁵ and R^{5*} are all hydrogen at the same time. Such 6′-substituted thio LNA nucleosides are disclosed in WO 2011/156202, which is hereby incorporated by reference. In certain aspects of such 6′-substituted thio LNA, R^{a} is alkyl, in particular methyl.

In some aspects, —X—Y— is —C(═CH₂)C(R^{a}R^{b})—, such as, W is oxygen, and R¹, R², R³, R⁵ and R^{5*} are all hydrogen at the same time. Such vinyl carbo LNA nucleosides are disclosed in WO 2008/154401 and WO 2009/067647, which are both hereby incorporated by reference.

In some aspects, —X—Y— is —N(OR^{a})—CH₂—, W is oxygen and R¹, R², R³, R⁵ and R^{5*} are all hydrogen at the same time. In some aspects, R^{a} is alkyl such as methyl. Such LNA nucleosides are also known as N substituted LNAs and are disclosed in WO 2008/150729, which is hereby incorporated by reference.

In some aspects, —X—Y— is —O—NCH₃— (Seth et al., J. Org. Chem 2010 op. cit.).

In some aspects, —X—Y— is ON(R^{a})—N(R^{a})—O—, —NR^{a}—CR^{a}R^{b}—CR^{a}R^{b}—, or —NR^{a}—CR^{a}R^{b}—, W is oxygen, and R¹, R², R³, R⁵ and R^{5*} are all hydrogen at the same time. In certain aspects, R^{a} is alkyl, such as methyl. (Seth et al., J. Org. Chem 2010 op. cit.).

In some aspects, R⁵ and R^{5*} are both hydrogen at the same time. In other aspects, one of R⁵ and R^{5*} is hydrogen and the other one is alkyl, such as methyl. In such aspects, R¹, R² and R³ can be in particular hydrogen and —X—Y— can be in particular —O—CH₂— or —O—CHC(R^{a})₃—, such as —O—CH(CH₃)—.

In some aspects, —X—Y— is —CR^{a}R^{b}—O—CR^{a}R^{b}—, such as —CH₂—O—CH₂—, W is oxygen and R¹, R², R³, R⁵ and R^{5*} are all hydrogen at the same time. In such aspects, R^{a} can be in particular alkyl such as methyl. Such LNA nucleosides are also known as conformationally restricted nucleotides (CRNs) and are disclosed in WO 2013/036868, which is hereby incorporated by reference.

In some aspects, —X—Y— is —O—CR^{a}R^{b}—O—CR^{a}R^{b}—, such as —O—CH₂—O—CH₂—, W is oxygen and R¹, R², R³, R⁵ and R^{5*} are all hydrogen at the same time. In certain aspects, R^{a} can be in particular alkyl such as methyl. Such LNA nucleosides are also known as COC nucleotides and are disclosed in Mitsuoka et al., Nucleic Acids Research 2009, 37(4), 1225-1238, which is hereby incorporated by reference.

It will be recognized than, unless specified, the LNA nucleosides may be in the beta-D or alpha-L stereoisoform.

Certain examples of LNA nucleosides are presented in Scheme 1.

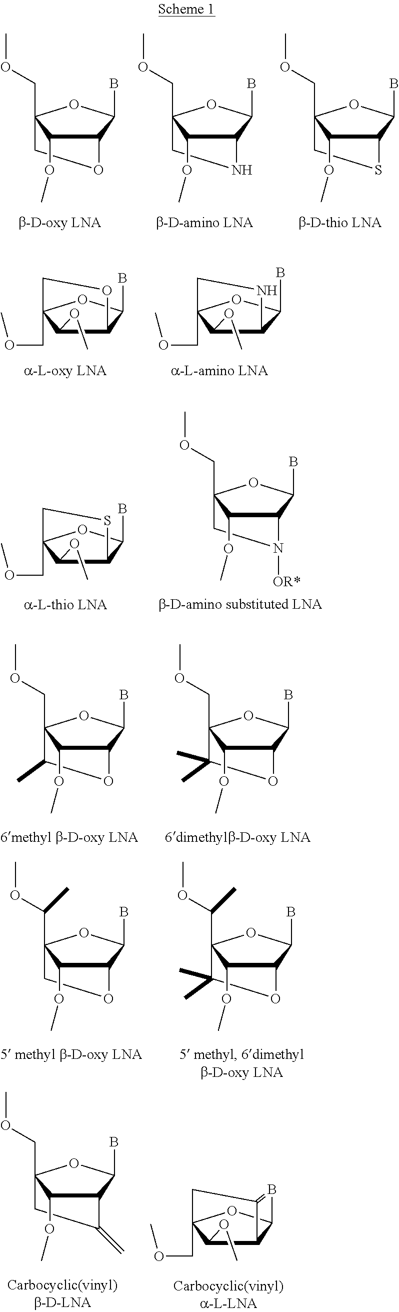

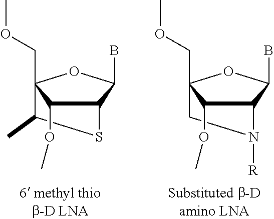

As illustrated elsewhere, in some aspects of the disclosure the LNA nucleosides in the oligonucleotides are beta-D-oxy-LNA nucleosides.

### III.E. Nuclease Mediated Degradation

Nuclease mediated degradation refers to an oligonucleotide capable of mediating degradation of a complementary nucleotide sequence when forming a duplex with such a sequence.

In some aspects, the oligonucleotide may function via nuclease mediated degradation of the target nucleic acid, where the oligonucleotides of the disclosure are capable of recruiting a nuclease, particularly and endonuclease, preferably endoribonuclease (RNase), such as RNase H. Examples of oligonucleotide designs which operate via nuclease mediated mechanisms are oligonucleotides which typically comprise a region of at least 5 or 6 DNA nucleosides and are flanked on one side or both sides by affinity enhancing nucleosides, for example gapmers.

### III.F. RNase H Activity and Recruitment

The RNase H activity of an antisense oligonucleotide refers to its ability to recruit RNase H when in a duplex with a complementary RNA molecule and induce degradation of the complementary RNA molecule. WO01/23613 provides in vitro methods for determining RNaseH activity, which may be used to determine the ability to recruit RNaseH. Typically, an oligonucleotide is deemed capable of recruiting RNase H if, when provided with a complementary target nucleic acid sequence, it has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers, with phosphorothioate linkages between all monomers in the oligonucleotide, and using the methodology provided by Example 91-95 of WO01/23613.

In some aspects, an oligonucleotide is deemed essentially incapable of recruiting RNaseH if, when provided with the complementary target nucleic acid, the RNaseH initial rate, as measured in pmol/l/min, is less than 20%, such as less than 10%,such as less than 5% of the initial rate determined when using a oligonucleotide having the same base sequence as the oligonucleotide being tested, but containing only DNA monomers, with no 2′ substitutions, with phosphorothioate linkages between all monomers in the oligonucleotide, and using the methodology provided by Example 91-95 of WO01/23613.

### III.G. ASO Design

The ASO of the disclosure can comprise a nucleotide sequence which comprises both nucleosides and nucleoside analogs, and can be in the form of a gapmer. Examples of configurations of a gapmer that can be used with the ASO of the disclosure are described in U.S. Patent Appl. Publ. No. 2012/0322851.

The term “gapmer” as used herein refers to an antisense oligonucleotide which comprises a region of RNase H recruiting oligonucleotides (gap) which is flanked 5′ and 3′ by one or more affinity enhancing modified nucleosides (flanks). The term “LNA gapmer” is a gapmer oligonucleotide wherein at least one of the affinity enhancing modified nucleosides is an LNA nucleoside. The term “mixed wing gapmer” refers to an LNA gapmer wherein the flank regions comprise at least one LNA nucleoside and at least one DNA nucleoside or non-LNA modified nucleoside, such as at least one 2′ substituted modified nucleoside, such as, for example, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, 2′-Fluro-DNA, arabino nucleic acid (ANA), and 2′-Fluoro-ANA nucleoside(s).

In some aspects, the ASO of the disclosure can be in the form of a mixmer. In some aspects, the ASO of the disclosure can be in the form of a totalmer. In some aspects, in addition to enhancing affinity of the ASO for the target region, some nucleoside analogs also mediate RNase (e.g., RNaseH) binding and cleavage. Since α-L-LNA monomers recruit RNaseH activity to a certain extent, in some aspects, gap regions (e.g., region B as referred to herein) of ASOs containing α-L-LNA monomers consist of fewer monomers recognizable and cleavable by the RNaseH, and more flexibility in the mixmer construction is introduced.

In some aspects, the ASO of the disclosure is a gapmer and comprises a contiguous stretch of nucleotides (e.g., one or more DNA) which is capable of recruiting an RNase, such as RNaseH, referred to herein in as region B (B), wherein region B is flanked at both 5′ and 3′ by regions of nucleoside analogs 5′ and 3′ to the contiguous stretch of nucleotides of region B—these regions are referred to as regions A (A) and C (C), respectively. In some aspects, the nucleoside analogs are sugar modified nucleosides (e.g., high affinity sugar modified nucleosides). In certain aspects, the sugar modified nucleosides of regions A and C enhance the affinity of the ASO for the target nucleic acid (i.e., affinity enhancing 2′ sugar modified nucleosides). In some aspects, the sugar modified nucleosides are 2′ sugar modified nucleosides, such as high affinity 2′ sugar modifications, such as LNA and/or 2′-MOE.

In a gapmer, the 5′ and 3′ most nucleosides of region B are DNA nucleosides, and are positioned adjacent to nucleoside analogs (e.g., high affinity sugar modified nucleosides) of regions A and C, respectively. In some aspects, regions A and C can be further defined by having nucleoside analogs at the end most distant from region B (i.e., at the 5′ end of region A and at the 3′ end of region C).

In some aspects, the ASOs of the present disclosure comprise a nucleotide sequence of formula (5′ to 3′) A-B-C, wherein: (A) (5′ region or a first wing sequence) comprises at least one nucleoside analog (e.g., 3-5 LNA units); (B) comprises at least four consecutive nucleosides (e.g., 4-24 DNA units), which are capable of recruiting RNase (when formed in a duplex with a complementary RNA molecule, such as the pre-mRNA or mRNA target); and (C) (3′ region or a second wing sequence) comprises at least one nucleoside analog (e.g., 3-5 LNA units).

In some aspects, region A comprises 3-5 nucleoside analogs, such as LNA, region B consists of 6-24 (e.g., 6, 7, 8, 9, 10, 11, 12, 13, or 14) DNA units, and region C consists of 3 or 4 nucleoside analogs, such as LNA. Such designs include (A-B-C) 3-14-3, 3-11-3, 3-12-3, 3-13-3, 4-9-4, 4-10-4, 4-11-4, 4-12-4, and 5-10-5. In some aspects, the ASO has a design of LLLDₙLLL, LLLLDₙLLLL, or LLLLLDₙLLLLL, wherein the L is a nucleoside analog, the D is DNA, and n can be any integer between 4 and 24. In some aspects, n can be any integer between 6 and 14. In some aspects, n can be any integer between 8 and 12. In some aspects, the ASO has a design of LLLMMDnMMLLL, LLLMDnMLLL, LLLLMMDnMMLLLL, LLLLMDnMLLLL, LLLLLLMMDnMMLLLLL, or LLLLLLMDnMLLLLL, wherein the D is DNA, n can be any integer between 3 and 15, the L is LNA, and the M is 2′MOE.

Further gapmer designs are disclosed in WO2004/046160, WO 2007/146511, and WO2008/113832, each of which is hereby incorporated by reference in its entirety.

### III.H. Internucleotide Linkages

The monomers of the ASOs described herein are coupled together via linkage groups. Suitably, each monomer is linked to the 3′ adjacent monomer via a linkage group.

The person having ordinary skill in the art would understand that, in the context of the present disclosure, the 5′ monomer at the end of an ASO does not comprise a 5′ linkage group, although it may or may not comprise a 5′ terminal group.

In some aspects, the contiguous nucleotide sequence comprises one or more modified internucleoside linkages. The terms “linkage group” or “internucleoside linkage” are intended to mean a group capable of covalently coupling together two nucleosides. Non-limiting examples include phosphate groups and phosphorothioate groups.

The nucleosides of the ASO of the disclosure or contiguous nucleosides sequence thereof are coupled together via linkage groups. Suitably, each nucleoside is linked to the 3′ adjacent nucleoside via a linkage group.

In some aspects, the internucleoside linkage is modified from its normal phosphodiester to one that is more resistant to nuclease attack, such as phosphorothioate, which is cleavable by RNaseH, also allows that route of antisense inhibition in reducing the expression of the target gene. In some aspects, at least 75%, at least 80%, at least 85%, at least 90%, at least 91%, at least 92%, at least 93%, at least 94%, at least 95%, at least 96%, at least 97%, at least 98%, at least 99%, or 100% of internucleoside linkages are modified.

### IV. Extracellular Vesicles, e.g., Exosomes

Disclosed herein are EVs, e.g., exosomes, comprising an ASO. The ASO can be any ASO described herein or a functional fragment thereof. In certain aspects, the ASO reduces the level of a STAT6 mRNA or a STAT6 protein in a target cell.

In some aspects, the EV, e.g., the exosome, targets a tumor cell, dendritic cell, T cell, B cell, macrophage, monocyte, neuron, hepatocyte, Kupffer cell, myeloid-lineage cell (e.g., a neutrophil, myeloid-derived suppressor cell (MDSC, e.g., a monocytic MDSC or a granulocytic MDSC), monocyte, macrophage, hematopoietic stem cell, basophil, neutrophil, or eosinophil), or any combination thereof. In some aspects, the EV, e.g., the exosome, targets a myeloid-lineage cell. In some aspects, the EV, e.g., the exosome, targets a macrophage. In certain aspects, the EV, e.g., the exosome, targets the liver, heart, lungs, brain, kidneys, central nervous system, peripheral nervous system, muscle, bone, joint, skin, intestine, bladder, pancreas, lymph nodes, spleen, blood, bone marrow, or any combination thereof.

In some aspects, the EV, e.g., the exosome, reduces the expression of one or more gene that is upregulated by the STAT6. In some aspects, the EV, e.g., the exosome, promotes differentiation of M2 macrophages. In some aspects, the EV, e.g., the exosome, reduces differentiation of M1 macrophages.

As described supra, EVs, e.g., exosomes, described herein are extracellular vesicles with a diameter between about 20-300 nm (e.g., between 40-200 nm). The size of the EV, e.g., exosome, described herein can be measured according to methods described, infra.

In some aspects, an EV, e.g., exosome, of the present disclosure comprises a bi-lipid membrane (“EV, e.g., exosome, membrane”), comprising an interior (luminal) surface and an exterior surface. In certain aspects, the interior (luminal) surface faces the inner core (i.e., lumen) of the EV, e.g., exosome. In certain aspects, the exterior surface can be in contact with the endosome, the multivesicular bodies, or the membrane/cytoplasm of a producer cell or a target cell

In some aspects, the EV, e.g., exosome, membrane comprises lipids and fatty acids. In some aspects, the EV, e.g., exosome, membrane comprises phospholipids, glycolipids, fatty acids, sphingolipids, phosphoglycerides, sterols, cholesterols, and phosphatidylserines.

In some aspects, the EV, e.g., exosome, membrane comprises an inner leaflet and an outer leaflet. The composition of the inner and outer leaflet can be determined by transbilayer distribution assays known in the art, see, e.g., Kuypers et al., Biohim Biophys Acta 1985 819:170. In some aspects, the composition of the outer leaflet is between approximately 70-90% choline phospholipids, between approximately 0-15% acidic phospholipids, and between approximately 5-30% phosphatidylethanolamine. In some aspects, the composition of the inner leaflet is between approximately 15-40% choline phospholipids, between approximately 10-50% acidic phospholipids, and between approximately 30-60% phosphatidylethanolamine.

In some aspects, the EV, e.g., exosome, membrane comprises one or more polysaccharide, such as glycan.

In some aspects, the EV, e.g., exosome, of the present disclosure comprises an ASO, wherein the ASO is linked to the EV via a scaffold moiety, either on the exterior surface of the EV or on the luminal surface of the EV.

In some aspects, the EV, e.g., exosome, comprising an ASO comprises an anchoring moiety, which optionally comprising a linker, between the ASO and the exosome membrane. Non-limiting examples of the linkers are disclosed elsewhere herein.

### IV.A. Anchoring Moieties

One or more anchoring moieties (AMs) can be used to anchor an ASO to the EV of the present disclosure. In some aspects, the ASO is linked directly to the anchoring moiety or via a linker. In some aspects, the ASO can be attached to an anchoring moiety or linker combination via reaction between a “reactive group” (RG; e.g., amine, thiol, hydroxy, carboxylic acid, or azide) with a “reactive moiety” (RM; e.g., maleimide, succinate, NHS). Several potential synthetic routes are envisioned, for example:
[AM]-/Reactive moiety/+/Reactive group/-[ASO]
[AM]-[Linker]n-/Reactive moiety/+/Reactive group/-[ASO]
[AM]-/Reactive moiety/+/Reactive group/-[Linker]n-[ASO]
[AM]-[Linker]n-/Reactive moiety/+/Reactive group/-[Linker]n-[ASO]

The anchoring moiety can insert into the lipid bilayer of an EV, e.g., an exosome, allowing the loading of the exosome with an ASO. Currently, a predominant obstacle to the commercialization of exosomes as a delivery vehicle for polar ASOs, is highly inefficient loading. This obstacle can be overcome by modifying polar ASOs, prior to loading them into exosomes. Thus, as described herein, modification of ASOs facilitates their loading into exosomes.

The methods of loading exosomes with modified polar ASOs set forth herein significantly improve loading efficiency as compared to the loading efficiency previously reported for introducing unmodified ASOs into exosomes by, for example, electroporation or cationic lipid transfection.

In some aspects, the modifications increase the hydrophobicity of the an ASO by at least about 1, at least about 2, at least about 3, at least about 4, at least about 5, at least about 6, at least about 7, at least about 8, at least about 9, or at least about 10 fold relative to native (non-modified) ASO. In some aspects, the modifications increase the hydrophobicity of the ASO by at least about 1, at least about 2, at least about 3, at least about 4, at least about 5, at least about 6, at least about 7, at least about 8, at least about 9, or at least about 10 orders of magnitude relative to native (non-modified) ASO.

In some aspects, the modifications increase the hydrophobicity of the ASO by at least about 10%, at least about 20%, at least about 30%, at least about 40%, at least about 50%, at least about 60%, at least about 70%, at least about 80%, at least about 90%, at least about 100%, at least about 125%, at least about 150%, at least about 175%, at least about 200%, at least about 250%, at least about 300%, at least about 350%, at least about 400%, at least about 450%, at least about 500%, at least about 600%, at least about 700%, at least about 800%, at least about 900%, or at least about 1000% relative to native (non-modified) ASO, e.g., the corresponding unmodified ASO. Increases in hydrophobicity can be assessed using any suitable method. For example, hydrophobicity can be determined by measuring the percentage solubility in an organic solvent, such as octanol, as compared to solubility in an aqueous solvent, such as water.

In some aspect, an anchoring moiety can be chemically conjugated to an ASO to enhance its hydrophobic character. In exemplary aspects, the anchoring moiety is a sterol (e.g., cholesterol), GM1, a lipid, a vitamin, a small molecule, a peptide, or a combination thereof. In some aspects, the moiety is a lipid. In some aspects, the anchoring moiety is a sterol, e.g., cholesterol. Additional hydrophobic moieties include, for example, phospholipids, lysophospholipids, fatty acids, or vitamins (e.g., vitamin D or vitamin E).

In some aspects, the anchoring moiety is conjugated at the termini of the ASO either directly or via one or more linkers (i.e., “terminal modification”). In other aspects, the anchoring moiety is conjugated to other portions of the ASO.

In some aspects, the ASO can include a detectable label. Exemplary labels include fluorescent labels and/or radioactive labels. In some aspects, where ASOs are fluorescently labeled, the detectable label can be, for example, Cy3. Adding a detectable label to ASOs can be used as a way of labeling exosomes, and following their biodistribution. In other aspects, a detectable label can be attached to exosomes directly, for example, by way of labeling an exosomal lipid and/or an exosomal peptide.

The different components of an ASO (i.e., anchoring moieties, linkers and linker combinations, and ASOs) can be linked by amide, ester, ether, thioether, disulfide, phosphoramidate, phosphotriester, phosphorodithioate, methyl phosphonate, phosphodiester, or phosphorothioate linkages or, alternatively any or other linkage.

In some aspects, the different components of an ASO can be linker using bifunctional linkers (i.e., linkers containing two functional groups), such as N-succinimidyl-3-(2-pyridyldithio)propionate, N-4-maleimide butyric acid, S-(2-pyridyldithio)cystamine, iodoacetoxysuccinimide, N-(4-maleimidebutyloxy) succinimide, N-[5-(3′-maleimide propylamide)-1-carboxypentyl]iminodiacetic acid, N-(5-aminopentyl)-iminodiacetic acid, and the like.

Suitable anchoring moieties capable of anchoring an ASO to the surface of an EV, e.g., an exosome, comprise for example sterols (e.g., cholesterol), lipids, lysophospholipids, fatty acids, or fat-soluble vitamins, as described in detail below.

In some aspects, the anchoring moiety can be a lipid. A lipid anchoring moiety can be any lipid known in the art, e.g., palmitic acid or glycosylphosphatidylinositols. In some aspects, the lipid, is a fatty acid, phosphatide, phospholipid (e.g., phosphatidyl choline, phosphatidyl serine, or phosphatidyl ethanolamine), or analogue thereof (e.g. phophatidylcholine, lecithin, phosphatidylethanolamine, cephalin, or phosphatidylserine or analogue or portion thereof, such as a partially hydrolyzed portion thereof).

Generally, anchoring moieties are chemically attached. However, an anchoring moiety can be attached to an ASO enzymatically. In some aspects, in the possible to attach an anchoring moiety to an ASO via modification of cell culture conditions. For example, by using a culture medium where myristic acid is limiting, some other fatty acids including shorter-chain and unsaturated, can be attached to an N-terminal glycine. For example, in BK channels, myristate has been reported to be attached posttranslationally to internal serine/threonine or tyrosine residues via a hydroxyester linkage.

The anchoring moiety can be conjugated to an ASO directly or indirectly via a linker combination, at any chemically feasible location, e.g., at the 5′ and/or 3′ end of the ASO. In one aspect, the anchoring moiety is conjugated only to the 3′ end of the ASO. In one aspect, the anchoring moiety is conjugated only to the 5′ end of the ASO. In one aspect, the anchoring moiety is conjugated at a location which is not the 3′ end or 5′ end of the ASO.

Some types of membrane anchors that can be used to practice the methods of the present disclosure presented in the following table:

| Modification | Modifying Group |
|---|---|
| S-Palmitoylation | 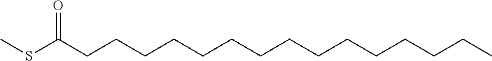 [SMILES: CCCCCCCCCCCCCCCC(=O)SC] |
| N-Palmitoylation | 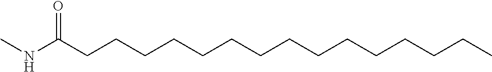 [SMILES: CCCCCCCCCCCCCCCC(=O)NC] |
| N-Myristoylation | 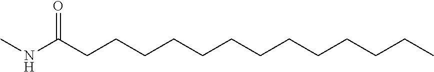 [SMILES: CCCCCCCCCCCCCC(=O)NC] |
| O-Acylation | 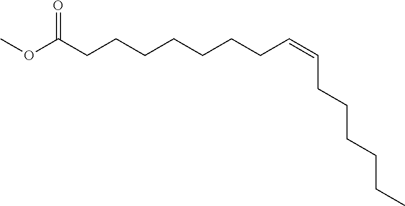 [SMILES: CCCCCC/C=C\CCCCCCCC(=O)OC] |
| Farnesylation | 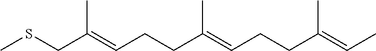 [SMILES: C/C=C(\C)CC/C=C(\C)CC/C=C(\C)CSC] |
| Geranylgeranylation | 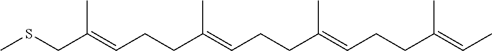 [SMILES: C/C=C(\C)CC/C=C(\C)CC/C=C(\C)CC/C=C(\C)CSC] |
| Cholesterol | 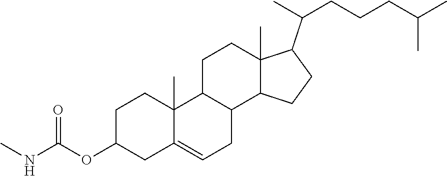 [SMILES: CNC(=O)OC1CCC2(C)C(=CCC3C2CCC2(C)C(C(C)CCCC(C)C)CCC32)C1] |

In some aspects, an anchoring moiety of the present disclosure can comprise two or more types of anchoring moieties disclosed herein. For example, in some aspects, an anchoring moiety can comprise two lipids, e.g., a phospholipids and a fatty acid, or two phospholipids, or two fatty acids, or a lipid and a vitamin, or cholesterol and a vitamin, etc. which taken together have 6-80 carbon atoms (i.e., an equivalent carbon number (ECN) of 6-80).

### IV.A.1. Cholesterol and Other Sterols

In some aspects, the anchoring moiety comprises a sterol, steroid, hopanoid, hydroxysteroid, secosteroid, or analog thereof with lipophilic properties. In some aspects, the anchoring moiety comprises a sterol, such as a phytosterol, mycosterol, or zoosterol. Exemplary zoosterols include cholesterol and 24S-hydroxycholesterol; exemplary phytosterols include ergosterol (mycosterol), campesterol, sitosterol, and stigmasterol. In some aspects, the sterol is selected from ergosterol, 7-dehydrocholesterol, cholesterol, 24S-hydroxycholesterol, lanosterol, cycloartenol, fucosterol, saringosterol, campesterol, β-sitosterol, sitostanol, coprostanol, avenasterol, or stigmasterol. Sterols may be found either as free sterols, acylated (sterol esters), alkylated (stearyl alkyl ethers), sulfated (sterol sulfate), or linked to a glycoside moiety (stearyl glycosides), which can be itself acylated (acylated sterol glycosides).

In some aspects, the anchoring moiety comprises a steroid. In some aspects, the steroid is selected from dihydrotestosterone, uvaol, hecigenin, diosgenin, progesterone, or cortisol.

For example, sterols may be conjugated to the ASO directly or via a linker combination at the available —OH group of the sterol. Exemplary sterols have the general skeleton shown below:

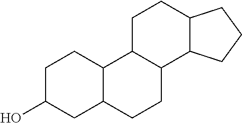 [SMILES: OC1CCC2C(CCC3C4CCCC4CCC23)C1]

As a further example, ergosterol has the structure below:

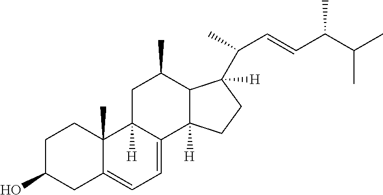

Cholesterol has the structure below:

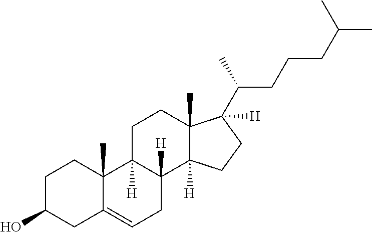

Accordingly, in some embodiments, the free —OH group of a sterol or steroid is used to conjugate the ASO directly or via a linker combination, to the sterol (e.g., cholesterol) or steroid.

### IV.A.2. Fatty Acids

In some aspects, the anchoring moiety is a fatty acid. In some aspects, the fatty acid is a short-chain, medium-chain, or long-chain fatty acid. In some aspects, the fatty acid is a saturated fatty acid. In some aspects, the fatty acid is an unsaturated fatty acid. In some aspects, the fatty acid is a monounsaturated fatty acid. In some aspects, the fatty acid is a polyunsaturated fatty acid, such as an omega-3 or omega-6 fatty acid.

In some aspects, the anchoring moiety comprises two fatty acids, each of which is independently selected from a fatty acid having a chain with any one of the foregoing ranges or numbers of carbon atoms. In some aspects, one of the fatty acids is independently a fatty acid with a C6-C21 chain and one is independently a fatty acid with a C12-C36 chain. In some embodiments, each fatty acid independently has a chain of 11, 12, 13, 14, 15, 16, or 17 carbon atoms.

Suitable fatty acids include saturated straight-chain fatty acids, saturated branched fatty acids, unsaturated fatty acids, hydroxy fatty acids, and polycarboxylic acids. In some aspects, such fatty acids have up to 32 carbon atoms.

### IV.A.3.Phospholipids

In some aspects, the anchoring moiety comprises a phospholipid. Phospholipids are a class of lipids that are a major component of all cell membranes. They can form lipid bilayers because of their amphiphilic characteristic. The structure of the phospholipid molecule generally consists of two hydrophobic fatty acid “tails” and a hydrophilic “head” consisting of a phosphate group. For example, a phospholipid can be a lipid according to the following formula:

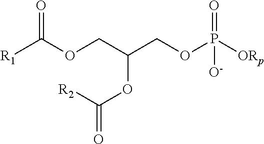

in which Rₚ represents a phospholipid moiety and R₁ and R₂ represent fatty acid moieties with or without unsaturation that may be the same or different.

A phospholipid moiety may be selected, for example, from the non-limiting group consisting of phosphatidyl choline, phosphatidyl ethanolamine, phosphatidyl glycerol, phosphatidyl serine, phosphatidic acid, 2 lysophosphatidyl choline, and a sphingomyelin.

### IV.A.4. Lysolipids (e.g., Lysophospholipids)

In some aspects, the anchoring moiety comprises a lysolipid, e.g., a lysophospholipid. Lysolipids are derivatives of a lipid in which one or both fatty acyl chains have been removed, generally by hydrolysis. Lysophospholipids are derivatives of a phospholipid in which one or both fatty acyl chains have been removed by hydrolysis.

In some aspects, the anchoring moiety comprises any of the phospholipids disclosed above, in which one or both acyl chains have been removed via hydrolysis, and therefore the resulting lysophospholipid comprises one or no fatty acid acyl chain.

In some aspects, the anchoring moiety comprises a lysoglycerophospholipid, a lysoglycosphingoliopid, a lysophosphatidylcholine, a lysophosphatidylethanolamine, a lysophosphatidylinositol, or a lysophosphatidylserine.

### IV.A. 5. Vitamins

In some aspects, the anchoring moiety comprises a lipophilic vitamin, e.g., folic acid, vitamin A, vitamin E, or vitamin K

In some aspects, the anchoring moiety comprises vitamin A. Vitamin A is a group of unsaturated nutritional organic compounds that includes retinol, retinal, retinoic acid, and several provitamin A carotenoids (most notably beta-carotene). In some aspects, the anchoring moiety comprises retinol. In some aspects, the anchoring moiety comprises a retinoid. Retinoids are a class of chemical compounds that are vitamers of vitamin A or are chemically related to it. In some aspects, the anchoring moiety comprises a first generation retinoid (e.g., retinol, tretinoin, isotretinoin, or alitretinoin), a second-generation retinoid (e.g., etretinate or acitretin), a third-generation retinoid (e.g., adapalene, bexarotene, or tazarotene), or any combination thereof.

### IV.B. Linker Combinations

In some aspects, an ASO is linked to a hydrophobic membrane anchoring moiety disclosed herein via a linker combination, which can comprise any combination of cleavable and/or non-cleavable linkers. The main function of a linker combination is to provide the optimal spacing between the anchoring moiety or moieties and the BAM target. For example, in the case of an ASO, the linker combination should reduce steric hindrances and position the ASO so it can interact with a target nucleic acid, e.g., a mRNA or a miRNA.

Linkers may be susceptible to cleavage (“cleavable linker”) thereby facilitating release of the biologically active molecule. Thus, in some aspects, a linker combination disclosed herein can comprise a cleavable linker. Such cleavable linkers may be susceptible, for example, to acid-induced cleavage, photo-induced cleavage, peptidase-induced cleavage, esterase-induced cleavage, and disulfide bond cleavage, at conditions under which the biologically active molecule remains active. Alternatively, linkers may be substantially resistant to cleavage (“non-cleavable linker”). In some aspects, the cleavable linker comprises a spacer. In some aspects the spacer is PEG.

In some aspects, a linker combination comprises at least 2, at least 3, at least 4, at least 5, or at least 6 or more different linkers disclosed herein. In some aspects, linkers in a linker combination can be linked by an ester linkage (e.g., phosphodiester or phosphorothioate ester).

In some aspects, the linker is direct bond between an anchoring moiety and a BAM, e.g., an ASO.

### IV.B.1.Non-Cleavable Linkers

In some aspects, the linker combination comprises a “non-cleavable liker.” Non-cleavable linkers are any chemical moiety capable of linking two or more components of a modified biologically active molecule of the present disclosure (e.g., a biologically active molecule and an anchoring moiety; a biologically active molecule and a cleavable linker; an anchoring moiety and a cleavable linker) in a stable, covalent manner and does not fall off under the categories listed above for cleavable linkers. Thus, non-cleavable linkers are substantially resistant to acid-induced cleavage, photo-induced cleavage, peptidase-induced cleavage, esterase-induced cleavage and disulfide bond cleavage.

Furthermore, non-cleavable refers to the ability of the chemical bond in the linker or adjoining to the linker to withstand cleavage induced by an acid, photolabile-cleaving agent, a peptidase, an esterase, or a chemical or physiological compound that cleaves a disulfide bond, at conditions under which a cyclic dinucleotide and/or the antibody does not lose its activity. In some aspects, the biologically active molecule is attached to the linker via another linker, e.g., a self-immolative linker.

In some aspects, the linker combination comprises a non-cleavable linker comprising, e.g., tetraethylene glycol (TEG), hexaethylene glycol (HEG), polyethylene glycol (PEG), succinimide, or any combination thereof. In some aspects, the non-cleavable linker comprises a spacer unit to link the biologically active molecule to the non-cleavable linker.

In some aspects, one or more non-cleavable linkers comprise smaller units (e.g., HEG, TEG, glycerol, C2 to C12 alkyl, and the like) linked together. In one aspect, the linkage is an ester linkage (e.g., phosphodiester or phosphorothioate ester) or other linkage.

IV.B.1.a. Ethylene Glycols (HEG, TEG, PEG)

In some aspects, the linker combination comprises a non-cleavable linker, wherein the non-cleavable linker comprises a polyethylene glycol (PEG) characterized by a formula R³—(O—CH₂—CH₂)ₙ— or R³—(O—CH₂—CH₂)ₙ—O— with R³ being hydrogen, methyl or ethyl and n having a value from 2 to 200. In some aspects, the linker comprises a spacer, wherein the spacer is PEG.

In some aspects, the PEG linker is an oligo-ethylene glycol, e.g., diethylene glycol, triethylene glycol, tetra ethylene glycol (TEG), pentaethylene glycol, or a hexaethylene glycol (HEG) linker.

In some aspects, n has a value of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 189, 181, 182, 183, 184, 185, 186, 187, 188, 189, 190, 191, 192, 193, 194, 195, 196, 197, 198, 199, or 200.

In some aspects, n is between 2 and 10, between 10 and 20, between 20 and 30, between 30 and 40, between 40 and 50, between 50 and 60, between 60 and 70, between 70 and 80, between 80 and 90, between 90 and 100, between 100 and 110, between 110 and 120, between 120 and 130, between 130 and 140, between 140 and 150, between 150 and 160, between 160 and 170, between 170 and 180, between 180 and 190, or between 190 and 200.

In some specific aspects, n has a value from 3 to 200, from 3 to 20, from 10 to 30, or from 9 to 45.

In some aspects, the PEG is a branched PEG. Branched PEGs have three to ten PEG chains emanating from a central core group.

In certain embodiments, the PEG moiety is a monodisperse polyethylene glycol. In the context of the present disclosure, a monodisperse polyethylene glycol (mdPEG) is a PEG that has a single, defined chain length and molecular weight. mdPEGs are typically generated by separation from the polymerization mixture by chromatography. In certain formulae, a monodisperse PEG moiety is assigned the abbreviation mdPEG.

In some aspects, the PEG is a Star PEG. Star PEGs have 10 to 100 PEG chains emanating from a central core group.

In some aspects, the PEG is a Comb PEGs. Comb PEGs have multiple PEG chains normally grafted onto a polymer backbone.

In certain aspects, the PEG has a molar mass between 100 g/mol and 3000 g/mol, particularly between 100 g/mol and 2500 g/mol, more particularly of approx. 100 g/mol to 2000 g/mol. In certain aspects, the PEG has a molar mass between 200 g/mol and 3000 g/mol, particularly between 300 g/mol and 2500 g/mol, more particularly of approx. 400 g/mol to 2000 g/mol.

In some aspects, the PEG is PEG₁₀₀, PEG₂₀₀, PEG₃₀₀, PEG₄₀₀, PEG₅₀₀, PEG₆₀₀, PEG₇₀₀, PEG₈₀₀, PEG₉₀₀, PEG₁₀₀₀, PEG₁₁₀₀, PEG₁₂₀₀, PEG₁₃₀₀, PEG₁₄₀₀, PEG₁₅₀₀, PEG₁₆₀₀, PEG₁₇₀₀, PEG₁₈₀₀, PEG₁₉₀₀, PEG₂₀₀₀, PEG₂₁₀₀, PEG₂₂₀₀, PEG₂₃₀₀, PEG₂₄₀₀, PEG₂₅₀₀, PEG₁₆₀₀, PEG₁₇₀₀, PEG₁₈₀₀, PEG₁₉₀₀, PEG₂₀₀₀, PEG₂₁₀₀, PEG₂₂₀₀, PEG₂₃₀₀, PEG₂₄₀₀, PEG₂₅₀₀, PEG₂₆₀₀, PEG₂₇₀₀, PEG₂₈₀₀, PEG₂₉₀₀, or PEG₃₀₀₀. In one particular aspect, the PEG is PEG₄₀₀. In another particular aspect, the PEG is PEG₂₀₀₀.

In some aspects, a linker combination of the present disclosure can comprise several PEG linkers, e.g., a cleavable linker flanked by PEG, HEG, or TEG linkers.

In some aspects, the linker combination comprises (HEG)n and/or (TEG)n, wherein n is an integer between 1 and 50, and each unit is connected, e.g., via a phosphate ester linker, a phosphorothioate ester linkage, or a combination thereof.

IV.B.1.b. Glycerol and Polyglycerols (PG)

In some aspects, the linker combination comprises a non-cleavable linker comprising a glycerol unit or a polyglycerol (PG) described by the formula ((R³—O—(CH₂—CHOH—CH₂O)ₙ—) with R3 being hydrogen, methyl or ethyl, and n having a value from 3 to 200. In some aspects, n has a value from 3 to 20. In some aspects, n has a value from 10 to 30.

In some aspects, the PG linker is a digylcerol, triglycerol, tetraglycerol (TG), pentaglycerol, or a hexaglycerol (HG) linker.

In some aspects, the linker combination comprises (glycerol)n, and/or (HG)n and/or (TG)n, wherein n is an integer between 1 and 50, and each unit is connected, e.g., via a phosphate ester linker, a phosphorothioate ester linkage, or a combination thereof.

In some aspects, the linker combination comprises at least one aliphatic (alkyl) linker, e.g., propyl, butyl, hexyl, or C2-C12 alkyl, such as C2-C10 alkyl or C2-C₆ alkyl.

### IV.B.2. Cleavable Linkers

In some aspects, different components of an ASO disclosed herein can be linker by a cleavable linker. The term cleavable linker refers to a linker comprising at least one linkage or chemical bond that can be broken or cleaved. As used herein, the term cleave refers to the breaking of one or more chemical bonds in a relatively large molecule in a manner that produces two or more relatively smaller molecules. Cleavage may be mediated, e.g., by a nuclease, peptidase, protease, phosphatase, oxidase, or reductase, for example, or by specific physicochemical conditions, e.g., redox environment, pH, presence of reactive oxygen species, or specific wavelengths of light.

In some aspects, the term “cleavable,” as used herein, refers, e.g., to rapidly degradable linkers, such as, e.g., phosphodiester and disulfides, while the term “non-cleavable” refers, e.g., to more stable linkages, such as, e.g., nuclease-resistant phosphorothioates.

In some aspects, the cleavable linker is a dinucleotide or trinucleotide linker, a disulfide, an imine, a thioketal, a val-cit dipeptide, or any combination thereof.

In some aspects, the cleavable linker comprises valine-alanine-p-aminobenzylcarbamate or valine-citrulline-p-aminobenzylcarbamate.

IV.B.2.a. Redox Cleavable Linkers

In some aspects, the linker combination comprises a redox cleavable linker. As a non-limiting example, one type of cleavable linker is a redox cleavable linking group that is cleaved upon reduction or upon oxidation.

In some aspects, the redox cleavable linker contains a disulfide bond, i.e., it is a disulfide cleavable linker.

Redox cleavable linkers can be reduced, e.g., by intracellular mercaptans, oxidases, or reductases.

IV.B.2.b. Reactive Oxygen Species (ROS) Cleavable Linkers

In some aspects, the linker combination can comprise a cleavable linker which may be cleaved by a reactive oxygen species (ROS), such as superoxide (Of) or hydrogen peroxide (H2O2), generated, e.g., by inflammation processes such as activated neutrophils. In some aspects, the ROS cleavable linker is a thioketal cleavable linker. See, e.g., U.S. Pat. No. 8,354,455B2, which is herein incorporated by reference in its entirety.

IV.B.2.c. pH Dependent Cleavable Linkers

In some aspects, the linker is an “acid labile linker” comprising an acid cleavable linking group, which is a linking group that is selectively cleaved under acidic conditions (pH<7).

As a non-limiting example, the acid cleavable linking group is cleaved in an acidic environment, e.g., about 6.0, 5.5, 5.0 or less. In some aspects, the pH is about 6.5 or less. In some aspects, the linker is cleaved by an agent such as an enzyme that can act as a general acid, e.g., a peptidase (which may be substrate specific) or a phosphatase. Within cells, certain low pH organelles, such as endosomes and lysosomes, can provide a cleaving environment to the acid cleavable linking group. Although the pH of human serum is 7.4, the average pH in cells is slightly lower, ranging from about 7.1 to 7.3. Endosomes also have an acidic pH, ranging from 5.5 to 6.0, and lysosomes are about 5.0 at an even more acidic pH. Accordingly, pH dependent cleavable linkers are sometimes called endosomically labile linkers in the art.

Further examples may be found in U.S. Pat. Nos. 9,790,494B2 and 8,137,695B2, the contents of which are incorporated herein by reference in their entireties.

IV.B.2.d. Enzymatic Cleavable Linkers

In some aspects, the linker combination can comprise a linker cleavable by intracellular or extracellular enzymes, e.g., proteases, esterases, nucleases, amidades. The range of enzymes that can cleave a specific linker in a linker combination depends on the specific bonds and chemical structure of the linker. Accordingly, peptidic linkers can be cleaved, e.g., by peptidases, linkers containing ester linkages can be cleaved, e.g., by esterases; linkers containing amide linkages can be cleaved, e.g., by amidades; etc.

IV.B.2.e. Protease Cleavable Linkers

In some aspects, the linker combination comprises a protease cleavable linker, i.e., a linker that can be cleaved by an endogenous protease. Only certain peptides are readily cleaved inside or outside cells. See, e.g., Trout et al., 79 Proc. Natl. Acad. Sci. USA, 626-629 (1982) and Umemoto et al. 43 Int. J. Cancer, 677-684 (1989). Cleavable linkers can contain cleavable sites composed of a-amino acid units and peptidic bonds, which chemically are amide bonds between the carboxylate of one amino acid and the amino group of a second amino acid. Other amide bonds, such as the bond between a carboxylate and the α-amino acid group of lysine, are understood not to be peptidic bonds and are considered non-cleavable.

IV.B.2.f. Esterase Cleavable Linkers

Some linkers are cleaved by esterases (“esterase cleavable linkers”). Only certain esters can be cleaved by esterases and amidases present inside or outside of cells. Esters are formed by the condensation of a carboxylic acid and an alcohol. Simple esters are esters produced with simple alcohols, such as aliphatic alcohols, and small cyclic and small aromatic alcohols. Examples of ester-based cleavable linking groups include, but are not limited to, esters of alkylene, alkenylene and alkynylene groups. The ester cleavable linking group has the general formula —C (O)O— or —OC(O)—.

IV.B.2.g. Phosphatase Cleavable Linkers

In some aspects, a linker combination can includes a phosphate-based cleavable linking group is cleaved by an agent that degrades or hydrolyzes phosphate groups. An example of an agent that cleaves intracellular phosphate groups is an enzyme such as intracellular phosphatase. Examples of phosphate-based linking groups are —O—P(O)(OR k)-O—, —O—P(S)(ORₖ)—O—, —O—P(S)(SRₖ)—O—, —S—P(O)(ORₖ)—O—, —O—P(O)(ORₖ)—S—, —S—P(O)(ORₖ)—S—, —O—P(S)(ORₖ)—S—, —SP(S)(ORₖ)—O—, —OP(O)(Rₖ)—O—, —OP(S)(Rₖ)—O—, —SP(O)(Rₖ)—O—, —SP(S)(Rₖ)—O—, —SP(O)(Rₖ)—S—, or —OP(S)(Rₖ)—S—.

In various aspects, Rₖ is any of the following: NH₂, BH₃, CH₃, C₁₋₆ alkyl, C₆₋₁₀ aryl, C₁₋₆ alkoxy and C₆₋₁₀ aryl-oxy. In some aspects, C₁₋₆ alkyl and C₆₋₁₀ aryl are unsubstituted. Further non-limiting examples are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —SP(S)(H)—O—, —SP(O)(H)—S—, —OP(S)(H)—S—, or —O—P(O)(OH)—O—.

IV.B.2.h. Photoactivated Cleavable Linkers

In some aspects, the combination linker comprises a photoactivated cleavable linker, e.g., a nitrobenzyl linker or a linker comprising a nitrobenzyl reactive group.

IV.B.2.i. Self-Immolative Linker

In some aspects, the linker combination comprises a self-immolative linker In some aspects, the self-immolative linker in the EV (e.g., exosome) of the present disclosure undergoes 1,4 elimination after the enzymatic cleavage of the protease-cleavable linker. In some aspects, the self-immolative linker in the EV (e.g., exosome) of the present disclosure undergoes 1,6 elimination after the enzymatic cleavage of the protease-cleavable linker. In some aspects, the self-immolative linker is, e.g., a p-aminobenzyl (pAB) derivative, such as a p-aminobenzyl carbamate (pABC), a p-amino benzyl ether (PABE), a p-amino benzyl carbonate, or a combination thereof.

In some aspects, the cleavable linker is valine-alanine-p-aminobenzylcarbamate or valine-citrulline-p-aminobenzylcarbamate.

### IV.B.3. Reactive Moieties (RM)

The ASOs of the present disclosure are generated either via chemical synthesis or via chemical reaction between their components. For example, in some aspects, an anchoring moiety comprising a reactive group (e.g., maleimide) can react with an ASO comprising a maleimide-reacting group, to yield a hydrophobically modified ASO of the present disclosure, where the anchoring moiety may insert into the lipid bilayer of the membrane of an exosome, thereby attaching the ASO to the surface of the exosome.

### IV.C. Scaffold Moieties

One or more scaffold moieties can be expressed in the EVs. In some aspects, one or more scaffold moieties are used to anchor an ASO to the EV of the present disclosure. In other aspects, one or more scaffold moieties are used to anchor a protein or a molecule to the EVs in addition to the ASOs. Therefore, an EV of the present disclosure comprises an anchoring moiety linking an ASO and a scaffold moiety linking a protein or a molecule, e.g., a targeting moiety. In some aspects, the ASO is linked to the scaffold moiety. In some aspects, the EV comprises more than one scaffold moiety. In some aspects, a first ASO is linked to a first scaffold moiety and a second ASO is linked to a second scaffold moiety. In some aspects, the first scaffold moiety and the second scaffold moiety are the same type of scaffold moiety, e.g., the first and second scaffold moieties are both a Scaffold X protein. In some aspects, the first scaffold moiety and the second scaffold moiety are different types of scaffold moiety, e.g., the first scaffold moiety is a Scaffold Y protein and the second scaffold moiety is a Scaffold X protein. In some aspects, the first scaffold moiety is a Scaffold Y, disclosed herein. In some aspects, the first scaffold moiety is a Scaffold X, disclosed herein. In some aspects, the second scaffold moiety is a Scaffold Y, disclosed herein. In some aspects, the second scaffold moiety is a Scaffold X, disclosed herein.

In some aspects, the EV comprises one or more scaffold moieties, which are capable of anchoring an ASO to the EV, e.g., exosome, (e.g., either on the luminal surface or on the exterior surface). In certain aspects, the scaffold moiety is a polypeptide (“scaffold protein”). In certain aspects, the scaffold protein comprises an exosome protein or a fragment thereof. In other aspects, scaffold moieties are non-polypeptide moieties. In some aspects, scaffold proteins include various membrane proteins, such as transmembrane proteins, integral proteins and peripheral proteins, enriched on the exosome membranes. They can include various CD proteins, transporters, integrins, lectins, and cadherins. In certain aspects, a scaffold moiety (e.g., scaffold protein) comprises Scaffold X. In other aspects, a scaffold moiety (e.g., exosome protein) comprises Scaffold Y. In further aspects, a scaffold moiety (e.g., exosome protein) comprises both a Scaffold X and a Scaffold Y.

In some aspects, EVs, e.g., exosomes, of the present disclosure comprise a membrane modified in its composition. For example, their membrane compositions can be modified by changing the protein, lipid, or glycan content of the membrane.

In some aspects, the surface-engineered EVs, e.g., exosomes, are generated by chemical and/or physical methods, such as PEG-induced fusion and/or ultrasonic fusion. In other aspects, the surface-engineered EVs, e.g., exosomes, are generated by genetic engineering. EVs, e.g., exosomes, produced from a genetically-modified producer cell or a progeny of the genetically-modified cell can contain modified membrane compositions. In some aspects, surface-engineered EVs, e.g., exosomes, have scaffold moiety (e.g., exosome protein, e.g., Scaffold X) at a higher or lower density (e.g., higher number) or include a variant or a fragment of the scaffold moiety.

For example, surface (e.g., Scaffold X)-engineered EVs, can be produced from a cell (e.g., HEK293 cells) transformed with an exogenous sequence encoding a scaffold moiety (e.g., exosome proteins, e.g., Scaffold X) or a variant or a fragment thereof. EVs including scaffold moiety expressed from the exogenous sequence can include modified membrane compositions.

Various modifications or fragments of the scaffold moiety can be used for the aspects of the present disclosure. For example, scaffold moiety modified to have enhanced affinity to a binding agent can be used for generating surface-engineered EV that can be purified using the binding agent. Scaffold moieties modified to be more effectively targeted to EVs and/or membranes can be used. Scaffold moieties modified to comprise a minimal fragment required for specific and effective targeting to exosome membranes can be also used.

Scaffold moieties can be engineered to be expressed as a fusion molecule, e.g., fusion molecule of Scaffold X to an ASO. For example, the fusion molecule can comprise a scaffold moiety disclosed herein (e.g., Scaffold X, e.g., PTGFRN, BSG, IGSF2, IGSF3, IGSF8, ITGB1, ITGA4, SLC3A2, ATP transporter, or a fragment or a variant thereof) linked to an ASO.

In some aspects, the Scaffold X comprises Prostaglandin F2 receptor negative regulator (the PTGFRN polypeptide). The PTGFRN protein can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor-associated protein, or CD315. The full length amino acid sequence of the human PTGFRN protein (Uniprot Accession No. Q9P2B2) is shown at Table 2 as SEQ ID NO: 301. The PTGFRN polypeptide contains a signal peptide (amino acids 1 to 25 of SEQ ID NO: 301), the extracellular domain (amino acids 26 to 832 of SEQ ID NO: 301), a transmembrane domain (amino acids 833 to 853 of SEQ ID NO: 301), and a cytoplasmic domain (amino acids 854 to 879 of SEQ ID NO: 301). The mature PTGFRN polypeptide consists of SEQ ID NO: 301 without the signal peptide, i.e., amino acids 26 to 879 of SEQ ID NO: 301.

TABLE 2
Exemplary Scaffold X Protein Sequences

| Protein | Sequence |
|---|---|
| The PTGFRN | MGRLASRPLLLALLSLALCRGRVVRVPTATLVRVVGTELVIPCNVSDYDGP |
| Protein | SEQNFDWSFSSLGSSFVELASTWEVGFPAQLYQERLQRGEILLRRTANDAV |
| (SEQ ID NO: | ELHIKNVQPSDQGHYKCSTPSTDATVQGNYEDTVQVKVLADSLHVGPSARP |
| 301) | PPSLSLREGEPFELRCTAASASPLHTHLALLWEVHRGPARRSVLALTHEGR |
| | FHPGLGYEQRYHSGDVRLDTVGSDAYRLSVSRALSADOGSYRCIVSEWIAE |
| | QGNWQEIQEKAVEVATVVIQPSVLRAAVPKNVSVAEGKELDLTCNITTDRA |
| | DDVRPEVTWSFSRMPDSTLPGSRVLARLDRDSLVHSSPHVALSHVDARSYH |
| | LLVRDVSKENSGYYYCHVSLWAPGHNRSWHKVAEAVSSPAGVGVTWLEPDY |
| | QVYLNASKVPGFADDPTELACRVVDTKSGEANVRFTVSWYYRMNRRSDNVV |
| | TSELLAVMDGDWTLKYGERSKQRAQDGDFIFSKEHTDTFNFRIQRTTEEDR |
| | GNYYCVVSAWTKQRNNSWVKSKDVFSKPVNIFWALEDSVLVVKARQPKPFF |
| | AAGNTFEMTCKVSSKNIKSPRYSVLIMAEKPVGDLSSPNETKYIISLDQDS |
| | VVKLENWTDASRVDGVVLEKVQEDEFRYRMYQTQVSDAGLYRCMVTAWSPV |
| | RGSLWREAATSLSNPIEIDFQTSGPIFNASVHSDTPSVIRGDLIKLFCIIT |
| | VEGAALDPDDMAFDVSWFAVHSFGLDKAPVLLSSLDRKGIVTTSRRDWKSD |
| | LSLERVSVLEFLLQVHGSEDQDFGNYYCSVTPWVKSPTGSWQKEAEIHSKP |
| | VFITVKMDVLNAFKYPLLIGVGLSTVIGLLSCLIGYCSSHWCCKKEVQETR |
| | RERRRLMSMEMD |
| The PTGFRN | GPIFNASVHSDTPSVIRGDLIKLFCIITVEGAALDPDDMAFDVSWFAVHSF |
| protein | GLDKAPVLLSSLDRKGIVTTSRRDWKSDLSLERVSVLEFLLQVHGSEDQDF |
| Fragment | GNYYCSVTPWVKSPTGSWQKEAEIHSKPVFITVKMDVLNAFKYPLLIGVGL |
| (SEQ ID NO: | STVIGLLSCLIGYCSSHWCCKKEVQETRRERRRLMSMEM |
| 302) | 687-878 of SEQ ID NO: 301 |

### IV.D. Targeting Moiety

In some aspects, the EV, e.g., exosome, comprises a targeting moiety, e.g., an exogenous targeting moiety. In some aspects, the exogenous targeting moiety comprises a peptide, an antibody or an antigen-binding fragment thereof, a chemical compound, an RNA aptamer, or any combination thereof. In some aspects, the targeting moiety comprises a microprotein, a designed ankyrin repeat protein (darpin), an anticalin, an adnectin, an aptamer, a peptide mimetic molecule, a natural ligand for a receptor, a camelid nanobody, or any combination thereof. In some aspects, the exogenous targeting moiety comprises a full-length antibody, a single domain antibody, a heavy chain only antibody (VHH), a single chain antibody, a shark heavy chain only antibody (VNAR), an scFv, a Fv, a Fab, a Fab′, a F(ab′)2, or any combination thereof. In some aspects, the antibody is a single chain antibody.

In some aspects, the targeting moiety targets the exosome to the liver, heart, lungs, brain, kidneys, central nervous system, peripheral nervous system, muscle, bone, joint, skin, intestine, bladder, pancreas, lymph nodes, spleen, blood, bone marrow, or any combination thereof. In some aspects, the targeting moiety targets the exosome to a tumor cell, dendritic cell, T cell, B cell, macrophage, neuron, hepatocyte, Kupffer cell, a myeloid-lineage cell (e.g., neutrophil, maonocyte, macrophage, or an MDSC (e.g., a monocytic MDSC or a granulocytic MDSC)), hematopoietic stem cell, or any combination thereof.

In some aspects, the targeting moiety is linked to the EV, e.g., the exosome, by a scaffold protein. In some aspects, the scaffold protein is any scaffold protein disclosed herein. In some aspects, the scaffold protein is a Scaffold X. In some aspects, the scaffold protein is a Scaffold Y.

### IV.E. Linkers

As described supra, extracellular vesicles (EVs) of the present disclosure (e.g., exosomes and nanovesicles) can comprises one or more linkers that link a molecule of interest (e.g., an ASO) to the EVs (e.g., to the exterior surface or on the luminal surface). In some aspects, an ASO is linked to the EVs directly or via a scaffold moiety (e.g., Scaffold X or Scaffold Y). In certain aspects, the ASO is linked to the scaffold moiety by a linker. In certain aspects, the ASO is linked to the second scaffold moiety by a linker.

In certain aspects, an ASO is linked to the exterior surface of an exosome via Scaffold X. In further aspects, an ASO is linked to the luminal surface of an exosome via Scaffold X or Scaffold Y. The linker can be any chemical moiety known in the art.

As used herein, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. When multiple linkers are present, each of the linkers can be the same or different. Generally, linkers provide flexibility or prevent/ameliorate steric hindrances. Linkers are not typically cleaved; however, in certain aspects, such cleavage can be desirable. Accordingly, in some aspects, a linker can comprise one or more protease-cleavable sites, which can be located within the sequence of the linker or flanking the linker at either end of the linker sequence.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids. v

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

Linkers can be susceptible to cleavage (“cleavable linker”) thereby facilitating release of the biologically active molecule (e.g., an ASO).

In some aspects, the linker is a “reduction-sensitive linker.” In some aspects, the reduction-sensitive linker contains a disulfide bond. In some aspects, the linker is an “acid labile linker.” In some aspects, the acid labile linker contains hydrazone. Suitable acid labile linkers also include, for example, a cis-aconitic linker, a hydrazide linker, a thiocarbamoyl linker, or any combination thereof.

In some aspects, the linker comprises a non-cleavable linker.

In some aspects, the linker comprises acrylic phosphoramidite (e.g., ACRYDITE™), adenylation, azide (NETS Ester), digoxigenin (NETS Ester), cholesterol-TEG, I-LINKER™, an amino modifier (e.g., amino modifier C6, amino modifier C12, amino modifier C6 dT, or Uni-Link™ amino modifier), alkyne, 5′ Hexynyl, 5-Octadiynyl dU, biotinylation (e.g., biotin, biotin (Azide), biotin dT, biotin-TEG, dual biotin, PC biotin, or desthiobiotin), thiol modification (thiol modifier C3 S—S, dithiol or thiol modifier C6 S—S), or any combination thereof.

In some aspects, the linker comprises a terpene such as nerolidol, farnesol, limonene, linalool, geraniol, carvone, fenchone, or menthol; a lipid such as palmitic acid or myristic acid; cholesterol; oleyl; retinyl; cholesteryl residues; cholic acid; adamantane acetic acid; 1-pyrene butyric acid; dihydrotestosterone; 1,3-Bis-O(hexadecyl)glycerol; geranyloxyhexyl group; hexadecylglycerol; borneol; 1,3-propanediol; heptadecyl group; O3-(oleoyl)lithocholic acid; O3-(oleoyl)cholenic acid; dimethoxytrityl; phenoxazine, a maleimide moiety, a glucorinidase type, a CL2A-SN38 type, folic acid; a carbohydrate; vitamin A; vitamin E; vitamin K, or any combination thereof.

### IV.F. Modified EVs Comprising Tropism Moieties

In some aspects, an EV, e.g., exosome, disclosed herein can be surface engineered to adjust its properties, e.g., biodistribution, e.g., via incorporation of immuno-affinity ligands or cognate receptor ligands. For example, EV, e.g., exosomes, disclosed herein can be surface engineered to direct them to a specific cellular type, e.g., Schwann cells, sensory neurons, motor neurons, meningeal macrophages, or a tumor cell, or can be surface engineered to enhance their migration to a specific compartment, e.g., to the CNS (in order to improve intrathecal compartment retention) or to a tumor microenvironment.

In some aspects, an EV, e.g., exosome, comprises (i) an ASO disclosed herein and (ii) a bio-distribution modifying agent or targeting moiety. In some aspects, the bio-distribution modifying agent or targeting moiety comprises a single-domain antigen-biding moiety, e.g., a VHH and/or a vNAR. As used here, the terms “bio-distribution modifying agent” and “targeting moiety” are used interchangeably and refer to an agent that can modify the distribution of extracellular vesicles (e.g., exosomes, nanovesicles) in vivo or in vitro (e.g., in a mixed culture of cells of different varieties). In some aspects, the targeting moiety alters the tropism of the EV (e.g., exosome), i.e., the target moiety is a “tropism moiety”. As used herein, the term “tropism moiety” refers to a targeting moiety that when expressed on an EV (e.g., exosome) alters and/or enhances the natural movement of the EV. For example, in some aspects, a tropism moiety can promote the EV (e.g., exosome) to be taken up by a particular cell, tissue, or organ.

EVs, e.g., exosomes, exhibit preferential uptake in discrete cell types and tissues, and their tropism can be directed by adding proteins to their surface that interact with receptors on the surface of target cells. The tropism moiety can comprise a biological molecule, such as a protein, a peptide, a lipid, or a carbohydrate, or a synthetic molecule. For example, in some aspects the tropism moiety can comprise an affinity ligand, e.g., an antibody (such as an anti-CD19 nanobody, an anti-CD22 nanobody, an anti-CLEC9A nanobody, or an anti-CD3 nanobody), a VHH domain, a phage display peptide, a fibronectin domain, a camelid nanobody, and/or a vNAR. In some aspects, the tropism moiety can comprise, e.g., a synthetic polymer (e.g., PEG), a natural ligand/molecule (e.g., CD40L, albumin, CD47, CD24, CD55, CD59), and/or a recombinant protein (e.g., XTEN).

### V. PD-1 Antagonists

Certain aspects of the present disclosure are directed to method of preventing or treating a disease or condition in a subject in need thereof, comprising administering to the subject (i) an extracellular vesicle comprising an ASO and (ii) a PD-1 antagonist. In some aspects, the PD-1 antagonist blocks or reduces PD-1 signaling. In certain aspects, the PD-1 antagonist inhibits the interaction between PD-1 and PD-L1. Any PD-1 antagonist can be used in the methods disclosed herein. In some aspects, the PD-1 antagonist comprises a polypeptide (e.g., an antibody, an antigen-binding portion of an antibody, a ligand, or any combination thereof), a small molecule, a nucleic acid molecule (e.g., a miRNA, an siRNA, an antisense oligonucleotide, or any combination thereof), or any combination thereof.

In certain aspects, the PD-1 antagonist comprises an antibody or an antigen-binding portion thereof that specifically binds PD-1, e.g., human PD-1. In certain aspects, the PD-1 antagonist comprises an antibody or an antigen-binding portion thereof that specifically binds PD-L1, e.g., human PD-L1. In certain aspects, the PD-1 antagonist comprises an antibody or an antigen-binding portion thereof that specifically binds PD-L2, e.g., human PD-L2. Ther are many therapeutic anti-PD-1 and anti-PD-L1 antibodies known in the art, any one or more of which is suitable for the methods disclosed herein. In certain aspects, the anti-PD-1 antibody comprises an antibody selected from nivolumab, pembrolizumab, PDR001, MEDI-0680, cemiplimab, JS001, BGB-A317, INCSHR1210, TSR-042, GLS-010, AM-0001, STI-1110, AGEN2034, MGA012, IBI308, and any combination thereof. In certain aspects, the anti-PD-1 antibody comprises nivolumab. In certain aspects, the anti-PD-1 antibody comprises pembrolizumab. In certain aspects, the anti-PD-1 antibody comprises PDR001. In certain aspects, the anti-PD-1 antibody comprises MEDI-0680. In certain aspects, the anti-PD-1 antibody comprises cemiplimab. In certain aspects, the anti-PD-1 antibody comprises JS001. In certain aspects, the anti-PD-1 antibody comprises BGB-A317. In certain aspects, the anti-PD-1 antibody comprises INCSHR1210. In certain aspects, the anti-PD-1 antibody comprises TSR-042. In certain aspects, the anti-PD-1 antibody comprises GLS-010. In certain aspects, the anti-PD-1 antibody comprises AM-0001. In certain aspects, the anti-PD-1 antibody comprises STI-1110. In certain aspects, the anti-PD-1 antibody comprises AGEN2034. In certain aspects, the anti-PD-1 antibody comprises MGA012. In certain aspects, the anti-PD-1 antibody comprises IBI308. In certain aspects, the anti-PD-1 antibody comprises a bispecific antibody comprising one or more of the anti-PD-1 antibodies listed herein.

In certain aspects, the anti-PD-L1 antibody comprises an antibody selected from atezolizumab, durvalumab, avelumab, STI-1014, CX-072, KN035, LY3300054, CK-301, BMS-936559, and any combination thereof. In certain aspects, the anti-PD-L1 antibody comprises atezolizumab. In certain aspects, the anti-PD-L1 antibody comprises durvalumab. In certain aspects, the anti-PD-L1 antibody comprises avelumab. In certain aspects, the anti-PD-L1 antibody comprises STI-1014. In certain aspects, the anti-PD-L1 antibody comprises CX-072. In certain aspects, the anti-PD-L1 antibody comprises KN035. In certain aspects, the anti-PD-L1 antibody comprises LY3300054. In certain aspects, the anti-PD-L1 antibody comprises CK-301. In certain aspects, the anti-PD-L1 antibody comprises BMS-936559. In certain aspects, the anti-PD-1 antibody comprises a bispecific antibody comprising one or more of the anti-PD-L1 antibodies listed herein.

In certain aspects, the extracellular vesicle and the PD-1 antagonist are administered concurrently. In some aspects, the extracellular vesicle and the PD-1 antagonist are administered sequentially. In some aspects, the extracellular vesicle and the PD-1 antagonist are administered on the same day. In some aspects, the extracellular vesicle and the PD-1 antagonist are administered on the different days.

In some aspects, the ASO and the PD-1 antagonist for the present disclosure can be administered in the same EV. In other aspects, the ASO and the PD-1 antagonist for the present disclosure are administered in different EVs. For example, the present disclosure includes a pharmaceutical composition comprising an EV comprising an ASO and an EV comprising a PD-1 antagonist. In some aspects, the pharmaceutical composition comprising the EV, e.g., exosome, is administered prior to administration of the PD-1 antagonist. In other aspects, the pharmaceutical composition comprising the EV, e.g., exosome, is administered after the administration of the PD-1 antagonist. In further aspects, the pharmaceutical composition comprising the EV, e.g., exosome, is administered concurrently with the PD-1 antagonist.

### VI. Pharmaceutical Compositions

Provided herein are pharmaceutical compositions comprising an EV, e.g., exosome, of the present disclosure having the desired degree of purity, and a pharmaceutically acceptable carrier or excipient, in a form suitable for administration to a subject. In some aspects, the pharmaceutical composition further comprises a PD-1 antagonist. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of extracellular vesicles. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

In some aspects, a pharmaceutical composition comprises one or more therapeutic agents, e.g., a PD-1 antagonist, and an exosome described herein. In certain aspects, the EVs, e.g., exosomes, are co-administered with one or more additional therapeutic agents, e.g., a PD-1 antagonist, in a pharmaceutically acceptable carrier.

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the extracellular vesicles described herein, use thereof in the compositions is contemplated.

In some aspects, the diluent comprises NaCl. In some aspects, the diluent comprises at least about 10 mM to at least about 200 mM NaCl, at least about 10 mM to at least about 175 mM NaCl, at least about 10 mM to at least about 150 mM NaCl, at least about 10 mM to at least about 125 mM NaCl, at least about 10 mM to at least about 100 mM NaCl, at least about 10 mM to at least about 75 mM NaCl, at least about 10 mM to at least about 50 mM NaCl, at least about 25 mM to at least about 200 mM NaCl, at least about 25 mM to at least about 175 mM NaCl, at least about 25 mM to at least about 150 mM NaCl, at least about 25 mM to at least about 125 mM NaCl, at least about 25 mM to at least about 100 mM NaCl, at least about 25 mM to at least about 75 mM NaCl, at least about 25 mM to at least about 50 mM NaCl, at least about 50 mM to at least about 200 mM NaCl, at least about 50 mM to at least about 175 mM NaCl, at least about 50 mM to at least about 150 mM NaCl, at least about 50 mM to at least about 125 mM NaCl, at least about 50 mM to at least about 100 mM NaCl, at least about 50 mM to at least about 75 mM NaCl, at least about 75 mM to at least about 200 mM NaCl, at least about 75 mM to at least about 175 mM NaCl, at least about 75 mM to at least about 150 mM NaCl, at least about 75 mM to at least about 125 mM NaCl, at least about 75 mM to at least about 100 mM NaCl, at least about 100 mM to at least about 200 mM NaCl, at least about 100 mM to at least about 175 mM NaCl, at least about 100 mM to at least about 150 mM NaCl, at least about 125 mM to at least about 200 mM NaCl, at least about 125 mM to at least about 175 mM NaCl, at least about 125 mM to at least about 150 mM NaCl, or at least about 150 mM to at least about 200 mM NaCl. In some aspects, the diluent comprises at least about 25 mM NaCl, at least about 30 mM NaCl, at least about 35 mM NaCl, at least about 40 mM NaCl, at least about 45 mM NaCl, at least about 50 mM NaCl, at least about 55 mM NaCl, at least about 60 mM NaCl, at least about 70 mM NaCl, at least about 75 mM NaCl, at least about 80 mM NaCl, at least about 90 mM NaCl, at least about 100 mM NaCl, at least about 110 mM NaCl, at least about 120 mM NaCl, at least about 125 mM NaCl, at least about 130 mM NaCl, at least about 135 mM NaCl, at least about 140 mM NaCl, at least about 145 mM NaCl, at least about 150 mM NaCl, at least about 155 mM NaCl, at least about 160 mM NaCl, at least about 165 mM NaCl, at least about 170 mM NaCl, at least about 175 mM NaCl, at least about 180 mM NaCl, at least about 190 mM NaCl, or at least about 200 mM NaCl. In some aspects, the diluent comprises about 50 mM NaCl. In some aspects, the diluent comprises about 150 mM NaCl. In some aspects, the diluent comprises about 155 mM NaCl.

In some aspects, the diluent comprises a phosphate (PO₄). In some aspects, the phosphate is a phosphate salt. In some aspects, the diluent comprises at least about 1 mM to at least about 50 mM phosphate. In some aspects, the diluent comprises at least about 1 mM to at least about 30 mM, at least about 1 mM to at least about 20 mM, at least about 1 mM to at least about 10 mM, at least about 1 mM to at least about 5 mM, at least about 2 mM to at least about 25 mM, at least about 3 mM to at least about 20 mM, at least about 5 mM to at least about 30 mM, at least about 5 mM to at least about 25 mM, at least about 5 mM to at least about 20 mM, at least about 10 mM to at least about 30 mM, at least about 10 mM to at least about 25 mM, at least about 10 mM to at least about 20 mM, at least about 15 mM to at least about 30 mM, at least about 15 mM to at least about 25 mM, at least about 15 mM to at least about 20 mM, at least about 20 mM to at least about 30 mM, at least about 20 mM to at least about 25 mM, at least about 2 mM to at least about 9 mM, at least about 3 mM to at least about 8 mM, at least about 4 mM to at least about 7 mM, at least about 4 mM to at least about 6 mM, or at least about 4 mM to at least about 5 mM phosphate. In some aspects, the diluent comprises at least about 1 mM, at least about 2 mM, at least about 3 mM, at least about 4 mM, at least about 4.1 mM, at least about 4.2 mM, at least about 4.3 mM, at least about 4.4 mM, at least about 4.5 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, at least about 15 mM, at least about 16 mM, at least about 17 mM, at least about 18 mM, at least about 19 mM, at least about 20 mM, at least about 21 mM, at least about 22 mM, at least about 23 mM, at least about 24 mM, at least about 25 mM, or at least about 30 mM phosphate. In some aspects, the diluent comprises at least about 4.2 mM phosphate. In some aspects, the diluent comprises at least about 20 mM phosphate. In some aspects, the diluent comprises at least about 15 mM phosphate. In some aspects, the diluent comprises at least about 10 mM phosphate. In some aspects, the diluent comprises at least about 5 mM phosphate.

In some aspects, the diluent comprises a saccharide. In some aspects, the saccharide present in the diluent is a monosaccharide, a disaccharide, a trisaccharide, or any other saccharide. In some aspects, the saccharide is a sucrose. In some aspects, the saccharide is a trehalose. In some aspects, the saccharide, e.g., sucrose or trehalose, is present in the diluent at a concentration from at least about 1% to at least about 10%, from at least about 2% to at least about 9%, from at least about 3% to at least about 8%, from at least about 4% to at least about 7%, from at least about 4% to at least about 6%, from at least about 3% to at least about 7%, from at least about 5% to at least about 10%, from at least about 5% to at least about 9%, from at least about 5% to at least about 8%, or from at least about 5% to at least about 7%. In some aspects, the saccharide, e.g., sucrose or trehalose, is present in the diluent at a concentration of at least about 1%. In some aspects, the saccharide, e.g., sucrose or trehalose, is present in the diluent at a concentration of at least about 2%. In some aspects, the saccharide, e.g., sucrose or trehalose, is present in the diluent at a concentration of at least about 3%. In some aspects, the saccharide, e.g., sucrose or trehalose, is present in the diluent at a concentration of at least about 4%. In some aspects, the saccharide, e.g., sucrose or trehalose, is present in the diluent at a concentration of at least about 5%. In some aspects, the saccharide, e.g., sucrose or trehalose, is present in the diluent at a concentration of at least about 6%. In some aspects, the saccharide, e.g., sucrose or trehalose, is present in the diluent at a concentration of at least about 7%. In some aspects, the saccharide, e.g., sucrose or trehalose, is present in the diluent at a concentration of at least about 8%. In some aspects, the saccharide, e.g., sucrose or trehalose, is present in the diluent at a concentration of at least about 9%. In some aspects, the saccharide, e.g., sucrose or trehalose, is present in the diluent at a concentration of at least about 10%.

In some aspects, the diluent comprises at least about 1% sucrose. In some aspects, the diluent comprises at least about 2% sucrose. In some aspects, the diluent comprises at least about 2.5% sucrose. In some aspects, the diluent comprises at least about 3% sucrose. In some aspects, the diluent comprises at least about 4% sucrose. In some aspects, the diluent comprises at least about 5% sucrose. In some aspects, the diluent comprises at least about 6% sucrose. In some aspects, the diluent comprises at least about 7% sucrose. In some aspects, the diluent comprises at least about 8% sucrose. In some aspects, the diluent comprises at least about 9% sucrose. In some aspects, the diluent comprises at least about 10% sucrose.

In some aspects, the diluent comprises at least about 10 mg/ml sucrose. In some aspects, the diluent comprises at least about 20 mg/ml sucrose. In some aspects, the diluent comprises at least about 25 mg/ml sucrose. In some aspects, the diluent comprises at least about 30 mg/ml sucrose. In some aspects, the diluent comprises at least about 40 mg/ml sucrose. In some aspects, the diluent comprises at least about 50 mg/ml sucrose. In some aspects, the diluent comprises at least about 60 mg/ml sucrose. In some aspects, the diluent comprises at least about 70 mg/ml sucrose. In some aspects, the diluent comprises at least about 80 mg/ml sucrose. In some aspects, the diluent comprises at least about 90 mg/ml sucrose. In some aspects, the diluent comprises at least about 100 mg/ml sucrose.

In some aspects, the diluent comprises phosphate buffered saline (PBS).

In some aspects, the diluent has a pH of at least about 6.5 to about 7.5. In some aspects, the diluent has a pH of about 6.5, about 6.6, about 6.7, about 6.8, about 6.9, about 7.0, about 7.1, about 7.2, about 7.3, about 7.4, or about 7.5. In some aspects, the diluent has a pH of about 7.2.

In some aspects, the osmolarity of the diluent is from about 250 mOsm/kg to about 450 mOsm/kg. In certain aspects, the osmolarity of the diluent is between about 275 mOsm/kg and about 450 mOsm/kg, between about 280 mOsm/kg and about 450 mOsm/kg, between about 300 mOsm/kg and about 450 mOsm/kg, between about 275 mOsm/kg and about 400 mOsm/kg, between about 280 mOsm/kg and about 400 mOsm/kg, between about 300 mOsm/kg and about 400 mOsm/kg, between about 275 mOsm/kg and about 380 mOsm/kg, between about 280 mOsm/kg and about 380 mOsm/kg, between about 300 mOsm/kg and about 380 mOsm/kg, between about 275 mOsm/kg and about 350 mOsm/kg, between about 280 mOsm/kg and about 350 mOsm/kg, between about 300 mOsm/kg and about 350 mOsm/kg, between about 275 mOsm/kg and about 310 mOsm/kg, between about 280 mOsm/kg and about 310 mOsm/kg, or between about 300 mOsm/kg and about 310 mOsm/kg.

In some aspects, the osmolarity of the diluent is about 360 mOsm/kg, about 370 mOsm/kg, about 380 mOsm/kg, about 390 mOsm/kg, about 395 mOsm/kg, or about 400 mOsm/kg. In some aspects, the osmolarity of the diluent is about 395 mOsm/kg.

Supplementary therapeutic agents can also be incorporated into the compositions. Typically, a pharmaceutical composition is formulated to be compatible with its intended route of administration. The EVs, e.g., exosomes, can be administered by parenteral, topical, intravenous, oral, subcutaneous, intra-arterial, intradermal, transdermal, rectal, intracranial, intraperitoneal, intranasal, intratumoral, intramuscular route or as inhalants. In certain aspects, the pharmaceutical composition comprising exosomes is administered intravenously, e.g. by injection. The EVs, e.g., exosomes, can optionally be administered in combination with other therapeutic agents that are at least partly effective in treating the disease, disorder or condition for which the EVs, e.g., exosomes, are intended.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as manitol, sorbitol, and sodium chloride can be added to the composition. Prolonged absorption of the injectable compositions can be brought about by including in the composition a compound which delays absorption, e.g., aluminum monostearate and gelatin.

Sterile injectable solutions can be prepared by incorporating the EVs, e.g., exosomes, in an effective amount and in an appropriate solvent with one or more ingredients enumerated herein or known in the art, as desired. Generally, dispersions are prepared by incorporating the EVs, e.g., exosomes, into a sterile vehicle that contains a basic dispersion medium and any desired other ingredients. In the case of sterile powders for the preparation of sterile injectable solutions, methods of preparation are vacuum drying and freeze-drying that yield a powder of the active ingredient plus any additional desired ingredient from a previously sterile-filtered solution thereof. The EVs, e.g., exosomes, can be administered in the form of a depot injection or implant preparation which can be formulated in such a manner to permit a sustained or pulsatile release of the EV, e.g., exosome.

Systemic administration of compositions comprising exosomes can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects the pharmaceutical composition comprising EVs, e.g., exosomes is administered intravenously into a subject that would benefit from the pharmaceutical composition. In certain other aspects, the composition is administered to the lymphatic system, e.g., by intralymphatic injection or by intranodal injection (see e.g., Senti et al., PNAS 105(46): 17908 (2008)), or by intramuscular injection, by subcutaneous administration, by intratumoral injection, by direct injection into the thymus, or into the liver.

In certain aspects, the pharmaceutical composition comprising exosomes is administered as a liquid suspension. In certain aspects, the pharmaceutical composition is administered as a formulation that is capable of forming a depot following administration. In certain preferred aspects, the depot slowly releases the EVs, e.g., exosomes, into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

In some aspects, the pharmaceutical compositions described herein comprise a pharmaceutically acceptable salt. In some aspects, the pharmaceutically acceptable salt comprises a sodium salt, a potassium salt, an ammonium salt, or any combination thereof.

In some aspects, the pharmaceutical compositions described herein comprise the EVs, e.g., exosomes, and PD-1 antagonists, described herein, and optionally an additional pharmaceutically active or therapeutic agent. The additional therapeutic agent can be a biological agent, a small molecule agent, or a nucleic acid agent. In some aspects, the additional therapeutic agent is an additional STAT6 antagonist. In some aspects, the STAT6 antagonist is any STAT6 antagonist disclosed herein. In some aspects, the additional STAT6 antagonist is an anti-STAT6 antibody. In some aspects, the additional STAT6 antagonist is a small molecule. In some aspects, the additional STAT6 antagonist is a small molecule.

Dosage forms are provided that comprise a pharmaceutical composition comprising the EVs, e.g., exosomes, described herein. In some aspects, the dosage form is formulated as a liquid suspension for intravenous injection. In some aspects, the dosage form is formulated as a liquid suspension for intratumoral injection.

In certain aspects, the preparation of exosomes is subjected to radiation, e.g., X rays, gamma rays, beta particles, alpha particles, neutrons, protons, elemental nuclei, UV rays in order to damage residual replication-competent nucleic acids.

In certain aspects, the preparation of exosomes is subjected to gamma irradiation using an irradiation dose of more than 1, 5, 10, 15, 20, 25, 30, 35, 40, 50, 60, 70, 80, 90, 100, or more than 100 kGy.

In certain aspects, the preparation of exosomes is subjected to X-ray irradiation using an irradiation dose of more than 0.1, 0.5, 1, 5, 10, 15, 20, 25, 30, 35, 40, 50, 60, 70, 80, 90, 100, 200, 300, 400, 500, 600, 700, 800, 900, 1000, 2000, 3000, 4000, 5000, 6000, 7000, 8000, 9000, 10000, or greater than 10000 mSv.

In some aspects, the pharmaceutical composition comprises:
a. an extracellular vesicle comprising an ASO, wherein the ASO comprises a contiguous nucleotide sequence of 10 to 30 nucleotides in length that is complementary to a nucleic acid sequence within a STAT6 transcript;
b. a sucrose at a concentration of about 5% w/v,
c. sodium chloride at a concentration of about 100 mM;
d. a potassium phosphate monobasic at a concentration of about 5 mM;
e. a sodium phosphate dibasic heptahydrate at a concentration of about 15 mM;
   wherein the pharmaceutical composition is in a solution at a pH of 7.2; and
   wherein the pharmaceutical composition comprises an osmolarity of about 365 to about 425 mOsm/kg.

In some aspects, the pharmaceutical composition comprises:
a. an extracellular vesicle comprising an ASO, wherein the ASO comprises a contiguous nucleotide sequence of 10 to 30 nucleotides in length that is complementary to a nucleic acid sequence within a STAT6 transcript;
b. a sucrose at a concentration of about 5% w/v,
c. sodium chloride at a concentration of about 100 mM;
d. a potassium phosphate monobasic at a concentration of about 5 mM;
e. a sodium phosphate dibasic heptahydrate at a concentration of about 15 mM;
   wherein the pharmaceutical composition is in a solution at a pH of 7.2; and
   wherein the pharmaceutical composition comprises an osmolarity of about 395 mOsm/kg.

In some aspects, the pharmaceutical composition comprises:
a. an extracellular vesicle comprising an ASO, wherein the ASO comprises a contiguous nucleotide sequence of 10 to 30 nucleotides in length that is complementary to a nucleic acid sequence within a STAT6 transcript;
b. a sucrose at a concentration of about 146 mM,
c. sodium chloride at a concentration of about 100 mM;
d. a potassium phosphate monobasic at a concentration of about 5 mM;
e. a sodium phosphate dibasic heptahydrate at a concentration of about 15 mM;
   wherein the pharmaceutical composition is in a solution at a pH of 7.2; and
   wherein the pharmaceutical composition comprises an osmolarity of about 365 to about 425 mOsm/kg.

In some aspects, the pharmaceutical composition comprises:
a. an extracellular vesicle comprising an ASO, wherein the ASO comprises a contiguous nucleotide sequence of 10 to 30 nucleotides in length that is complementary to a nucleic acid sequence within a STAT6 transcript;
b. a sucrose at a concentration of about 146 mM,
c. sodium chloride at a concentration of about 100 mM;
d. a potassium phosphate monobasic at a concentration of about 5 mM;
e. a sodium phosphate dibasic heptahydrate at a concentration of about 15 mM;
   wherein the pharmaceutical composition is in a solution at a pH of 7.2; and
   wherein the pharmaceutical composition comprises an osmolarity of about 395 mOsm/kg.

In some aspects, the pharmaceutical composition comprises:
a. an extracellular vesicle comprising an ASO, wherein the ASO comprises a nucleic acid sequence selected from SEQ ID NOs: 91-193;
b. a sucrose at a concentration of about 146 mM,
c. sodium chloride at a concentration of about 100 mM;
d. a potassium phosphate monobasic at a concentration of about 5 mM;
e. a sodium phosphate dibasic heptahydrate at a concentration of about 15 mM;
   wherein the pharmaceutical composition is in a solution at a pH of 7.2; and
   wherein the pharmaceutical composition comprises an osmolarity of about 395 mOsm/kg.

In some aspects, the pharmaceutical composition comprises:
a. an extracellular vesicle comprising an ASO, wherein the ASO comprises the nucleic acid sequence GAAAGGTTCCGTCGGGC (SEQ ID NO: 144);
b. a sucrose at a concentration of about 146 mM,
c. sodium chloride at a concentration of about 100 mM;
d. a potassium phosphate monobasic at a concentration of about 5 mM;
e. a sodium phosphate dibasic heptahydrate at a concentration of about 15 mM;
   wherein the pharmaceutical composition is in a solution at a pH of 7.2; and
   wherein the pharmaceutical composition comprises an osmolarity of about 395 mOsm/kg.

In some aspects, the pharmaceutical composition comprises:
a. an extracellular vesicle comprising an ASO, wherein the ASO comprises the nucleic acid sequence CTGAGTCGCTGAAGCGG (SEQ ID NO: 145);
b. a sucrose at a concentration of about 146 mM,
c. sodium chloride at a concentration of about 100 mM;
d. a potassium phosphate monobasic at a concentration of about 5 mM;
e. a sodium phosphate dibasic heptahydrate at a concentration of about 15 mM;
   wherein the pharmaceutical composition is in a solution at a pH of 7.2; and
   wherein the pharmaceutical composition comprises an osmolarity of about 395 mOsm/kg.

In some aspects, the pharmaceutical composition comprises:
a. an extracellular vesicle comprising an ASO, wherein the ASO comprises the nucleic acid sequence GCCCTTGTACTTTTGCATAG (SEQ ID NO: 193);
b. a sucrose at a concentration of about 146 mM,
c. sodium chloride at a concentration of about 100 mM;
d. a potassium phosphate monobasic at a concentration of about 5 mM;
e. a sodium phosphate dibasic heptahydrate at a concentration of about 15 mM;
   wherein the pharmaceutical composition is in a solution at a pH of 7.2; and
   wherein the pharmaceutical composition comprises an osmolarity of about 395 mOsm/kg.

In some aspects, the pharmaceutical composition comprises:
a. an extracellular vesicle comprising an ASO, wherein the ASO comprises the nucleic acid sequence GCAAGATCCCGGATTCGGTC (SEQ ID NO: 185);
b. a sucrose at a concentration of about 146 mM,
c. sodium chloride at a concentration of about 100 mM;
d. a potassium phosphate monobasic at a concentration of about 5 mM;
e. a sodium phosphate dibasic heptahydrate at a concentration of about 15 mM;
wherein the pharmaceutical composition is in a solution at a pH of 7.2; and
wherein the pharmaceutical composition comprises an osmolarity of about 395 mOsm/kg.

In some aspects, the ASO comprises a nucleic acid sequence selected from SEQ ID NOs: 91-193. In some aspects, the ASO comprises the nucleic acid sequence GAAAGGTTCCGTCGGGC (SEQ ID NO: 144). In some aspects, the ASO comprises the nucleic acid sequence CTGAGTCGCTGAAGCGG (SEQ ID NO: 145). In some aspects, the ASO comprises the nucleic acid sequence GCCCTTGTACTTTTGCATAG (SEQ ID NO: 193). In some aspects, the ASO comprises the nucleic acid sequence GCAAGATCCCGGATTCGGTC (SEQ ID NO: 185).

In certain aspects, the pharmaceutical composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose;
(c) Sodium chloride;
(d) Potassium phosphate monobasic at a concentration of about 5 mM;
(e) Sodium phosphate dibasic at a concentration of about 15 mM, wherein the pH of the pharmaceutical composition is about 7.2;
wherein the sucrose is at a concentration selected from about 73 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 146 mM, and about 150 mM;
   wherein the sodium chloride is at a concentration selected from about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 146 mM, and about 150 mM; and
wherein the pharmaceutical composition comprises an osmolarity of about 395 mOsm/kg.

In certain aspects, the pharmaceutical composition comprises:
(a) Extracellular vesicles comprising an ASO;
(b) Sucrose;
(c) Sodium chloride;
(d) Potassium phosphate monobasic at a concentration of about 5 mM;
(e) Sodium phosphate dibasic at a concentration of about 15 mM,
(f) wherein the pH of the pharmaceutical composition is about 7.2;
wherein the sucrose is at a concentration selected from about 73 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 146 mM, and about 150 mM;
wherein the sodium chloride is at a concentration selected from about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 146 mM, and about 150 mM; and
wherein the pharmaceutical composition comprises an osmolarity of about 395 mOsm/kg.

In some aspects, the ASO comprises a nucleic acid sequence selected from SEQ ID NOs: 91-193.

In some aspects, the pharmaceutical composition comprises:
(a) Extracellular vesicles comprising an ASO;
(b) Sucrose;
(c) Sodium chloride;
(d) Potassium phosphate monobasic at a concentration of about 5 mM;
(e) Sodium phosphate dibasic at a concentration of about 15 mM,
(f) wherein the pH of the pharmaceutical composition is about 7.2;
wherein the sucrose is at a concentration selected from about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, and about 5.0%;
wherein the sodium chloride is at a concentration selected from about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 146 mM, and about 150 mM; and
wherein the pharmaceutical composition comprises an osmolarity of about 395 mOsm/kg.

In some aspects, the ASO comprises a nucleic acid sequence selected from SEQ ID NOs: 91-193.

In certain aspects, the pharmaceutical composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose at a concentration of about 73 mM to about 146 mM;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In some aspects, the pharmaceutical composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose at a concentration of about 2.5% to about 5%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In certain aspects, the pharmaceutical composition comprises:
(a) Extracellular vesicles comprising an ASO, wherein the ASO comprises a nucleic acid sequence selected from SEQ ID NOs: 91-193;
(b) Sucrose at a concentration of about 73 mM to about 146 mM;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In some aspects, the pharmaceutical composition comprises:
(a) Extracellular vesicles comprising an ASO, wherein the ASO comprises a nucleic acid sequence selected from SEQ ID NOs: 91-193;
(b) Sucrose at a concentration of about 2.5% to about 5%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In some aspects, the pharmaceutical composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose at a concentration of about 5%;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In some aspects, the pharmaceutical composition comprises:
(a) Extracellular vesicles comprising an ASO, wherein the ASO comprises a contiguous nucleotide sequence of 10 to 30 nucleotides in length that is complementary to a nucleic acid sequence within a STAT6 transcript;
(b) Sucrose at a concentration of about 2.5%;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In some aspects, the ASO comprises a nucleic acid sequence selected from SEQ ID NOs: 91-93.

In certain aspects, the pharmaceutical composition comprises:
(a) Extracellular vesicles comprising an ASO, wherein the ASO comprises a nucleic acid sequence selected from SEQ ID NOs: 91-193;
(b) Sucrose at a concentration of about 146 mM;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In some aspects, the composition comprises:
(a) Extracellular vesicles comprising an ASO, wherein the ASO comprises a nucleic acid sequence selected from SEQ ID NOs: 91-193;
(b) Sucrose at a concentration of about 5%;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In some aspects, the pharmaceutical composition comprises:
(a) Extracellular vesicles comprising an ASO, wherein the ASO comprises a nucleic acid sequence selected from SEQ ID NOs: 91-193;
(b) Sucrose at a concentration of about 4.5%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In some aspects, the pharmaceutical composition comprises:
(a) Extracellular vesicles comprising an ASO, wherein the ASO comprises a nucleic acid sequence selected from SEQ ID NOs: 91-193;
(b) Sucrose at a concentration of about 4%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In some aspects, the pharmaceutical composition comprises:
(a) Extracellular vesicles comprising an ASO, wherein the ASO comprises a nucleic acid sequence selected from SEQ ID NOs: 91-193;
(b) Sucrose at a concentration of about 3.5%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In some aspects, the pharmaceutical composition comprises:
(a) Extracellular vesicles comprising an ASO, wherein the ASO comprises a nucleic acid sequence selected from SEQ ID NOs: 91-193;
(b) Sucrose at a concentration of about 3%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In some aspects, the pharmaceutical composition comprises:
(a) Extracellular vesicles comprising an ASO, wherein the ASO comprises a nucleic acid sequence selected from SEQ ID NOs: 91-193;
(b) Sucrose at a concentration of about 2.5%;
(c) Sodium chloride at a concentration of about 50 mM to about 150 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In certain aspects, the pharmaceutical composition comprises:
(a) Extracellular vesicles comprising an ASO, wherein the ASO comprises the nucleic acid sequence GAAAGGTTCCGTCGGGC (SEQ ID NO: 144);
(b) Sucrose at a concentration of about 146 mM;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In some aspects, the pharmaceutical composition comprises:
(a) Extracellular vesicles comprising an ASO, wherein the ASO comprises the nucleic acid sequence GAAAGGTTCCGTCGGGC (SEQ ID NO: 144);
(b) Sucrose at a concentration of about 5%;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In certain aspects, the pharmaceutical composition comprises:
(a) Extracellular vesicles comprising an ASO, wherein the ASO comprises the nucleic acid sequence CTGAGTCGCTGAAGCGG (SEQ ID NO: 145);
(b) Sucrose at a concentration of about 146 mM;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 4395 mOsm/kg.

In some aspects, the pharmaceutical composition comprises:
(a) Extracellular vesicles comprising an ASO, wherein the ASO comprises the nucleic acid sequence CTGAGTCGCTGAAGCGG (SEQ ID NO: 145);
(b) Sucrose at a concentration of about 5%;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In certain aspects, the pharmaceutical composition comprises:
(a) Extracellular vesicles comprising an ASO, wherein the ASO comprises the nucleic acid sequence GCCCTTGTACTTTTGCATAG (SEQ ID NO: 193);
(b) Sucrose at a concentration of about 146 mM;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In some aspects, the pharmaceutical composition comprises:
(a) Extracellular vesicles comprising an ASO, wherein the ASO comprises the nucleic acid sequence GCCCTTGTACTTTTGCATAG (SEQ ID NO: 193);
(b) Sucrose at a concentration of about 5%;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In certain aspects, the pharmaceutical composition comprises:
(a) Extracellular vesicles comprising an ASO, wherein the ASO comprises the nucleic acid sequence GCAAGATCCCGGATTCGGTC (SEQ ID NO: 185);
(b) Sucrose at a concentration of about 146 mM;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In some aspects, the pharmaceutical composition comprises:
(a) Extracellular vesicles comprising an ASO, wherein the ASO comprises the nucleic acid sequence GCAAGATCCCGGATTCGGTC (SEQ ID NO: 185);
(b) Sucrose at a concentration of about 5%;
(c) Sodium chloride at a concentration of about 100 mM;
(d) Potassium phosphate monobasic at a concentration of about 5 mM;
(e) Sodium phosphate dibasic at a concentration of about 15 mM,
(f) wherein the pH of the pharmaceutical composition is about 7.2; and
wherein the pharmaceutical composition comprises an osmolarity of about 395 mOsm/kg.

In some aspects, the pharmaceutical composition is lyophilized.

### VII. Kits

Also provided herein are kits comprising one or more exosomes described herein and one or more PD-1 antagonist disclosed herein. In some aspects, provided herein is a pharmaceutical pack or kit comprising one or more containers filled with one or more of the ingredients of the pharmaceutical compositions described herein, such as one or more exosomes provided herein, optional an instruction for use. In some aspects, the kits contain a pharmaceutical composition described herein and any prophylactic or therapeutic agent, such as those described herein. In some aspects, the kit further comprises instructions to administer the EV according to any method disclosed herein. In some aspects, the kit is for use in the treatment of a disease or condition associated with hematopoiesis. In some aspects, the kit is a diagnostic kit.

All of the references cited above, as well as all references cited herein, are incorporated herein by reference in their entireties.

The following examples are offered by way of illustration and not by way of limitation.

### EXAMPLES

### Example 1: In Vivo Administration of Exosomes with STAT6 ASO in Combination with a PD1

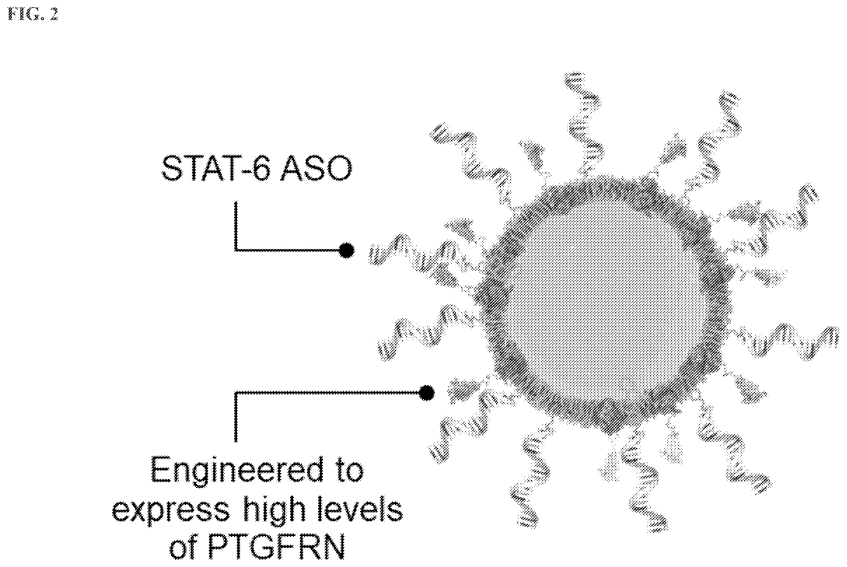

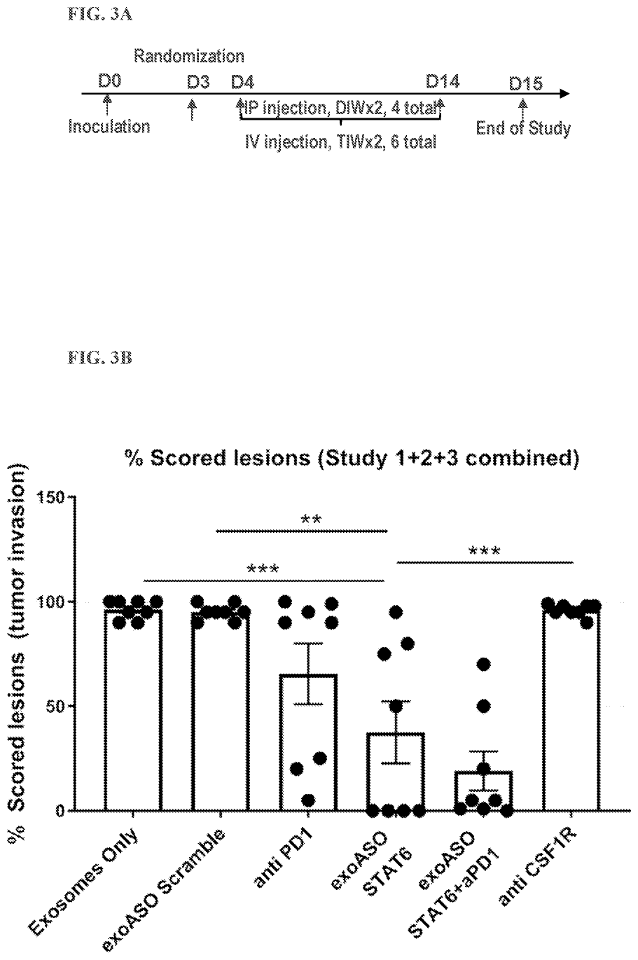

To determine the anti-tumor activity, exoASO-STAT6 and control exosome were administered as single agents and in combination with an anti PD-1 monoclonal antibody evaluated in the orthotopic Hepa1-6 murine hepatocellular carcinoma model. Hepa1-6 cells were injected into spleen of C57BL/6 mice to induce the tumor development in the liver (n=7-8 per group). At day 3 post inoculation, body weight was measured and mice were divided into 6 treatment groups (Table 3; FIG. 3A). Mice in group 1 were administered 1×10¹¹ empty exosomes by intravenous (IV) injection on days 4, 6, 8, 10, 12, and 14 post inoculation. Mice in group 2 were administered 2×10¹¹ exosomes loaded with Scramble ASO on days 4, 6, 8, 10, 12, and 14 post inoculation by IV. Mice in group 3 were administered 10 mg/kg of an anti-PD1 antibody (aPD-1) by intraperitoneal (IP) injection on days 4, 7, 10, and 13 post inoculation. Mice in group 4 were administered 2×10¹¹ exosomes loaded with STAT6 ASO on days 4, 6, 8, 10, 12, and 14 post inoculation by IV. Mice in group 5 were administered 2×10¹¹ exosomes loaded with STAT6 ASO on days 4, 6, 8, 10, 12, and 14 post inoculation by IV, and 10 mg/kg of an aPD-1 by IP injection on days 4, 7, 10, and 13 post inoculation. Mice in group 6 were administered 10 mg/kg of an anti-CSF1R antibody (aCSF1R) by IP injection on days 4, 7, 10, and 13 post inoculation. Mice were sacrificed at day 15 post inoculation, and livers were collected to score the visible tumor lesions.

TABLE 3
In vivo efficacy experimental design.

| Grp. | Drug | Dose | Route | # Doses | Dosing | N |
|---|---|---|---|---|---|---|
| 1 | Exosomes | 1 × 10¹¹ | IV | 6 | q2d | 8 |
| 2 | exoASO Scramble | 2 × 10¹¹ | IV | 6 | q2d | 8 |
| 3 | aPD-1 | 10 mg/kg | IP | 4 | TW | 8 |
| 4 | exoASO STAT6 | 2 × 10¹¹ | IV | 6 | q2d | 8 |
| 5 | exoASO STAT6 | 2 × 10¹¹ | IV | 6 | q2d | 8 |
| | aPD-1 | 10 mg/kg | IP | 4 | TW | |
| 6 | aCSF1R | 10 mg/kg | IP | 11 | q2d | 8 |

IV = intravenous injection;
IP = intraperitoneal injection;
q2d = every 2 days;
TW = twice per week.

Efficacy was determined by percentage of macroscopically scored visible tumor lesions at end point, as shown in FIG. 3B. Mice treated with an anti-PD-1 antibody alone (about 75%) or exoASO STAT6 alone (about 50%) showed a moderate decrease in the percent of scored lesions, whereas mice treated with a combination of an anti-PD-1 antibody and exoASO STAT6 had less than 25% scored lesions, relative to exosome only controls. exoASO-STAT6 monotherapy and in combination with an anti PD-1 monoclonal antibody resulted in complete tumor remissions (CR) in ≥50% of mice in each group, significantly more than in any other treatment group. (*** P<0.0005; ** P<0.005 one-way ANOVA). Further, 5 out of 7 mice treated with a combination of exoASO STAT6 and an anti-PD-1 antibody showed complete responses, whereas only 1 mouse out of 8 mice treated with an anti-PD-1 antibody alone and 4 out of 8 mice treated with exoASO STAT6 alone showed complete responses. Conversely, no mice treated with an anti-CSF1R antibody showed any change in the percent of scored lesions, and none of the anti-CDF1R-treated mice showed a complete response.

### Example 2: Delivery of STA T6 Antisense Oligonucleotides to Tumor Associated Macrophages (TAMs) Using Engineered Exosomes

Exosome-mediated delivery enhances ASO uptake in human M2 macrophages in vitro by 2× compared to free ASO. Enhanced ASO delivery by exosomes was also observed in vivo after IV administration. An 11× increase in uptake of ASO was observed in monocytes and MDSCs in the blood and up to 5.8-fold increase in TAMs and MDSCs in the tumor compared to free ASO. The exoASO-STAT6 treatment resulted in a 2× increase in STAT6 silencing as compared to free STAT6 ASO in human M2-polarized macrophages. Target gene knockdown induced by exoASO-STAT6 was persistent for up to 10 days in mouse, cynomolgus monkey and human M2 macrophages. Importantly, exoASO-STAT6 treatment induced macrophage M2 to M1 reprogramming in vitro, as demonstrated by a 7× decrease in M2 marker CD163 and a 25× increase in pro-inflammatory cytokines such as IL-12 or TNFα. Efficacy studies in the CT26 tumor model showed potent dose-dependent single agent activity of exoASO-STAT6 with 94% tumor growth inhibition and 80% complete responses at the efficacious dose.

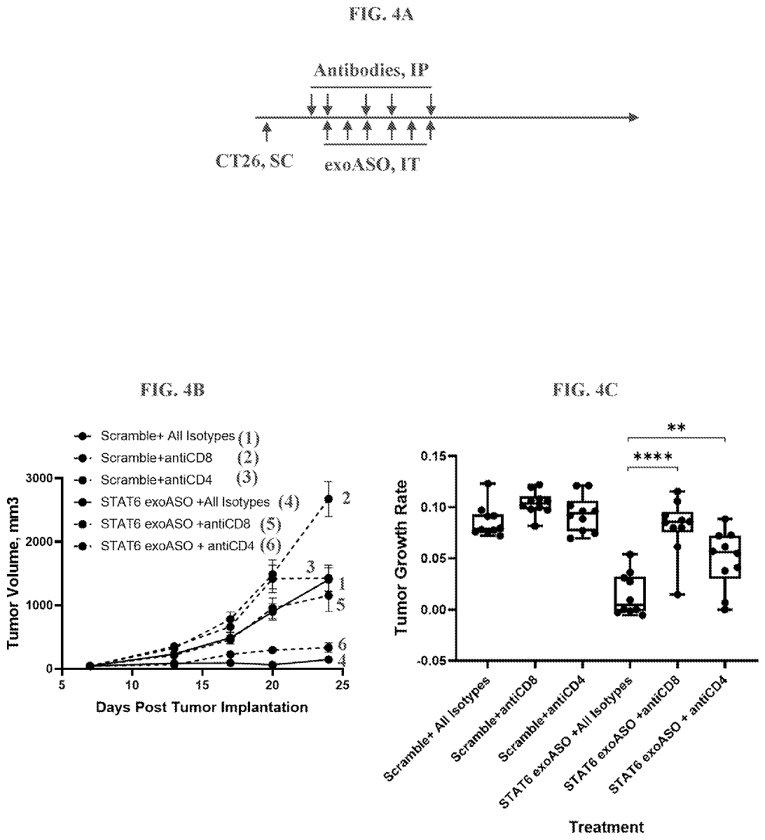

exoASO-STAT6 anti-tumoral activity was impaired by CD8 T-cell depletion and no tumor growth was observed after tumor cell re-challenge of responder animals, demonstrating the induction of immunological memory. Mice bearing subcutaneous CT26 tumors were treated with exoASO-STAT6 or exoASO-Scramble and injected intraperitoneal with CD4 (clone GK15) or CD8 (clone 53-6.7) depleting antibodies (or isotype control antibodies) (FIG. 4A). Depletion of CD8 T cells completely abrogated the anti-tumoral efficacy of exoASO-STAT6, while depletion of CD4 T cells partially but significantly inhibited the response to exoASO-STAT6 (FIGS. 4B-4C). Mice bearing sc CT26 tumors were treated it with exoASO-STAT6 either TIW or q3d for a total of 6 doses (6 ug/dose). Complete responder mice in which tumors were completely cleared were rechallenged with CT26 tumor cells on the opposite flank and tumor growth was recorded. Naïve mice with no previous tumors were used as control.

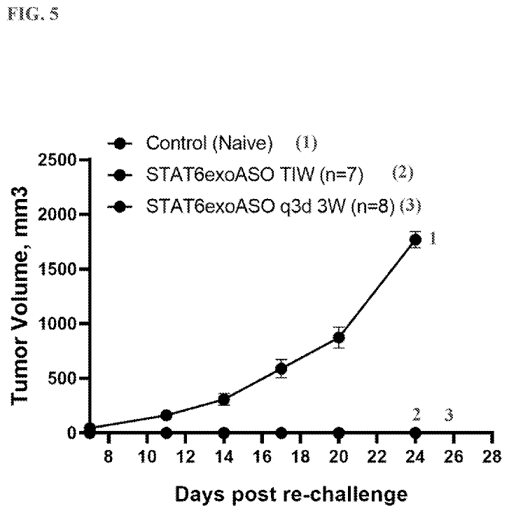

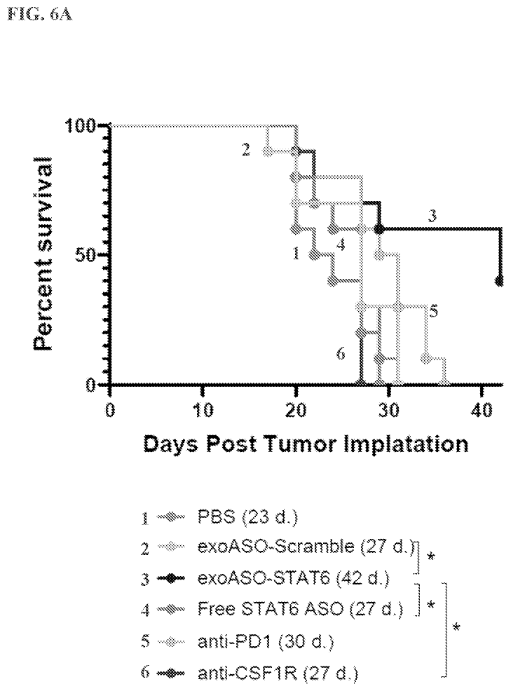

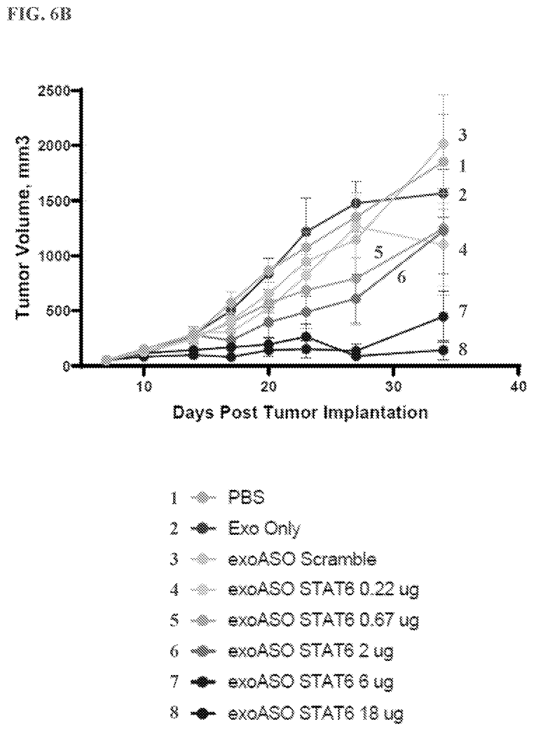

exoASO-STAT6 treatment significantly prolonged the survival by day 42 (P=0.0073 vs PBS group) (FIG. 6A). To optimize the dose and dose regimen of exoASO-STAT6 we compared the efficacy of three times a week (TIW) dosing for two weeks to twice a week dosing (BIW) for 3 weeks. Both doses resulted in comparable tumor growth inhibition resulting in 7/10 and 8/10 CRs, respectively. The less frequent dosing was chosen based on the duration of STAT6 KD in tumor CD11b cells, which peaks at 48 h following IT dosing. Next, we evaluated the dose response from 0.22 to 18 μg/dose of exoASO-STAT6 in the CT26 model. A dose dependent decrease in tumor volume of the injected tumor was observed following treatment with exoASO-STAT6 (FIG. 6B). Empty exosomes or control ASO containing exosomes failed to control tumor growth. Minimal tumor growth inhibition was observed at the 0.2 μg dose. At intermediate dose levels (0.6 and 2 μg BIW), CR was observed in one out of 10 animals (10%) with modest anti-tumor activity. At the higher dose levels (6 μg or 18 μg BIW) CR was observed in 5 and 6 out of 10 animals, respectively (50% to 60% CR) with a significant reduction in tumor growth rate (P<0.001).

Mice that were complete responders to exoASO-STAT6 were resistant to tumor rechallenge, demonstrating that treatment with exoASO-STAT6 induces immunological memory (FIG. 5).

The requirement for CD4⁺ T cells, CD8⁺ T cells in mediating antitumor responses was further characterized with antibody depletion studies. Following tumor implantation, the CD4⁺ T cells, CD8⁺ T cells, were depleted by treatment with depleting antibodies two days prior to the initiation of exoASO-STAT6 IT treatment. Robust tumor growth inhibition (P<0.0001) was observed in the exoASO-STAT6 treated isotype control group resulting in CR in 6 out of 10 mice (60% CR). In contrast, no CR was observed in the anti-CD8 antibody treated group and the tumor growth rate was comparable to control groups. The exoASO-STAT6-treated group in the presence of anti-CD4 showed modest reduction in tumor growth. Complete tumor remission was observed only in 2 out of 10 mice treated with exoASO-STAT6 in the presence of anti-CD4 antibody. These data demonstrate the critical role played by CD8+ T cells in mediating exo-STAT6 anti-tumor immunity. This study represents the first macrophage targeted therapy with single agent anti-tumor activity.

Systemic IV administration of exoASO-STAT6 showed significant single agent activity in an orthotopic model of HCC unresponsive to other immunotherapies (61% reduction in tumor mass and complete elimination of tumor lesions in 50% of treated mice). Gene expression analysis showed a decrease in M2 markers (e.g., Tgfb1 and Ccl17) and an increase in M1 markers such as IL1b induced by exoASO-STAT6 treatment. A significant increase in interferon and cytotoxic T-cell gene signatures was also observed, as well as a decrease in pathways involved in tumor progression such as angiogenesis and cell proliferation.

In summary, exoASO-STAT6 selectively silences STAT6 in TAMS, resulting in effective reprogramming that converts immunosuppressive macrophages to a pro-inflammatory phenotype, which in turns translates into potent single agent anti-tumor activity in multiple checkpoint refractory tumor models.

### Example 3: Effective Remodeling of the TME by exoASO STAT6 in the CT26 Tumor Model

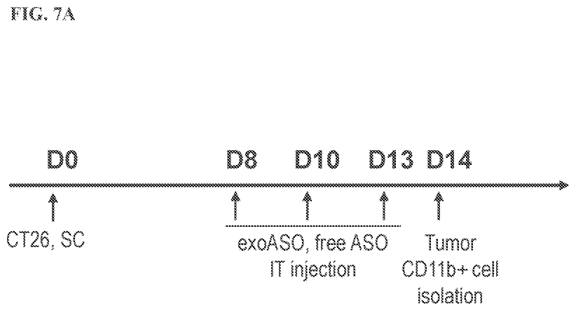

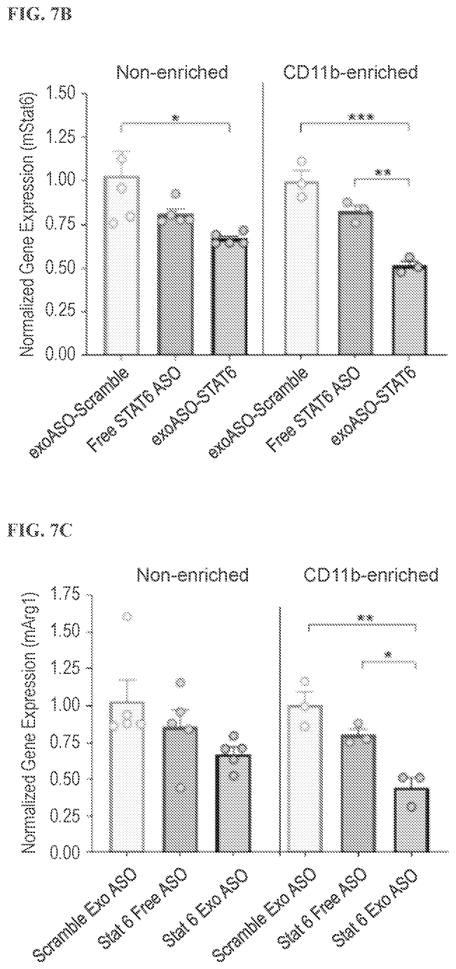

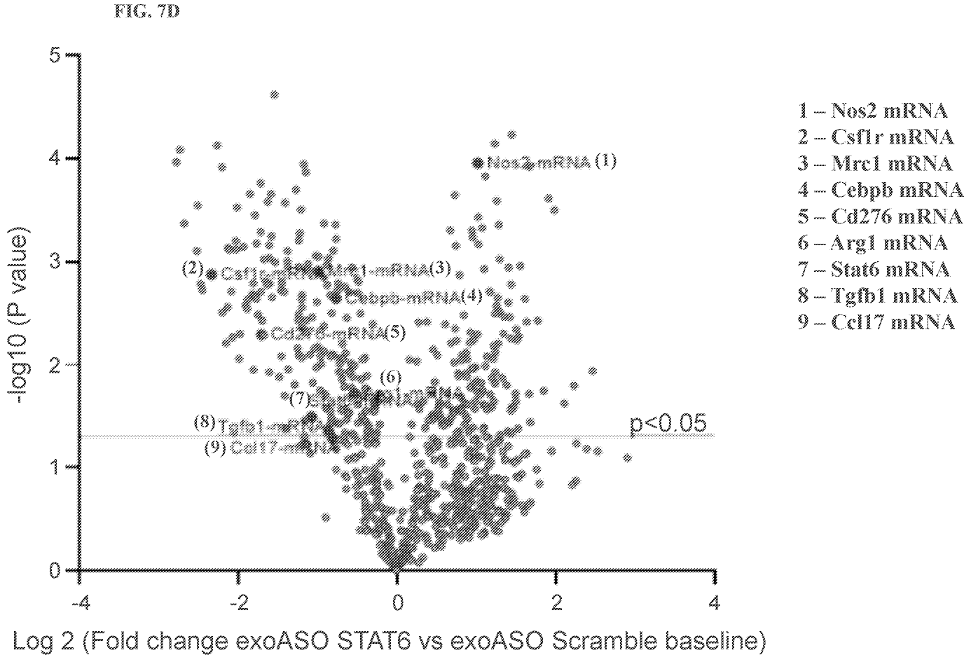

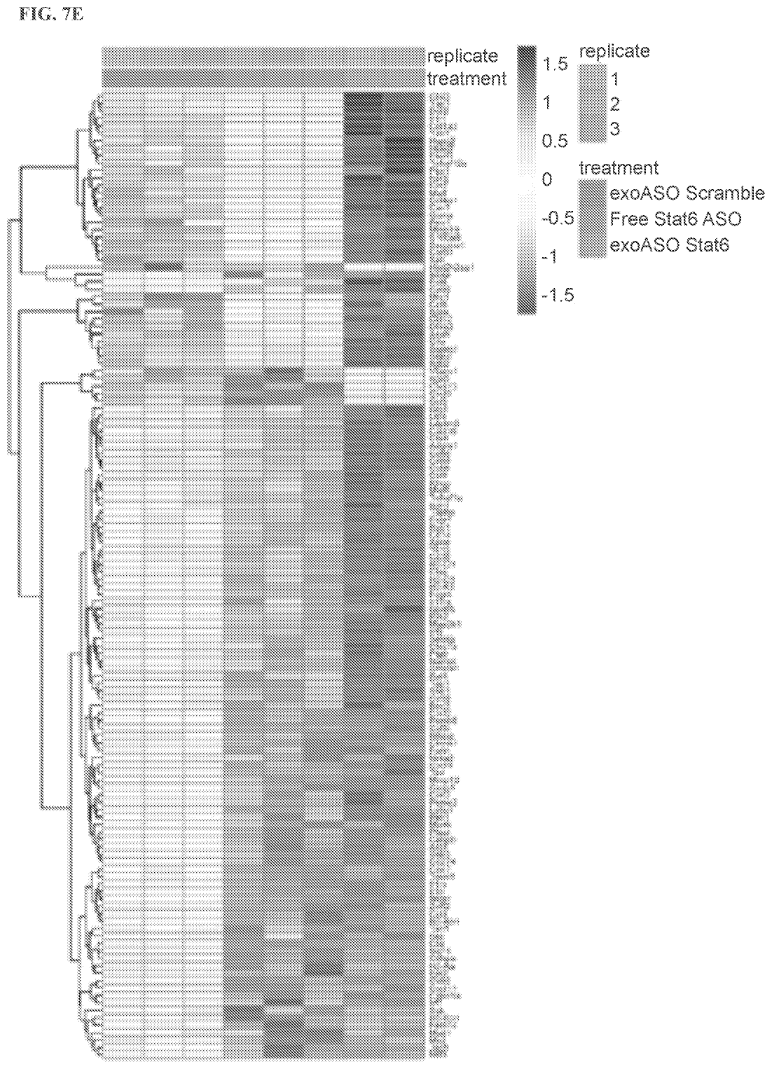

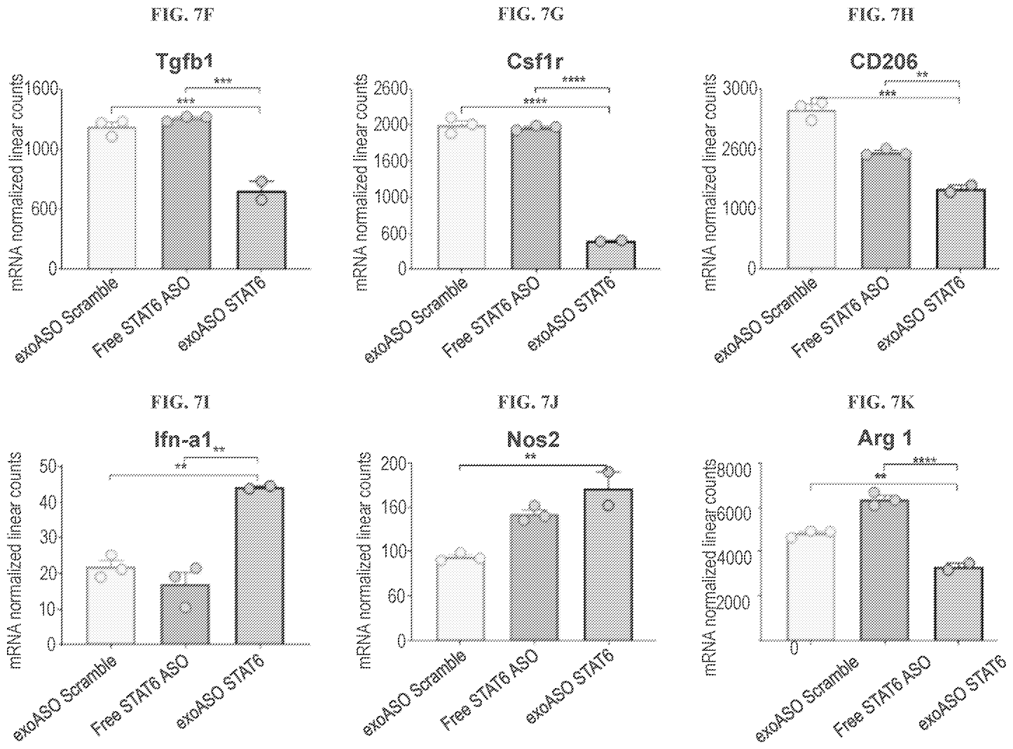

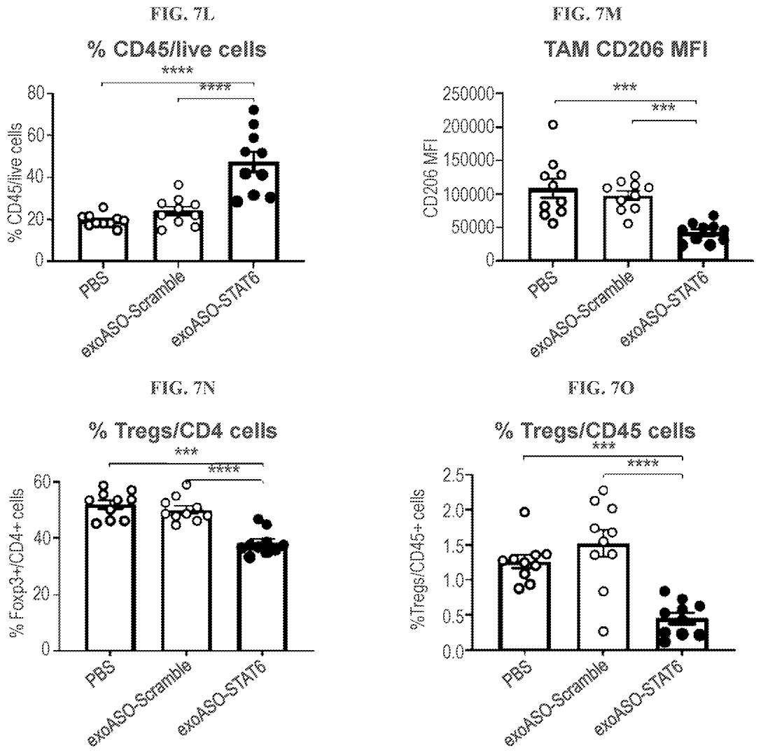

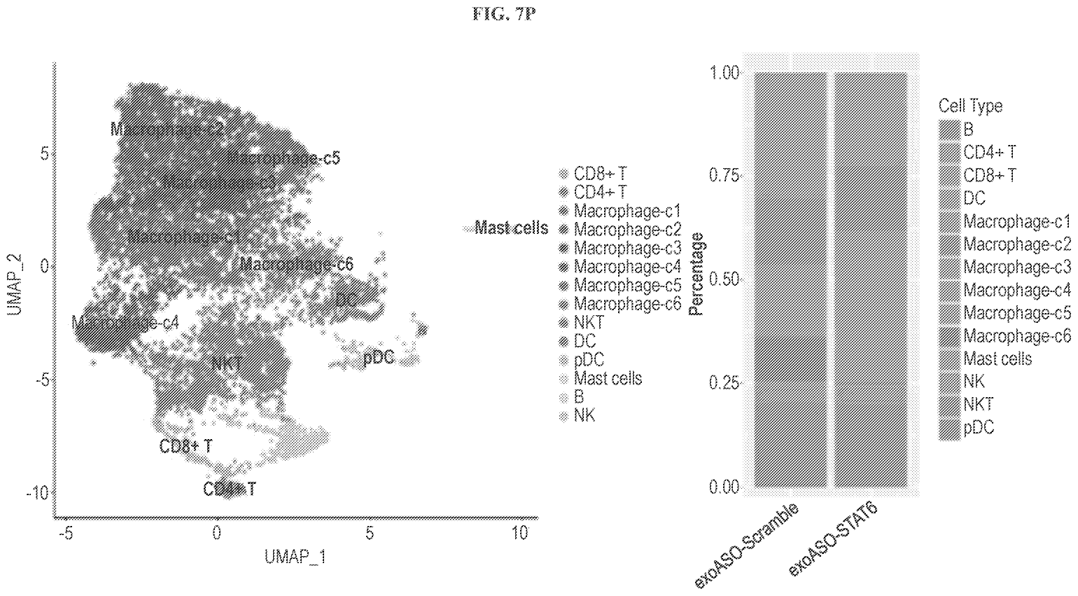

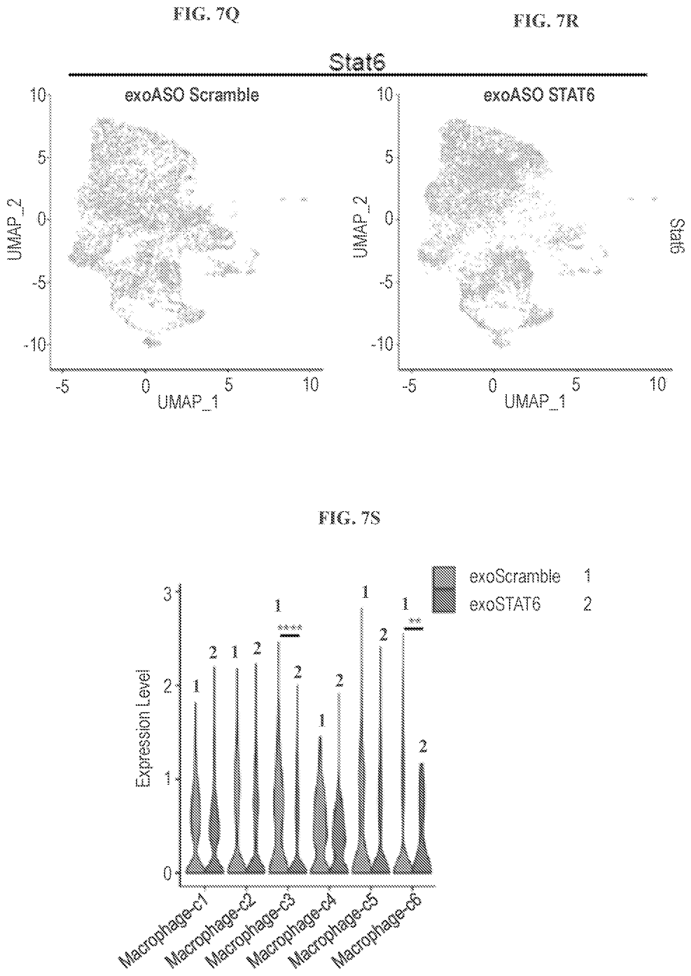

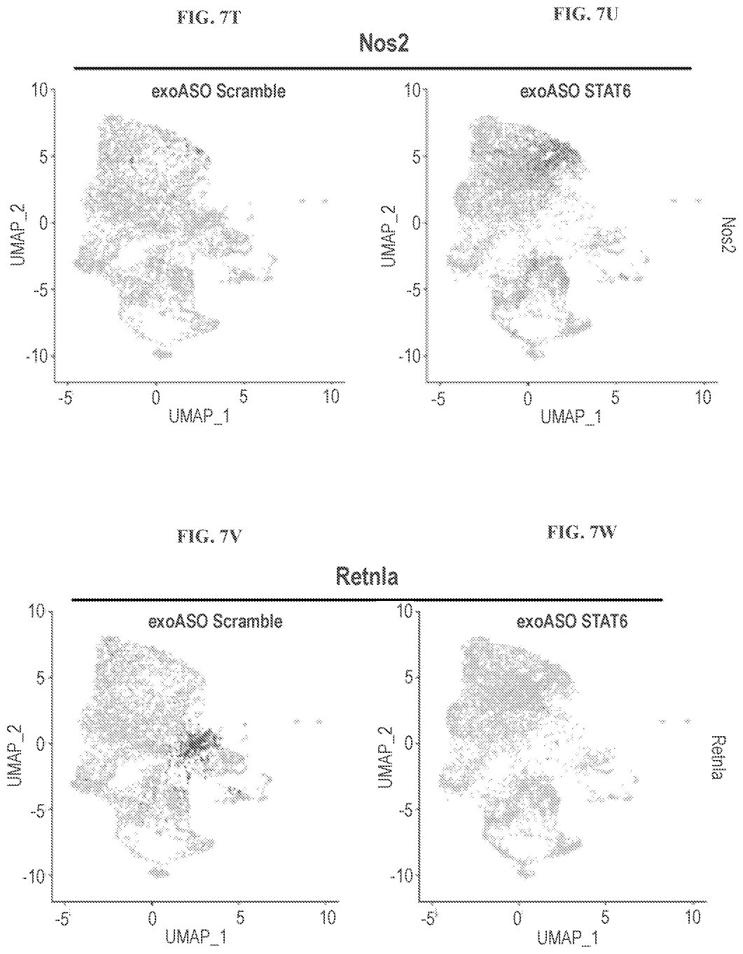

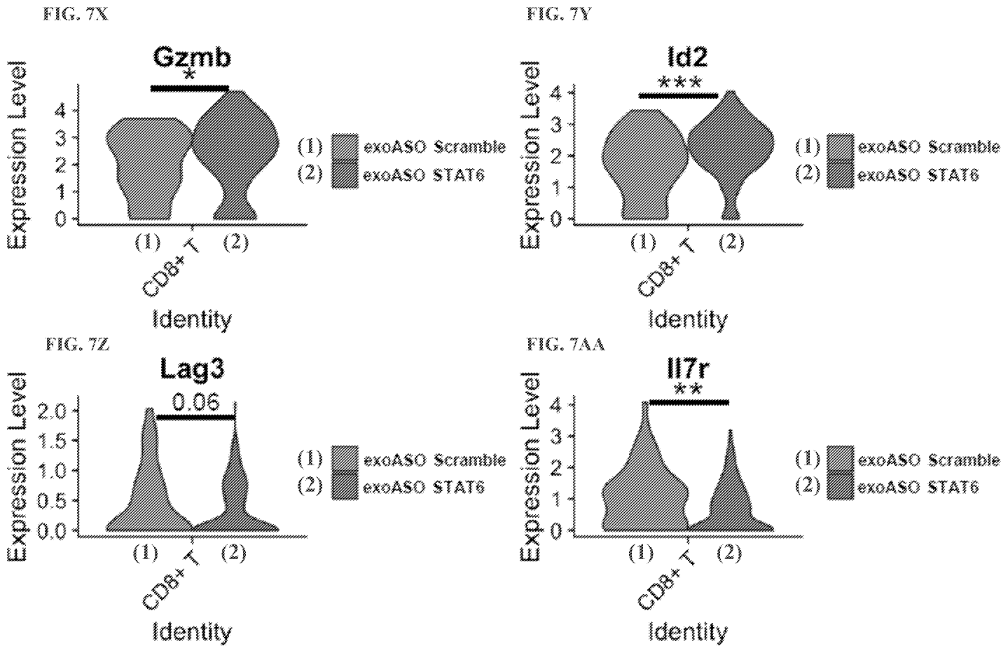

The pharmacodynamic effect of exoASO-STAT6 treatment was investigated in the CT26 tumor model. CT26 tumor bearing mice were treated with three IT doses of exoASO STAT6 (4 μg) or controls as indicated in FIG. 7A. The tumor was dissected, and the macrophages were enriched by CD11b positive cell sorting. The enrichment of CD11b was confirmed by flow cytometry. Treatment with exoASO-STAT6 induced about a 50% reduction of Stat6 mRNA in the CD11b enriched cells, while only 17% KD was observed after treatment with free STAT6 ASO (FIGS. 7B-7C). Moreover, enriched CD11b cells showed a 56% reduction in the expression of the M2 gene Arg1 whereas free STAT6 ASO-mediated reduction of Arg1 expression was significantly less (20%).

To assess in more detail the impact of Stat6 knockdown on the TME myeloid compartment, gene-expression analysis was performed using Nanostring platform. Genes associated with M2 macrophage phenotype such as Csf1r were down regulated by up to 80% in the exoASO-STAT6 treatment group as compared to the control exoASO group. Similarly, a 2-fold increase in M1 related genes such as Nos2 was observed in the exoASO-STAT6 group, suggesting an effective reprogramming of these TAMs following exoASO-STAT6 treatment (FIGS. 7D-7K). An equivalent dose of free STAT6 ASO treatment did not result in measurable modulation of M2 or M1 related genes demonstrating that exosome-mediated delivery is required in vivo to induce effective reprogramming of TAMs.

Next, we evaluated the effect of exoASO-STAT6 treatment on the composition of the tumor immune infiltrate. Immunophenotyping of CT26 tumors after IT treatment with exoASO-STAT6 or control exoASO as described in FIG. 7A was performed to evaluate global changes in the immune cells from the TME. Cytofluorometric analyses of digested tumors showed a significant 2-fold increase in the immune infiltrate (% CD45+ cells) in the exoASO-STAT6 vs control exoASO group (FIGS. 7L-7O). Despite the increase in total immune cells, the frequency of total macrophages and total T cells in the tumor remained unchanged, reflecting the expansion of new myeloid populations. Importantly, the expression of the M2 marker CD206 was reduced by 60% in F4/80+ TAMs by exoASO-STAT6 (FIG. 7M), supporting the results from the gene expression analysis (FIG. 7H). Analysis of the T cell infiltrate showed no changes in the frequency of CD8 T cells. However, a significant decrease (P<0.0001) in the proportion of Tregs within the CD4 compartment was observed (38% vs 50% exoASO-STAT6 vs control exoASO, respectively), as well as a 3.3-fold reduction in the frequency of Tregs in the immune infiltrate (FIGS. 7N-70).

Assessment of gene expression of tumor infiltrating cells by single cell RNA sequencing (scRNAseq) confirmed that exoASO-STAT6 causes significant changes in the monocyte/macrophage populations in the tumor (FIGS. 7P-7S). Unbiased analysis identified six monocyte/macrophage populations with distinct gene expression profile within the CT26 tumors (FIG. 7P; Table 4A). Clusters c3 and c6 showed significant reduction of Stat6 expression induced by exoASO-STAT6 compared to the control exoASO treatment (P<0.0001 and P<0.01 for c3 and c6, respectively) (FIGS. 7Q-7S). Two subsets, c3 and c5, expanded in response to exoASO-STAT6 (FIG. 7P). Importantly, levels of the M1 marker Nos2 increased significantly in the exoASO-STAT6 treatment group, in particular in the expanded clusters c3 and c5 (FIGS. 7T-7U; Tables XXB-XXc). Nos2 induction has been associated with macrophage reprogramming and anti-tumor immune response to exoIL-12 therapy. In contrast, clusters cl and c6 were reduced by exoASO-STAT6 treatment (FIG. 7P). Cluster c6 is represented by cells expressing high levels of CD163 and Retnla (Fizz1), a population predominantly associated with M2 polarized macrophages (FIGS. 7V-7W). Fizz1 has been previously reported to be regulated by IL-4 and STAT6 signaling pathway. exoASO-STAT6 treatment profoundly reduced the levels of this population as compared to control exoASO. The cluster 1 is represented by high levels of complement proteins C1q, an important mediator of innate inflammation that is induced by M2 reprograming cytokines like IL-4. exoASO-STAT6 treatment resulted in significant reduction of this population. These results confirm a reduction in the monocyte/macrophage cells with M2 polarized markers and an increase in the M1 polarized population. Furthermore, the scRNAseq study also revealed that exoASO-STAT6 induced activation of the tumor-infiltrating CD8 T cells, which showed increased expression of effector/activation genes such as Gzmb and Id2, and decrease of exhaustion markers such as Lag3 (FIGS. 7X-7AA).

TABLE 4A
Clusters identified by single cell RNAseq in the CT26 tumor infiltrate

| | clustero | cluster1 | cluster2 | cluster3 | cluster4 | cluster5 | cluster6 |
|---|---|---|---|---|---|---|---|
| 1 | Thbs1 | Mgp | Timp1 | C1qa | Ube2c | Gzmb | Gm42418 |
| 2 | Arg1 | Ogn | Lgals7 | C1qb | Cenpf | Il2rb | Lgmn |
| 3 | Ly6c2 | Dcn | S100a4 | C1qc | Top2a | Gzma | Psap |
| 4 | Slpi | Aspn | Spp1 | Apoe | Prc1 | Nkg7 | Gatm |
| 5 | Gm15056 | Tmem176b | Hmga2 | Ccl12 | Tubb5 | Prf1 | Ctsb |
| 6 | Il1b | Mmp3 | Rhox5 | Lyz2 | Hmgb2 | Ms4a4b | Ccl6 |
| 7 | Hmox1 | Tmem176a | Igfbp6 | Ms4a7 | Tubb4b | Trbc1 | Ccl8 |
| 8 | Ms4a4c | Cebpd | Lxn | Ctsc | Anln | Gzmc | C1qc |
| 9 | Cd14 | Cp | Tm4sf1 | Ccl7 | Hist1h1b | Trbc2 | Crip1 |
| 10 | Malt1 | Apod | Prkg2 | Ch25h | Cks1b | Irf8 | Cotl1 |
| 11 | Plac8 | Ccn1 | Phgdh | Plau | Cks2 | AW1120 | Laptm5 |
| | | | | | | 10 | |
| 12 | F10 | Sox4 | Asns | Cxcl9 | Cdk1 | Cd3g | C1qa |
| 13 | Cybb | C1s1 | Hspd1 | Gatm | Birc5 | Satb1 | Mafb |
| 14 | Id2 | Fn1 | Rpl391 | Ctss | Stmn1 | Serpinb9 | Gm26917 |
| 15 | Chil3 | Pcolce | Baiap2l1 | Msr1 | Smc4 | C1qa | Maf |
| 16 | Ptgs2 | Fosb | Med10 | Ccl2 | Racgap1 | C1qc | Ccl24 |
| 17 | Ms4a6d | Spp1 | Mt2 | Mrc1 | Tm4sf1 | Ccl5 | Grn |
| 18 | Mafb | Baiap2l1 | Tnfrsf12a | Trem2 | H2afz | Thy1 | Ccl7 |
| 19 | Ccr2 | Col1a2 | 1700012B | Grn | Ube2s | Ccl8 | Mrc1 |
| | | | 09Rik | | | | |
| 20 | Fcgr1 | Lima1 | Tpm1 | Cxcl16 | Pbk | Cd52 | Apoe |
| 21 | Ccl9 | Cxcl1 | Rpl12 | Fcgr3 | Tpx2 | Apoe | Marcks |
| 22 | Ms4a6c | Cald1 | Cald1 | Fcgr2b | Selenoh | Klrd1 | Sdc3 |
| 23 | Cxcl2 | Klf9 | Ccnd1 | Lgmn | Smc2 | C1qb | Wfdc17 |
| 24 | Ccl6 | Cavin2 | Oaf | C3ar1 | Ran | Xist | Ccl9 |
| 25 | Cd74 | Rarres2 | Cd34 | Dab2 | Tuba1b | Icos | Aif1 |
| 26 | Psap | Sema5a | Anxa3 | Fcer1g | Pclaf | Vps37b | Arpc5 |
| 27 | Tgfbi | Prrx1 | Atf5 | Cd68 | 1700012 | Ccl7 | Efhd2 |
| | | | | | B09Rik | | |
| 28 | H2-Aa | Nr2f1 | Rarres1 | Unc93b1 | Hmgb1 | Ptprc | BC005 |
| | | | | | | | 537 |
| 29 | Zeb2 | Mt1 | Rps18 | Cd74 | Rrm2 | Serpinb | F13a1 |
| | | | | | | 6b | |
| 30 | Srgn | Igfbp6 | Rpl28 | Aif1 | Cdc20 | Laptm5 | Pf4 |
| 31 | Vcan | Igfbp4 | Cavin3 | Stab1 | Phgdh | Lcp1 | Clec4a1 |
| 32 | Lst1 | F830016 | Pcolce | Adgre1 | Tmpo | Ccl12 | Selenop |
| | | B08Rik | | | | | |
| 33 | Hilpda | Pbx1 | Cav1 | Cfp | Timp1 | Pf4 | Glul |
| 34 | Ctss | Slit2 | Ercc1 | Pld4 | Hmga2 | Coro1a | Sirpa |
| 35 | H2-Ab1 | Serping1 | S100a6 | Cd72 | Cenpe | Ikzf2 | Zfp703 |
| 36 | Plin2 | Serpine1 | Gas5 | Ifi30 | H2afx | Sh2d2a | Creg1 |
| 37 | Adam8 | Errfi1 | Esm1 | Fcgr4 | Prkg2 | Rac2 | Junb |
| 38 | Rsad2 | Cd9 | Lgals1 | Clec12a | S100a4 | Cd3d | Dazap2 |
| 39 | Il1a | Cd63 | Npm1 | Ctsb | Nusap1 | Runx3 | Fabp5 |
| 40 | Wfdc17 | Maged1 | Eef1b2 | Ctsa | Ccnb1 | Rgs1 | Ap2a2 |
| 41 | Slfn2 | Itgb5 | Cavin2 | Csf1r | Cenpa | Nabp1 | Gda |
| 42 | Itgam | Tgfbr3 | Rps2 | Ctsz | Rpl39l | Cemip2 | Hal |
| 43 | Cdkn1a | Nedd4 | Hspe1 | Ms4a6c | Tnfrsf12a | Ptpn18 | Naaa |
| 44 | Clec4d | Dnajb1 | Hspa9 | Hexa | Dut | Lyz2 | Cd83 |
| 45 | Cxcl3 | Rabac1 | Esd | Ms4a6d | Ercc1 | Mrc1 | Kctd12 |
| 46 | Ccl24 | Anxa1 | Rpl22l1 | Cd93 | Hmgn1 | Dennd4a | Cltc |
| 47 | Fabp5 | Jun | Ybx3 | Cotl1 | H2afv | Lgmn | Ckb |
| 48 | Mmp12 | Hes1 | Eif4ebp1 | Atp1b3 | Ccna2 | Id2 | Fcgrt |
| 49 | Pf4 | Neat1 | Csrp2 | Mpeg1 | Cavin3 | Tnfrsf9 | Atp6v0a1 |
| 50 | Inhba | Tm4sf1 | Serpinh1 | Tyrobp | Cdca3 | Gzmf | Hmox1 |

| cluster7 | cluster8 | cluster9 | cluster10 | cluster11 | cluster12 | cluster13 |
|---|---|---|---|---|---|---|
| S100a9 | Mki67 | Rps26 | C1s1 | Cdc20 | Gzma | Ccr7 |
| S100a8 | Arl6ip1 | Rpl13 | Sema5a | Pttg1 | Gzmb | Ccl22 |
| G0s2 | Birc5 | Rps3a1 | Slit2 | Cenpa | Nkg7 | Ramp3 |
| Ccl3 | Pclaf | Rpl30 | Nr2f1 | Kif20a | Prf1 | Cd209a |
| Hcar2 | Cdca8 | Rpsa | Col1a2 | Ccnb2 | Il2rb | Cbfa2t3 |
| Cstdc4 | Ube2c | Rps4x | Ogn | Cdca3 | Ms4a4b | Timd4 |
| Acod1 | Slamf9 | Rpl41 | Cp | Cenpe | Ctla2a | Sept3 |
| CcrI2 | Cdca3 | Rplp0 | C1qb | Anln | Serpinb9 | H2-DMb2 |
| Hdc | Cenpe | S100a6 | Sox4 | Hmgb3 | Txk | Pkib |
| Cxc12 | Ccna2 | Rpl6 | Apod | Racgap1 | Ncr1 | Mmp25 |
| Stfa211 | Ccnb2 | Lgals1 | C1qa | Selenoh | Klrd1 | Lsp1 |
| Slfn4 | Tpx2 | Rpl7 | C1qc | Tpx2 | Satb1 | H2-Eb1 |
| Rsad2 | C1qb | Rps12 | Ccl12 | Dynll1 | Serpinb6b | Cytip |
| Clec4e | Pttg1 | Rps2 | Scd1 | Ube2s | Ctsw | H2-Aa |
| Il1b | Gatm | Rpl37 | Apoe | Rpl391 | Trbc1 | Syngr2 |
| Srgn | C1qc | Rpl24 | Ank | Birc5 | Klrk1 | Cd74 |
| Mxd1 | Top2a | Rpl28 | Tmem176a | Esm1 | Sept1 | Napsa |
| Il1r2 | C1qa | Rps5 | Vcam1 | Ddx39 | Gimap4 | St8sia4 |
| Slc7a11 | Clec12a | Eef1b2 | Hmga2 | Prkg2 | Gimap3 | H2-Ab1 |
| Il1rn | Cenpa | Rpl34 | Tmem176b | Ran | Ptprcap | Tbc1d4 |
| Trem1 | Smc2 | S100a4 | Lima1 | Hdgf | Sh2d2a | Mgl2 |
| Il1f9 | Stmn1 | Rpl36 | Mgp | Serpinh1 | Gem | Cd83 |
| Samsn1 | Pld4 | Rpl36a | Mmp3 | Cks1b | Car2 | Cst3 |
| Csf3r | Tuba1b | Rps18 | Axl | 1700012B09Rik | Skap1 | Myo1g |
| Mirt1 | Ctsc | Npm1 | Pcolce | Fst | Eomes | Tmsb4x |
| Cd24a | Plau | Rpl21 | Lyz2 | Cks2 | Thy1 | Rel |
| Marck | Tagln2 | Rpl5 | Cebpd | Tnfrsf12a | Klra3 | Crip1 |
| sl1 | | | | | | |
| Cxcr2 | Trem2 | Rpl35 | Fn1 | Hmgb1 | Klre1 | Ifi30 |
| Clec4d | Tubb5 | Ran | mt-Co2 | Lmna | Selplg | H2-DMb1 |
| Plek | Cxcl16 | Rpl4 | Timp2 | Phgdh | Irf8 | H2-DMa |
| Asprv1 | Ifi30 | H2afz | Baiap2l1 | Oaf | AW112010 | Etv3 |
| Nfkbia | Ch25h | Tubb5 | Mt2 | Hmgn1 | Hcst | Atox1 |
| Pnrc1 | H2afx | Ube2c | Pcbp2 | Lgals1 | Ptprc | Wdfy4 |
| Ets2 | Cfp | Gas5 | Ccnd1 | Lyar | Vps37b | Dennd4a |
| Isg15 | Tgfbi | Snrpd1 | mt-Cytb | Asns | Rac2 | Tnip3 |
| Tnfaip | Msr1 | Hmgb1 | Plec | Cacybp | Dennd4a | Gm2a |
| 2 | | | | | | |
| Cd274 | Hist1h1b | AY036118 | ligp1 | Csrp2 | Xcl1 | Plbd1 |
| Ptgs2 | Unc93b1 | Rpl26 | mt-Nd1 | Tm4sf1 | Cd52 | Bcl2a1b |
| Gadd | Hmgb2 | Rhox5 | #N/A | Cct6a | Ifngr1 | Tmem123 |
| 45b | | | | | | |
| Ppp1r | Aif1 | Rpl39I | #N/A | Rhox5 | Ugcg | Serpinb6b |
| 15a | | | | | | |
| Slfn1 | Ccl12 | 1810037I17Rik | #N/A | H2afz | Bcl2 | Irf8 |
| Ccl4 | Ms4a7 | Tubb4b | #N/A | S100a11 | Gzmc | Naaa |
| Msrb1 | Grn | Nap1l1 | #N/A | Hspd1 | Ets1 | Malt1 |
| Cxcl3 | Cxcl9 | Phgdh | #N/A | Med10 | Nabp1 | Samsn1 |
| Lmnb1 | Xist | Rpa3 | #N/A | Lgals7 | Runx3 | Traf1 |
| Resf1 | Lyz2 | Anp32b | #N/A | Hmgb2 | Serpinb9b | Nr4a2 |
| Isg20 | Ighm | 1700012B09Rik | #N/A | Ppp1r14b | Cemip2 | Ighm |
| Csf1 | Ccl7 | Cks2 | #N/A | Ccnd1 | Ccl5 | Serpinb9 |
| Icam1 | Ccl2 | 2410006H16Rik | #N/A | Baiap2l1 | Tnfrsf9 | Ccl5 |
| Ifit1 | Atp1b3 | Hmgb2 | #N/A | Ncl | Gzmd | Ifitm1 |

| cluster14 | cluster15 | cluster16 | cluster17 | cluster18 |
|---|---|---|---|---|
| Cd3g | Bgn | Klk1 | Igfbp7 | Mcpt4 |
| Trbc2 | Tnc | Ccr9 | Igfbp3 | Tpsb2 |
| Ms4a4b | Fstl1 | Siglech | Plvap | Cpa3 |
| Cd3d | Igfbp7 | Cox6a2 | Gpihbp1 | Cyp11a1 |
| Cd3e | Timp3 | Rnase6 | Ubd | Mcpt8 |
| Ctla4 | Col6a3 | Cyb561a3 | Timp3 | Fcer1a |
| Icos | Mfap5 | Bcl11a | Ramp2 | Hs3st1 |
| Trac | Col5a2 | Ighm | Egfl7 | Cd200r3 |
| Tnfrsf4 | Meg3 | Iglc3 | Cdh5 | Ms4a2 |
| Cd2 | Postn | Atp1b1 | Esam | Il13 |
| Cd8b1 | Col12a1 | Klk1b27 | Emcn | Mrgprb1 |
| Thy1 | Fbn1 | Cd7 | Sparcl1 | Tph1 |
| Gimap3 | I133 | Fcrla | Trp53i11 | Il4 |
| Cxcr6 | Tnfaip6 | Pacsin1 | Mcam | Tpsab1 |
| Cd28 | Ccl11 | Mctp2 | Car4 | Ctsg |
| Il2rb | Col6a2 | Mzb1 | Adgrf5 | Cma1 |
| Tnfrsf18 | Col5a3 | Spib | Rbp7 | Hdc |
| Il2ra | Lum | St8sia4 | Cldn5 | Gata2 |
| Cd8a | Gpx3 | Gpr171 | Depp1 | Serpinb1a |
| Ltb | Loxl1 | Mef2c | Icam2 | Hgf |
| Ptprcap | Cxcl12 | Tspan13 | Mgll | Cish |
| Lck | Lox | Slpi | Stc1 | Cyp4f18 |
| Cd27 | Cpxm1 | Sell | Tmem252 | Ctse |
| Sh2d2a | Plpp3 | P2ry14 | Lrg1 | Srgn |
| Pdcd1 | Mmp2 | Il7r | S1pr1 | Il6 |
| Lat | Ebf1 | Ly6c2 | Myct1 | Gzmb |
| Gimap4 | Thy1 | Selplg | Plpp3 | Csf2rb2 |
| Sept1 | Crabp1 | Blnk | Mmrn2 | Furin |
| Ablim1 | Ctsk | Irf8 | Fabp4 | Csf2rb |
| Skap1 | Nid1 | Pltp | Flt1 | ler3 |
| Itk | Adamts2 | Pld4 | Kdr | Socs2 |
| Trbc1 | Acta2 | Runx2 | Pecam1 | Il1rl1 |
| Nkg7 | Aebp1 | Lair1 | Ctla2a | Rgs1 |
| Satb1 | Rarres2 | Abhd17b | Gng11 | Lilrb4a |
| Ikzf2 | Serping1 | Tcf4 | Sparc | Gm20186 |
| Klrd1 | Col5a1 | Mpeg1 | Ecscr | Lilr4b |
| Ets1 | Col6a1 | Ctsh | Rbp1 | Gcnt1 |
| Gzmb | Col3a1 | Ncf1 | Col4a2 | Tax1bp1 |
| Rac2 | Col1a1 | Dnajc7 | Col4a1 | Ctla2a |
| Vps37b | Sparc | Rabgap1I | Ly6c1 | Ccl3 |
| Prf1 | Serpinh1 | Rell1 | Fabp5 | Osm |
| Cd52 | Serpinf1 | Nucb2 | Cav1 | Ccl4 |
| Akap13 | Dcn | Xbp1 | Pdlim1 | Frmd4b |
| Ptpn22 | Col1a2 | Xist | Crip2 | Ifitm1 |
| Tnfrsf9 | Fbln2 | Cybb | Fkbp1a | Ccl9 |
| VgII4 | Cxcl14 | Stk17b | Cxcl9 | Ccl6 |
| Ifngr1 | Serpine1 | Plac8 | Cd93 | Acod1 |
| Il7r | Col4a1 | Tyrobp | Ece1 | Plk3 |
| Uhrf2 | Timp1 | Smc6 | Ly6a | Csf1 |
| Ccl5 | Saa3 | Bst2 | Rhoc | Gm12840 |

TABLE 4B
Differentially expressed genes between exoASO-STAT6 and
control exoASO groups in cluster 3 and cluster 5.

| | | | | |
|---|---|---|---|---|
| p_val | avg_logFC | pct.1 | pct.2 | p_val_adj |

C3

| | | | | | |
|---|---|---|---|---|---|
| Cxcl9 | 2.56E−28 | 1.387886 | 0.634 | 0.266 | 5.29E−24 |
| Rps12 | 1.44E−23 | 0.407943 | 1 | 0.994 | 2.96E−19 |
| Ftl1 | 6.64E−23 | −0.41809 | 1 | 1 | 1.37E−18 |
| Rpl23 | 3.87E−22 | 0.372373 | 0.993 | 0.977 | 8.00E−18 |
| Rps24 | 1.84E−20 | 0.373368 | 1 | 0.979 | 3.80E−16 |
| Cstb | 6.49E−20 | −0.41189 | 0.959 | 0.988 | 1.34E−15 |
| Rpl18a | 1.81E−19 | 0.396118 | 0.979 | 0.951 | 3.74E−15 |
| Igfbp4 | 1.84E−19 | 0.503659 | 0.324 | 0.085 | 3.80E−15 |
| Hmox1 | 5.57E−19 | −0.68678 | 0.766 | 0.952 | 1.15E−14 |
| Rps4x | 6.59E−19 | 0.435325 | 0.993 | 0.964 | 1.36E−14 |
| Rpl13a | 3.58E−18 | 0.442749 | 0.972 | 0.949 | 7.39E−14 |
| Fth1 | 4.16E−18 | −0.64596 | 1 | 1 | 8.59E−14 |
| Rps20 | 6.04E−18 | 0.376904 | 0.993 | 0.987 | 1.25E−13 |
| Apoe | 6.99E−18 | 0.780726 | 0.952 | 0.781 | 1.44E−13 |
| Rpl13 | 8.04E−18 | 0.38878 | 0.986 | 0.979 | 1.66E−13 |
| Grina | 1.75E−16 | −0.52504 | 0.766 | 0.925 | 3.61E−12 |
| H2-Ab1 | 1.88E−16 | 0.821725 | 0.876 | 0.78 | 3.87E−12 |
| Rps19 | 1.96E−16 | 0.362926 | 0.979 | 0.966 | 4.05E−12 |
| Rpl27a | 2.08E−16 | 0.323732 | 1 | 0.987 | 4.30E−12 |
| Atp1b3 | 3.93E−16 | 0.532755 | 0.89 | 0.892 | 8.11E−12 |
| Cd86 | 4.52E−16 | 0.429905 | 0.448 | 0.335 | 9.34E−12 |
| Rpl30 | 7.22E−16 | 0.347924 | 0.986 | 0.968 | 1.49E−11 |
| Clec4d | 1.07E−15 | −0.70028 | 0.531 | 0.787 | 2.20E−11 |
| Rps11 | 1.32E−15 | 0.41841 | 0.993 | 0.975 | 2.72E−11 |
| Eef1a1 | 1.59E−15 | 0.27884 | 1 | 0.997 | 3.29E−11 |
| Tpt1 | 1.85E−15 | 0.245561 | 1 | 1 | 3.81E−11 |
| Trf | 2.01E−15 | 0.581443 | 0.531 | 0.339 | 4.15E−11 |
| Por | 2.20E−15 | −0.4174 | 0.503 | 0.825 | 4.55E−11 |
| Ccnd1 | 2.31E−15 | 0.608515 | 0.462 | 0.303 | 4.78E−11 |
| Rpl19 | 4.48E−15 | 0.299707 | 0.993 | 0.981 | 9.25E−11 |
| Rps16 | 4.94E−15 | 0.326395 | 1 | 0.988 | 1.02E−10 |
| Rps10 | 8.27E−15 | 0.356878 | 0.986 | 0.98 | 1.71E−10 |
| Ell2 | 9.12E−15 | −0.55826 | 0.31 | 0.639 | 1.88E−10 |
| Rps15 | 9.54E−15 | 0.409783 | 0.986 | 0.965 | 1.97E−10 |
| Rpl28 | 9.88E−15 | 0.372546 | 0.993 | 0.96 | 2.04E−10 |
| Rps9 | 1.43E−14 | 0.337039 | 0.986 | 0.996 | 2.96E−10 |
| Rpl6 | 1.77E−14 | 0.326065 | 0.993 | 0.96 | 3.65E−10 |
| Rpl23a | 3.35E−14 | 0.374767 | 0.966 | 0.933 | 6.92E−10 |
| Rpl10a | 7.38E−14 | 0.403634 | 0.966 | 0.922 | 1.52E−09 |
| Lgals3 | 8.43E−14 | −0.58529 | 0.945 | 0.981 | 1.74E−09 |
| Rps27a | 9.95E−14 | 0.293781 | 1 | 0.981 | 2.05E−09 |
| Gm10076 | 1.18E−13 | 0.292342 | 0.545 | 0.505 | 2.44E−09 |
| Glrx | 1.74E−13 | −0.56945 | 0.655 | 0.876 | 3.59E−09 |
| Cd81 | 1.82E−13 | 0.410449 | 0.345 | 0.116 | 3.75E−09 |
| Cd36 | 2.42E−13 | −0.94507 | 0.193 | 0.449 | 4.99E−09 |
| Rps5 | 2.57E−13 | 0.360872 | 0.986 | 0.976 | 5.30E−09 |
| Rps7 | 2.64E−13 | 0.356396 | 1 | 0.96 | 5.44E−09 |
| Eef1b2 | 3.26E−13 | 0.370191 | 0.952 | 0.881 | 6.72E−09 |
| Lilr4b | 7.02E−13 | −0.42412 | 0.793 | 0.927 | 1.45E−08 |
| Rpl32 | 1.34E−12 | 0.37917 | 0.986 | 0.967 | 2.77E−08 |
| Rplp2 | 1.46E−12 | 0.320473 | 1 | 0.976 | 3.01E−08 |
| Cox7a2l | 1.60E−12 | 0.360855 | 0.752 | 0.716 | 3.30E−08 |
| Gas5 | 1.85E−12 | 0.321564 | 0.503 | 0.477 | 3.83E−08 |
| Rplp1 | 2.59E−12 | 0.245388 | 0.993 | 0.997 | 5.35E−08 |
| Rpl17 | 6.35E−12 | 0.350173 | 0.993 | 0.971 | 1.31E−07 |
| Cd83 | 7.20E−12 | 0.572638 | 0.669 | 0.548 | 1.49E−07 |
| Atp5g2 | 8.16E−12 | 0.334846 | 0.91 | 0.893 | 1.68E−07 |
| Ctsl | 8.24E−12 | −0.64894 | 0.966 | 0.992 | 1.70E−07 |
| Rps3 | 8.41E−12 | 0.321921 | 0.979 | 0.944 | 1.74E−07 |
| Blvrb | 1.02E−11 | −0.49678 | 0.745 | 0.882 | 2.11E−07 |
| Eif3f | 1.40E−11 | 0.357628 | 0.91 | 0.86 | 2.89E−07 |
| Fau | 1.60E−11 | 0.264386 | 1 | 0.99 | 3.30E−07 |
| Heatr5a | 1.82E−11 | 0.192735 | 0.269 | 0.17 | 3.76E−07 |
| Rpl37a | 2.66E−11 | 0.329926 | 1 | 0.988 | 5.50E−07 |
| Rp15 | 2.89E−11 | 0.380935 | 0.945 | 0.874 | 5.96E−07 |
| Il10ra | 3.32E−11 | 0.365333 | 0.634 | 0.546 | 6.86E−07 |
| Twf2 | 3.47E−11 | 0.263309 | 0.579 | 0.565 | 7.16E−07 |
| Rps3a1 | 3.94E−11 | 0.273536 | 1 | 0.987 | 8.14E−07 |
| Rpl36a | 4.69E−11 | 0.335942 | 0.897 | 0.849 | 9.68E−07 |
| H2-Eb1 | 5.17E−11 | 0.728322 | 0.821 | 0.687 | 1.07E−06 |
| Slc48a1 | 5.59E−11 | −0.47072 | 0.49 | 0.75 | 1.15E−06 |
| Cd274 | 6.77E−11 | −0.48477 | 0.717 | 0.893 | 1.40E−06 |
| Dnase1l3 | 6.84E−11 | 0.361288 | 0.228 | 0.083 | 1.41E−06 |
| Palld | 6.95E−11 | 0.296046 | 0.366 | 0.22 | 1.44E−06 |
| C1qb | 7.15E−11 | 0.654351 | 0.662 | 0.594 | 1.48E−06 |
| Prdx6 | 7.40E−11 | −0.52589 | 0.476 | 0.697 | 1.53E−06 |
| Ctsd | 8.17E−11 | −0.43154 | 0.952 | 0.992 | 1.69E−06 |
| Rack1 | 8.66E−11 | 0.28781 | 0.972 | 0.97 | 1.79E−06 |
| Rpl3 | 1.01E−10 | 0.331619 | 0.993 | 0.952 | 2.08E−06 |
| Sod2 | 1.11E−10 | −0.50337 | 0.786 | 0.909 | 2.29E−06 |
| Rpl12 | 1.56E−10 | 0.324756 | 0.972 | 0.948 | 3.21E−06 |
| Rps25 | 2.16E−10 | 0.284324 | 0.931 | 0.937 | 4.45E−06 |
| Smpdl3a | 2.28E−10 | −0.47752 | 0.476 | 0.716 | 4.71E−06 |
| H2-Aa | 2.74E−10 | 0.742458 | 0.89 | 0.771 | 5.65E−06 |
| Lyz2 | 2.83E−10 | 0.492306 | 0.972 | 0.915 | 5.85E−06 |
| R3hcc1l | 3.51E−10 | 0.103478 | 0.117 | 0.117 | 7.24E−06 |
| Eif4e | 3.56E−10 | −0.37911 | 0.538 | 0.777 | 7.34E−06 |
| Rpl7 | 3.92E−10 | 0.296474 | 1 | 0.972 | 8.10E−06 |
| Commd4 | 4.58E−10 | 0.116971 | 0.379 | 0.419 | 9.47E−06 |
| Gm14636 | 4.73E−10 | −0.66941 | 0.228 | 0.486 | 9.77E−06 |
| Ly86 | 4.77E−10 | 0.397399 | 0.731 | 0.625 | 9.86E−06 |
| Cndp2 | 5.79E−10 | −0.3295 | 0.91 | 0.963 | 1.20E−05 |
| AA467197 | 6.27E−10 | −0.64409 | 0.166 | 0.363 | 1.30E−05 |
| Rps29 | 6.75E−10 | 0.184879 | 1 | 0.992 | 1.39E−05 |
| Cd74 | 6.99E−10 | 0.65164 | 0.966 | 0.884 | 1.44E−05 |
| Prdx1 | 7.07E−10 | −0.58071 | 0.979 | 0.992 | 1.46E−05 |
| Rpl18 | 7.13E−10 | 0.306328 | 0.966 | 0.963 | 1.47E−05 |
| Ly6c2 | 7.19E−10 | −0.64411 | 0.407 | 0.68 | 1.48E−05 |
| Serinc3 | 7.30E−10 | 0.343362 | 0.883 | 0.859 | 1.51E−05 |
| Csf1r | 7.40E−10 | 0.34238 | 0.848 | 0.806 | 1.53E−05 |
| H2-DMb1 | 8.31E−10 | 0.520577 | 0.683 | 0.473 | 1.72E−05 |
| Oip5os1 | 8.51E−10 | 0.186252 | 0.434 | 0.444 | 1.76E−05 |
| Pdlim5 | 1.16E−09 | 0.149774 | 0.372 | 0.415 | 2.39E−05 |
| Tecr | 1.18E−09 | 0.231068 | 0.469 | 0.353 | 2.44E−05 |
| Rpl27 | 1.23E−09 | 0.301668 | 0.986 | 0.966 | 2.54E−05 |
| Rgl1 | 1.49E−09 | −0.40273 | 0.724 | 0.881 | 3.07E−05 |
| mt-Nd4 | 1.80E−09 | 0.28365 | 0.966 | 0.974 | 3.71E−05 |
| mt-Cytb | 2.01E−09 | 0.356525 | 0.979 | 0.987 | 4.16E−05 |
| Rab4b | 2.51E−09 | 0.212385 | 0.517 | 0.472 | 5.19E−05 |
| Rpl34 | 2.62E−09 | 0.249859 | 0.986 | 0.964 | 5.41E−05 |
| Oat | 2.62E−09 | 0.104066 | 0.4 | 0.473 | 5.42E−05 |
| Vcam1 | 2.96E−09 | 0.630449 | 0.352 | 0.138 | 6.12E−05 |
| Isoc1 | 2.97E−09 | 0.219888 | 0.4 | 0.407 | 6.13E−05 |
| mt-Nd1 | 2.99E−09 | 0.278939 | 0.966 | 0.976 | 6.16E−05 |
| Jaml | 3.32E−09 | 0.394598 | 0.648 | 0.521 | 6.85E−05 |
| Dock11 | 3.80E−09 | 0.206049 | 0.324 | 0.256 | 7.84E−05 |
| Sdf4 | 3.85E−09 | 0.174238 | 0.593 | 0.632 | 7.95E−05 |
| Ptma | 4.31E−09 | 0.227132 | 1 | 0.986 | 8.90E−05 |
| Upp1 | 4.36E−09 | −0.57258 | 0.324 | 0.555 | 9.00E−05 |
| Saa3 | 4.68E−09 | −0.89862 | 0.083 | 0.313 | 9.67E−05 |
| Sh3bp5 | 4.98E−09 | −0.39675 | 0.331 | 0.573 | 0.000103 |
| Wfdc17 | 5.13E−09 | −0.56827 | 0.869 | 0.94 | 0.000106 |
| Fcgr2b | 5.39E−09 | 0.278309 | 0.931 | 0.906 | 0.000111 |
| mt-Atp6 | 5.72E−09 | 0.281694 | 0.979 | 0.993 | 0.000118 |
| Txn1 | 5.80E−09 | −0.35411 | 0.903 | 0.962 | 0.00012 |
| Rpl39 | 5.83E−09 | 0.234376 | 1 | 0.965 | 0.00012 |
| H2-DMa | 6.25E−09 | 0.363502 | 0.862 | 0.769 | 0.000129 |
| Sulf2 | 6.78E−09 | 0.184732 | 0.152 | 0.054 | 0.00014 |
| Nop53 | 7.76E−09 | 0.187211 | 0.538 | 0.553 | 0.00016 |
| Ednrb | 7.99E−09 | −0.66374 | 0.145 | 0.384 | 0.000165 |
| Plpp3 | 8.19E−09 | −0.2474 | 0 | 0.138 | 0.000169 |
| Adam8 | 8.47E−09 | −0.46694 | 0.579 | 0.801 | 0.000175 |
| Tspo | 8.53E−09 | −0.28265 | 0.979 | 0.987 | 0.000176 |
| Tbc1d22a | 8.59E−09 | 0.186284 | 0.262 | 0.198 | 0.000177 |
| Slc16a3 | 8.68E−09 | −0.44971 | 0.531 | 0.739 | 0.000179 |
| Uri1 | 8.89E−09 | 0.137253 | 0.179 | 0.156 | 0.000183 |
| Ubb | 9.56E−09 | −0.36582 | 0.938 | 0.961 | 0.000197 |
| Rnf128 | 1.03E−08 | −0.36361 | 0.09 | 0.302 | 0.000213 |
| Eif3e | 1.31E−08 | 0.230467 | 0.662 | 0.649 | 0.00027 |
| Coro2a | 1.36E−08 | −0.25649 | 0.152 | 0.398 | 0.00028 |
| Pdia4 | 1.56E−08 | −0.3206 | 0.51 | 0.588 | 0.000322 |
| Ctsc | 1.60E−08 | 0.281436 | 0.972 | 0.953 | 0.000331 |
| Arl4a | 1.64E−08 | 0.127691 | 0.248 | 0.253 | 0.000339 |
| F10 | 1.67E−08 | −0.49429 | 0.241 | 0.503 | 0.000345 |
| Rps28 | 1.70E−08 | 0.246963 | 1 | 0.984 | 0.000352 |
| Cd68 | 1.81E−08 | −0.3849 | 0.862 | 0.933 | 0.000374 |
| B2m | 1.89E−08 | −0.21964 | 1 | 1 | 0.000391 |
| Esd | 2.01E−08 | −0.39661 | 0.669 | 0.85 | 0.000416 |
| C1qa | 2.09E−08 | 0.592959 | 0.745 | 0.621 | 0.000433 |
| Cadm1 | 2.14E−08 | 0.36125 | 0.324 | 0.118 | 0.000442 |
| Sp110 | 2.50E−08 | 0.21259 | 0.579 | 0.632 | 0.000516 |
| Pfdn5 | 2.58E−08 | 0.257302 | 0.91 | 0.897 | 0.000532 |
| Pnn | 2.61E−08 | 0.125198 | 0.262 | 0.281 | 0.00054 |
| Tppp3 | 2.75E−08 | −0.41012 | 0.2 | 0.446 | 0.000567 |
| Samm50 | 2.81E−08 | 0.219159 | 0.455 | 0.35 | 0.00058 |
| Usp48 | 2.85E−08 | 0.171512 | 0.276 | 0.232 | 0.000588 |
| Mdm2 | 3.02E−08 | −0.43529 | 0.552 | 0.747 | 0.000624 |
| Ccl2 | 3.19E−08 | −0.62072 | 0.834 | 0.929 | 0.000658 |
| Ppp1r21 | 3.59E−08 | 0.206143 | 0.359 | 0.289 | 0.000741 |
| Rpl8 | 3.64E−08 | 0.197088 | 0.993 | 0.98 | 0.000752 |
| Cd300lf | 3.71E−08 | −0.43749 | 0.49 | 0.688 | 0.000766 |
| Ubc | 4.04E−08 | −0.37066 | 0.876 | 0.951 | 0.000833 |
| Cpsf4 | 4.08E−08 | 0.109321 | 0.234 | 0.255 | 0.000842 |
| Anxa4 | 4.24E−08 | −0.31439 | 0.759 | 0.889 | 0.000875 |
| Tgfbr2 | 4.54E−08 | 0.225531 | 0.469 | 0.433 | 0.000937 |
| Slc40a1 | 4.55E−08 | 0.183359 | 0.069 | 0.119 | 0.000939 |
| Anapc1 | 4.59E−08 | 0.160148 | 0.324 | 0.284 | 0.000947 |
| Slpi | 4.95E−08 | −0.90625 | 0.2 | 0.398 | 0.001021 |
| Ankrd11 | 4.97E−08 | 0.306525 | 0.662 | 0.539 | 0.001026 |
| Gpr132 | 5.35E−08 | 0.317914 | 0.448 | 0.387 | 0.001104 |
| Tnf | 5.65E−08 | −0.49004 | 0.469 | 0.701 | 0.001166 |
| C6 | 5.99E−08 | 0.21951 | 0.103 | 0.009 | 0.001237 |
| Arfgap3 | 6.29E−08 | 0.109269 | 0.207 | 0.217 | 0.001299 |
| Tmem59 | 6.47E−08 | 0.182429 | 0.628 | 0.667 | 0.001336 |
| Hs6st1 | 6.51E−08 | 0.170633 | 0.145 | 0.106 | 0.001345 |
| Ccar1 | 6.87E−08 | 0.163016 | 0.359 | 0.346 | 0.001419 |
| Tuba1c | 7.11E−08 | −0.27446 | 0.621 | 0.836 | 0.001468 |
| Inhba | 7.30E−08 | −0.77315 | 0.11 | 0.323 | 0.001508 |
| Glipr2 | 7.39E−08 | −0.333 | 0.324 | 0.58 | 0.001526 |
| Synrg | 7.50E−08 | 0.156323 | 0.241 | 0.185 | 0.001548 |
| Rhob | 7.83E−08 | −0.24056 | 0.517 | 0.762 | 0.001616 |
| Utp11 | 9.69E−08 | 0.126404 | 0.324 | 0.347 | 0.002002 |
| Sowahc | 9.82E−08 | 0.301563 | 0.428 | 0.448 | 0.002027 |
| A930007l19Rik | 1.08E−07 | −0.25333 | 0.145 | 0.366 | 0.002238 |
| Irf7 | 1.21E−07 | −0.20512 | 0.793 | 0.929 | 0.002506 |
| Crip1 | 1.24E−07 | −0.6451 | 0.924 | 0.954 | 0.00256 |
| Slc9a9 | 1.34E−07 | 0.235153 | 0.29 | 0.165 | 0.002759 |
| Rps14 | 1.34E−07 | 0.223498 | 0.986 | 0.982 | 0.002762 |
| Mbnl1 | 1.41E−07 | 0.306969 | 0.814 | 0.699 | 0.002918 |
| Rps21 | 1.42E−07 | 0.224764 | 0.993 | 0.978 | 0.002924 |
| Cttnbp2nl | 1.54E−07 | 0.221724 | 0.49 | 0.422 | 0.003187 |
| Fuca1 | 1.60E−07 | 0.199508 | 0.538 | 0.516 | 0.003308 |
| Zfand5 | 1.74E−07 | −0.32951 | 0.745 | 0.895 | 0.003588 |
| Sectm1a | 1.76E−07 | 0.261268 | 0.29 | 0.211 | 0.003642 |
| Rps13 | 1.83E−07 | 0.257596 | 0.979 | 0.962 | 0.003769 |
| Utp14b | 1.87E−07 | 0.124301 | 0.117 | 0.048 | 0.003867 |
| Clptm1 | 1.91E−07 | 0.133777 | 0.434 | 0.456 | 0.003939 |
| Socs1 | 1.94E−07 | −0.29097 | 0.393 | 0.646 | 0.004006 |
| Akap8 | 1.99E−07 | 0.189974 | 0.29 | 0.175 | 0.004112 |
| Cxcl2 | 2.03E−07 | −0.61621 | 0.786 | 0.9 | 0.004202 |
| Stx11 | 2.13E−07 | −0.21156 | 0.255 | 0.497 | 0.004391 |
| Atp6v1a | 2.15E−07 | −0.2689 | 0.724 | 0.897 | 0.004438 |
| Clock | 2.15E−07 | 0.145801 | 0.269 | 0.245 | 0.00444 |
| Slc25a1 | 2.18E−07 | 0.106117 | 0.262 | 0.318 | 0.004503 |
| Ifitm6 | 2.31E−07 | −0.32974 | 0.103 | 0.309 | 0.00477 |
| Pcmtd1 | 2.34E−07 | 0.232332 | 0.483 | 0.395 | 0.004838 |
| Laptm5 | 2.35E−07 | 0.228208 | 0.979 | 0.98 | 0.004851 |
| Mark2 | 2.36E−07 | 0.102561 | 0.359 | 0.395 | 0.004867 |
| S100a8 | 2.56E−07 | −0.58237 | 0.221 | 0.465 | 0.005288 |
| Eef2 | 2.59E−07 | 0.274754 | 0.924 | 0.906 | 0.00535 |
| Ifi30 | 2.62E−07 | 0.317628 | 0.924 | 0.883 | 0.005415 |
| Tarm1 | 2.77E−07 | −0.28328 | 0.193 | 0.396 | 0.005722 |
| Creg1 | 2.97E−07 | −0.41984 | 0.821 | 0.904 | 0.006126 |
| Tifab | 3.05E−07 | 0.231532 | 0.455 | 0.371 | 0.006301 |
| 2410006H16Rik | 3.16E−07 | 0.267389 | 0.455 | 0.37 | 0.006524 |
| Tcf4 | 3.18E−07 | 0.30895 | 0.662 | 0.558 | 0.006573 |
| Acod1 | 3.19E−07 | −0.54614 | 0.593 | 0.772 | 0.006577 |
| Nos2 | 3.22E−07 | −0.70414 | 0.166 | 0.349 | 0.006645 |
| Rps18 | 3.25E−07 | 0.245493 | 0.848 | 0.825 | 0.006713 |
| mt-Nd2 | 3.38E−07 | 0.336176 | 0.952 | 0.938 | 0.006987 |
| Rpl9 | 3.40E−07 | 0.244205 | 0.993 | 0.963 | 0.007027 |
| Fbxl5 | 3.51E−07 | −0.45623 | 0.538 | 0.705 | 0.007249 |
| Plekhm1 | 3.54E−07 | −0.26274 | 0.193 | 0.427 | 0.007311 |
| Trappc3 | 3.71E−07 | 0.139541 | 0.393 | 0.403 | 0.007666 |
| H2-DMb2 | 3.80E−07 | 0.227344 | 0.331 | 0.191 | 0.007843 |
| Igf2r | 3.97E−07 | −0.14298 | 0.138 | 0.32 | 0.008198 |
| Atp6v1c1 | 4.13E−07 | −0.27817 | 0.552 | 0.764 | 0.008519 |
| Eif3k | 4.36E−07 | 0.24139 | 0.883 | 0.84 | 0.008997 |
| mt-Co2 | 4.43E−07 | 0.217088 | 0.979 | 0.993 | 0.009141 |
| Top2b | 4.65E−07 | 0.20273 | 0.386 | 0.318 | 0.009612 |
| Itgb7 | 4.78E−07 | −0.37717 | 0.31 | 0.535 | 0.009877 |
| Tagln2 | 4.81E−07 | −0.40653 | 0.772 | 0.871 | 0.009929 |
| Lilrb4a | 4.90E−07 | −0.32507 | 0.834 | 0.937 | 0.010114 |
| Evi2a | 5.42E−07 | 0.206481 | 0.641 | 0.641 | 0.011199 |
| Stag2 | 5.83E−07 | 0.191359 | 0.428 | 0.403 | 0.012045 |
| Rpl22l1 | 5.98E−07 | 0.266161 | 0.752 | 0.742 | 0.012356 |
| Mtmr6 | 6.04E−07 | 0.126478 | 0.331 | 0.354 | 0.012464 |
| Il6ra | 6.22E−07 | 0.286241 | 0.6 | 0.454 | 0.012839 |
| Txnrd1 | 6.53E−07 | −0.28853 | 0.455 | 0.674 | 0.013474 |
| Rpsa | 6.71E−07 | 0.27361 | 0.993 | 0.974 | 0.013848 |
| Rpl11 | 6.73E−07 | 0.251423 | 0.966 | 0.965 | 0.013889 |
| Irf1 | 6.77E−07 | −0.29562 | 0.8 | 0.932 | 0.013975 |
| Rnh1 | 6.84E−07 | −0.30913 | 0.697 | 0.861 | 0.014122 |
| Ppp3ca | 7.31E−07 | 0.291134 | 0.655 | 0.562 | 0.015094 |
| Klf6 | 7.45E−07 | −0.3064 | 0.945 | 0.986 | 0.015373 |
| Lhfpl2 | 7.46E−07 | −0.38135 | 0.207 | 0.399 | 0.015398 |
| Hltf | 7.46E−07 | 0.125651 | 0.2 | 0.152 | 0.015399 |
| Ifi203 | 7.74E−07 | 0.331131 | 0.703 | 0.73 | 0.015984 |
| Cysltr1 | 7.81E−07 | 0.254476 | 0.51 | 0.395 | 0.016129 |
| Bst1 | 7.93E−07 | −0.28952 | 0.579 | 0.767 | 0.01637 |
| Ltb4r1 | 8.05E−07 | −0.40386 | 0.31 | 0.54 | 0.016624 |
| Hhex | 8.19E−07 | 0.193466 | 0.255 | 0.177 | 0.016902 |
| Mpp1 | 8.34E−07 | −0.24921 | 0.524 | 0.746 | 0.017222 |
| Nek7 | 8.52E−07 | 0.254742 | 0.49 | 0.394 | 0.017598 |
| Ikbkb | 8.89E−07 | 0.164143 | 0.51 | 0.519 | 0.018354 |
| Rpl35a | 8.92E−07 | 0.197547 | 1 | 0.979 | 0.01842 |
| Smim13 | 9.07E−07 | 0.16776 | 0.193 | 0.116 | 0.018728 |
| Il11ra1 | 9.15E−07 | 0.113397 | 0.152 | 0.163 | 0.018898 |
| Ccpg1 | 9.24E−07 | 0.206631 | 0.255 | 0.164 | 0.01908 |
| Rps17 | 9.31E−07 | 0.222579 | 0.945 | 0.911 | 0.019215 |
| Rpl7a | 9.59E−07 | 0.254176 | 0.966 | 0.949 | 0.019794 |
| Azin1 | 1.02E−06 | −0.26634 | 0.566 | 0.726 | 0.020997 |
| Phf14 | 1.08E−06 | 0.104487 | 0.248 | 0.239 | 0.022198 |
| Rps15a | 1.10E−06 | 0.24102 | 0.986 | 0.976 | 0.022648 |
| Axin1 | 1.17E−06 | 0.104187 | 0.214 | 0.227 | 0.024261 |
| Hmbox1 | 1.18E−06 | 0.204998 | 0.393 | 0.316 | 0.024268 |
| Pde7b | 1.18E−06 | 0.163294 | 0.186 | 0.115 | 0.024398 |
| Fnbp1 | 1.22E−06 | 0.15645 | 0.393 | 0.38 | 0.025144 |
| Retnla | 1.26E−06 | 0.446774 | 0.138 | 0.027 | 0.025955 |
| Gla | 1.27E−06 | −0.22064 | 0.462 | 0.689 | 0.026198 |
| Tnfrsf23 | 1.27E−06 | −0.16254 | 0.097 | 0.201 | 0.026324 |
| Itm2b | 1.28E−06 | 0.281859 | 1 | 0.986 | 0.026384 |
| Trps1 | 1.29E−06 | 0.317946 | 0.676 | 0.483 | 0.026617 |
| Mgl2 | 1.29E−06 | 0.275552 | 0.11 | 0.02 | 0.026672 |
| Aebp2 | 1.31E−06 | 0.162709 | 0.366 | 0.358 | 0.027102 |
| Smc1a | 1.32E−06 | 0.100084 | 0.359 | 0.406 | 0.027222 |
| Snrnp25 | 1.32E−06 | 0.106946 | 0.234 | 0.264 | 0.027245 |
| Ifitm3 | 1.42E−06 | −0.21009 | 0.959 | 0.992 | 0.029368 |
| Csf3 | 1.46E−06 | −0.35535 | 0.014 | 0.133 | 0.030113 |
| Foxj3 | 1.55E−06 | 0.117252 | 0.186 | 0.174 | 0.031975 |
| Ywhag | 1.64E−06 | −0.27273 | 0.641 | 0.821 | 0.03388 |
| Kat2b | 1.65E−06 | 0.19448 | 0.434 | 0.426 | 0.034068 |
| Map3k14 | 1.65E−06 | 0.145114 | 0.166 | 0.135 | 0.034135 |
| Mospd2 | 1.66E−06 | 0.128445 | 0.345 | 0.357 | 0.034277 |
| Ssr4 | 1.67E−06 | 0.221009 | 0.724 | 0.708 | 0.034385 |
| Ssh2 | 1.73E−06 | 0.267003 | 0.414 | 0.277 | 0.035667 |
| Nfxl1 | 1.81E−06 | 0.228992 | 0.331 | 0.229 | 0.037342 |
| Slc31a2 | 1.84E−06 | −0.26744 | 0.455 | 0.682 | 0.037988 |
| Iah1 | 1.86E−06 | 0.159135 | 0.31 | 0.221 | 0.038329 |
| Ccl7 | 1.89E−06 | −0.59592 | 0.814 | 0.909 | 0.039107 |
| Cst3 | 1.97E−06 | 0.285426 | 0.972 | 0.963 | 0.040774 |
| 3830406C13Rik | 2.04E−06 | 0.114469 | 0.29 | 0.313 | 0.042066 |
| Srxn1 | 2.05E−06 | −0.22794 | 0.214 | 0.435 | 0.042249 |
| Mdm4 | 2.10E−06 | 0.15077 | 0.276 | 0.223 | 0.043314 |
| mt-Nd4l | 2.21E−06 | 0.220066 | 0.524 | 0.481 | 0.045555 |
| Sec62 | 2.39E−06 | 0.138322 | 0.524 | 0.543 | 0.049387 |
| Mospd3 | 2.48E−06 | 0.127617 | 0.228 | 0.2 | 0.051225 |
| Rnf13 | 2.51E−06 | 0.220272 | 0.497 | 0.43 | 0.051903 |
| Trim30d | 2.56E−06 | 0.195907 | 0.414 | 0.469 | 0.052787 |
| Kmt5a | 2.56E−06 | −0.2368 | 0.297 | 0.525 | 0.052834 |
| Ezr | 2.70E−06 | −0.28172 | 0.497 | 0.705 | 0.055703 |
| Card19 | 2.72E−06 | −0.31668 | 0.752 | 0.879 | 0.056111 |
| Slfn2 | 2.74E−06 | −0.23088 | 0.855 | 0.946 | 0.056542 |
| Npm1 | 2.76E−06 | 0.1519 | 0.855 | 0.899 | 0.056967 |
| Slc9a3r1 | 2.84E−06 | −0.28237 | 0.421 | 0.58 | 0.058623 |
| S100a9 | 2.90E−06 | −0.7131 | 0.172 | 0.385 | 0.059899 |
| Rbm26 | 2.91E−06 | 0.125358 | 0.283 | 0.262 | 0.060113 |
| Shisa5 | 2.91E−06 | −0.2417 | 0.779 | 0.917 | 0.060181 |
| Hnrnpll | 2.93E−06 | 0.157489 | 0.372 | 0.368 | 0.060434 |
| Map2k3 | 2.98E−06 | −0.28509 | 0.455 | 0.668 | 0.061487 |
| Pgam1 | 2.99E−06 | −0.23518 | 0.938 | 0.966 | 0.061812 |
| Ms4a6c | 3.09E−06 | 0.236084 | 0.945 | 0.943 | 0.063768 |
| Arg2 | 3.17E−06 | −0.35976 | 0.614 | 0.77 | 0.065448 |
| Ankrd13c | 3.23E−06 | 0.186603 | 0.352 | 0.274 | 0.066606 |
| Tnfaip2 | 3.27E−06 | −0.48421 | 0.752 | 0.863 | 0.067512 |
| Mrps14 | 3.31E−06 | 0.232294 | 0.69 | 0.608 | 0.068423 |
| Psmc1 | 3.38E−06 | −0.26826 | 0.448 | 0.634 | 0.069813 |
| Zfp36l1 | 3.49E−06 | 0.420038 | 0.876 | 0.836 | 0.072094 |
| Acsl4 | 3.50E−06 | 0.125995 | 0.4 | 0.447 | 0.07225 |
| Basp1 | 3.51E−06 | −0.39968 | 0.793 | 0.912 | 0.072474 |
| Tmem258 | 3.52E−06 | 0.129983 | 0.634 | 0.697 | 0.072666 |
| Oasl1 | 3.75E−06 | −0.16206 | 0.476 | 0.691 | 0.077351 |
| Gatm | 3.80E−06 | 0.274281 | 0.903 | 0.75 | 0.078463 |
| Timm22 | 3.91E−06 | 0.125793 | 0.221 | 0.227 | 0.080695 |
| Cxcl16 | 3.98E−06 | 0.279081 | 0.869 | 0.72 | 0.08216 |
| Pde4dip | 4.05E−06 | 0.17476 | 0.166 | 0.092 | 0.083693 |
| Ptrhd1 | 4.13E−06 | 0.14515 | 0.262 | 0.212 | 0.085357 |
| Sgms2 | 4.15E−06 | −0.23828 | 0.131 | 0.32 | 0.08578 |
| Igbp1 | 4.19E−06 | 0.215552 | 0.428 | 0.3 | 0.0866 |
| 2210016L21Rik | 4.26E−06 | 0.106605 | 0.214 | 0.218 | 0.088059 |
| Gnaq | 4.29E−06 | 0.212322 | 0.517 | 0.465 | 0.088678 |
| Cd24a | 4.35E−06 | −0.35282 | 0.09 | 0.261 | 0.089791 |
| Dennd1b | 4.53E−06 | 0.241731 | 0.434 | 0.336 | 0.093601 |
| Rps23 | 4.78E−06 | 0.17853 | 0.972 | 0.962 | 0.098748 |
| Snrpe | 4.90E−06 | 0.180256 | 0.759 | 0.777 | 0.101137 |
| Ero1l | 5.06E−06 | −0.37581 | 0.455 | 0.638 | 0.104445 |
| Slco3a1 | 5.14E−06 | 0.231816 | 0.276 | 0.179 | 0.106222 |
| Rps8 | 5.20E−06 | 0.234555 | 1 | 0.983 | 0.107351 |
| D1Ertd622e | 5.23E−06 | 0.1644 | 0.345 | 0.328 | 0.107947 |
| Hnrnpa1 | 5.25E−06 | 0.215325 | 0.703 | 0.653 | 0.108401 |
| Slc25a36 | 5.30E−06 | 0.151345 | 0.324 | 0.298 | 0.109463 |
| Ankrd55 | 5.31E−06 | −0.24836 | 0.034 | 0.179 | 0.109592 |
| Bicra | 5.38E−06 | 0.128442 | 0.166 | 0.126 | 0.11109 |
| Ccl3 | 5.44E−06 | −0.60077 | 0.462 | 0.671 | 0.112339 |
| Rgs10 | 5.51E−06 | 0.221772 | 0.593 | 0.543 | 0.113764 |
| Ccni | 5.51E−06 | 0.160712 | 0.434 | 0.407 | 0.113807 |
| Rps6 | 5.68E−06 | 0.233181 | 0.931 | 0.915 | 0.117216 |
| Yipf6 | 5.73E−06 | 0.144028 | 0.228 | 0.18 | 0.118264 |
| Cd44 | 5.87E−06 | −0.26198 | 0.855 | 0.93 | 0.12131 |
| Rpl37 | 6.00E−06 | 0.21501 | 0.972 | 0.97 | 0.1239 |
| Relb | 6.16E−06 | 0.154457 | 0.366 | 0.348 | 0.127282 |
| Mif4gd | 6.21E−06 | 0.169488 | 0.331 | 0.278 | 0.128208 |
| H2-Q6 | 6.28E−06 | −0.18955 | 0.372 | 0.596 | 0.129647 |
| Tapbp | 6.52E−06 | −0.22734 | 0.931 | 0.982 | 0.134629 |
| Hnrnpul2 | 6.54E−06 | 0.109042 | 0.386 | 0.419 | 0.135138 |
| Man2b1 | 6.58E−06 | 0.220369 | 0.6 | 0.564 | 0.13579 |
| Rcsd1 | 6.72E−06 | 0.165333 | 0.221 | 0.147 | 0.138663 |
| Arl8a | 6.77E−06 | −0.21523 | 0.607 | 0.798 | 0.139724 |
| Pid1 | 7.54E−06 | 0.359607 | 0.683 | 0.506 | 0.155744 |
| Traf7 | 7.67E−06 | 0.141017 | 0.324 | 0.276 | 0.158284 |
| Itgb2 | 7.68E−06 | −0.27705 | 0.69 | 0.821 | 0.158621 |
| Mgat4a | 8.09E−06 | 0.185205 | 0.303 | 0.212 | 0.16697 |
| Slc12a6 | 8.16E−06 | 0.194986 | 0.324 | 0.228 | 0.168537 |
| Ap4s1 | 8.27E−06 | 0.10643 | 0.193 | 0.186 | 0.170805 |
| Tmsb10 | 8.50E−06 | −0.40143 | 0.945 | 0.965 | 0.175415 |
| Tmem140 | 8.55E−06 | −0.27737 | 0.352 | 0.555 | 0.176628 |
| Cdc34 | 8.65E−06 | −0.17839 | 0.255 | 0.471 | 0.178519 |
| Cxcl10 | 8.95E−06 | 0.68793 | 0.572 | 0.449 | 0.184746 |
| Tanc2 | 9.15E−06 | 0.175337 | 0.2 | 0.094 | 0.18893 |
| Bst2 | 9.78E−06 | −0.22539 | 0.628 | 0.819 | 0.201901 |
| Rapgef1 | 9.99E−06 | 0.112524 | 0.221 | 0.185 | 0.206256 |
| Nr4a3 | 1.03E−05 | 0.280178 | 0.31 | 0.263 | 0.212843 |
| Gltp | 1.04E−05 | 0.219746 | 0.634 | 0.604 | 0.215487 |
| Arcn1 | 1.05E−05 | −0.14704 | 0.434 | 0.645 | 0.215908 |
| Mycbp2 | 1.06E−05 | 0.246848 | 0.669 | 0.621 | 0.218249 |
| Ccdc71l | 1.06E−05 | −0.2104 | 0.359 | 0.58 | 0.218675 |
| Gm13822 | 1.06E−05 | −0.23257 | 0.083 | 0.249 | 0.219662 |
| Cap1 | 1.07E−05 | −0.21383 | 0.917 | 0.962 | 0.221263 |
| Acer3 | 1.12E−05 | 0.246226 | 0.655 | 0.546 | 0.230681 |
| Ube2m | 1.13E−05 | −0.21768 | 0.379 | 0.599 | 0.233689 |
| Pfn1 | 1.14E−05 | −0.15964 | 0.993 | 0.999 | 0.236072 |
| Rpl21 | 1.14E−05 | 0.252477 | 0.903 | 0.831 | 0.236325 |
| Glcci1 | 1.15E−05 | 0.159252 | 0.166 | 0.067 | 0.237842 |
| Ifih1 | 1.16E−05 | 0.20341 | 0.51 | 0.519 | 0.239139 |
| C1qc | 1.20E−05 | 0.422089 | 0.724 | 0.628 | 0.246895 |
| Pkm | 1.20E−05 | −0.23071 | 0.972 | 0.985 | 0.247561 |
| Cdk2ap2 | 1.22E−05 | 0.286166 | 0.69 | 0.654 | 0.251683 |
| Rtraf | 1.22E−05 | 0.118106 | 0.71 | 0.778 | 0.252724 |
| Stat6 | 1.23E−05 | 0.235881 | 0.503 | 0.327 | 0.254898 |
| Fam49b | 1.33E−05 | 0.245233 | 0.959 | 0.862 | 0.274699 |
| Anxa2 | 1.34E−05 | −0.31108 | 0.862 | 0.952 | 0.277461 |
| Kdm3b | 1.35E−05 | 0.108282 | 0.207 | 0.144 | 0.277891 |
| Rpl14 | 1.45E−05 | 0.259667 | 0.938 | 0.913 | 0.298821 |
| Gas6 | 1.48E−05 | 0.357942 | 0.303 | 0.167 | 0.306537 |
| Gsr | 1.49E−05 | −0.26225 | 0.469 | 0.669 | 0.307751 |
| Cxcl3 | 1.51E−05 | 0.78881 | 0.138 | 0.323 | 0.310963 |
| Axl | 1.52E−05 | 0.262186 | 0.29 | 0.159 | 0.314446 |
| D6Wsu163e | 1.54E−05 | 0.109174 | 0.159 | 0.119 | 0.317215 |
| Zdhhc14 | 1.54E−05 | 0.146207 | 0.166 | 0.051 | 0.317353 |
| Ggh | 1.58E−05 | −0.23658 | 0.386 | 0.579 | 0.32667 |
| Wbp2 | 1.60E−05 | −0.16477 | 0.372 | 0.584 | 0.330558 |
| Vim | 1.63E−05 | −0.30016 | 0.972 | 0.987 | 0.336238 |
| Zfp518a | 1.67E−05 | 0.110343 | 0.138 | 0.116 | 0.344005 |
| Rtp4 | 1.71E−05 | −0.26499 | 0.621 | 0.791 | 0.35207 |
| Eef1d | 1.73E−05 | 0.188754 | 0.648 | 0.63 | 0.357191 |
| Uqcrh | 1.74E−05 | 0.208815 | 0.883 | 0.892 | 0.358564 |
| Ndufv1 | 1.75E−05 | 0.10242 | 0.303 | 0.336 | 0.360366 |
| Erp29 | 1.76E−05 | 0.186863 | 0.855 | 0.846 | 0.363253 |
| Slc11a1 | 1.86E−05 | −0.24373 | 0.503 | 0.704 | 0.385091 |
| Psmb5 | 1.90E−05 | −0.25471 | 0.607 | 0.766 | 0.391336 |
| Helz2 | 1.98E−05 | −0.24873 | 0.276 | 0.472 | 0.408751 |
| Nsd3 | 2.01E−05 | 0.230117 | 0.614 | 0.528 | 0.41449 |
| Rpl10 | 2.05E−05 | 0.21622 | 0.91 | 0.927 | 0.424026 |
| Ccndbp1 | 2.12E−05 | 0.108956 | 0.393 | 0.425 | 0.437023 |
| H2-D1 | 2.17E−05 | −0.11858 | 1 | 0.998 | 0.447955 |
| Tbc1d2b | 2.21E−05 | 0.118652 | 0.262 | 0.255 | 0.456762 |
| Pnrc2 | 2.25E−05 | 0.102046 | 0.228 | 0.213 | 0.463744 |
| Tmem37 | 2.25E−05 | 0.120479 | 0.221 | 0.214 | 0.465383 |
| Eml4 | 2.26E−05 | 0.129184 | 0.317 | 0.314 | 0.46699 |
| Sqstm1 | 2.26E−05 | −0.33774 | 0.745 | 0.855 | 0.467435 |
| Gm4951 | 2.28E−05 | 0.130932 | 0.4 | 0.47 | 0.47044 |
| Trem1 | 2.28E−05 | −0.30104 | 0.11 | 0.286 | 0.471366 |
| Igf2bp2 | 2.29E−05 | −0.2074 | 0.097 | 0.268 | 0.47203 |
| Psmd8 | 2.34E−05 | −0.20459 | 0.683 | 0.845 | 0.482702 |
| Trim12c | 2.34E−05 | −0.20038 | 0.228 | 0.359 | 0.482732 |
| Irf2bp2 | 2.40E−05 | 0.276248 | 0.814 | 0.76 | 0.495841 |
| Naca | 2.41E−05 | 0.198923 | 0.959 | 0.92 | 0.498396 |
| Msn | 2.45E−05 | −0.20257 | 0.869 | 0.958 | 0.505995 |
| EgIn3 | 2.57E−05 | −0.23296 | 0.124 | 0.305 | 0.529815 |
| Cct8 | 2.59E−05 | −0.15407 | 0.586 | 0.767 | 0.534417 |
| Isg20 | 2.62E−05 | −0.21534 | 0.228 | 0.429 | 0.540712 |
| Nfkb2 | 2.62E−05 | 0.109112 | 0.379 | 0.423 | 0.541705 |
| Ppia | 2.68E−05 | 0.197711 | 0.986 | 0.978 | 0.55254 |
| Eif3m | 2.74E−05 | 0.132742 | 0.531 | 0.552 | 0.565509 |
| Fabp5 | 2.76E−05 | −0.64138 | 0.69 | 0.819 | 0.569674 |
| Fbxw17 | 2.86E−05 | 0.181933 | 0.255 | 0.185 | 0.590546 |
| Rragc | 2.88E−05 | −0.13588 | 0.4 | 0.597 | 0.595393 |
| Pdcd6 | 2.90E−05 | −0.11286 | 0.497 | 0.686 | 0.597937 |
| Dock8 | 2.90E−05 | 0.211716 | 0.407 | 0.339 | 0.598578 |
| Sgk3 | 2.92E−05 | 0.111505 | 0.455 | 0.504 | 0.603704 |
| Atxn1 | 2.94E−05 | 0.173614 | 0.317 | 0.272 | 0.607763 |
| Anxa6 | 3.00E−05 | −0.28476 | 0.524 | 0.678 | 0.619812 |
| Arid4a | 3.01E−05 | 0.238762 | 0.517 | 0.371 | 0.621607 |
| Lilra6 | 3.09E−05 | −0.14204 | 0.193 | 0.371 | 0.638835 |
| Phf11b | 3.09E−05 | 0.15611 | 0.448 | 0.515 | 0.639046 |
| Vwa8 | 3.14E−05 | 0.120898 | 0.145 | 0.084 | 0.64773 |
| Klrk1 | 3.38E−05 | 0.213006 | 0.393 | 0.316 | 0.698465 |
| Clec4e | 3.43E−05 | −0.31688 | 0.441 | 0.648 | 0.708036 |
| Mindy3 | 3.52E−05 | 0.194588 | 0.421 | 0.267 | 0.725817 |
| Gngt2 | 3.54E−05 | −0.29106 | 0.434 | 0.556 | 0.730061 |
| Vegfa | 3.58E−05 | 0.311704 | 0.441 | 0.436 | 0.73829 |
| Mef2c | 3.67E−05 | 0.216711 | 0.324 | 0.174 | 0.758501 |
| Rsad2 | 3.69E−05 | −0.33017 | 0.579 | 0.77 | 0.761672 |
| Adap2 | 3.72E−05 | 0.210307 | 0.462 | 0.363 | 0.768657 |
| Nsd2 | 3.79E−05 | 0.173949 | 0.324 | 0.263 | 0.783221 |
| Ifrd1 | 3.84E−05 | −0.23967 | 0.752 | 0.898 | 0.79216 |
| Tcerg1 | 3.87E−05 | 0.140866 | 0.386 | 0.378 | 0.799835 |
| Atp5h | 3.88E−05 | 0.201998 | 0.869 | 0.866 | 0.801954 |
| Was | 3.91E−05 | 0.107989 | 0.428 | 0.459 | 0.80824 |
| Wdr45b | 3.91E−05 | 0.177159 | 0.441 | 0.411 | 0.808293 |
| Trp53bp2 | 3.93E−05 | −0.10072 | 0.055 | 0.181 | 0.812341 |
| Plin3 | 4.01E−05 | −0.18007 | 0.372 | 0.581 | 0.82836 |
| Pik3r1 | 4.09E−05 | 0.182432 | 0.434 | 0.398 | 0.84498 |
| Fpr1 | 4.16E−05 | −0.23933 | 0.069 | 0.22 | 0.859598 |
| Spata5 | 4.17E−05 | 0.100736 | 0.124 | 0.081 | 0.860446 |
| Fam76a | 4.30E−05 | −0.15556 | 0.372 | 0.578 | 0.887502 |
| Anxa1 | 4.36E−05 | −0.35468 | 0.538 | 0.683 | 0.899333 |
| Rhou | 4.38E−05 | −0.21091 | 0.186 | 0.377 | 0.905134 |
| S100a10 | 4.39E−05 | −0.30462 | 0.924 | 0.952 | 0.907522 |
| Xrn1 | 4.45E−05 | 0.154347 | 0.421 | 0.384 | 0.919024 |
| Cox4i1 | 4.46E−05 | 0.18138 | 0.966 | 0.942 | 0.919998 |
| Zfp992 | 4.47E−05 | −0.10361 | 0.062 | 0.194 | 0.922396 |
| Ugp2 | 4.48E−05 | −0.16558 | 0.503 | 0.697 | 0.92408 |
| Pafah1b1 | 4.65E−05 | −0.1778 | 0.69 | 0.855 | 0.959546 |
| Txnl1 | 4.75E−05 | −0.19886 | 0.517 | 0.715 | 0.981413 |
| Ctsk | 4.78E−05 | −0.34499 | 0.069 | 0.181 | 0.987813 |
| Larp4b | 4.86E−05 | 0.159198 | 0.634 | 0.658 | 1 |
| Ergic3 | 4.87E−05 | 0.173118 | 0.455 | 0.378 | 1 |
| P2ry10b | 4.91E−05 | −0.15759 | 0.152 | 0.326 | 1 |
| Csf1 | 4.93E−05 | −0.44234 | 0.124 | 0.266 | 1 |
| Dicer1 | 4.96E−05 | 0.11834 | 0.255 | 0.202 | 1 |
| Myo9a | 4.98E−05 | 0.182265 | 0.338 | 0.259 | 1 |
| Ndufs4 | 5.01E−05 | 0.104337 | 0.538 | 0.593 | 1 |
| Ensa | 5.03E−05 | 0.158252 | 0.552 | 0.546 | 1 |
| Tmem189 | 5.06E−05 | −0.12817 | 0.614 | 0.796 | 1 |
| Cd200r1 | 5.09E−05 | −0.24197 | 0.366 | 0.565 | 1 |
| Fopnl | 5.10E−05 | 0.130181 | 0.248 | 0.21 | 1 |
| Sra1 | 5.11E−05 | −0.19669 | 0.366 | 0.572 | 1 |
| Rfk | 5.50E−05 | −0.11885 | 0.186 | 0.361 | 1 |
| Ptpn2 | 5.60E−05 | −0.20034 | 0.379 | 0.541 | 1 |
| Use1 | 5.99E−05 | 0.187507 | 0.497 | 0.418 | 1 |
| Dusp3 | 6.14E−05 | −0.25738 | 0.497 | 0.671 | 1 |
| Gabarap | 6.23E−05 | −0.11563 | 0.993 | 0.989 | 1 |
| Pitpnm1 | 6.23E−05 | 0.125663 | 0.234 | 0.19 | 1 |
| Slc35f6 | 6.28E−05 | −0.18689 | 0.393 | 0.598 | 1 |
| Pphln1 | 6.41E−05 | 0.11212 | 0.228 | 0.222 | 1 |
| Ctsh | 6.42E−05 | −0.24646 | 0.869 | 0.927 | 1 |
| Cd40 | 6.47E−05 | 0.175034 | 0.414 | 0.412 | 1 |
| Impact | 6.63E−05 | −0.22029 | 0.393 | 0.598 | 1 |
| Sec61g | 6.91E−05 | −0.22663 | 0.855 | 0.924 | 1 |
| Rps26 | 6.91E−05 | 0.166241 | 0.986 | 0.984 | 1 |
| Gm45716 | 7.04E−05 | 0.138145 | 0.138 | 0.068 | 1 |
| Ifit3 | 7.05E−05 | 0.279425 | 0.434 | 0.465 | 1 |
| Pnpla2 | 7.10E−05 | 0.152838 | 0.386 | 0.333 | 1 |
| Psap | 7.13E−05 | −0.32024 | 1 | 0.999 | 1 |
| mt-Co3 | 7.21E−05 | 0.225491 | 0.993 | 0.996 | 1 |
| Coq5 | 7.23E−05 | −0.11923 | 0.131 | 0.288 | 1 |
| AI504432 | 7.38E−05 | 0.115701 | 0.2 | 0.222 | 1 |
| Nectin2 | 7.39E−05 | 0.123034 | 0.152 | 0.117 | 1 |
| Dtx2 | 7.40E−05 | −0.16801 | 0.145 | 0.321 | 1 |
| H2-Q7 | 7.41E−05 | −0.25677 | 0.641 | 0.799 | 1 |
| Pla2g15 | 7.50E−05 | 0.137792 | 0.324 | 0.332 | 1 |
| Brd2 | 7.54E−05 | −0.25174 | 0.759 | 0.874 | 1 |
| Hspa1a | 7.62E−05 | 0.120164 | 0.234 | 0.327 | 1 |
| Trmt2b | 7.85E−05 | 0.10632 | 0.124 | 0.066 | 1 |
| Mmp19 | 8.03E−05 | −0.29773 | 0.262 | 0.421 | 1 |
| Ash1l | 8.17E−05 | 0.166303 | 0.49 | 0.482 | 1 |
| Mrc1 | 8.19E−05 | 0.360542 | 0.897 | 0.798 | 1 |
| Rab8b | 8.39E−05 | −0.23361 | 0.717 | 0.861 | 1 |
| Btg2 | 8.46E−05 | 0.262565 | 0.793 | 0.84 | 1 |
| Cdc42ep2 | 8.52E−05 | −0.21987 | 0.241 | 0.429 | 1 |
| Ciita | 8.55E−05 | 0.235937 | 0.331 | 0.184 | 1 |
| Ubl3 | 8.92E−05 | 0.237984 | 0.772 | 0.703 | 1 |
| Mcfd2 | 8.97E−05 | 0.134123 | 0.455 | 0.482 | 1 |
| Slfn1 | 9.02E−05 | −0.18201 | 0.51 | 0.705 | 1 |
| E230032D23Rik | 9.06E−05 | −0.11952 | 0.034 | 0.138 | 1 |
| Kpna3 | 9.09E−05 | 0.161523 | 0.352 | 0.324 | 1 |
| I17r | 9.10E−05 | −0.45938 | 0.221 | 0.391 | 1 |
| Ddhd1 | 9.27E−05 | 0.116802 | 0.269 | 0.277 | 1 |
| Fchsd2 | 9.35E−05 | 0.276353 | 0.345 | 0.194 | 1 |
| Ctsb | 9.64E−05 | −0.28802 | 0.993 | 0.999 | 1 |
| Ptafr | 9.65E−05 | −0.22345 | 0.634 | 0.806 | 1 |
| Itm2c | 9.66E−05 | 0.165142 | 0.683 | 0.705 | 1 |
| Slk | 9.83E−05 | 0.150905 | 0.531 | 0.531 | 1 |
| Serp1 | 0.000101 | −0.2573 | 0.724 | 0.854 | 1 |
| Baiap2 | 0.000102 | 0.124094 | 0.228 | 0.246 | 1 |
| Brpf1 | 0.000103 | 0.157611 | 0.372 | 0.316 | 1 |
| Rgcc | 0.000104 | −0.39295 | 0.193 | 0.331 | 1 |
| Runx3 | 0.000106 | 0.286605 | 0.552 | 0.441 | 1 |
| Eif1 | 0.00011 | −0.18613 | 0.993 | 0.992 | 1 |
| Tuba4a | 0.000112 | −0.17166 | 0.179 | 0.361 | 1 |
| Gm26917 | 0.000115 | −0.33093 | 0.469 | 0.624 | 1 |
| Klf2 | 0.000115 | −0.36393 | 0.786 | 0.873 | 1 |
| Rab31 | 0.000116 | −0.21382 | 0.538 | 0.719 | 1 |
| Gm10260 | 0.000117 | 0.1151 | 0.386 | 0.377 | 1 |
| Nfkbib | 0.000117 | −0.11088 | 0.317 | 0.499 | 1 |
| Sell | 0.000117 | −0.28234 | 0.255 | 0.438 | 1 |
| Cpne2 | 0.000118 | −0.21282 | 0.352 | 0.549 | 1 |
| Endod1 | 0.000119 | 0.156469 | 0.228 | 0.17 | 1 |
| Ndufa2 | 0.00012 | 0.212574 | 0.876 | 0.824 | 1 |
| Gcnt1 | 0.00012 | 0.134631 | 0.172 | 0.132 | 1 |
| Khdc4 | 0.000121 | −0.11702 | 0.159 | 0.318 | 1 |
| Rpl31 | 0.000125 | 0.211195 | 0.897 | 0.87 | 1 |
| Id3 | 0.000126 | −0.30302 | 0.193 | 0.255 | 1 |
| Iqsec1 | 0.00013 | −0.11724 | 0.159 | 0.315 | 1 |
| Atf1 | 0.000131 | −0.18143 | 0.269 | 0.442 | 1 |
| Mir22hg | 0.000135 | −0.23009 | 0.517 | 0.702 | 1 |
| Arg1 | 0.000136 | −0.72256 | 0.607 | 0.664 | 1 |
| Psmd2 | 0.000138 | −0.20508 | 0.703 | 0.837 | 1 |
| Hdac7 | 0.000144 | −0.11205 | 0.028 | 0.122 | 1 |
| Trim25 | 0.000147 | −0.17784 | 0.51 | 0.696 | 1 |
| Hal | 0.000147 | −0.54003 | 0.428 | 0.57 | 1 |
| Etv6 | 0.000148 | 0.147892 | 0.448 | 0.427 | 1 |
| 1110008P14Rik | 0.00015 | −0.12645 | 0.11 | 0.267 | 1 |
| Lmbrd2 | 0.000155 | 0.107333 | 0.255 | 0.226 | 1 |
| Wdr33 | 0.000158 | 0.131526 | 0.338 | 0.304 | 1 |
| Pink1 | 0.000158 | 0.127658 | 0.186 | 0.134 | 1 |
| Lsm4 | 0.000158 | 0.113001 | 0.497 | 0.523 | 1 |
| Clcn7 | 0.000161 | −0.2053 | 0.241 | 0.401 | 1 |
| Rnaset2a | 0.000163 | −0.13783 | 0.345 | 0.486 | 1 |
| Ciao2a | 0.000165 | −0.1866 | 0.641 | 0.79 | 1 |
| Cd300c2 | 0.000165 | 0.179846 | 0.71 | 0.72 | 1 |
| Zfhx3 | 0.000167 | 0.222053 | 0.331 | 0.202 | 1 |
| Atp6v1d | 0.00017 | −0.10349 | 0.538 | 0.708 | 1 |
| Tmbim4 | 0.00017 | −0.12395 | 0.586 | 0.761 | 1 |
| Acot7 | 0.000173 | −0.14324 | 0.09 | 0.229 | 1 |
| Rpl36 | 0.000173 | 0.220503 | 0.945 | 0.91 | 1 |
| Got1 | 0.000175 | 0.163654 | 0.517 | 0.498 | 1 |
| Eif1a | 0.000178 | −0.18842 | 0.49 | 0.68 | 1 |
| Stk10 | 0.000179 | 0.128743 | 0.221 | 0.168 | 1 |
| Peli1 | 0.000184 | −0.1129 | 0.579 | 0.749 | 1 |
| Ptbp1 | 0.000184 | 0.120625 | 0.469 | 0.472 | 1 |
| Msmo1 | 0.000185 | 0.129317 | 0.228 | 0.202 | 1 |
| Rae1 | 0.000187 | −0.10993 | 0.166 | 0.325 | 1 |
| Orai2 | 0.000187 | −0.12432 | 0.11 | 0.251 | 1 |
| Sgsh | 0.000191 | 0.151301 | 0.221 | 0.181 | 1 |
| Fcgrt | 0.000191 | 0.334108 | 0.683 | 0.567 | 1 |
| Dab2 | 0.000195 | 0.282144 | 0.897 | 0.836 | 1 |
| Runx1 | 0.0002 | 0.203616 | 0.628 | 0.589 | 1 |
| Prdm1 | 0.000202 | 0.212206 | 0.372 | 0.354 | 1 |
| Itga9 | 0.000203 | 0.214578 | 0.283 | 0.184 | 1 |
| Flrt2 | 0.000203 | −0.23579 | 0.262 | 0.416 | 1 |
| Il15 | 0.000204 | 0.180245 | 0.379 | 0.353 | 1 |
| Il18rap | 0.000206 | −0.14109 | 0.055 | 0.183 | 1 |
| Rhov | 0.000206 | −0.15316 | 0.069 | 0.204 | 1 |
| Psma7 | 0.000207 | −0.22137 | 0.897 | 0.938 | 1 |
| Naa10 | 0.000208 | 0.124217 | 0.241 | 0.183 | 1 |
| Srebf2 | 0.000209 | 0.159224 | 0.317 | 0.228 | 1 |
| Gpalpp1 | 0.000212 | 0.182607 | 0.421 | 0.367 | 1 |
| Plin2 | 0.000214 | −0.2857 | 0.917 | 0.951 | 1 |
| Tacc1 | 0.000215 | 0.13956 | 0.469 | 0.463 | 1 |
| Lrrfip2 | 0.000218 | −0.19525 | 0.338 | 0.517 | 1 |
| Hist3h2a | 0.000221 | 0.114343 | 0.166 | 0.124 | 1 |
| Gna12 | 0.000221 | 0.174366 | 0.469 | 0.432 | 1 |
| Gas7 | 0.000221 | −0.20935 | 0.338 | 0.53 | 1 |
| Gpx1 | 0.000224 | −0.22355 | 0.966 | 0.945 | 1 |
| S100a13 | 0.000226 | 0.148261 | 0.683 | 0.685 | 1 |
| Rpl35 | 0.000229 | 0.198305 | 0.862 | 0.844 | 1 |
| Calm3 | 0.000231 | −0.2061 | 0.738 | 0.872 | 1 |
| Tubb6 | 0.000232 | −0.22676 | 0.607 | 0.769 | 1 |
| Zc3hav1 | 0.000232 | −0.18351 | 0.586 | 0.757 | 1 |
| Eif4ebp2 | 0.000235 | 0.110064 | 0.428 | 0.455 | 1 |
| Zswim4 | 0.000235 | −0.15872 | 0.186 | 0.341 | 1 |
| Cggbp1 | 0.000237 | −0.1889 | 0.517 | 0.7 | 1 |
| Macf1 | 0.000239 | 0.185884 | 0.572 | 0.52 | 1 |
| Nfe211 | 0.000242 | 0.105977 | 0.372 | 0.368 | 1 |
| Frmd4b | 0.000246 | 0.227334 | 0.545 | 0.475 | 1 |
| Dyrk1a | 0.000247 | 0.115172 | 0.317 | 0.308 | 1 |
| Hsp90aa1 | 0.00025 | −0.24384 | 0.772 | 0.882 | 1 |
| Derl1 | 0.000251 | −0.13272 | 0.476 | 0.657 | 1 |
| Mocs2 | 0.000256 | −0.1002 | 0.359 | 0.533 | 1 |
| Camk1d | 0.000261 | 0.238348 | 0.531 | 0.459 | 1 |
| Timp2 | 0.000266 | 0.277581 | 0.676 | 0.626 | 1 |
| Sec22b | 0.000267 | 0.123039 | 0.338 | 0.307 | 1 |
| Luc7l2 | 0.000268 | 0.187817 | 0.703 | 0.671 | 1 |
| Hk3 | 0.000268 | −0.20638 | 0.607 | 0.773 | 1 |
| Pira2 | 0.00027 | −0.24748 | 0.641 | 0.787 | 1 |
| Lrrc58 | 0.00027 | 0.132385 | 0.538 | 0.558 | 1 |
| Rnase4 | 0.000272 | 0.170484 | 0.366 | 0.309 | 1 |
| Raph1 | 0.000274 | −0.22182 | 0.352 | 0.541 | 1 |
| Myh9 | 0.00028 | 0.115898 | 0.669 | 0.725 | 1 |
| Mrpl21 | 0.000282 | 0.100824 | 0.352 | 0.363 | 1 |
| Snx2 | 0.000286 | 0.14189 | 0.772 | 0.833 | 1 |
| Nup93 | 0.000287 | 0.127038 | 0.255 | 0.225 | 1 |
| Mdh2 | 0.000289 | −0.14564 | 0.669 | 0.824 | 1 |
| Mcl1 | 0.00029 | −0.13676 | 0.979 | 0.992 | 1 |
| Ly6c1 | 0.000295 | −0.34112 | 0.269 | 0.422 | 1 |
| Ly96 | 0.000297 | −0.16383 | 0.29 | 0.445 | 1 |
| G0s2 | 0.000297 | −0.27003 | 0.124 | 0.272 | 1 |
| Jkamp | 0.000301 | 0.15318 | 0.379 | 0.292 | 1 |
| Tle3 | 0.000303 | 0.109202 | 0.441 | 0.47 | 1 |
| Ranbp3 | 0.000304 | 0.111922 | 0.255 | 0.199 | 1 |
| Ankrd44 | 0.000304 | 0.154475 | 0.324 | 0.273 | 1 |
| Phlda1 | 0.000305 | −0.32084 | 0.393 | 0.58 | 1 |
| Mllt6 | 0.000308 | 0.128073 | 0.283 | 0.259 | 1 |
| Rnf4 | 0.000309 | 0.128723 | 0.469 | 0.459 | 1 |
| Cd38 | 0.000311 | −0.29826 | 0.366 | 0.54 | 1 |
| Hebp1 | 0.000312 | 0.246774 | 0.559 | 0.46 | 1 |
| Dusp6 | 0.000314 | 0.23688 | 0.366 | 0.239 | 1 |
| Slc39a11 | 0.000323 | 0.115647 | 0.152 | 0.101 | 1 |
| Ttc1 | 0.000328 | −0.16466 | 0.276 | 0.444 | 1 |
| Slc25a4 | 0.000329 | 0.204607 | 0.483 | 0.417 | 1 |
| Pag1 | 0.00033 | −0.13388 | 0.11 | 0.232 | 1 |
| Fcgr3 | 0.00033 | 0.201096 | 0.931 | 0.93 | 1 |
| Mafg | 0.00033 | −0.17062 | 0.545 | 0.723 | 1 |
| Flna | 0.000331 | −0.20039 | 0.703 | 0.844 | 1 |
| Rpl15 | 0.000341 | 0.213559 | 0.959 | 0.941 | 1 |
| Maf1 | 0.000344 | 0.15817 | 0.49 | 0.448 | 1 |
| Rac2 | 0.000345 | −0.24463 | 0.669 | 0.779 | 1 |
| Ap3s1 | 0.000346 | −0.21239 | 0.607 | 0.763 | 1 |
| Ptpro | 0.000346 | 0.166225 | 0.386 | 0.359 | 1 |
| Oas1a | 0.000352 | −0.12609 | 0.138 | 0.295 | 1 |
| Maml2 | 0.000353 | 0.12387 | 0.131 | 0.067 | 1 |
| Cbl | 0.000354 | 0.187158 | 0.634 | 0.62 | 1 |
| Ethe1 | 0.000364 | −0.1209 | 0.324 | 0.497 | 1 |
| H1f0 | 0.000366 | 0.133332 | 0.338 | 0.377 | 1 |
| Tgoln1 | 0.000368 | −0.20106 | 0.655 | 0.804 | 1 |
| Ier3ip1 | 0.000369 | −0.15727 | 0.641 | 0.801 | 1 |
| Ccl4 | 0.000373 | −0.43959 | 0.786 | 0.897 | 1 |
| Prrc2c | 0.000377 | 0.117672 | 0.69 | 0.733 | 1 |
| Ctnnbip1 | 0.00038 | 0.138876 | 0.372 | 0.336 | 1 |
| Atp6v1h | 0.000381 | −0.16546 | 0.414 | 0.593 | 1 |
| Iscu | 0.000389 | 0.112024 | 0.503 | 0.528 | 1 |
| Irf2bpl | 0.000392 | −0.11503 | 0.324 | 0.492 | 1 |
| Dock7 | 0.000393 | 0.124207 | 0.331 | 0.285 | 1 |
| Lrmp | 0.000395 | 0.176514 | 0.379 | 0.261 | 1 |
| Rpl4 | 0.000395 | 0.194356 | 0.793 | 0.79 | 1 |
| Selenof | 0.000396 | 0.170472 | 0.772 | 0.751 | 1 |
| Sertad1 | 0.000397 | −0.19965 | 0.372 | 0.543 | 1 |
| Eif3i | 0.000397 | 0.129114 | 0.621 | 0.638 | 1 |
| Gnb1 | 0.000398 | −0.1545 | 0.91 | 0.974 | 1 |
| Leprotl1 | 0.000401 | −0.18551 | 0.503 | 0.666 | 1 |
| Wdr82 | 0.000408 | 0.147495 | 0.324 | 0.207 | 1 |
| Hilpda | 0.000414 | −0.30413 | 0.531 | 0.707 | 1 |
| Chchd2 | 0.000415 | −0.10752 | 0.959 | 0.992 | 1 |
| Cdk6 | 0.000423 | 0.169799 | 0.379 | 0.334 | 1 |
| Tax1bp1 | 0.000425 | −0.21772 | 0.8 | 0.874 | 1 |
| Plekhm2 | 0.000435 | −0.19253 | 0.317 | 0.497 | 1 |
| Fkbp4 | 0.000438 | 0.126756 | 0.262 | 0.219 | 1 |
| Rbm3 | 0.000439 | 0.174511 | 0.966 | 0.971 | 1 |
| Nucks1 | 0.000441 | 0.151222 | 0.503 | 0.504 | 1 |
| Stat3 | 0.000441 | 0.203739 | 0.862 | 0.74 | 1 |
| Man1a2 | 0.000441 | 0.118643 | 0.262 | 0.219 | 1 |
| Capg | 0.000443 | −0.20536 | 0.793 | 0.906 | 1 |
| Ube2g1 | 0.000443 | −0.16519 | 0.166 | 0.309 | 1 |
| Lasp1 | 0.000444 | −0.21437 | 0.628 | 0.776 | 1 |
| Ndufb2 | 0.000449 | −0.12845 | 0.393 | 0.572 | 1 |
| Errfi1 | 0.00045 | −0.3651 | 0.276 | 0.382 | 1 |
| Lims1 | 0.000452 | −0.10272 | 0.517 | 0.678 | 1 |
| Abcg1 | 0.000453 | −0.20495 | 0.6 | 0.761 | 1 |
| Slc27a4 | 0.000475 | −0.11639 | 0.076 | 0.167 | 1 |
| Atp6v0e | 0.000476 | −0.15097 | 0.876 | 0.949 | 1 |
| Abhd17a | 0.000507 | 0.135281 | 0.4 | 0.368 | 1 |
| Gdi2 | 0.000515 | 0.169642 | 0.91 | 0.914 | 1 |
| S100a11 | 0.00052 | −0.21058 | 0.828 | 0.92 | 1 |
| Emc10 | 0.000525 | −0.18465 | 0.379 | 0.531 | 1 |
| Oas3 | 0.000527 | −0.1526 | 0.469 | 0.646 | 1 |
| Fam214b | 0.000535 | −0.16188 | 0.221 | 0.389 | 1 |
| Dr1 | 0.000536 | 0.101613 | 0.317 | 0.335 | 1 |
| Hcar2 | 0.000536 | −0.37984 | 0.338 | 0.506 | 1 |
| Mirt2 | 0.00054 | −0.11523 | 0.034 | 0.142 | 1 |
| Atp5d | 0.000541 | 0.111592 | 0.772 | 0.823 | 1 |
| Zfp148 | 0.000543 | 0.12 | 0.393 | 0.388 | 1 |
| Hnrnpm | 0.000546 | 0.135435 | 0.759 | 0.796 | 1 |
| Park7 | 0.000548 | 0.132283 | 0.641 | 0.653 | 1 |
| Oaz1 | 0.000554 | 0.163403 | 0.993 | 0.984 | 1 |
| Slc8b1 | 0.000561 | −0.18489 | 0.283 | 0.418 | 1 |
| Pcbp2 | 0.000561 | 0.164036 | 0.931 | 0.937 | 1 |
| Specc1l | 0.000563 | 0.153168 | 0.366 | 0.315 | 1 |
| Nfkbiz | 0.000565 | −0.24705 | 0.703 | 0.841 | 1 |
| Taok1 | 0.000569 | 0.105306 | 0.559 | 0.6 | 1 |
| Nfatc2 | 0.000574 | 0.151963 | 0.317 | 0.228 | 1 |
| Arhgef7 | 0.00058 | 0.118775 | 0.262 | 0.222 | 1 |
| Prnp | 0.000581 | −0.14576 | 0.152 | 0.281 | 1 |
| Gmps | 0.000597 | 0.135162 | 0.317 | 0.272 | 1 |
| Pom121 | 0.000602 | −0.12744 | 0.124 | 0.235 | 1 |
| Tcp11l1 | 0.000615 | −0.10999 | 0.138 | 0.28 | 1 |
| Snx17 | 0.000617 | 0.135361 | 0.572 | 0.574 | 1 |
| Slc4a7 | 0.000619 | 0.154621 | 0.338 | 0.267 | 1 |
| Ptges3 | 0.000621 | −0.19245 | 0.614 | 0.762 | 1 |
| Suclg2 | 0.000623 | 0.142999 | 0.262 | 0.196 | 1 |
| Iqgap1 | 0.000625 | −0.14779 | 0.945 | 0.968 | 1 |
| Tnfrsf1b | 0.000629 | −0.24517 | 0.614 | 0.754 | 1 |
| Srd5a3 | 0.00064 | 0.106611 | 0.441 | 0.484 | 1 |
| Pik3r6 | 0.000641 | 0.115591 | 0.207 | 0.143 | 1 |
| Mefv | 0.000643 | −0.11733 | 0.214 | 0.37 | 1 |
| Mafb | 0.00066 | 0.257898 | 0.924 | 0.945 | 1 |
| Preb | 0.000668 | −0.15186 | 0.179 | 0.319 | 1 |
| Pik3cg | 0.000672 | 0.132144 | 0.283 | 0.218 | 1 |
| Cfb | 0.000673 | −0.2615 | 0.71 | 0.831 | 1 |
| Gm44751 | 0.000675 | 0.214471 | 0.434 | 0.307 | 1 |
| Hmg20b | 0.000679 | 0.103235 | 0.331 | 0.32 | 1 |
| Hmgn1 | 0.000688 | 0.193798 | 0.428 | 0.308 | 1 |
| Numa1 | 0.000699 | 0.159909 | 0.359 | 0.266 | 1 |
| Ndufa12 | 0.000702 | 0.136769 | 0.579 | 0.585 | 1 |
| Emsy | 0.000703 | 0.127081 | 0.262 | 0.228 | 1 |
| Qk | 0.000703 | 0.159734 | 0.821 | 0.839 | 1 |
| Bnip3l | 0.000705 | −0.21751 | 0.628 | 0.753 | 1 |
| Diaph2 | 0.000705 | 0.185536 | 0.448 | 0.349 | 1 |
| Qdpr | 0.000707 | 0.165607 | 0.338 | 0.234 | 1 |
| Dnajc8 | 0.000707 | 0.124079 | 0.579 | 0.592 | 1 |
| Lipa | 0.000713 | −0.22756 | 0.531 | 0.632 | 1 |
| Oasl2 | 0.000716 | −0.18173 | 0.6 | 0.761 | 1 |
| Ms4a6b | 0.000721 | 0.198133 | 0.807 | 0.829 | 1 |
| Casd1 | 0.000722 | 0.141838 | 0.2 | 0.121 | 1 |
| 2900097C17Rik | 0.000722 | −0.1113 | 0.579 | 0.702 | 1 |
| Rpl38 | 0.000723 | 0.119225 | 0.993 | 0.975 | 1 |
| Manf | 0.000733 | −0.1729 | 0.628 | 0.771 | 1 |
| Elf2 | 0.000739 | 0.14915 | 0.414 | 0.368 | 1 |
| Plk2 | 0.000744 | −0.25748 | 0.545 | 0.712 | 1 |
| Rnf7 | 0.000751 | 0.111628 | 0.662 | 0.707 | 1 |
| Myo9b | 0.000751 | 0.109854 | 0.483 | 0.488 | 1 |
| Eps8 | 0.000754 | −0.25851 | 0.476 | 0.622 | 1 |
| Idh2 | 0.000758 | 0.149764 | 0.393 | 0.361 | 1 |
| Zfp277 | 0.00076 | 0.106903 | 0.269 | 0.231 | 1 |
| Cers2 | 0.000763 | −0.17953 | 0.628 | 0.772 | 1 |
| Psmd13 | 0.000781 | −0.16862 | 0.462 | 0.636 | 1 |
| Fam20b | 0.000787 | 0.143349 | 0.331 | 0.274 | 1 |
| Tollip | 0.000787 | −0.11817 | 0.338 | 0.497 | 1 |
| Cers6 | 0.000792 | 0.166628 | 0.552 | 0.535 | 1 |
| 1810058124Rik | 0.000794 | −0.17224 | 0.545 | 0.715 | 1 |
| Vps53 | 0.000815 | 0.106916 | 0.29 | 0.245 | 1 |
| Bmt2 | 0.000832 | −0.15185 | 0.103 | 0.235 | 1 |
| Gm48065 | 0.000832 | 0.114587 | 0.159 | 0.086 | 1 |
| Snap23 | 0.000855 | −0.14444 | 0.497 | 0.634 | 1 |
| Mrpl13 | 0.000856 | 0.176546 | 0.49 | 0.386 | 1 |
| Psmc4 | 0.00086 | −0.13575 | 0.414 | 0.589 | 1 |
| Slc16a1 | 0.000867 | −0.11205 | 0.055 | 0.171 | 1 |
| Il2rg | 0.000867 | 0.161557 | 0.621 | 0.599 | 1 |
| 1810013L24Rik | 0.000871 | −0.14065 | 0.276 | 0.444 | 1 |
| Ghitm | 0.000873 | −0.12563 | 0.503 | 0.664 | 1 |
| Bri3 | 0.000883 | −0.16306 | 0.91 | 0.96 | 1 |
| Ifi27l2a | 0.000887 | −0.34146 | 0.924 | 0.96 | 1 |
| Gapdh | 0.000889 | −0.24136 | 0.979 | 0.976 | 1 |
| Wars | 0.000893 | 0.121859 | 0.386 | 0.374 | 1 |
| Asprv1 | 0.000895 | −0.12386 | 0.028 | 0.116 | 1 |
| Smim7 | 0.0009 | 0.124916 | 0.366 | 0.347 | 1 |
| Gclm | 0.000902 | −0.21321 | 0.31 | 0.485 | 1 |
| Acot8 | 0.000912 | −0.11078 | 0.076 | 0.202 | 1 |
| Prr13 | 0.000912 | −0.19325 | 0.655 | 0.786 | 1 |
| Casp1 | 0.000916 | −0.11522 | 0.434 | 0.598 | 1 |
| Ipo7 | 0.000934 | 0.107177 | 0.276 | 0.263 | 1 |
| Sec13 | 0.00094 | 0.15644 | 0.545 | 0.49 | 1 |
| Il10rb | 0.000942 | −0.13678 | 0.614 | 0.77 | 1 |
| Ifitm1 | 0.000948 | −0.1244 | 0.207 | 0.352 | 1 |
| Prpf38b | 0.00095 | 0.142621 | 0.538 | 0.522 | 1 |
| Fam20c | 0.000951 | −0.17995 | 0.166 | 0.298 | 1 |
| Pon2 | 0.000959 | 0.13977 | 0.324 | 0.272 | 1 |
| Actn4 | 0.000963 | −0.16579 | 0.586 | 0.729 | 1 |
| Taf15 | 0.000966 | 0.122714 | 0.31 | 0.276 | 1 |
| Rpl22 | 0.000973 | 0.204646 | 0.972 | 0.943 | 1 |
| Il1a | 0.000973 | −0.48754 | 0.269 | 0.411 | 1 |
| Tm6sf1 | 0.000978 | 0.165453 | 0.586 | 0.568 | 1 |
| Rplp0 | 0.000985 | 0.208415 | 0.972 | 0.955 | 1 |
| Cox16 | 0.000987 | 0.103498 | 0.414 | 0.427 | 1 |
| Lbh | 0.000989 | 0.147682 | 0.497 | 0.519 | 1 |
| Larp4 | 0.001004 | 0.122354 | 0.434 | 0.426 | 1 |
| Tnpo3 | 0.001005 | −0.14791 | 0.276 | 0.425 | 1 |
| Nme2 | 0.001024 | 0.225714 | 0.917 | 0.851 | 1 |
| Fnip2 | 0.001026 | −0.19228 | 0.393 | 0.567 | 1 |
| Cd53 | 0.001028 | −0.18992 | 0.945 | 0.954 | 1 |
| Fpr2 | 0.001044 | −0.29477 | 0.4 | 0.543 | 1 |
| Rab5if | 0.001047 | −0.19084 | 0.69 | 0.799 | 1 |
| Etf1 | 0.001067 | −0.16671 | 0.648 | 0.797 | 1 |
| Bak1 | 0.00107 | −0.19146 | 0.6 | 0.718 | 1 |
| Morrbid | 0.001078 | −0.121 | 0.076 | 0.196 | 1 |
| C3 | 0.00108 | 0.252926 | 0.683 | 0.694 | 1 |
| Sub1 | 0.001107 | 0.197155 | 0.903 | 0.879 | 1 |
| Nfkbia | 0.001131 | −0.26413 | 0.903 | 0.954 | 1 |
| Ftl1-ps1 | 0.001133 | −0.13488 | 0.241 | 0.385 | 1 |
| Mrps15 | 0.001142 | 0.107778 | 0.434 | 0.439 | 1 |
| Minpp1 | 0.001146 | 0.1042 | 0.255 | 0.239 | 1 |
| Pitpna | 0.001152 | −0.2026 | 0.883 | 0.914 | 1 |
| Ccdc6 | 0.001164 | 0.113722 | 0.179 | 0.134 | 1 |
| Plekha2 | 0.001185 | −0.13749 | 0.352 | 0.513 | 1 |
| Ncf4 | 0.00119 | 0.208055 | 0.793 | 0.706 | 1 |
| Epb41l2 | 0.001202 | 0.16295 | 0.648 | 0.652 | 1 |
| Tom1 | 0.001224 | −0.1147 | 0.159 | 0.308 | 1 |
| Ppt1 | 0.001228 | 0.154894 | 0.69 | 0.678 | 1 |
| Klf7 | 0.001254 | 0.162244 | 0.566 | 0.602 | 1 |
| Nav1 | 0.00126 | 0.138056 | 0.255 | 0.175 | 1 |
| Ezh1 | 0.001264 | 0.112246 | 0.214 | 0.173 | 1 |
| Irf2 | 0.001278 | 0.155767 | 0.786 | 0.791 | 1 |
| Med21 | 0.001308 | −0.19783 | 0.393 | 0.533 | 1 |
| Lyst | 0.001308 | −0.14712 | 0.283 | 0.444 | 1 |
| Rgl2 | 0.001312 | 0.115528 | 0.241 | 0.197 | 1 |
| Pgls | 0.001314 | 0.137832 | 0.634 | 0.632 | 1 |
| Wdr26 | 0.001333 | −0.15528 | 0.717 | 0.85 | 1 |
| Metap2 | 0.001334 | −0.1641 | 0.566 | 0.727 | 1 |
| Immp1l | 0.001343 | −0.12495 | 0.152 | 0.298 | 1 |
| Abracl | 0.001352 | 0.167553 | 0.786 | 0.792 | 1 |
| Hivep3 | 0.001368 | 0.143518 | 0.214 | 0.139 | 1 |
| Csrp1 | 0.001376 | 0.166214 | 0.352 | 0.274 | 1 |
| Fbxo33 | 0.001393 | −0.16651 | 0.186 | 0.341 | 1 |
| Lrpap1 | 0.001403 | −0.18063 | 0.414 | 0.579 | 1 |
| Rassf3 | 0.001408 | 0.19577 | 0.441 | 0.341 | 1 |
| Plgrkt | 0.00141 | −0.20756 | 0.517 | 0.672 | 1 |
| Smox | 0.001412 | −0.27113 | 0.441 | 0.574 | 1 |
| Itga4 | 0.001418 | 0.197718 | 0.372 | 0.293 | 1 |
| Myadm | 0.001425 | −0.16657 | 0.276 | 0.427 | 1 |
| Spg21 | 0.001431 | 0.117585 | 0.676 | 0.691 | 1 |
| Gipc1 | 0.00145 | −0.13332 | 0.262 | 0.418 | 1 |
| Ppp1r7 | 0.001461 | −0.10483 | 0.214 | 0.366 | 1 |
| Znrf1 | 0.001469 | −0.12718 | 0.186 | 0.327 | 1 |
| Bbip1 | 0.001471 | −0.14135 | 0.359 | 0.53 | 1 |
| Arhgef2 | 0.001481 | 0.107874 | 0.559 | 0.571 | 1 |
| Dpagt1 | 0.001481 | 0.10475 | 0.221 | 0.192 | 1 |
| Zfp644 | 0.001482 | 0.119919 | 0.324 | 0.292 | 1 |
| Rnase6 | 0.001492 | 0.154117 | 0.159 | 0.103 | 1 |
| Dcaf8 | 0.001525 | 0.125096 | 0.352 | 0.255 | 1 |
| Myd88 | 0.001537 | −0.16975 | 0.434 | 0.585 | 1 |
| Adrm1 | 0.001538 | −0.14652 | 0.262 | 0.427 | 1 |
| Rab11fip1 | 0.001547 | −0.23823 | 0.317 | 0.475 | 1 |
| Hnrnpd | 0.001548 | 0.109513 | 0.662 | 0.697 | 1 |
| Eno1 | 0.001549 | −0.23963 | 0.821 | 0.87 | 1 |
| Gm2a | 0.001562 | 0.168703 | 0.848 | 0.874 | 1 |
| Ly9 | 0.001565 | −0.1156 | 0.172 | 0.315 | 1 |
| Igf1 | 0.001576 | 0.224197 | 0.207 | 0.121 | 1 |
| Ctsz | 0.001578 | −0.15036 | 0.952 | 0.987 | 1 |
| Syngr2 | 0.001582 | 0.110122 | 0.71 | 0.741 | 1 |
| Psmb2 | 0.001584 | −0.13488 | 0.697 | 0.836 | 1 |
| Qsox1 | 0.001594 | 0.123279 | 0.393 | 0.389 | 1 |
| Rybp | 0.001602 | −0.19288 | 0.297 | 0.443 | 1 |
| Eif3h | 0.001603 | 0.135829 | 0.779 | 0.766 | 1 |
| F3 | 0.001613 | −0.21472 | 0.055 | 0.149 | 1 |
| Inppl1 | 0.001621 | −0.13605 | 0.269 | 0.401 | 1 |
| Cmtm6 | 0.001623 | 0.160687 | 0.531 | 0.461 | 1 |
| Rab11a | 0.001631 | −0.17074 | 0.772 | 0.884 | 1 |
| Trib1 | 0.001645 | −0.1626 | 0.579 | 0.739 | 1 |
| Psmd11 | 0.001661 | −0.14771 | 0.566 | 0.726 | 1 |
| Tlr13 | 0.001668 | −0.16754 | 0.386 | 0.536 | 1 |
| Slfn4 | 0.001674 | −0.35719 | 0.379 | 0.531 | 1 |
| Fam217b | 0.001674 | 0.105447 | 0.145 | 0.103 | 1 |
| Cln5 | 0.001686 | −0.14696 | 0.207 | 0.361 | 1 |
| Ist1 | 0.001689 | −0.12848 | 0.593 | 0.733 | 1 |
| Bdp1 | 0.001701 | 0.113505 | 0.345 | 0.335 | 1 |
| Unc93b1 | 0.001704 | 0.162156 | 0.959 | 0.937 | 1 |
| Ucp2 | 0.001757 | 0.175977 | 0.924 | 0.924 | 1 |
| Rer1 | 0.001771 | −0.11576 | 0.552 | 0.702 | 1 |
| Sirpa | 0.00178 | 0.124923 | 0.917 | 0.957 | 1 |
| Slc12a9 | 0.001787 | 0.114511 | 0.262 | 0.242 | 1 |
| Rpl24 | 0.001804 | 0.173651 | 0.966 | 0.965 | 1 |
| Mrpl30 | 0.001812 | 0.12854 | 0.6 | 0.601 | 1 |
| Gbp4 | 0.001814 | −0.24237 | 0.772 | 0.877 | 1 |
| Stx8 | 0.001824 | 0.136579 | 0.428 | 0.368 | 1 |
| Aprt | 0.001828 | −0.17333 | 0.841 | 0.926 | 1 |
| mt-Atp8 | 0.001847 | 0.102299 | 0.234 | 0.203 | 1 |
| Cyth4 | 0.001855 | 0.163767 | 0.717 | 0.703 | 1 |
| Foxn3 | 0.00186 | 0.157511 | 0.366 | 0.265 | 1 |
| Svbp | 0.0019 | 0.167947 | 0.503 | 0.45 | 1 |
| Irak3 | 0.00192 | −0.13908 | 0.159 | 0.288 | 1 |
| Crk | 0.001925 | 0.124064 | 0.566 | 0.551 | 1 |
| Hyou1 | 0.001932 | −0.13505 | 0.214 | 0.357 | 1 |
| Usp9x | 0.001948 | 0.11775 | 0.531 | 0.54 | 1 |
| Mndal | 0.001949 | 0.176136 | 0.724 | 0.741 | 1 |
| Xrcc1 | 0.001951 | 0.123794 | 0.207 | 0.113 | 1 |
| Il6st | 0.001963 | −0.22039 | 0.317 | 0.449 | 1 |
| Slc7a11 | 0.001966 | −0.32426 | 0.228 | 0.379 | 1 |
| Lrp12 | 0.001968 | −0.15473 | 0.207 | 0.361 | 1 |
| Dnaaf3 | 0.001976 | 0.111839 | 0.166 | 0.102 | 1 |
| Saysd1 | 0.002004 | 0.100939 | 0.166 | 0.121 | 1 |
| Samsn1 | 0.002011 | −0.26814 | 0.297 | 0.438 | 1 |
| Il18bp | 0.002029 | −0.22103 | 0.669 | 0.794 | 1 |
| Camk1 | 0.002071 | 0.121385 | 0.524 | 0.518 | 1 |
| Med8 | 0.002073 | −0.11202 | 0.317 | 0.476 | 1 |
| Zup1 | 0.002083 | 0.160651 | 0.448 | 0.393 | 1 |
| Ppp3r1 | 0.002086 | −0.13549 | 0.359 | 0.512 | 1 |
| Stx16 | 0.002125 | 0.137302 | 0.324 | 0.229 | 1 |
| Tmem51 | 0.002125 | 0.137688 | 0.421 | 0.41 | 1 |
| Herc2 | 0.002137 | 0.157803 | 0.359 | 0.26 | 1 |
| Timm13 | 0.002154 | −0.12559 | 0.662 | 0.773 | 1 |
| Gnas | 0.002155 | −0.13137 | 0.683 | 0.815 | 1 |
| Cnot6l | 0.002166 | 0.141125 | 0.428 | 0.383 | 1 |
| Ctla2b | 0.002186 | 0.143243 | 0.159 | 0.062 | 1 |
| Gm15056 | 0.002202 | −0.68717 | 0.31 | 0.452 | 1 |
| Idi1 | 0.00229 | 0.117855 | 0.152 | 0.093 | 1 |
| Tmem251 | 0.002292 | −0.15791 | 0.317 | 0.478 | 1 |
| Dglucy | 0.002311 | 0.114485 | 0.131 | 0.064 | 1 |
| Nlrp3 | 0.002338 | −0.24675 | 0.566 | 0.72 | 1 |
| Cd93 | 0.002338 | 0.206017 | 0.545 | 0.518 | 1 |
| Clpp | 0.002355 | −0.12435 | 0.097 | 0.221 | 1 |
| Eno2 | 0.002378 | −0.10412 | 0.138 | 0.266 | 1 |
| Xaf1 | 0.002392 | −0.12156 | 0.428 | 0.59 | 1 |
| Naglu | 0.002395 | −0.12538 | 0.269 | 0.422 | 1 |
| Tcp1 | 0.002406 | 0.10125 | 0.572 | 0.601 | 1 |
| Rpl26 | 0.002415 | 0.173255 | 0.766 | 0.765 | 1 |
| Tnfrsf26 | 0.002428 | −0.17093 | 0.159 | 0.301 | 1 |
| Mfsd7a | 0.002435 | −0.11568 | 0.097 | 0.208 | 1 |
| Spata2 | 0.002442 | −0.1127 | 0.117 | 0.219 | 1 |
| Arl4c | 0.00246 | 0.239512 | 0.703 | 0.609 | 1 |
| Furin | 0.002462 | −0.14477 | 0.676 | 0.798 | 1 |
| Capns1 | 0.002494 | −0.15602 | 0.703 | 0.82 | 1 |
| Tes | 0.002504 | −0.14526 | 0.221 | 0.372 | 1 |
| Tut7 | 0.002554 | 0.138431 | 0.676 | 0.67 | 1 |
| Ube2a | 0.002582 | −0.13129 | 0.455 | 0.617 | 1 |
| Fgd2 | 0.002595 | 0.161274 | 0.276 | 0.207 | 1 |
| Kxd1 | 0.002597 | 0.115727 | 0.483 | 0.463 | 1 |
| Chmp1b | 0.002599 | −0.10777 | 0.324 | 0.48 | 1 |
| Emc6 | 0.002626 | 0.101337 | 0.448 | 0.448 | 1 |
| Jmjd1c | 0.002628 | 0.122221 | 0.628 | 0.658 | 1 |
| Ndufb9 | 0.002628 | −0.15826 | 0.766 | 0.874 | 1 |
| Ralgds | 0.002629 | −0.19463 | 0.414 | 0.56 | 1 |
| Pik3ca | 0.002633 | −0.15134 | 0.234 | 0.383 | 1 |
| Shoc2 | 0.002664 | −0.12071 | 0.262 | 0.417 | 1 |
| Arl6ip1 | 0.00267 | −0.1453 | 0.841 | 0.915 | 1 |
| Emilin1 | 0.002683 | 0.118574 | 0.221 | 0.169 | 1 |
| Mir155hg | 0.002687 | −0.15417 | 0.076 | 0.171 | 1 |
| Jag1 | 0.002689 | −0.19204 | 0.09 | 0.181 | 1 |
| Gak | 0.00269 | −0.14811 | 0.331 | 0.474 | 1 |
| Cfl1 | 0.002715 | −0.13856 | 0.979 | 0.983 | 1 |
| Man1a | 0.002745 | 0.201275 | 0.559 | 0.503 | 1 |
| Fdps | 0.002762 | 0.112479 | 0.241 | 0.142 | 1 |
| Svil | 0.002784 | 0.143599 | 0.386 | 0.301 | 1 |
| Cd72 | 0.002798 | 0.182452 | 0.421 | 0.277 | 1 |
| Cd48 | 0.002802 | 0.17774 | 0.579 | 0.491 | 1 |
| Mthfsl | 0.002809 | −0.12346 | 0.214 | 0.341 | 1 |
| Colec12 | 0.002811 | 0.187434 | 0.607 | 0.53 | 1 |
| Ep400 | 0.002842 | 0.123914 | 0.407 | 0.363 | 1 |
| Acadm | 0.002845 | 0.140573 | 0.359 | 0.304 | 1 |
| Cd300ld | 0.002848 | −0.14612 | 0.159 | 0.296 | 1 |
| Klhl9 | 0.002938 | −0.10084 | 0.248 | 0.395 | 1 |
| Gch1 | 0.002967 | −0.15242 | 0.51 | 0.669 | 1 |
| Epb41 | 0.00301 | 0.106505 | 0.4 | 0.405 | 1 |
| Rtf1 | 0.003024 | −0.11339 | 0.317 | 0.471 | 1 |
| Ggnbp2 | 0.003034 | −0.10538 | 0.476 | 0.604 | 1 |
| Xylt2 | 0.003097 | −0.10912 | 0.097 | 0.214 | 1 |
| Arhgap18 | 0.003111 | 0.144558 | 0.29 | 0.226 | 1 |
| Gadd45a | 0.003131 | −0.23247 | 0.138 | 0.267 | 1 |
| Ophn1 | 0.003147 | 0.112478 | 0.179 | 0.086 | 1 |
| U2af2 | 0.003174 | −0.14304 | 0.366 | 0.528 | 1 |
| Atrx | 0.003192 | 0.130358 | 0.503 | 0.486 | 1 |
| Pdpn | 0.003228 | −0.32738 | 0.297 | 0.418 | 1 |
| Myl12b | 0.003232 | 0.139097 | 0.876 | 0.875 | 1 |
| Tiparp | 0.003249 | −0.23312 | 0.614 | 0.734 | 1 |
| Itgam | 0.00325 | 0.24647 | 0.807 | 0.886 | 1 |
| Tpr | 0.003256 | 0.144636 | 0.738 | 0.756 | 1 |
| Ufm1 | 0.003381 | −0.10144 | 0.434 | 0.581 | 1 |
| Arl5c | 0.003401 | 0.184739 | 0.4 | 0.343 | 1 |
| Elovl1 | 0.003411 | −0.10343 | 0.31 | 0.457 | 1 |
| Wdr3 | 0.003463 | 0.102755 | 0.228 | 0.186 | 1 |
| Pgd | 0.003476 | −0.148 | 0.572 | 0.724 | 1 |
| Stx12 | 0.003511 | −0.10207 | 0.4 | 0.54 | 1 |
| Ms4a7 | 0.00357 | 0.277516 | 0.807 | 0.762 | 1 |
| Gk | 0.00358 | −0.16257 | 0.207 | 0.354 | 1 |
| Rrp36 | 0.003604 | −0.10514 | 0.069 | 0.175 | 1 |
| Galnt7 | 0.003633 | 0.151411 | 0.4 | 0.347 | 1 |
| Map3k2 | 0.003686 | 0.109245 | 0.407 | 0.405 | 1 |
| Ank2 | 0.003714 | 0.180861 | 0.29 | 0.214 | 1 |
| Cycs | 0.003724 | −0.19852 | 0.697 | 0.808 | 1 |
| Lmnb1 | 0.003753 | −0.22449 | 0.276 | 0.426 | 1 |
| Tob1 | 0.003817 | −0.13901 | 0.207 | 0.325 | 1 |
| Gng2 | 0.003827 | −0.16404 | 0.676 | 0.761 | 1 |
| Tpi1 | 0.003842 | −0.23757 | 0.89 | 0.938 | 1 |
| Sde2 | 0.003862 | −0.16943 | 0.421 | 0.576 | 1 |
| Lpl | 0.003882 | −0.51118 | 0.186 | 0.27 | 1 |
| Ccl8 | 0.003924 | 0.13618 | 0.841 | 0.889 | 1 |
| Klf10 | 0.003995 | −0.1756 | 0.255 | 0.401 | 1 |
| Agtpbp1 | 0.004038 | −0.10313 | 0.166 | 0.296 | 1 |
| Ubr4 | 0.004087 | −0.12064 | 0.4 | 0.519 | 1 |
| Gm49774 | 0.004089 | −0.18325 | 0.179 | 0.305 | 1 |
| Eid1 | 0.004107 | 0.131592 | 0.621 | 0.609 | 1 |
| Wfdc21 | 0.004156 | −0.12782 | 0.034 | 0.112 | 1 |
| Creb5 | 0.004202 | 0.218895 | 0.572 | 0.518 | 1 |
| Atp5md | 0.004203 | −0.10677 | 0.69 | 0.808 | 1 |
| B4galt1 | 0.004221 | 0.142309 | 0.434 | 0.417 | 1 |
| Son | 0.004227 | 0.135201 | 0.752 | 0.763 | 1 |
| Alcam | 0.00424 | 0.175254 | 0.352 | 0.266 | 1 |
| Cxcr4 | 0.00425 | 0.215574 | 0.628 | 0.612 | 1 |
| Galk2 | 0.004297 | 0.100267 | 0.29 | 0.269 | 1 |
| Tmem123 | 0.004301 | 0.160293 | 0.586 | 0.557 | 1 |
| Gpr171 | 0.004309 | 0.147837 | 0.138 | 0.075 | 1 |
| BC031181 | 0.004321 | −0.13459 | 0.428 | 0.576 | 1 |
| Tpm4 | 0.004428 | 0.129976 | 0.683 | 0.686 | 1 |
| AC149090.1 | 0.004474 | 0.127103 | 0.324 | 0.316 | 1 |
| Capn1 | 0.004518 | −0.14414 | 0.31 | 0.464 | 1 |
| Mib1 | 0.00457 | 0.112702 | 0.345 | 0.331 | 1 |
| Cnn3 | 0.004581 | 0.127477 | 0.559 | 0.58 | 1 |
| Smc6 | 0.004616 | 0.129601 | 0.497 | 0.48 | 1 |
| Akap9 | 0.004626 | 0.115866 | 0.29 | 0.245 | 1 |
| Cdkn2d | 0.004646 | −0.13022 | 0.159 | 0.269 | 1 |
| Ldha | 0.004649 | −0.13174 | 0.869 | 0.949 | 1 |
| Ptgr1 | 0.004692 | −0.17542 | 0.276 | 0.356 | 1 |
| Etnk1 | 0.004699 | 0.164418 | 0.49 | 0.437 | 1 |
| Ndufb1-ps | 0.004702 | −0.17189 | 0.814 | 0.847 | 1 |
| Pim1 | 0.004807 | −0.14899 | 0.91 | 0.965 | 1 |
| Ctdspl2 | 0.004819 | 0.100082 | 0.221 | 0.169 | 1 |
| Vps37b | 0.004871 | −0.14041 | 0.331 | 0.484 | 1 |
| Scp2 | 0.004874 | 0.150173 | 0.71 | 0.687 | 1 |
| Mgst1 | 0.004929 | −0.19555 | 0.434 | 0.589 | 1 |
| Cript | 0.005026 | −0.10383 | 0.51 | 0.655 | 1 |
| Srgn | 0.00503 | −0.37106 | 0.959 | 0.954 | 1 |
| Zfas1 | 0.00505 | 0.184829 | 0.379 | 0.291 | 1 |
| S100a6 | 0.005077 | 0.16825 | 0.952 | 0.989 | 1 |
| Nup62 | 0.005094 | −0.11658 | 0.159 | 0.29 | 1 |
| Tet3 | 0.005095 | 0.148556 | 0.559 | 0.519 | 1 |
| Psmb6 | 0.005159 | −0.15904 | 0.766 | 0.874 | 1 |
| Tmem234 | 0.005193 | 0.108785 | 0.738 | 0.76 | 1 |
| Rnf141 | 0.005238 | −0.11532 | 0.166 | 0.297 | 1 |
| Ch25h | 0.00531 | 0.324207 | 0.297 | 0.218 | 1 |
| Cd180 | 0.005316 | −0.15759 | 0.338 | 0.49 | 1 |
| Lamtor1 | 0.005335 | −0.11338 | 0.703 | 0.831 | 1 |
| Scimp | 0.005342 | 0.177182 | 0.462 | 0.425 | 1 |
| Hist1h1c | 0.005377 | 0.132458 | 0.138 | 0.122 | 1 |
| Itpr2 | 0.005474 | 0.134011 | 0.262 | 0.194 | 1 |
| Ubxn4 | 0.005474 | −0.14356 | 0.49 | 0.642 | 1 |
| Midn | 0.005489 | −0.12288 | 0.338 | 0.482 | 1 |
| Pecam1 | 0.005497 | −0.12602 | 0.221 | 0.347 | 1 |
| Atp13a3 | 0.00551 | 0.100218 | 0.4 | 0.411 | 1 |
| Micall1 | 0.005542 | −0.11176 | 0.11 | 0.219 | 1 |
| Rab7 | 0.005551 | −0.18385 | 0.8 | 0.855 | 1 |
| Cmc1 | 0.005555 | 0.133089 | 0.441 | 0.379 | 1 |
| Alas1 | 0.005558 | 0.117384 | 0.269 | 0.217 | 1 |
| Rab7b | 0.005625 | −0.18164 | 0.441 | 0.582 | 1 |
| Abca1 | 0.005652 | 0.14454 | 0.593 | 0.639 | 1 |
| Cenpa | 0.005672 | 0.143775 | 0.228 | 0.164 | 1 |
| Hadhb | 0.005674 | 0.105464 | 0.421 | 0.421 | 1 |
| Uhrf2 | 0.005676 | 0.100943 | 0.366 | 0.379 | 1 |
| Ikbke | 0.00572 | −0.14659 | 0.386 | 0.524 | 1 |
| Timm8a1 | 0.005733 | −0.10852 | 0.145 | 0.27 | 1 |
| Mcemp1 | 0.005766 | −0.17143 | 0.131 | 0.251 | 1 |
| Pycard | 0.005781 | 0.18486 | 0.676 | 0.797 | 1 |
| 5-Mar | 0.005819 | 0.101007 | 0.545 | 0.583 | 1 |
| Vezf1 | 0.005824 | 0.108284 | 0.29 | 0.261 | 1 |
| Dmac2 | 0.005863 | 0.104623 | 0.159 | 0.075 | 1 |
| 2310022A10Rik | 0.005874 | −0.14031 | 0.29 | 0.419 | 1 |
| Car13 | 0.006023 | −0.15076 | 0.207 | 0.347 | 1 |
| H13 | 0.006043 | 0.108979 | 0.559 | 0.579 | 1 |
| Timm17a | 0.006092 | −0.10263 | 0.324 | 0.469 | 1 |
| Gbp2 | 0.00619 | −0.22948 | 0.848 | 0.894 | 1 |
| Snhg3 | 0.006205 | 0.104826 | 0.4 | 0.388 | 1 |
| Abce1 | 0.006359 | −0.13315 | 0.2 | 0.339 | 1 |
| Gm16286 | 0.006395 | −0.15806 | 0.593 | 0.728 | 1 |
| Clec4b1 | 0.006504 | −0.16274 | 0.048 | 0.117 | 1 |
| Bola3 | 0.006523 | 0.106322 | 0.338 | 0.32 | 1 |
| Snx18 | 0.006678 | −0.17033 | 0.628 | 0.76 | 1 |
| Ctdsp2 | 0.006826 | 0.100145 | 0.448 | 0.462 | 1 |
| Ctss | 0.006839 | −0.16714 | 1 | 0.998 | 1 |
| Apbb1ip | 0.006848 | −0.18812 | 0.572 | 0.703 | 1 |
| Tmem106a | 0.006852 | −0.14071 | 0.731 | 0.838 | 1 |
| Fam102b | 0.00687 | −0.14518 | 0.366 | 0.514 | 1 |
| Pak2 | 0.006912 | −0.11547 | 0.724 | 0.832 | 1 |
| Il1r2 | 0.006913 | 0.17912 | 0.131 | 0.254 | 1 |
| Rreb1 | 0.006923 | 0.130549 | 0.407 | 0.362 | 1 |
| Pde3b | 0.006928 | 0.104282 | 0.159 | 0.093 | 1 |
| Cstdc4 | 0.006948 | −0.43339 | 0.097 | 0.196 | 1 |
| Vopp1 | 0.006954 | 0.120025 | 0.283 | 0.245 | 1 |
| Rnase2a | 0.007018 | 0.236189 | 0.152 | 0.064 | 1 |
| Tlnrd1 | 0.00709 | −0.13118 | 0.331 | 0.478 | 1 |
| Cks2 | 0.007101 | 0.120864 | 0.207 | 0.176 | 1 |
| Tnrc6b | 0.007157 | 0.160945 | 0.483 | 0.352 | 1 |
| Nr4a2 | 0.007219 | 0.287441 | 0.421 | 0.329 | 1 |
| Hnrnpk | 0.007275 | 0.121056 | 0.917 | 0.931 | 1 |
| Mpp6 | 0.007394 | 0.123327 | 0.29 | 0.255 | 1 |
| Fam104a | 0.007425 | −0.11293 | 0.538 | 0.682 | 1 |
| Mxd4 | 0.007522 | 0.120347 | 0.255 | 0.176 | 1 |
| Eva1b | 0.007675 | 0.117462 | 0.29 | 0.245 | 1 |
| Malat1 | 0.007679 | 0.122545 | 0.972 | 0.981 | 1 |
| Zfp281 | 0.007733 | −0.11297 | 0.241 | 0.375 | 1 |
| Zyx | 0.007761 | −0.21285 | 0.71 | 0.786 | 1 |
| Rgs2 | 0.007789 | 0.1778 | 0.586 | 0.496 | 1 |
| Neurl3 | 0.007794 | 0.176112 | 0.352 | 0.246 | 1 |
| Calhm6 | 0.007948 | 0.199037 | 0.51 | 0.526 | 1 |
| Kpna4 | 0.007948 | −0.12948 | 0.586 | 0.727 | 1 |
| Hsd3b7 | 0.008136 | −0.16309 | 0.352 | 0.471 | 1 |
| Cd9 | 0.00814 | −0.26127 | 0.262 | 0.395 | 1 |
| Oser1 | 0.008171 | −0.12863 | 0.407 | 0.556 | 1 |
| Bcl2a1b | 0.00819 | 0.201222 | 0.641 | 0.644 | 1 |
| Med28 | 0.008225 | −0.15921 | 0.503 | 0.63 | 1 |
| Mrpl48 | 0.008278 | 0.106893 | 0.317 | 0.293 | 1 |
| Fyb | 0.008312 | 0.115867 | 0.862 | 0.843 | 1 |
| Polr3gl | 0.008315 | 0.106971 | 0.234 | 0.183 | 1 |
| Nipbl | 0.008338 | 0.137477 | 0.614 | 0.535 | 1 |
| Il18 | 0.008343 | −0.11492 | 0.248 | 0.374 | 1 |
| Clcn5 | 0.008362 | −0.14077 | 0.434 | 0.459 | 1 |
| Spop | 0.008414 | 0.124979 | 0.648 | 0.652 | 1 |
| Lmna | 0.008414 | −0.22997 | 0.752 | 0.823 | 1 |
| Psma3 | 0.008432 | −0.16049 | 0.793 | 0.877 | 1 |
| Sirt2 | 0.008467 | −0.12853 | 0.434 | 0.571 | 1 |
| Mmp8 | 0.008522 | −0.117 | 0.069 | 0.111 | 1 |
| Bod1l | 0.008688 | 0.106475 | 0.386 | 0.355 | 1 |
| Lat2 | 0.008764 | −0.15975 | 0.379 | 0.495 | 1 |
| Zmiz1 | 0.008786 | 0.112379 | 0.545 | 0.562 | 1 |
| Cdc42se2 | 0.0088 | 0.102591 | 0.545 | 0.556 | 1 |
| Cbr2 | 0.008825 | 0.240019 | 0.455 | 0.33 | 1 |
| Rcn2 | 0.008854 | 0.136509 | 0.359 | 0.284 | 1 |
| Klf13 | 0.008906 | 0.129246 | 0.745 | 0.718 | 1 |
| Atpif1 | 0.008951 | 0.108529 | 0.703 | 0.74 | 1 |
| Prune2 | 0.008998 | 0.113816 | 0.221 | 0.134 | 1 |
| Ipo5 | 0.009055 | −0.10858 | 0.214 | 0.316 | 1 |
| Ms4a4b | 0.00909 | 0.117435 | 0.159 | 0.102 | 1 |
| Tnfaip8 | 0.009215 | 0.138883 | 0.593 | 0.562 | 1 |
| Pold4 | 0.009323 | 0.122665 | 0.517 | 0.497 | 1 |
| Cmpk2 | 0.009326 | 0.130709 | 0.303 | 0.38 | 1 |
| Rps2 | 0.009362 | 0.132035 | 0.972 | 0.938 | 1 |
| B4galt3 | 0.009407 | −0.11823 | 0.145 | 0.213 | 1 |
| Fosb | 0.00941 | 0.201556 | 0.724 | 0.762 | 1 |
| Ccr2 | 0.009423 | 0.294106 | 0.724 | 0.606 | 1 |
| Daglb | 0.00948 | 0.129602 | 0.352 | 0.298 | 1 |
| Il17ra | 0.009527 | 0.138717 | 0.317 | 0.242 | 1 |
| Gpr65 | 0.009544 | 0.198249 | 0.503 | 0.405 | 1 |
| Hint1 | 0.009585 | −0.15844 | 0.786 | 0.869 | 1 |
| Sltm | 0.009607 | 0.105998 | 0.441 | 0.44 | 1 |
| Plscr3 | 0.009614 | 0.118165 | 0.483 | 0.454 | 1 |
| Acvrl1 | 0.009633 | −0.10972 | 0.317 | 0.459 | 1 |
| Osgep | 0.009635 | 0.151106 | 0.421 | 0.365 | 1 |
| Sdc3 | 0.009647 | −0.13672 | 0.979 | 0.976 | 1 |
| Ginm1 | 0.009765 | −0.10298 | 0.159 | 0.272 | 1 |
| Vat1 | 0.009961 | −0.17769 | 0.517 | 0.657 | 1 |
| Exoc3 | 0.009973 | 0.102159 | 0.366 | 0.341 | 1 |
| Cxcl1 | 0.011744 | −0.3829 | 0.483 | 0.61 | 1 |
| Selenop | 0.012637 | 0.265057 | 0.828 | 0.772 | 1 |
| Gm42418 | 0.015663 | 0.299093 | 1 | 0.999 | 1 |
| Il1rn | 0.021633 | −0.27738 | 0.745 | 0.811 | 1 |
| Stfa211 | 0.028301 | −0.27895 | 0.062 | 0.137 | 1 |
| Pf4 | 0.037953 | 0.302184 | 0.731 | 0.664 | 1 |
| Ly6a | 0.044421 | −0.27206 | 0.724 | 0.803 | 1 |
| Ccl5 | 0.052407 | 0.427119 | 0.448 | 0.387 | 1 |
| Ptgs2 | 0.063505 | −0.25859 | 0.614 | 0.701 | 1 |
| Mmp12 | 0.085238 | −0.57109 | 0.117 | 0.156 | 1 |
| Ccl6 | 0.089763 | −0.26793 | 0.924 | 0.897 | 1 |
| Ccl24 | 0.224172 | −0.26021 | 0.324 | 0.338 | 1 |

C5

| | | | | | |
|---|---|---|---|---|---|
| Nos2 | 3.55E−23 | −1.0948 | 0.333 | 0.73 | 7.33E−19 |
| Rps3a1 | 4.91E−23 | 0.391922 | 0.995 | 0.975 | 1.01E−18 |
| Rpl12 | 5.91E−22 | 0.552619 | 0.949 | 0.863 | 1.22E−17 |
| Gm15056 | 1.15E−20 | −1.75618 | 0.081 | 0.412 | 2.37E−16 |
| Rpl32 | 1.64E−20 | 0.504434 | 0.98 | 0.967 | 3.39E−16 |
| Rpl3 | 4.26E−20 | 0.451053 | 0.944 | 0.875 | 8.80E−16 |
| Rpl13a | 2.00E−19 | 0.480926 | 0.955 | 0.919 | 4.13E−15 |
| Rps12 | 3.39E−19 | 0.462887 | 0.995 | 0.982 | 7.00E−15 |
| Eef1a1 | 2.99E−18 | 0.322594 | 1 | 0.99 | 6.18E−14 |
| Lyz2 | 4.09E−18 | 0.81 | 0.899 | 0.766 | 8.44E−14 |
| Rps4x | 4.42E−18 | 0.412588 | 0.97 | 0.964 | 9.14E−14 |
| Rps20 | 5.11E−18 | 0.379528 | 0.995 | 0.975 | 1.05E−13 |
| Rps8 | 5.87E−18 | 0.388401 | 0.995 | 0.969 | 1.21E−13 |
| Rps19 | 1.52E−17 | 0.427067 | 0.98 | 0.947 | 3.14E−13 |
| Socs1 | 1.95E−17 | −0.8792 | 0.263 | 0.527 | 4.02E−13 |
| Rpl10a | 2.54E−17 | 0.506779 | 0.949 | 0.845 | 5.24E−13 |
| Tapbp | 5.17E−17 | −0.65621 | 0.596 | 0.789 | 1.07E−12 |
| Rpl13 | 5.76E−17 | 0.419681 | 0.985 | 0.962 | 1.19E−12 |
| Zbp1 | 5.92E−17 | −0.5998 | 0.283 | 0.646 | 1.22E−12 |
| Grina | 7.17E−17 | −0.59042 | 0.798 | 0.941 | 1.48E−12 |
| Rps27a | 7.29E−17 | 0.383932 | 0.995 | 0.964 | 1.50E−12 |
| Sod2 | 7.74E−17 | −0.70217 | 0.753 | 0.896 | 1.60E−12 |
| Tmsb10 | 1.02E−16 | −0.72184 | 0.909 | 0.969 | 2.11E−12 |
| Rps5 | 1.45E−16 | 0.432161 | 0.96 | 0.952 | 3.00E−12 |
| Ccl6 | 1.88E−16 | 0.641943 | 0.924 | 0.718 | 3.88E−12 |
| Mrc1 | 1.89E−16 | 0.891925 | 0.813 | 0.534 | 3.90E−12 |
| B2m | 6.25E−16 | −0.38365 | 1 | 1 | 1.29E−11 |
| Rp15 | 7.23E−16 | 0.536326 | 0.909 | 0.753 | 1.49E−11 |
| Rack1 | 7.79E−16 | 0.447237 | 0.97 | 0.898 | 1.61E−11 |
| Rpl14 | 1.09E−15 | 0.464316 | 0.929 | 0.83 | 2.24E−11 |
| Ccl9 | 1.27E−15 | 0.540685 | 0.909 | 0.69 | 2.62E−11 |
| AW112010 | 2.54E−15 | −1.08653 | 0.46 | 0.702 | 5.25E−11 |
| Cstb | 4.05E−15 | −0.3851 | 0.97 | 0.992 | 8.37E−11 |
| Rps11 | 4.19E−15 | 0.397807 | 0.98 | 0.957 | 8.65E−11 |
| Rpl18a | 4.70E−15 | 0.399111 | 0.985 | 0.934 | 9.71E−11 |
| Pf4 | 4.71E−15 | 0.840589 | 0.818 | 0.537 | 9.72E−11 |
| Rpsa | 5.26E−15 | 0.373682 | 0.955 | 0.934 | 1.09E−10 |
| Rps7 | 6.14E−15 | 0.365886 | 0.98 | 0.939 | 1.27E−10 |
| Glrx | 7.38E−15 | −0.77414 | 0.677 | 0.832 | 1.52E−10 |
| Rps14 | 8.72E−15 | 0.327626 | 0.975 | 0.954 | 1.80E−10 |
| Rplp1 | 9.84E−15 | 0.393351 | 0.985 | 0.99 | 2.03E−10 |
| Cebpb | 1.15E−14 | 0.383075 | 0.995 | 0.908 | 2.37E−10 |
| Rps13 | 1.17E−14 | 0.324799 | 0.985 | 0.947 | 2.42E−10 |
| Rps15 | 1.89E−14 | 0.415933 | 0.965 | 0.921 | 3.91E−10 |
| Rps24 | 2.14E−14 | 0.343876 | 0.99 | 0.972 | 4.43E−10 |
| Timp2 | 6.78E−14 | 0.741964 | 0.535 | 0.277 | 1.40E−09 |
| Rps10 | 8.40E−14 | 0.347008 | 0.995 | 0.967 | 1.74E−09 |
| Rpl17 | 1.74E−13 | 0.367938 | 0.975 | 0.944 | 3.59E−09 |
| Prdx6 | 2.09E−13 | −0.64006 | 0.747 | 0.911 | 4.32E−09 |
| Il1rn | 3.22E−13 | −0.6375 | 0.783 | 0.931 | 6.65E−09 |
| Irf7 | 5.17E−13 | −0.54559 | 0.495 | 0.781 | 1.07E−08 |
| S100a4 | 6.35E−13 | 0.575561 | 0.884 | 0.735 | 1.31E−08 |
| Rpl27a | 6.92E−13 | 0.305083 | 0.98 | 0.98 | 1.43E−08 |
| Rps15a | 1.14E−12 | 0.318098 | 0.975 | 0.977 | 2.35E−08 |
| Rps3 | 1.47E−12 | 0.298836 | 0.98 | 0.941 | 3.04E−08 |
| Rpl23 | 1.75E−12 | 0.323427 | 0.995 | 0.982 | 3.61E−08 |
| Eef1b2 | 2.51E−12 | 0.468619 | 0.869 | 0.743 | 5.19E−08 |
| AA467197 | 3.05E−12 | −1.02935 | 0.182 | 0.438 | 6.30E−08 |
| Npc2 | 3.49E−12 | −0.37602 | 0.98 | 0.997 | 7.20E−08 |
| Selenop | 4.36E−12 | 0.636955 | 0.475 | 0.214 | 9.01E−08 |
| Slamf7 | 6.97E−12 | −0.61454 | 0.157 | 0.417 | 1.44E−07 |
| Prdx5 | 8.66E−12 | −0.50848 | 0.909 | 0.975 | 1.79E−07 |
| Fth1 | 9.28E−12 | −0.44774 | 1 | 1 | 1.92E−07 |
| H2-T23 | 9.91E−12 | −0.50225 | 0.444 | 0.669 | 2.05E−07 |
| mt-Nd1 | 1.06E−11 | 0.539582 | 0.899 | 0.85 | 2.19E−07 |
| Slc31a1 | 1.26E−11 | −0.53055 | 0.399 | 0.656 | 2.60E−07 |
| Rsad2 | 1.33E−11 | −0.89516 | 0.318 | 0.623 | 2.74E−07 |
| Slc31a2 | 1.36E−11 | −0.40679 | 0.333 | 0.618 | 2.82E−07 |
| Upp1 | 1.38E−11 | −0.4806 | 0.399 | 0.705 | 2.86E−07 |
| Gbp2b | 1.59E−11 | −0.55761 | 0.333 | 0.618 | 3.28E−07 |
| Gbp2 | 1.67E−11 | −0.60797 | 0.343 | 0.641 | 3.44E−07 |
| Rpl7 | 1.84E−11 | 0.337044 | 0.99 | 0.962 | 3.81E−07 |
| Rpl8 | 2.27E−11 | 0.253696 | 0.949 | 0.962 | 4.70E−07 |
| Cd274 | 2.56E−11 | −0.56469 | 0.677 | 0.835 | 5.28E−07 |
| Fcgrt | 5.45E−11 | 0.551011 | 0.323 | 0.145 | 1.12E−06 |
| Rpl9 | 5.79E−11 | 0.298479 | 0.985 | 0.969 | 1.20E−06 |
| Rpl22 | 6.47E−11 | 0.322706 | 0.919 | 0.901 | 1.34E−06 |
| Calm3 | 6.69E−11 | −0.4285 | 0.677 | 0.758 | 1.38E−06 |
| Rplp0 | 7.43E−11 | 0.339448 | 0.975 | 0.936 | 1.53E−06 |
| Cd300lf | 9.30E−11 | −0.60384 | 0.586 | 0.763 | 1.92E−06 |
| Rpl27 | 1.03E−10 | 0.310947 | 0.975 | 0.952 | 2.12E−06 |
| Rpl28 | 1.04E−10 | 0.357823 | 0.97 | 0.962 | 2.15E−06 |
| Cd200 | 1.06E−10 | −0.62371 | 0.086 | 0.333 | 2.19E−06 |
| Rpl7a | 1.29E−10 | 0.301745 | 0.944 | 0.898 | 2.67E−06 |
| Rps25 | 1.42E−10 | 0.277815 | 0.939 | 0.919 | 2.93E−06 |
| Hk3 | 1.59E−10 | −0.47896 | 0.384 | 0.628 | 3.29E−06 |
| Ly6c2 | 1.73E−10 | −0.69741 | 0.187 | 0.466 | 3.57E−06 |
| Rnf149 | 2.04E−10 | −0.42902 | 0.389 | 0.611 | 4.20E−06 |
| Slfn5 | 2.68E−10 | −0.62098 | 0.359 | 0.595 | 5.53E−06 |
| Rps26 | 3.20E−10 | 0.302229 | 0.97 | 0.947 | 6.62E−06 |
| Mdm2 | 3.88E−10 | −0.53187 | 0.672 | 0.809 | 8.01E−06 |
| Rpl11 | 4.25E−10 | 0.295459 | 0.965 | 0.944 | 8.77E−06 |
| Rps16 | 6.55E−10 | 0.276385 | 0.99 | 0.985 | 1.35E−05 |
| Cfb | 7.49E−10 | −0.59157 | 0.278 | 0.499 | 1.55E−05 |
| Inhba | 1.04E−09 | −0.71707 | 0.53 | 0.758 | 2.15E−05 |
| Dtx2 | 1.14E−09 | −0.36577 | 0.111 | 0.321 | 2.36E−05 |
| Ifi44 | 1.15E−09 | −0.67336 | 0.293 | 0.534 | 2.38E−05 |
| Plac8 | 1.35E−09 | −0.74649 | 0.505 | 0.738 | 2.79E−05 |
| Gas6 | 1.35E−09 | 0.435777 | 0.167 | 0.038 | 2.79E−05 |
| Psme2 | 1.46E−09 | −0.47641 | 0.571 | 0.72 | 3.01E−05 |
| Fcgr1 | 1.89E−09 | −0.54998 | 0.601 | 0.789 | 3.91E−05 |
| Ppia | 2.06E−09 | 0.284314 | 0.955 | 0.962 | 4.25E−05 |
| Ifi204 | 2.19E−09 | −0.45734 | 0.54 | 0.766 | 4.52E−05 |
| F10 | 2.34E−09 | −0.49392 | 0.571 | 0.799 | 4.83E−05 |
| H2-K1 | 2.57E−09 | −0.33027 | 0.985 | 1 | 5.30E−05 |
| Slfn4 | 2.58E−09 | −0.57845 | 0.172 | 0.433 | 5.33E−05 |
| Ifi47 | 2.97E−09 | −0.49892 | 0.222 | 0.483 | 6.13E−05 |
| Cd83 | 3.11E−09 | 0.550536 | 0.581 | 0.374 | 6.43E−05 |
| Cd24a | 3.74E−09 | −0.45491 | 0.141 | 0.389 | 7.72E−05 |
| Igfbp4 | 4.26E−09 | 0.306683 | 0.182 | 0.046 | 8.80E−05 |
| Dab2 | 5.92E−09 | 0.465928 | 0.889 | 0.794 | 0.000122 |
| Slc9a9 | 7.25E−09 | 0.243812 | 0.136 | 0.013 | 0.00015 |
| Rpl36a | 7.75E−09 | 0.343753 | 0.859 | 0.789 | 0.00016 |
| Snx2 | 7.77E−09 | 0.388496 | 0.657 | 0.565 | 0.00016 |
| Gda | 7.80E−09 | 0.435237 | 0.672 | 0.461 | 0.000161 |
| Isg15 | 8.37E−09 | −0.65661 | 0.379 | 0.621 | 0.000173 |
| Eef2 | 8.61E−09 | 0.314368 | 0.894 | 0.827 | 0.000178 |
| Rtp4 | 9.35E−09 | −0.44881 | 0.227 | 0.476 | 0.000193 |
| Rpl23a | 9.58E−09 | 0.357193 | 0.939 | 0.86 | 0.000198 |
| Ptges | 1.14E−08 | −0.73361 | 0.091 | 0.267 | 0.000235 |
| Oas3 | 1.18E−08 | −0.36197 | 0.116 | 0.3 | 0.000243 |
| C3 | 1.19E−08 | −0.29617 | 0.298 | 0.565 | 0.000246 |
| Rgl1 | 1.28E−08 | −0.47245 | 0.672 | 0.819 | 0.000264 |
| Folr2 | 1.30E−08 | 0.439604 | 0.379 | 0.17 | 0.000268 |
| Rnase4 | 1.46E−08 | 0.369706 | 0.343 | 0.191 | 0.000302 |
| Car13 | 1.64E−08 | −0.31685 | 0.227 | 0.42 | 0.000339 |
| H3f3a | 1.65E−08 | −0.34294 | 0.955 | 0.977 | 0.000342 |
| Psmb9 | 1.69E−08 | −0.37042 | 0.545 | 0.756 | 0.00035 |
| Gbp4 | 1.76E−08 | −0.58034 | 0.263 | 0.509 | 0.000364 |
| Rplp2 | 1.84E−08 | 0.293448 | 0.98 | 0.954 | 0.00038 |
| Aff1 | 2.04E−08 | −0.36493 | 0.298 | 0.455 | 0.000422 |
| Gm4951 | 2.33E−08 | −0.3524 | 0.071 | 0.229 | 0.000482 |
| Ctss | 2.45E−08 | −0.36062 | 0.949 | 0.997 | 0.000506 |
| Slpi | 2.47E−08 | −0.99025 | 0.328 | 0.573 | 0.00051 |
| Rps6 | 2.47E−08 | 0.317194 | 0.889 | 0.855 | 0.000511 |
| Zdhhc14 | 2.50E−08 | 0.164524 | 0.157 | 0.046 | 0.000516 |
| Serpinb6a | 2.72E−08 | 0.480587 | 0.364 | 0.216 | 0.000562 |
| Trf | 3.02E−08 | 0.620052 | 0.399 | 0.224 | 0.000624 |
| Laptm5 | 3.28E−08 | 0.321098 | 0.904 | 0.903 | 0.000677 |
| Ly6c1 | 3.38E−08 | −0.39324 | 0.066 | 0.244 | 0.000698 |
| Epb41l1 | 3.92E−08 | 0.215337 | 0.197 | 0.071 | 0.000809 |
| Ifi27l2a | 4.12E−08 | −0.47059 | 0.813 | 0.896 | 0.000851 |
| Ccdc115 | 4.43E−08 | 0.233905 | 0.293 | 0.232 | 0.000914 |
| Tnfrsf1b | 5.11E−08 | −0.43039 | 0.631 | 0.753 | 0.001056 |
| Rps23 | 5.31E−08 | 0.27708 | 0.949 | 0.941 | 0.001097 |
| Met | 5.41E−08 | −0.28756 | 0.172 | 0.389 | 0.001116 |
| Cd38 | 5.42E−08 | −0.42139 | 0.162 | 0.361 | 0.001119 |
| mt-Cytb | 5.53E−08 | 0.417152 | 0.97 | 0.934 | 0.001141 |
| Fn1 | 6.01E−08 | 0.762499 | 0.657 | 0.514 | 0.001241 |
| Apoe | 6.09E−08 | 0.43547 | 0.803 | 0.677 | 0.001258 |
| Plpp3 | 6.41E−08 | −0.49026 | 0.187 | 0.407 | 0.001325 |
| Rpl18 | 6.43E−08 | 0.296077 | 0.97 | 0.954 | 0.001328 |
| Rps9 | 8.47E−08 | 0.245806 | 1 | 0.99 | 0.001749 |
| Comt | 9.80E−08 | 0.205825 | 0.217 | 0.127 | 0.002023 |
| Pde3b | 1.04E−07 | 0.293506 | 0.258 | 0.115 | 0.002154 |
| Tpt1 | 1.35E−07 | 0.144965 | 1 | 0.995 | 0.002779 |
| Plau | 1.38E−07 | 0.601371 | 0.586 | 0.425 | 0.002855 |
| Rps21 | 1.42E−07 | 0.207241 | 0.99 | 0.962 | 0.002925 |
| mt-Nd4 | 1.55E−07 | 0.365023 | 0.914 | 0.919 | 0.003209 |
| ligp1 | 1.80E−07 | −0.71447 | 0.152 | 0.361 | 0.003717 |
| S100a8 | 2.03E−07 | −0.76386 | 0.273 | 0.483 | 0.004193 |
| Rps18 | 2.04E−07 | 0.342766 | 0.823 | 0.735 | 0.004203 |
| Ms4a4c | 2.09E−07 | −0.38299 | 0.263 | 0.504 | 0.004309 |
| Ybx1 | 2.21E−07 | 0.24816 | 0.929 | 0.898 | 0.00457 |
| Tap1 | 2.35E−07 | −0.3726 | 0.515 | 0.682 | 0.004852 |
| Rpl6 | 2.61E−07 | 0.266011 | 0.949 | 0.949 | 0.005395 |
| Atp5g2 | 2.70E−07 | 0.29313 | 0.843 | 0.786 | 0.005577 |
| Kmt5a | 2.87E−07 | −0.35284 | 0.328 | 0.55 | 0.005926 |
| Rgs10 | 3.15E−07 | 0.30282 | 0.495 | 0.354 | 0.006495 |
| Npm1 | 3.24E−07 | 0.287231 | 0.874 | 0.832 | 0.006692 |
| Ptma | 3.30E−07 | 0.296421 | 0.975 | 0.947 | 0.006806 |
| Plin2 | 3.84E−07 | −0.3708 | 0.914 | 0.977 | 0.007932 |
| 1110038F14Rik | 4.11E−07 | −0.18827 | 0.167 | 0.254 | 0.008494 |
| H2-T22 | 4.70E−07 | −0.3651 | 0.495 | 0.616 | 0.009698 |
| Ell2 | 4.72E−07 | −0.36987 | 0.369 | 0.608 | 0.009752 |
| Cfp | 4.92E−07 | 0.381575 | 0.727 | 0.55 | 0.010162 |
| Rpl39 | 5.00E−07 | 0.234487 | 0.965 | 0.949 | 0.01033 |
| Rpl35 | 5.31E−07 | 0.2344 | 0.864 | 0.733 | 0.010964 |
| Rps17 | 5.70E−07 | 0.269756 | 0.869 | 0.819 | 0.011776 |
| Il7r | 5.94E−07 | −0.40664 | 0.717 | 0.865 | 0.012269 |
| Rapgef2 | 6.24E−07 | −0.47042 | 0.439 | 0.593 | 0.012882 |
| Irf1 | 6.49E−07 | −0.49164 | 0.581 | 0.723 | 0.01341 |
| Saa3 | 6.68E−07 | −1.62843 | 0.056 | 0.209 | 0.013792 |
| Fam168a | 6.80E−07 | 0.137339 | 0.258 | 0.267 | 0.014049 |
| Daglb | 6.87E−07 | 0.18372 | 0.222 | 0.135 | 0.014178 |
| Rpl10 | 7.00E−07 | 0.263651 | 0.914 | 0.916 | 0.014453 |
| Lgals3 | 7.32E−07 | −0.12765 | 0.985 | 0.992 | 0.015121 |
| Cd81 | 7.56E−07 | 0.356567 | 0.308 | 0.122 | 0.015619 |
| Psmb8 | 8.03E−07 | −0.34445 | 0.692 | 0.802 | 0.016589 |
| Cyb5b | 9.13E−07 | 0.135842 | 0.177 | 0.135 | 0.018862 |
| Nr4a1 | 9.21E−07 | 0.529843 | 0.556 | 0.387 | 0.019021 |
| Xaf1 | 1.00E−06 | −0.28425 | 0.177 | 0.392 | 0.020734 |
| Hspa8 | 1.04E−06 | 0.380006 | 0.965 | 0.936 | 0.02156 |
| Mef2c | 1.07E−06 | 0.293097 | 0.268 | 0.092 | 0.022011 |
| Fos | 1.15E−06 | 0.356499 | 0.874 | 0.919 | 0.023838 |
| Cat | 1.18E−06 | 0.28181 | 0.338 | 0.176 | 0.024277 |
| Cct2 | 1.35E−06 | 0.243684 | 0.48 | 0.354 | 0.027889 |
| Ctsh | 1.36E−06 | −0.34692 | 0.712 | 0.847 | 0.02815 |
| Ski | 1.45E−06 | 0.272548 | 0.424 | 0.336 | 0.029934 |
| Scaf8 | 1.45E−06 | 0.177446 | 0.182 | 0.158 | 0.030003 |
| C1qa | 1.54E−06 | 0.736931 | 0.48 | 0.308 | 0.031772 |
| Plec | 1.56E−06 | 0.16778 | 0.495 | 0.545 | 0.032295 |
| Rpl30 | 1.61E−06 | 0.227803 | 0.965 | 0.959 | 0.033217 |
| Lamp1 | 1.72E−06 | 0.248966 | 0.97 | 0.969 | 0.035508 |
| Ifitm3 | 1.77E−06 | −0.31641 | 0.929 | 0.977 | 0.036566 |
| Ass1 | 1.90E−06 | −0.30642 | 0.131 | 0.328 | 0.039252 |
| Kdm1a | 1.95E−06 | 0.271397 | 0.389 | 0.27 | 0.040255 |
| Fmnl2 | 2.11E−06 | −0.29051 | 0.374 | 0.509 | 0.043629 |
| Stx11 | 2.14E−06 | −0.31171 | 0.293 | 0.433 | 0.044137 |
| Dtx3l | 2.31E−06 | −0.32028 | 0.227 | 0.435 | 0.047715 |
| Rpl35a | 2.60E−06 | 0.179422 | 0.975 | 0.98 | 0.053763 |
| Rpl4 | 2.63E−06 | 0.2647 | 0.722 | 0.669 | 0.054347 |
| C1qc | 2.99E−06 | 0.640487 | 0.465 | 0.316 | 0.061662 |
| Atp2c1 | 3.11E−06 | 0.124977 | 0.207 | 0.198 | 0.064222 |
| Zranb3 | 3.45E−06 | 0.130833 | 0.141 | 0.069 | 0.071304 |
| Fkbp1a | 3.47E−06 | 0.266805 | 0.788 | 0.753 | 0.071679 |
| Ly6i | 3.49E−06 | −0.24818 | 0.025 | 0.155 | 0.072056 |
| Gbp6 | 3.50E−06 | −0.33379 | 0.116 | 0.244 | 0.072181 |
| Ubb | 3.58E−06 | −0.20862 | 0.955 | 0.975 | 0.073856 |
| Ccl5 | 3.66E−06 | −1.10073 | 0.106 | 0.282 | 0.075659 |
| Srgn | 3.94E−06 | −0.38888 | 0.99 | 0.972 | 0.08144 |
| Acod1 | 4.10E−06 | −0.25192 | 0.48 | 0.7 | 0.084697 |
| Nrp1 | 4.16E−06 | 0.275088 | 0.237 | 0.109 | 0.085932 |
| Spg7 | 4.30E−06 | 0.147077 | 0.157 | 0.135 | 0.088704 |
| Tcp11l2 | 4.43E−06 | 0.278662 | 0.293 | 0.176 | 0.091419 |
| Hcar2 | 4.65E−06 | −0.39147 | 0.308 | 0.527 | 0.095931 |
| Ptafr | 4.89E−06 | −0.38782 | 0.49 | 0.687 | 0.100976 |
| Trim47 | 4.97E−06 | 0.150737 | 0.131 | 0.061 | 0.102607 |
| Ncf1 | 5.20E−06 | 0.360422 | 0.49 | 0.364 | 0.107421 |
| Ssr3 | 5.48E−06 | −0.3254 | 0.657 | 0.768 | 0.113107 |
| LTO1 | 5.81E−06 | 0.103705 | 0.131 | 0.104 | 0.119986 |
| Psme1 | 5.92E−06 | −0.30207 | 0.677 | 0.809 | 0.122242 |
| Pitpnc1 | 6.47E−06 | 0.346048 | 0.394 | 0.234 | 0.133535 |
| mt-Nd2 | 6.59E−06 | 0.362287 | 0.793 | 0.746 | 0.136006 |
| Cd52 | 6.82E−06 | −0.38167 | 0.687 | 0.824 | 0.140851 |
| Itm2b | 6.89E−06 | 0.248282 | 0.924 | 0.936 | 0.142278 |
| Ntpcr | 6.99E−06 | 0.108637 | 0.187 | 0.122 | 0.144332 |
| Mapk3 | 7.00E−06 | 0.18844 | 0.54 | 0.532 | 0.144497 |
| Tspo | 7.00E−06 | −0.2652 | 0.914 | 0.939 | 0.144543 |
| Txn1 | 7.41E−06 | −0.28481 | 0.899 | 0.962 | 0.153087 |
| Rpl37 | 8.17E−06 | 0.198732 | 0.97 | 0.952 | 0.168679 |
| Rp9 | 8.44E−06 | 0.256958 | 0.52 | 0.438 | 0.174204 |
| Stxbp5 | 8.60E−06 | 0.20242 | 0.217 | 0.137 | 0.177491 |
| Ifit1 | 8.77E−06 | −0.41726 | 0.096 | 0.267 | 0.18101 |
| Ak2 | 8.78E−06 | −0.28717 | 0.374 | 0.527 | 0.181298 |
| Slfn2 | 8.98E−06 | −0.25404 | 0.788 | 0.913 | 0.185517 |
| Gng2 | 8.99E−06 | 0.281464 | 0.662 | 0.601 | 0.185656 |
| Akap13 | 9.28E−06 | −0.43739 | 0.586 | 0.733 | 0.191718 |
| Rcbtb2 | 9.41E−06 | 0.297642 | 0.419 | 0.244 | 0.194243 |
| Clk1 | 9.74E−06 | 0.237196 | 0.636 | 0.573 | 0.201205 |
| Gna12 | 1.02E−05 | 0.180525 | 0.313 | 0.254 | 0.209597 |
| Sec61g | 1.02E−05 | −0.23578 | 0.874 | 0.936 | 0.21095 |
| Igtp | 1.03E−05 | −0.34555 | 0.197 | 0.394 | 0.212025 |
| Smox | 1.04E−05 | −0.33022 | 0.662 | 0.827 | 0.214958 |
| Slc25a3 | 1.04E−05 | 0.200112 | 0.864 | 0.789 | 0.215025 |
| Mospd3 | 1.05E−05 | 0.177424 | 0.146 | 0.081 | 0.216533 |
| Unc119 | 1.05E−05 | 0.127842 | 0.162 | 0.142 | 0.217351 |
| Ggta1 | 1.07E−05 | −0.29927 | 0.217 | 0.415 | 0.220408 |
| Gnaq | 1.19E−05 | 0.305206 | 0.52 | 0.389 | 0.246186 |
| Cadm1 | 1.19E−05 | 0.222636 | 0.278 | 0.125 | 0.246575 |
| Rps28 | 1.21E−05 | 0.216047 | 0.98 | 0.967 | 0.248974 |
| Kctd6 | 1.25E−05 | 0.196739 | 0.212 | 0.109 | 0.258267 |
| Rpl19 | 1.25E−05 | 0.230762 | 0.985 | 0.969 | 0.258293 |
| Nampt | 1.29E−05 | −0.32435 | 0.793 | 0.855 | 0.266166 |
| Carhsp1 | 1.34E−05 | −0.29637 | 0.242 | 0.445 | 0.277325 |
| Tarm1 | 1.34E−05 | −0.30885 | 0.222 | 0.422 | 0.277328 |
| Tmbim4 | 1.54E−05 | −0.27415 | 0.475 | 0.667 | 0.317136 |
| Cd86 | 1.59E−05 | 0.208562 | 0.384 | 0.321 | 0.3284 |
| Cox7a2l | 1.59E−05 | 0.272301 | 0.702 | 0.634 | 0.328842 |
| Trafd1 | 1.61E−05 | −0.32367 | 0.399 | 0.59 | 0.332241 |
| Snx10 | 1.62E−05 | −0.2943 | 0.328 | 0.529 | 0.335207 |
| Dhrs9 | 1.66E−05 | 0.172699 | 0.197 | 0.102 | 0.343459 |
| Ifi44l | 1.69E−05 | −0.24918 | 0.106 | 0.272 | 0.34923 |
| Ecm1 | 1.72E−05 | −0.3943 | 0.52 | 0.656 | 0.3557 |
| Rpl37a | 1.81E−05 | 0.237618 | 0.99 | 0.987 | 0.373763 |
| Gna13 | 1.86E−05 | −0.281 | 0.874 | 0.916 | 0.384327 |
| Osm | 1.87E−05 | 0.490156 | 0.672 | 0.578 | 0.385747 |
| Sptbn1 | 1.88E−05 | 0.284281 | 0.389 | 0.282 | 0.387708 |
| Serpina3g | 1.90E−05 | −0.51438 | 0.131 | 0.3 | 0.392864 |
| Plbd2 | 1.91E−05 | 0.178738 | 0.354 | 0.308 | 0.393811 |
| Daxx | 1.95E−05 | −0.31398 | 0.237 | 0.41 | 0.403059 |
| Fosb | 2.07E−05 | 0.384415 | 0.662 | 0.621 | 0.428362 |
| Fcgr4 | 2.15E−05 | −0.37557 | 0.495 | 0.669 | 0.443794 |
| Tnfaip8 | 2.16E−05 | 0.164025 | 0.333 | 0.333 | 0.447038 |
| Trem2 | 2.18E−05 | 0.348349 | 0.672 | 0.522 | 0.450931 |
| Wwp1 | 2.19E−05 | 0.254099 | 0.283 | 0.173 | 0.452987 |
| Zfhx3 | 2.20E−05 | 0.184823 | 0.273 | 0.109 | 0.454776 |
| Il18bp | 2.21E−05 | −0.29984 | 0.202 | 0.384 | 0.455364 |
| Gm10076 | 2.22E−05 | 0.277242 | 0.556 | 0.41 | 0.459107 |
| Tent5c | 2.29E−05 | −0.34606 | 0.242 | 0.366 | 0.472919 |
| Socs3 | 2.34E−05 | −0.36949 | 0.712 | 0.784 | 0.483831 |
| Eif3h | 2.37E−05 | 0.241668 | 0.747 | 0.684 | 0.48992 |
| Cbl | 2.53E−05 | 0.298868 | 0.652 | 0.534 | 0.522871 |
| Glud1 | 2.54E−05 | 0.249211 | 0.596 | 0.506 | 0.524207 |
| Tor1aip1 | 2.57E−05 | −0.28438 | 0.444 | 0.593 | 0.531394 |
| Rilpl2 | 2.59E−05 | −0.38235 | 0.429 | 0.621 | 0.535318 |
| Helz2 | 2.68E−05 | −0.26987 | 0.157 | 0.326 | 0.553983 |
| Puf60 | 2.82E−05 | 0.14703 | 0.328 | 0.321 | 0.583106 |
| Hint1 | 2.89E−05 | 0.28969 | 0.742 | 0.636 | 0.596533 |
| Rpl21 | 2.94E−05 | 0.182158 | 0.879 | 0.812 | 0.606443 |
| Mbnl1 | 3.02E−05 | 0.274741 | 0.5 | 0.346 | 0.624249 |
| Cox4i1 | 3.18E−05 | 0.196396 | 0.904 | 0.908 | 0.656927 |
| Cbr2 | 3.26E−05 | 0.284606 | 0.177 | 0.053 | 0.672153 |
| Vkorc1 | 3.39E−05 | 0.157798 | 0.192 | 0.081 | 0.69938 |
| Fam129a | 3.60E−05 | 0.18318 | 0.167 | 0.074 | 0.743366 |
| Ahr | 3.67E−05 | 0.27759 | 0.232 | 0.125 | 0.757172 |
| Pnpla2 | 3.73E−05 | 0.187895 | 0.247 | 0.188 | 0.769675 |
| Alcam | 3.81E−05 | 0.309037 | 0.54 | 0.384 | 0.786393 |
| Ly6a | 3.90E−05 | −0.6488 | 0.389 | 0.539 | 0.805655 |
| Gbp5 | 4.01E−05 | −0.25981 | 0.141 | 0.305 | 0.827361 |
| Rps2 | 4.02E−05 | 0.233595 | 0.934 | 0.896 | 0.829146 |
| Coro2a | 4.03E−05 | −0.23905 | 0.162 | 0.336 | 0.832658 |
| Dock2 | 4.04E−05 | 0.21166 | 0.475 | 0.397 | 0.834006 |
| Eif4e3 | 4.29E−05 | 0.108471 | 0.197 | 0.186 | 0.886047 |
| Rnf213 | 4.35E−05 | −0.34371 | 0.348 | 0.481 | 0.898292 |
| Ms4a8a | 4.39E−05 | −0.14144 | 0.146 | 0.29 | 0.906795 |
| Trim30a | 4.39E−05 | −0.31468 | 0.409 | 0.585 | 0.907373 |
| Herc6 | 4.78E−05 | −0.32995 | 0.126 | 0.29 | 0.987548 |
| Rab11fip1 | 4.92E−05 | −0.14039 | 0.333 | 0.511 | 1 |
| Csf2rb2 | 4.94E−05 | −0.2671 | 0.273 | 0.453 | 1 |
| Actg1 | 5.05E−05 | 0.140408 | 0.99 | 0.997 | 1 |
| Fcer1g | 5.11E−05 | −0.26331 | 0.97 | 0.98 | 1 |
| Stat2 | 5.13E−05 | −0.28891 | 0.253 | 0.44 | 1 |
| Zc3h12d | 5.19E−05 | −0.13466 | 0.076 | 0.168 | 1 |
| Gclm | 5.58E−05 | 0.243851 | 0.374 | 0.425 | 1 |
| Mirt2 | 5.61E−05 | −0.14839 | 0.02 | 0.117 | 1 |
| Parp10 | 5.63E−05 | −0.21578 | 0.136 | 0.308 | 1 |
| Gbp7 | 5.72E−05 | −0.46417 | 0.354 | 0.517 | 1 |
| Nkap | 5.86E−05 | 0.142127 | 0.157 | 0.112 | 1 |
| Timp1 | 5.90E−05 | −0.59295 | 0.242 | 0.282 | 1 |
| Lasp1 | 6.23E−05 | −0.29002 | 0.707 | 0.835 | 1 |
| Itgb7 | 6.33E−05 | −0.30285 | 0.389 | 0.578 | 1 |
| Gpcpd1 | 6.58E−05 | 0.219486 | 0.323 | 0.239 | 1 |
| Ifi202b | 6.76E−05 | −0.28919 | 0.667 | 0.733 | 1 |
| Ctla2b | 7.20E−05 | 0.165532 | 0.146 | 0.051 | 1 |
| H2-Q7 | 7.29E−05 | −0.32409 | 0.591 | 0.735 | 1 |
| Ftl1 | 7.53E−05 | −0.12639 | 1 | 0.997 | 1 |
| Cpne3 | 7.56E−05 | 0.151945 | 0.298 | 0.262 | 1 |
| Irgm1 | 7.84E−05 | −0.31883 | 0.212 | 0.387 | 1 |
| Parp14 | 7.87E−05 | −0.34218 | 0.333 | 0.461 | 1 |
| Slc16a3 | 8.17E−05 | −0.30534 | 0.616 | 0.702 | 1 |
| Ahcyl2 | 8.36E−05 | 0.229361 | 0.242 | 0.148 | 1 |
| Ndufb9 | 8.41E−05 | −0.20803 | 0.672 | 0.715 | 1 |
| Cd93 | 9.42E−05 | 0.309263 | 0.601 | 0.44 | 1 |
| Ifi205 | 9.42E−05 | −0.20019 | 0.045 | 0.168 | 1 |
| Tgm2 | 9.50E−05 | −0.43226 | 0.641 | 0.733 | 1 |
| Rragd | 9.81E−05 | 0.137736 | 0.146 | 0.038 | 1 |
| Tex2 | 9.82E−05 | 0.17219 | 0.242 | 0.186 | 1 |
| Bst2 | 0.0001 | −0.31353 | 0.49 | 0.603 | 1 |
| Slc28a2 | 0.000101 | −0.30441 | 0.187 | 0.313 | 1 |
| Atp5c1 | 0.000102 | 0.227429 | 0.732 | 0.628 | 1 |
| Mndal | 0.000102 | −0.354 | 0.419 | 0.601 | 1 |
| Plbd1 | 0.000105 | −0.32915 | 0.53 | 0.646 | 1 |
| Malt1 | 0.000106 | −0.43818 | 0.682 | 0.748 | 1 |
| Ranbp2 | 0.000107 | −0.25956 | 0.399 | 0.578 | 1 |
| Nme2 | 0.000113 | 0.278258 | 0.778 | 0.707 | 1 |
| Ghitm | 0.000114 | 0.213946 | 0.611 | 0.534 | 1 |
| Ifit2 | 0.000115 | −0.57631 | 0.086 | 0.226 | 1 |
| Fpr2 | 0.000115 | −0.36119 | 0.177 | 0.326 | 1 |
| Hsp90b1 | 0.000116 | −0.28084 | 0.864 | 0.88 | 1 |
| Dtd2 | 0.00012 | 0.110901 | 0.106 | 0.061 | 1 |
| mt-Nd3 | 0.000121 | 0.339195 | 0.621 | 0.532 | 1 |
| Tmem8 | 0.000123 | 0.272542 | 0.172 | 0.112 | 1 |
| Tubb2a | 0.000125 | 0.200518 | 0.283 | 0.244 | 1 |
| Samhd1 | 0.000128 | −0.3602 | 0.606 | 0.69 | 1 |
| Adss | 0.000131 | 0.313627 | 0.5 | 0.389 | 1 |
| Smad1 | 0.000131 | 0.178852 | 0.192 | 0.112 | 1 |
| C1qb | 0.000132 | 0.523571 | 0.434 | 0.28 | 1 |
| Hivep1 | 0.000132 | 0.201007 | 0.288 | 0.216 | 1 |
| Cst3 | 0.000133 | 0.1776 | 0.924 | 0.908 | 1 |
| Agfg1 | 0.000134 | 0.134553 | 0.404 | 0.389 | 1 |
| Pmm2 | 0.000134 | 0.113485 | 0.146 | 0.122 | 1 |
| Gars | 0.000135 | 0.103173 | 0.237 | 0.219 | 1 |
| Ptprs | 0.000145 | 0.148306 | 0.126 | 0.043 | 1 |
| Anpep | 0.000146 | −0.26608 | 0.242 | 0.374 | 1 |
| Tsn | 0.000148 | 0.157342 | 0.409 | 0.377 | 1 |
| Tor3a | 0.000149 | −0.29078 | 0.354 | 0.496 | 1 |
| Sod1 | 0.00015 | −0.14127 | 0.313 | 0.351 | 1 |
| Nlrc5 | 0.000152 | −0.18388 | 0.086 | 0.209 | 1 |
| Jade1 | 0.000152 | 0.154378 | 0.162 | 0.079 | 1 |
| Ankrd55 | 0.000154 | −0.13771 | 0.066 | 0.181 | 1 |
| Stat1 | 0.000159 | −0.31605 | 0.419 | 0.583 | 1 |
| Osgin2 | 0.000163 | −0.29042 | 0.247 | 0.303 | 1 |
| Dcxr | 0.000164 | 0.116788 | 0.172 | 0.122 | 1 |
| Tax1bp1 | 0.000166 | −0.26852 | 0.737 | 0.85 | 1 |
| Manf | 0.000167 | −0.29334 | 0.652 | 0.662 | 1 |
| Brd2 | 0.000171 | −0.29281 | 0.687 | 0.812 | 1 |
| Rhbdf2 | 0.000173 | −0.25863 | 0.298 | 0.461 | 1 |
| Rpl36 | 0.000173 | 0.250651 | 0.919 | 0.873 | 1 |
| Calm1 | 0.000173 | −0.22604 | 0.99 | 0.972 | 1 |
| Ppm1a | 0.000175 | 0.107236 | 0.374 | 0.374 | 1 |
| Tln1 | 0.000178 | 0.242283 | 0.848 | 0.758 | 1 |
| Atp6v0a1 | 0.000179 | 0.223504 | 0.253 | 0.168 | 1 |
| Arl4c | 0.00018 | 0.339445 | 0.697 | 0.565 | 1 |
| Ifi211 | 0.00018 | −0.27769 | 0.162 | 0.323 | 1 |
| Uvrag | 0.000182 | 0.121074 | 0.247 | 0.216 | 1 |
| Gmps | 0.0002 | −0.14578 | 0.172 | 0.201 | 1 |
| Il13ra1 | 0.000207 | −0.28254 | 0.338 | 0.517 | 1 |
| Hnrnph3 | 0.000208 | −0.18979 | 0.157 | 0.267 | 1 |
| AI504432 | 0.000211 | 0.113496 | 0.283 | 0.303 | 1 |
| Mfsd6 | 0.000212 | 0.135384 | 0.126 | 0.031 | 1 |
| Mia2 | 0.000224 | 0.224058 | 0.369 | 0.249 | 1 |
| Foxk1 | 0.000227 | 0.125198 | 0.136 | 0.092 | 1 |
| Mett19 | 0.000232 | −0.31218 | 0.384 | 0.547 | 1 |
| Asxl2 | 0.000236 | 0.179612 | 0.278 | 0.183 | 1 |
| Ctdsp2 | 0.000239 | 0.229757 | 0.364 | 0.305 | 1 |
| Clec4e | 0.000239 | −0.37304 | 0.621 | 0.761 | 1 |
| Glis3 | 0.00024 | −0.23101 | 0.02 | 0.112 | 1 |
| Rpl34 | 0.000244 | 0.180508 | 0.975 | 0.972 | 1 |
| Reep3 | 0.000246 | 0.169299 | 0.556 | 0.57 | 1 |
| Fkbp3 | 0.000247 | 0.110586 | 0.242 | 0.211 | 1 |
| Igf1r | 0.000248 | 0.170696 | 0.197 | 0.132 | 1 |
| Sri | 0.000249 | −0.23947 | 0.535 | 0.672 | 1 |
| Bbc3 | 0.000252 | 0.125995 | 0.131 | 0.094 | 1 |
| Slc15a3 | 0.000252 | −0.21778 | 0.697 | 0.824 | 1 |
| Pspc1 | 0.000252 | 0.122091 | 0.162 | 0.104 | 1 |
| Nrg4 | 0.000255 | −0.14565 | 0.02 | 0.115 | 1 |
| Nucks1 | 0.000256 | 0.225124 | 0.414 | 0.254 | 1 |
| Ndfip2 | 0.00026 | −0.26791 | 0.439 | 0.595 | 1 |
| Igf1 | 0.000265 | 0.209944 | 0.106 | 0.028 | 1 |
| Jak1 | 0.000279 | −0.26287 | 0.636 | 0.733 | 1 |
| Psmb5 | 0.00028 | −0.26036 | 0.394 | 0.56 | 1 |
| Kpna4 | 0.000283 | 0.103229 | 0.566 | 0.634 | 1 |
| Tnfaip2 | 0.00029 | −0.35898 | 0.652 | 0.789 | 1 |
| Tbc1d2b | 0.000291 | 0.134369 | 0.227 | 0.183 | 1 |
| Btf3 | 0.000292 | 0.187412 | 0.944 | 0.924 | 1 |
| Btg2 | 0.000294 | 0.341793 | 0.783 | 0.753 | 1 |
| R3hdm2 | 0.000299 | 0.158434 | 0.338 | 0.293 | 1 |
| Rps29 | 0.0003 | 0.139966 | 0.995 | 0.977 | 1 |
| Creld2 | 0.00032 | −0.29069 | 0.177 | 0.321 | 1 |
| Ifi35 | 0.000321 | −0.22284 | 0.202 | 0.323 | 1 |
| Tcp1 | 0.000322 | 0.219675 | 0.515 | 0.433 | 1 |
| Trappc4 | 0.000324 | 0.148828 | 0.414 | 0.387 | 1 |
| Cryl1 | 0.000325 | 0.11746 | 0.131 | 0.036 | 1 |
| Mettl16 | 0.000327 | 0.112544 | 0.101 | 0.066 | 1 |
| Impa2 | 0.000337 | −0.20588 | 0.273 | 0.448 | 1 |
| Acap2 | 0.000338 | 0.114642 | 0.369 | 0.364 | 1 |
| Nr1d2 | 0.000339 | 0.183181 | 0.141 | 0.059 | 1 |
| Sdf2l1 | 0.000354 | −0.16607 | 0.303 | 0.328 | 1 |
| Tmem256 | 0.000368 | −0.24226 | 0.707 | 0.819 | 1 |
| Camk1 | 0.000369 | 0.194918 | 0.364 | 0.272 | 1 |
| Midn | 0.000386 | 0.181262 | 0.273 | 0.216 | 1 |
| Dnaja1 | 0.000386 | 0.33109 | 0.571 | 0.562 | 1 |
| Anxa3 | 0.000408 | 0.229383 | 0.434 | 0.387 | 1 |
| Lrmp | 0.00041 | 0.135048 | 0.136 | 0.064 | 1 |
| Pls3 | 0.000417 | 0.126362 | 0.141 | 0.059 | 1 |
| Atl3 | 0.000424 | 0.148313 | 0.283 | 0.221 | 1 |
| Sh3glb1 | 0.000435 | 0.17445 | 0.904 | 0.916 | 1 |
| Mmp8 | 0.000435 | −0.43409 | 0.202 | 0.366 | 1 |
| Gstm1 | 0.000437 | 0.270164 | 0.389 | 0.224 | 1 |
| Ifit3 | 0.00044 | −0.25133 | 0.081 | 0.206 | 1 |
| Hivep3 | 0.000441 | −0.28887 | 0.101 | 0.234 | 1 |
| Tra2a | 0.000445 | −0.31339 | 0.556 | 0.712 | 1 |
| Pcnt | 0.000446 | 0.128601 | 0.146 | 0.074 | 1 |
| Dram1 | 0.000454 | −0.20847 | 0.136 | 0.272 | 1 |
| Eif3i | 0.000463 | 0.19857 | 0.53 | 0.351 | 1 |
| Akr1b8 | 0.000471 | 0.159289 | 0.136 | 0.076 | 1 |
| Cd63 | 0.000471 | 0.19986 | 0.768 | 0.756 | 1 |
| Top2b | 0.000475 | 0.146305 | 0.227 | 0.145 | 1 |
| Lamp2 | 0.000477 | 0.200631 | 0.879 | 0.87 | 1 |
| Sem1 | 0.000481 | −0.12127 | 0.975 | 0.949 | 1 |
| Sdc1 | 0.000484 | −0.31492 | 0.182 | 0.338 | 1 |
| B3gat3 | 0.000487 | −0.13729 | 0.076 | 0.163 | 1 |
| 1810026B05Rik | 0.000488 | 0.127435 | 0.217 | 0.201 | 1 |
| Pgls | 0.000493 | 0.190174 | 0.556 | 0.453 | 1 |
| B4galt6 | 0.000494 | 0.21925 | 0.293 | 0.224 | 1 |
| 9130230L23Rik | 0.000496 | −0.21454 | 0.081 | 0.209 | 1 |
| Elob | 0.000513 | −0.20278 | 0.859 | 0.891 | 1 |
| Traf1 | 0.000519 | −0.39099 | 0.419 | 0.58 | 1 |
| Spcs2 | 0.000547 | −0.2433 | 0.5 | 0.664 | 1 |
| Fut11 | 0.000557 | 0.122268 | 0.131 | 0.059 | 1 |
| Ap2a2 | 0.000562 | 0.112657 | 0.364 | 0.389 | 1 |
| Gas5 | 0.000576 | 0.26622 | 0.535 | 0.486 | 1 |
| Fbx15 | 0.000592 | −0.34964 | 0.566 | 0.7 | 1 |
| Pou2f2 | 0.000597 | −0.14503 | 0.03 | 0.125 | 1 |
| Pcbp2 | 0.000599 | 0.145657 | 0.869 | 0.901 | 1 |
| Rsrp1 | 0.000603 | −0.20454 | 0.662 | 0.809 | 1 |
| Abca1 | 0.000604 | 0.267632 | 0.646 | 0.641 | 1 |
| 11-Sep | 0.000605 | 0.23355 | 0.348 | 0.229 | 1 |
| Slfn1 | 0.000608 | −0.23361 | 0.288 | 0.455 | 1 |
| Ezr | 0.000623 | −0.28639 | 0.48 | 0.639 | 1 |
| Npepl1 | 0.00063 | 0.123609 | 0.152 | 0.081 | 1 |
| Atp1b3 | 0.000631 | 0.239024 | 0.753 | 0.743 | 1 |
| Slc7a2 | 0.000632 | −0.49455 | 0.54 | 0.616 | 1 |
| Tmem140 | 0.000655 | −0.27757 | 0.268 | 0.372 | 1 |
| Igsf6 | 0.000658 | −0.28079 | 0.571 | 0.667 | 1 |
| Ciapin1 | 0.000666 | −0.1626 | 0.182 | 0.333 | 1 |
| Eprs | 0.000683 | 0.201047 | 0.424 | 0.29 | 1 |
| Lmbrd1 | 0.000693 | 0.143792 | 0.434 | 0.412 | 1 |
| Pgrmc2 | 0.000699 | 0.168946 | 0.192 | 0.122 | 1 |
| Akt3 | 0.0007 | 0.113527 | 0.182 | 0.094 | 1 |
| Hsp90ab1 | 0.000703 | 0.193761 | 0.929 | 0.888 | 1 |
| Tra2b | 0.000704 | −0.27185 | 0.48 | 0.621 | 1 |
| Cmpk2 | 0.000705 | −0.16273 | 0.091 | 0.211 | 1 |
| Ms4a7 | 0.000708 | −0.39988 | 0.46 | 0.567 | 1 |
| S100a11 | 0.000733 | −0.22632 | 0.909 | 0.906 | 1 |
| Ndrg1 | 0.000741 | 0.306713 | 0.96 | 0.962 | 1 |
| Ogfr | 0.000742 | −0.22988 | 0.258 | 0.394 | 1 |
| Glipr2 | 0.000744 | −0.28259 | 0.253 | 0.407 | 1 |
| Cysltr1 | 0.000745 | 0.201627 | 0.333 | 0.252 | 1 |
| Ap3s1 | 0.000745 | −0.24958 | 0.763 | 0.865 | 1 |
| Rpl26 | 0.000746 | 0.217403 | 0.737 | 0.707 | 1 |
| Ccl24 | 0.00078 | 0.398518 | 0.313 | 0.168 | 1 |
| ler3ip1 | 0.000817 | −0.22518 | 0.687 | 0.771 | 1 |
| Mmp12 | 0.000817 | 0.341452 | 0.687 | 0.517 | 1 |
| Fcna | 0.000818 | 0.15387 | 0.101 | 0.038 | 1 |
| Emsy | 0.000827 | 0.113236 | 0.162 | 0.132 | 1 |
| Mt2 | 0.00083 | 0.453841 | 0.545 | 0.425 | 1 |
| Iah1 | 0.000831 | 0.106301 | 0.136 | 0.099 | 1 |
| Bcl6 | 0.000839 | −0.18008 | 0.101 | 0.211 | 1 |
| Phf11b | 0.000857 | −0.12746 | 0.146 | 0.275 | 1 |
| Arpc5 | 0.000866 | −0.20315 | 0.899 | 0.941 | 1 |
| Rsu1 | 0.000868 | 0.176813 | 0.399 | 0.321 | 1 |
| Ddit4 | 0.000881 | 0.173214 | 0.273 | 0.318 | 1 |
| Rnf4 | 0.000886 | 0.116774 | 0.303 | 0.295 | 1 |
| Sh3bgrl | 0.000892 | 0.224947 | 0.742 | 0.656 | 1 |
| Smndc1 | 0.000919 | −0.13033 | 0.283 | 0.443 | 1 |
| Kcnq1ot1 | 0.00093 | 0.217503 | 0.227 | 0.104 | 1 |
| Flii | 0.000962 | −0.15255 | 0.328 | 0.496 | 1 |
| Eif3f | 0.000966 | 0.191484 | 0.788 | 0.776 | 1 |
| Park7 | 0.00097 | 0.215599 | 0.601 | 0.501 | 1 |
| Eif4e | 0.000972 | −0.26016 | 0.606 | 0.723 | 1 |
| Cd68 | 0.000987 | −0.22126 | 0.894 | 0.957 | 1 |
| Slc9a3r1 | 0.000988 | −0.25007 | 0.449 | 0.575 | 1 |
| Ly6e | 0.000994 | −0.31918 | 0.535 | 0.69 | 1 |
| Hck | 0.001012 | −0.20765 | 0.242 | 0.361 | 1 |
| Spp1 | 0.00102 | 0.110936 | 0.859 | 0.718 | 1 |
| Tanc2 | 0.001021 | 0.136229 | 0.157 | 0.056 | 1 |
| Clec4n | 0.001022 | 0.417609 | 0.591 | 0.486 | 1 |
| Rnf31 | 0.001022 | −0.10281 | 0.045 | 0.117 | 1 |
| Cpne2 | 0.001024 | −0.22374 | 0.177 | 0.3 | 1 |
| Laptm4a | 0.001064 | −0.18647 | 0.606 | 0.763 | 1 |
| Srf | 0.001089 | −0.11997 | 0.076 | 0.132 | 1 |
| Arid3a | 0.001094 | −0.18174 | 0.182 | 0.226 | 1 |
| Ubl3 | 0.001102 | 0.191097 | 0.46 | 0.458 | 1 |
| Capza1 | 0.00111 | 0.115903 | 0.606 | 0.628 | 1 |
| Adam15 | 0.001113 | 0.229565 | 0.46 | 0.361 | 1 |
| Atp6v1e1 | 0.00112 | −0.21216 | 0.621 | 0.756 | 1 |
| Il17ra | 0.001121 | −0.23892 | 0.131 | 0.265 | 1 |
| Selenom | 0.001168 | −0.10093 | 0.197 | 0.198 | 1 |
| Srsf2 | 0.001168 | −0.19008 | 0.631 | 0.784 | 1 |
| Slc9a8 | 0.001204 | −0.10206 | 0.045 | 0.142 | 1 |
| Nsa2 | 0.001209 | 0.169813 | 0.707 | 0.702 | 1 |
| Tmpo | 0.00121 | −0.22427 | 0.217 | 0.344 | 1 |
| Ddx58 | 0.001213 | −0.1893 | 0.106 | 0.232 | 1 |
| Ap1b1 | 0.001272 | 0.252595 | 0.389 | 0.28 | 1 |
| Olfm1 | 0.001288 | 0.222958 | 0.278 | 0.188 | 1 |
| Ptpn18 | 0.001312 | 0.234552 | 0.626 | 0.494 | 1 |
| Otulin | 0.001328 | 0.119671 | 0.278 | 0.254 | 1 |
| Epc1 | 0.00133 | −0.11813 | 0.222 | 0.26 | 1 |
| Gpatch8 | 0.001335 | 0.122181 | 0.263 | 0.204 | 1 |
| Rpl15 | 0.001335 | 0.200092 | 0.929 | 0.924 | 1 |
| Dock8 | 0.001337 | 0.117796 | 0.222 | 0.173 | 1 |
| Pabpc1 | 0.001339 | −0.17182 | 0.889 | 0.896 | 1 |
| Actr3 | 0.001359 | −0.19854 | 0.854 | 0.929 | 1 |
| Trim25 | 0.001386 | −0.26856 | 0.535 | 0.664 | 1 |
| Ing3 | 0.001396 | 0.101945 | 0.141 | 0.127 | 1 |
| Mt1 | 0.001397 | 0.294898 | 0.934 | 0.913 | 1 |
| Gsr | 0.001404 | −0.21997 | 0.364 | 0.529 | 1 |
| Gbp3 | 0.001413 | −0.22061 | 0.162 | 0.308 | 1 |
| Sp100 | 0.001428 | −0.23453 | 0.414 | 0.573 | 1 |
| Mindy2 | 0.00144 | 0.171373 | 0.515 | 0.478 | 1 |
| Myd88 | 0.001453 | −0.12552 | 0.141 | 0.27 | 1 |
| Phf21a | 0.00147 | 0.124108 | 0.217 | 0.198 | 1 |
| Alox5ap | 0.001478 | 0.162898 | 0.914 | 0.868 | 1 |
| Hsp90aa1 | 0.001486 | 0.213523 | 0.848 | 0.88 | 1 |
| Syk | 0.001497 | −0.22184 | 0.657 | 0.735 | 1 |
| Vcl | 0.001502 | 0.215797 | 0.253 | 0.165 | 1 |
| Gk | 0.001506 | −0.17836 | 0.136 | 0.275 | 1 |
| Bri3 | 0.001526 | −0.19522 | 0.914 | 0.959 | 1 |
| Imp3 | 0.001539 | 0.192794 | 0.293 | 0.186 | 1 |
| Fpr1 | 0.00154 | −0.14269 | 0.04 | 0.107 | 1 |
| Ldlrap1 | 0.001543 | −0.15187 | 0.177 | 0.316 | 1 |
| Rcan1 | 0.001547 | 0.264036 | 0.288 | 0.178 | 1 |
| Lmna | 0.001548 | 0.176878 | 0.758 | 0.796 | 1 |
| Hsph1 | 0.001553 | 0.362998 | 0.253 | 0.142 | 1 |
| Tcf4 | 0.001564 | 0.168597 | 0.49 | 0.389 | 1 |
| mt-Nd4l | 0.001583 | 0.16221 | 0.333 | 0.188 | 1 |
| Gpalpp1 | 0.001589 | 0.165288 | 0.374 | 0.288 | 1 |
| Mef2a | 0.001606 | 0.202152 | 0.586 | 0.542 | 1 |
| D10Wsu102e | 0.00161 | 0.213641 | 0.167 | 0.107 | 1 |
| Sdha | 0.001615 | 0.203446 | 0.359 | 0.285 | 1 |
| Tnip1 | 0.00162 | −0.19852 | 0.187 | 0.3 | 1 |
| Parp12 | 0.001644 | −0.19924 | 0.157 | 0.27 | 1 |
| Tbc1d9 | 0.001673 | −0.24265 | 0.47 | 0.578 | 1 |
| Isg20 | 0.001683 | −0.15901 | 0.081 | 0.196 | 1 |
| Ccr1 | 0.001697 | −0.22275 | 0.727 | 0.802 | 1 |
| Tbcb | 0.001717 | −0.21592 | 0.303 | 0.445 | 1 |
| Wnk1 | 0.001741 | 0.203699 | 0.722 | 0.702 | 1 |
| Lgals3bp | 0.001745 | −0.25658 | 0.404 | 0.511 | 1 |
| Utp14b | 0.00177 | 0.103676 | 0.111 | 0.097 | 1 |
| Tmem168 | 0.001785 | 0.153559 | 0.222 | 0.158 | 1 |
| Rasa4 | 0.001788 | −0.23397 | 0.374 | 0.499 | 1 |
| Suclg2 | 0.001802 | 0.139803 | 0.177 | 0.107 | 1 |
| Amdhd2 | 0.001844 | −0.17791 | 0.419 | 0.489 | 1 |
| Hspa5 | 0.001858 | −0.39572 | 0.874 | 0.911 | 1 |
| Cd36 | 0.001861 | 0.244052 | 0.465 | 0.323 | 1 |
| Shtn1 | 0.001872 | 0.156789 | 0.207 | 0.107 | 1 |
| Psma4 | 0.001881 | −0.19778 | 0.576 | 0.664 | 1 |
| Pla2g7 | 0.00189 | −0.26957 | 0.652 | 0.72 | 1 |
| Gadd45a | 0.001974 | −0.13198 | 0.268 | 0.415 | 1 |
| Pdcd10 | 0.001978 | −0.16604 | 0.53 | 0.692 | 1 |
| mt-Co3 | 0.001984 | 0.223482 | 1 | 0.992 | 1 |
| Ap2b1 | 0.001999 | −0.14106 | 0.323 | 0.476 | 1 |
| Sptlc1 | 0.002043 | 0.13209 | 0.197 | 0.145 | 1 |
| Ciao2b | 0.002054 | −0.18865 | 0.374 | 0.506 | 1 |
| Cenpa | 0.00208 | 0.131571 | 0.247 | 0.275 | 1 |
| Lars | 0.00215 | 0.158698 | 0.258 | 0.178 | 1 |
| Ptpn1 | 0.002162 | −0.17259 | 0.561 | 0.567 | 1 |
| Prdm1 | 0.002187 | −0.18738 | 0.278 | 0.435 | 1 |
| Rbck1 | 0.002194 | 0.109471 | 0.207 | 0.181 | 1 |
| Calr | 0.002204 | −0.27319 | 0.763 | 0.852 | 1 |
| Rnf19a | 0.002206 | 0.120512 | 0.242 | 0.196 | 1 |
| Ifi214 | 0.002272 | −0.17324 | 0.051 | 0.15 | 1 |
| Rab28 | 0.002295 | 0.112903 | 0.192 | 0.115 | 1 |
| Rhou | 0.002322 | −0.20866 | 0.131 | 0.262 | 1 |
| Dhrs1 | 0.002354 | −0.15432 | 0.232 | 0.303 | 1 |
| Tbck | 0.002404 | 0.107314 | 0.141 | 0.099 | 1 |
| Psmb10 | 0.002489 | −0.20527 | 0.409 | 0.478 | 1 |
| Slamf8 | 0.002512 | −0.30863 | 0.212 | 0.313 | 1 |
| Hadh | 0.002543 | 0.117705 | 0.126 | 0.051 | 1 |
| Csf1r | 0.002558 | 0.228354 | 0.727 | 0.667 | 1 |
| Sap30 | 0.002563 | 0.196527 | 0.525 | 0.575 | 1 |
| Gpat3 | 0.00257 | −0.2216 | 0.404 | 0.552 | 1 |
| Kdr | 0.002604 | −0.24806 | 0.081 | 0.181 | 1 |
| Gipr | 0.002608 | −0.13843 | 0.035 | 0.122 | 1 |
| Plgrkt | 0.002624 | −0.2567 | 0.369 | 0.504 | 1 |
| Slc25a45 | 0.002642 | 0.109588 | 0.126 | 0.076 | 1 |
| Rpl41 | 0.002693 | 0.117928 | 1 | 0.995 | 1 |
| Egr3 | 0.002713 | 0.114771 | 0.101 | 0.033 | 1 |
| Lrrc25 | 0.002718 | −0.18628 | 0.278 | 0.433 | 1 |
| Pank3 | 0.002724 | 0.194308 | 0.328 | 0.247 | 1 |
| Pnp | 0.002798 | −0.26679 | 0.455 | 0.575 | 1 |
| Ap2m1 | 0.002807 | 0.205316 | 0.616 | 0.539 | 1 |
| Hist1h1c | 0.002854 | 0.352032 | 0.359 | 0.229 | 1 |
| Slbp | 0.002873 | −0.10688 | 0.303 | 0.443 | 1 |
| Ifnar2 | 0.002924 | −0.20256 | 0.712 | 0.799 | 1 |
| Smim13 | 0.002939 | 0.100906 | 0.106 | 0.031 | 1 |
| mt-Atp6 | 0.00296 | 0.20922 | 1 | 0.99 | 1 |
| Diaph2 | 0.002977 | 0.193652 | 0.333 | 0.219 | 1 |
| Anxa4 | 0.002978 | −0.18297 | 0.697 | 0.72 | 1 |
| Elmsan1 | 0.002984 | 0.122122 | 0.146 | 0.112 | 1 |
| Gm17056 | 0.003007 | 0.144677 | 0.182 | 0.13 | 1 |
| Sgms2 | 0.003039 | −0.22793 | 0.217 | 0.349 | 1 |
| 5-Mar | 0.003057 | −0.17271 | 0.237 | 0.341 | 1 |
| Hbs1l | 0.003059 | 0.119096 | 0.152 | 0.081 | 1 |
| Arf5 | 0.003069 | −0.17685 | 0.742 | 0.855 | 1 |
| Cct8 | 0.003113 | 0.136778 | 0.515 | 0.494 | 1 |
| Rpl31 | 0.003126 | 0.209066 | 0.848 | 0.794 | 1 |
| H2-Q6 | 0.003151 | −0.21945 | 0.404 | 0.478 | 1 |
| Cltc | 0.003175 | 0.203989 | 0.783 | 0.781 | 1 |
| Gdi2 | 0.003194 | 0.187982 | 0.859 | 0.791 | 1 |
| Ifi207 | 0.003212 | −0.25093 | 0.515 | 0.659 | 1 |
| Uck2 | 0.003217 | 0.1849 | 0.258 | 0.214 | 1 |
| AC149090.1 | 0.003226 | 0.168545 | 0.217 | 0.204 | 1 |
| St8sia4 | 0.003262 | 0.263184 | 0.551 | 0.407 | 1 |
| Eid1 | 0.003356 | 0.21184 | 0.591 | 0.514 | 1 |
| Kif16b | 0.003362 | 0.110417 | 0.162 | 0.071 | 1 |
| Atp6v0e | 0.0034 | −0.1892 | 0.864 | 0.929 | 1 |
| Acvrl1 | 0.003428 | −0.21786 | 0.354 | 0.489 | 1 |
| Mafb | 0.003428 | 0.326399 | 0.929 | 0.883 | 1 |
| Irgm2 | 0.003436 | −0.1791 | 0.106 | 0.226 | 1 |
| Vma21 | 0.003459 | 0.163647 | 0.495 | 0.405 | 1 |
| Clec4d | 0.003496 | −0.24045 | 0.864 | 0.926 | 1 |
| Ssr4 | 0.003536 | 0.201656 | 0.662 | 0.608 | 1 |
| Lyst | 0.003536 | −0.25109 | 0.369 | 0.489 | 1 |
| Oas12 | 0.003567 | −0.22131 | 0.308 | 0.453 | 1 |
| Rpl38 | 0.003576 | 0.156762 | 0.97 | 0.962 | 1 |
| Hmgb2 | 0.00367 | −0.21624 | 0.586 | 0.728 | 1 |
| Arl13b | 0.003691 | −0.12602 | 0.071 | 0.163 | 1 |
| F7 | 0.003694 | −0.18107 | 0.167 | 0.3 | 1 |
| Nat9 | 0.003732 | −0.14078 | 0.152 | 0.224 | 1 |
| Otulinl | 0.003756 | 0.153502 | 0.197 | 0.107 | 1 |
| Egr1 | 0.003767 | 0.37151 | 0.823 | 0.807 | 1 |
| Dnajc7 | 0.003768 | 0.182302 | 0.343 | 0.272 | 1 |
| Aprt | 0.003789 | 0.189501 | 0.874 | 0.863 | 1 |
| Akr1b3 | 0.003839 | 0.189884 | 0.323 | 0.201 | 1 |
| Rrbp1 | 0.003844 | −0.19845 | 0.848 | 0.883 | 1 |
| Rnf19b | 0.003858 | −0.25215 | 0.712 | 0.766 | 1 |
| Hpgds | 0.003871 | 0.10999 | 0.141 | 0.084 | 1 |
| Etv1 | 0.003875 | 0.23052 | 0.237 | 0.155 | 1 |
| Gm20559 | 0.003899 | −0.19235 | 0.146 | 0.27 | 1 |
| Fam120a | 0.003913 | 0.206446 | 0.414 | 0.3 | 1 |
| Ndufa3 | 0.003955 | −0.2116 | 0.354 | 0.489 | 1 |
| Cebpg | 0.00398 | 0.126526 | 0.515 | 0.473 | 1 |
| Clec4a1 | 0.003992 | −0.19212 | 0.429 | 0.463 | 1 |
| Cox17 | 0.004004 | −0.21944 | 0.485 | 0.621 | 1 |
| Ifngr1 | 0.004009 | 0.149327 | 0.444 | 0.425 | 1 |
| Tmed7 | 0.00401 | −0.20747 | 0.465 | 0.59 | 1 |
| Hprt | 0.004035 | 0.181175 | 0.515 | 0.417 | 1 |
| Rsbn1 | 0.004071 | 0.217452 | 0.384 | 0.321 | 1 |
| Gcsh | 0.004074 | 0.128927 | 0.202 | 0.145 | 1 |
| Cxcl16 | 0.004132 | −0.25743 | 0.636 | 0.656 | 1 |
| Vps4b | 0.004171 | −0.12167 | 0.46 | 0.603 | 1 |
| Egr2 | 0.004182 | 0.269086 | 0.48 | 0.45 | 1 |
| Psma7 | 0.004239 | −0.18572 | 0.828 | 0.86 | 1 |
| Spsb2 | 0.004259 | 0.132578 | 0.172 | 0.13 | 1 |
| Foxp1 | 0.004301 | 0.233085 | 0.343 | 0.247 | 1 |
| Rassf3 | 0.004323 | 0.191927 | 0.359 | 0.272 | 1 |
| Mrpl52 | 0.004334 | −0.16854 | 0.677 | 0.735 | 1 |
| Sp140 | 0.004379 | −0.19423 | 0.354 | 0.494 | 1 |
| Cmtm4 | 0.004381 | 0.119135 | 0.172 | 0.107 | 1 |
| Selenof | 0.004429 | 0.141899 | 0.606 | 0.562 | 1 |
| Hist1h4i | 0.004437 | 0.293982 | 0.121 | 0.087 | 1 |
| Bcl3 | 0.004462 | −0.1236 | 0.182 | 0.303 | 1 |
| Mrpl33 | 0.004541 | 0.171723 | 0.606 | 0.537 | 1 |
| Tgfbr2 | 0.004542 | 0.178083 | 0.328 | 0.254 | 1 |
| Med31 | 0.004573 | −0.10345 | 0.157 | 0.272 | 1 |
| Rhov | 0.004604 | −0.35754 | 0.237 | 0.366 | 1 |
| Ddhd2 | 0.004629 | 0.126798 | 0.162 | 0.087 | 1 |
| Sidt2 | 0.004645 | 0.173897 | 0.273 | 0.178 | 1 |
| Tardbp | 0.004705 | 0.128752 | 0.354 | 0.336 | 1 |
| Acp5 | 0.004735 | −0.14509 | 0.045 | 0.132 | 1 |
| Ptpn7 | 0.004735 | 0.130286 | 0.197 | 0.148 | 1 |
| Clcn3 | 0.004766 | 0.220094 | 0.328 | 0.209 | 1 |
| Irf2 | 0.004828 | −0.22775 | 0.505 | 0.623 | 1 |
| Snx6 | 0.004856 | 0.216291 | 0.601 | 0.547 | 1 |
| Hexa | 0.004871 | 0.142093 | 0.742 | 0.73 | 1 |
| Man2b1 | 0.004935 | 0.165848 | 0.348 | 0.3 | 1 |
| Aopep | 0.005064 | 0.233739 | 0.475 | 0.346 | 1 |
| Smpdl3a | 0.005064 | 0.214375 | 0.5 | 0.43 | 1 |
| Colgalt1 | 0.00516 | 0.133599 | 0.288 | 0.186 | 1 |
| Gtf2a2 | 0.005273 | −0.10838 | 0.222 | 0.354 | 1 |
| Fabp4 | 0.005294 | 0.312743 | 0.207 | 0.099 | 1 |
| Tmem37 | 0.005298 | 0.140724 | 0.197 | 0.107 | 1 |
| Usp34 | 0.005333 | 0.144897 | 0.333 | 0.305 | 1 |
| Tet3 | 0.005339 | 0.175896 | 0.354 | 0.293 | 1 |
| Naca | 0.005386 | 0.183956 | 0.899 | 0.86 | 1 |
| Ufm1 | 0.005468 | −0.17782 | 0.399 | 0.494 | 1 |
| Sbno2 | 0.005491 | −0.15296 | 0.414 | 0.565 | 1 |
| Ndufb1-ps | 0.005508 | −0.1552 | 0.636 | 0.644 | 1 |
| Lilr4b | 0.005547 | −0.15843 | 0.899 | 0.957 | 1 |
| Cd40 | 0.00557 | −0.13192 | 0.131 | 0.242 | 1 |
| Atp6v1g1 | 0.005632 | −0.11193 | 0.864 | 0.947 | 1 |
| Sms | 0.005633 | 0.196952 | 0.384 | 0.267 | 1 |
| Hspa9 | 0.005698 | 0.216005 | 0.646 | 0.573 | 1 |
| Trim56 | 0.005709 | −0.13413 | 0.096 | 0.204 | 1 |
| Atp6v0c | 0.005758 | −0.1084 | 0.99 | 0.985 | 1 |
| B4galt1 | 0.005779 | 0.150948 | 0.444 | 0.417 | 1 |
| Ptgs2 | 0.005827 | −0.42912 | 0.687 | 0.789 | 1 |
| Fam8a1 | 0.005956 | −0.14199 | 0.101 | 0.206 | 1 |
| Asap1 | 0.006005 | 0.143549 | 0.222 | 0.191 | 1 |
| Lpxn | 0.006044 | −0.20206 | 0.343 | 0.45 | 1 |
| Cmas | 0.006051 | 0.139338 | 0.338 | 0.209 | 1 |
| Mbtd1 | 0.006058 | 0.109736 | 0.197 | 0.163 | 1 |
| Acsl1 | 0.006139 | −0.22998 | 0.242 | 0.303 | 1 |
| Vps72 | 0.006264 | 0.107133 | 0.162 | 0.117 | 1 |
| A930007119Rik | 0.006361 | 0.22015 | 0.273 | 0.402 | 1 |
| Trps1 | 0.00637 | 0.264855 | 0.389 | 0.298 | 1 |
| Phf20 | 0.006401 | 0.160055 | 0.308 | 0.229 | 1 |
| Shisa5 | 0.006401 | −0.21612 | 0.667 | 0.748 | 1 |
| Rasa1 | 0.006431 | 0.125717 | 0.247 | 0.17 | 1 |
| Ago2 | 0.006528 | 0.180055 | 0.606 | 0.59 | 1 |
| Pla2g15 | 0.006544 | 0.135322 | 0.157 | 0.084 | 1 |
| Eif2a | 0.006855 | 0.131187 | 0.263 | 0.188 | 1 |
| 5031425E22Rik | 0.006866 | −0.16421 | 0.162 | 0.288 | 1 |
| Sumo3 | 0.006922 | −0.16976 | 0.258 | 0.379 | 1 |
| Gpnmb | 0.006969 | −0.44605 | 0.091 | 0.191 | 1 |
| Cnn3 | 0.007172 | −0.21505 | 0.242 | 0.346 | 1 |
| Tmem123 | 0.00718 | 0.147778 | 0.434 | 0.417 | 1 |
| Agap1 | 0.007308 | 0.121883 | 0.136 | 0.074 | 1 |
| Atxn1 | 0.007394 | 0.171792 | 0.273 | 0.229 | 1 |
| Srp19 | 0.007408 | −0.17364 | 0.313 | 0.443 | 1 |
| Furin | 0.007463 | −0.20676 | 0.798 | 0.832 | 1 |
| Celf2 | 0.007485 | 0.171616 | 0.48 | 0.427 | 1 |
| Dhx9 | 0.007511 | −0.1097 | 0.207 | 0.247 | 1 |
| Necap2 | 0.007564 | 0.139403 | 0.5 | 0.471 | 1 |
| Hnrnpa1 | 0.007758 | 0.200688 | 0.631 | 0.537 | 1 |
| Nipsnap3b | 0.007844 | −0.11997 | 0.328 | 0.466 | 1 |
| Rhobtb1 | 0.007892 | 0.108705 | 0.136 | 0.056 | 1 |
| Ppp3ca | 0.00797 | 0.192402 | 0.51 | 0.382 | 1 |
| Mir22hg | 0.007991 | −0.20736 | 0.581 | 0.674 | 1 |
| Bzw1 | 0.008066 | 0.190636 | 0.722 | 0.631 | 1 |
| Nr4a2 | 0.008114 | 0.272 | 0.298 | 0.201 | 1 |
| Cast | 0.008135 | 0.130942 | 0.581 | 0.598 | 1 |
| Tsc22d3 | 0.008156 | −0.26745 | 0.49 | 0.555 | 1 |
| Cd48 | 0.008238 | −0.15407 | 0.207 | 0.298 | 1 |
| Tle3 | 0.008318 | −0.11021 | 0.192 | 0.313 | 1 |
| Fmnl1 | 0.008338 | 0.128413 | 0.227 | 0.183 | 1 |
| Blnk | 0.008381 | 0.133946 | 0.167 | 0.081 | 1 |
| Stk24 | 0.008393 | −0.13701 | 0.394 | 0.514 | 1 |
| Lgals1 | 0.008396 | 0.188873 | 0.98 | 0.967 | 1 |
| Picalm | 0.008474 | 0.209011 | 0.894 | 0.863 | 1 |
| Lmbrd2 | 0.008511 | 0.132351 | 0.192 | 0.125 | 1 |
| Trappc6a | 0.008556 | 0.165628 | 0.419 | 0.361 | 1 |
| Me2 | 0.008557 | −0.20634 | 0.293 | 0.369 | 1 |
| Sphk1 | 0.008583 | −0.11465 | 0.126 | 0.234 | 1 |
| Smap2 | 0.008608 | 0.142219 | 0.394 | 0.366 | 1 |
| Prdm2 | 0.008698 | −0.18364 | 0.237 | 0.351 | 1 |
| Irak3 | 0.008716 | −0.16506 | 0.136 | 0.244 | 1 |
| Pum3 | 0.008718 | −0.12244 | 0.116 | 0.193 | 1 |
| Eif3c | 0.00884 | 0.197385 | 0.662 | 0.565 | 1 |
| Ssh2 | 0.008885 | 0.164549 | 0.227 | 0.122 | 1 |
| Cnbp | 0.008904 | 0.199491 | 0.581 | 0.45 | 1 |
| Rhog | 0.008959 | −0.11954 | 0.505 | 0.646 | 1 |
| Arhgap26 | 0.008987 | 0.124715 | 0.116 | 0.059 | 1 |
| Pias4 | 0.008996 | −0.12 | 0.172 | 0.295 | 1 |
| Ppp1r12c | 0.008998 | 0.161216 | 0.328 | 0.282 | 1 |
| Gm11361 | 0.00902 | 0.145513 | 0.253 | 0.168 | 1 |
| Otud4 | 0.009048 | 0.137854 | 0.192 | 0.122 | 1 |
| Srpk1 | 0.009288 | −0.16012 | 0.237 | 0.369 | 1 |
| Pcna | 0.009327 | −0.17118 | 0.525 | 0.613 | 1 |
| Elavl1 | 0.009332 | 0.155858 | 0.379 | 0.328 | 1 |
| Drg1 | 0.009472 | 0.104148 | 0.232 | 0.191 | 1 |
| Baz2a | 0.009483 | −0.16812 | 0.182 | 0.267 | 1 |
| Tnfrsf21 | 0.009593 | −0.12265 | 0.283 | 0.29 | 1 |
| Camkk2 | 0.009686 | 0.116801 | 0.177 | 0.168 | 1 |
| Slc38a2 | 0.009834 | 0.236361 | 0.626 | 0.55 | 1 |
| 2410006H16Rik | 0.009847 | 0.259469 | 0.571 | 0.466 | 1 |
| Ccl12 | 0.011194 | −0.36577 | 0.369 | 0.394 | 1 |
| Srxn1 | 0.01322 | 0.264291 | 0.343 | 0.326 | 1 |
| Gm26917 | 0.01415 | 0.331176 | 0.429 | 0.466 | 1 |
| Il1a | 0.014206 | −0.43741 | 0.49 | 0.593 | 1 |
| Csf3 | 0.01721 | −0.34481 | 0.061 | 0.13 | 1 |
| Pdia4 | 0.020784 | −0.25082 | 0.419 | 0.491 | 1 |
| Tgfbi | 0.022342 | −0.25257 | 0.667 | 0.646 | 1 |
| Marcks | 0.027391 | −0.26705 | 0.566 | 0.677 | 1 |
| Hspa1b | 0.028676 | 0.449513 | 0.338 | 0.26 | 1 |
| Pmp22 | 0.040704 | 0.285964 | 0.394 | 0.313 | 1 |
| Hspa1a | 0.042292 | 0.502038 | 0.429 | 0.392 | 1 |
| Flrt3 | 0.053561 | −0.31999 | 0.278 | 0.313 | 1 |
| Fabp5 | 0.05607 | −0.33509 | 0.783 | 0.822 | 1 |
| Neat1 | 0.066655 | 0.250635 | 0.929 | 0.885 | 1 |
| Ctsk | 0.070278 | −0.30266 | 0.146 | 0.221 | 1 |
| Atf3 | 0.075045 | 0.250055 | 0.788 | 0.756 | 1 |
| Prdx1 | 0.080583 | 0.25051 | 0.864 | 0.913 | 1 |
| Zfp36l1 | 0.094184 | 0.25153 | 0.763 | 0.743 | 1 |
| Chil3 | 0.254227 | −0.2843 | 0.126 | 0.183 | 1 |
| Cxcl10 | 0.367352 | 0.253808 | 0.202 | 0.198 | 1 |

In sum, profound changes were observed in the macrophages and other infiltrating immune cells in CT26 tumors treated with exoASO-STAT6, resulting in the remodeling of the TME towards immune activation and anti-tumor immunity.

### Example 4: Systemic Administration of exoASO-STAT6 Results in Potent Monotherapy Anti-Tumor Response in an HCC Model

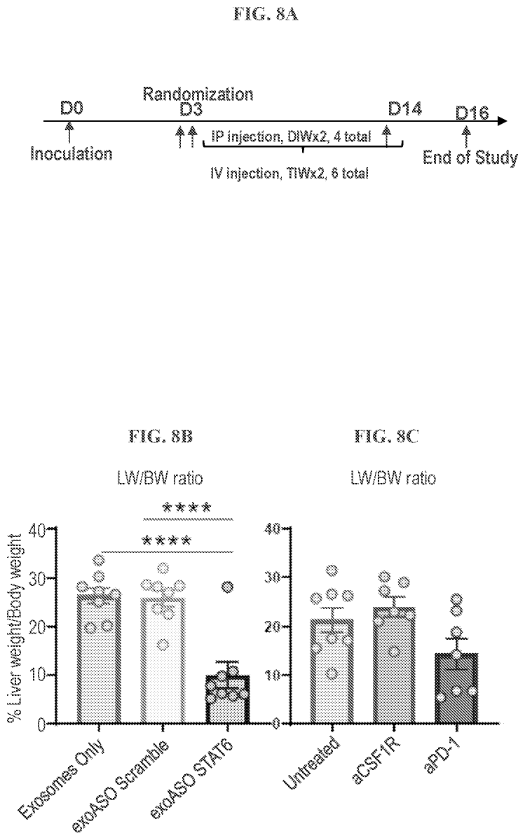

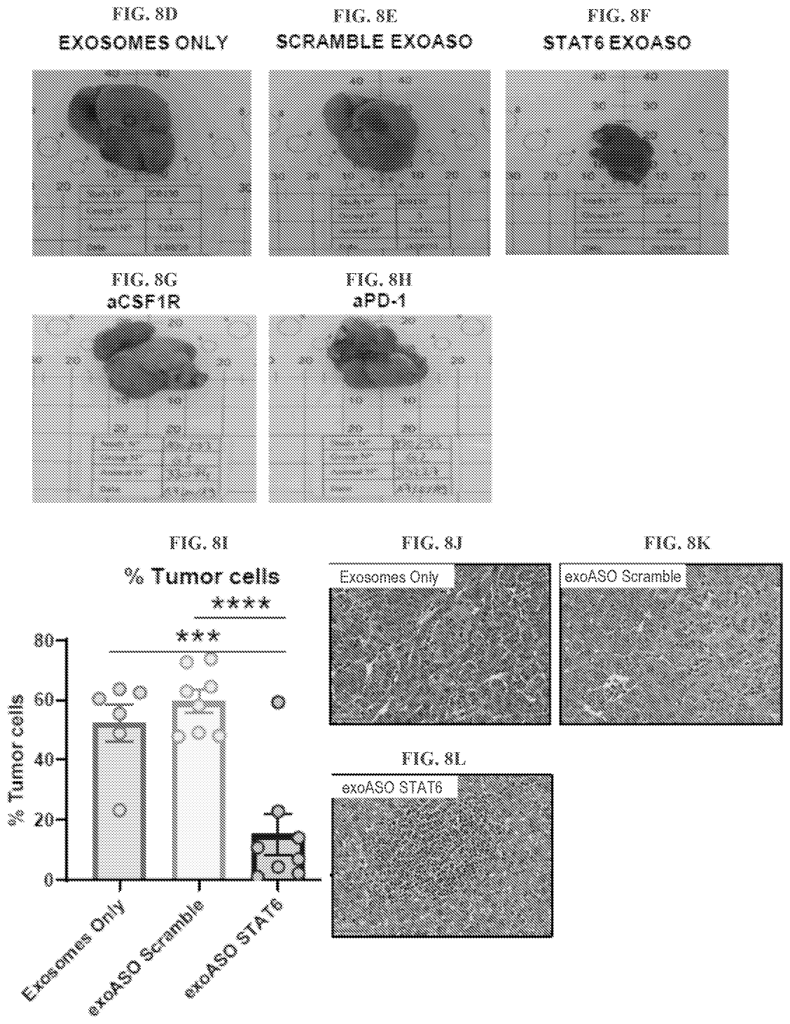

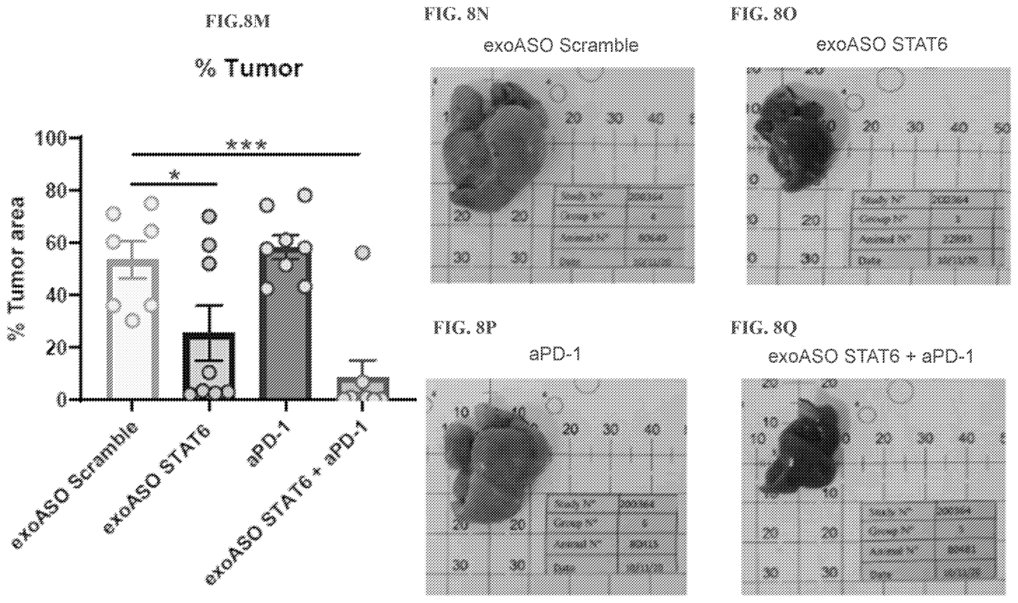

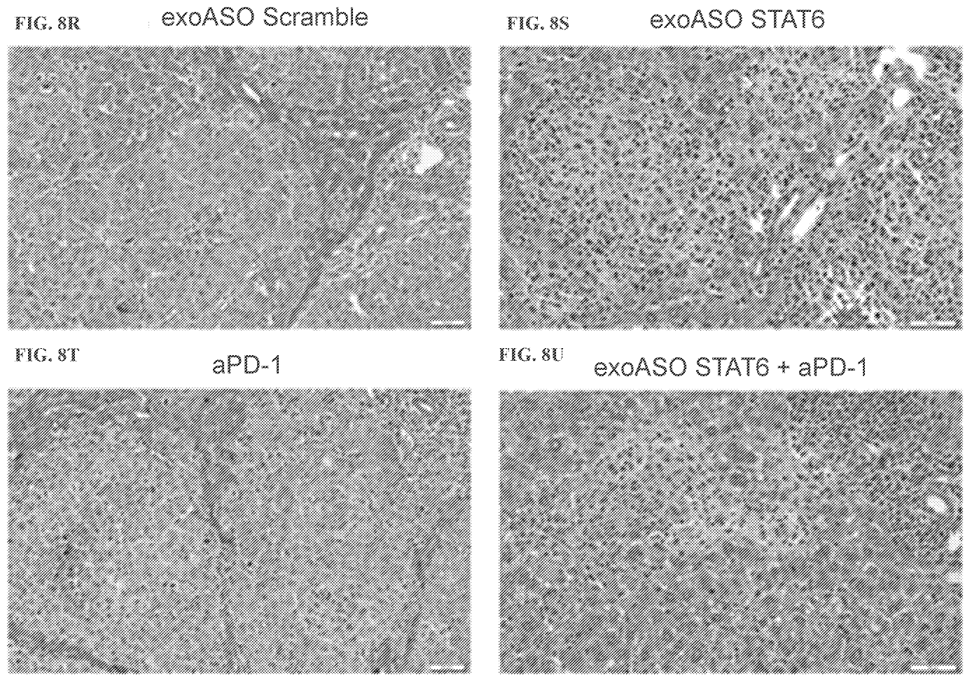

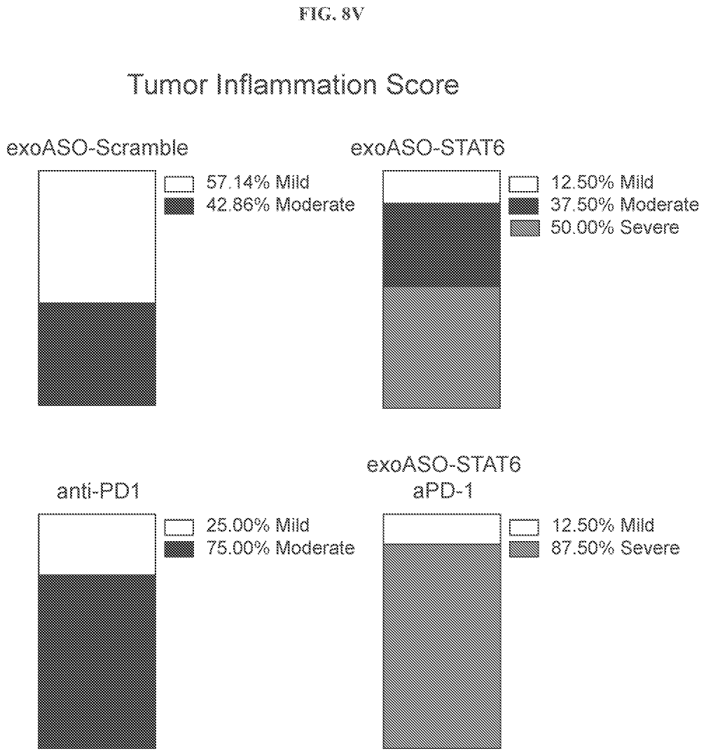

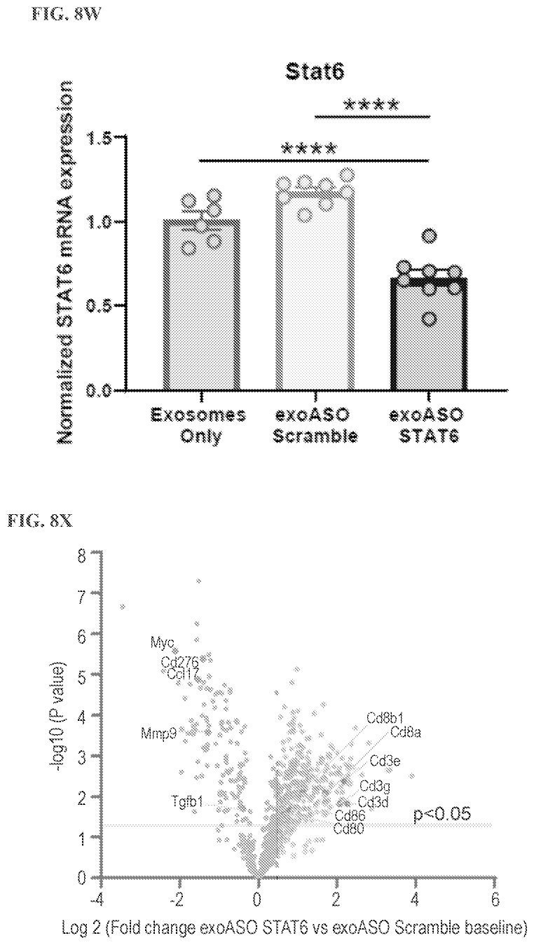

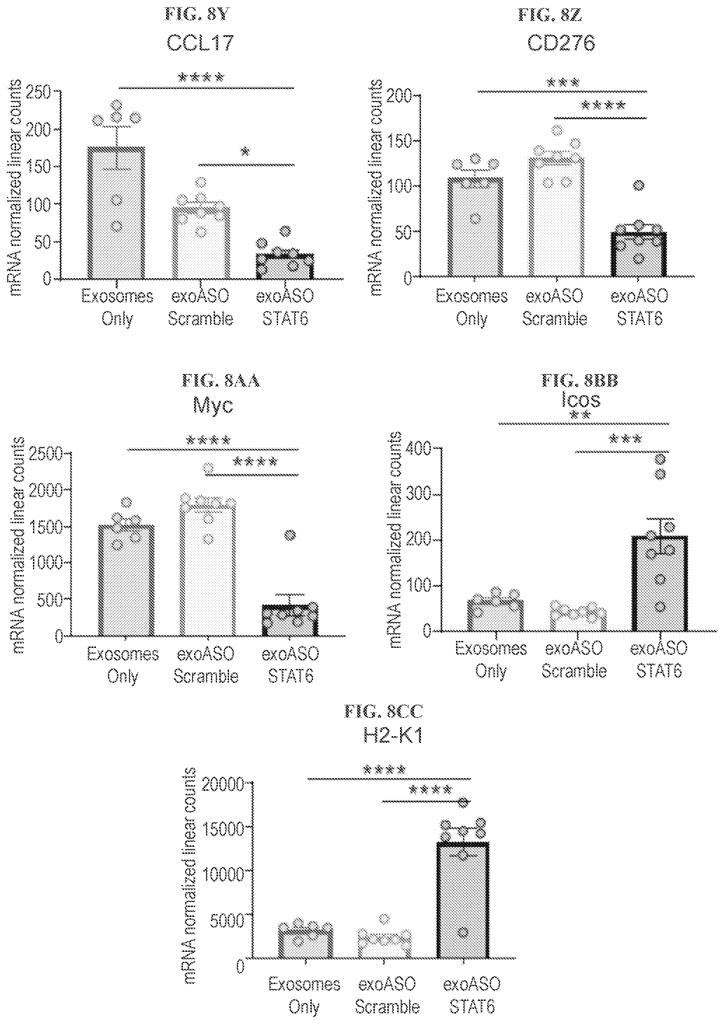

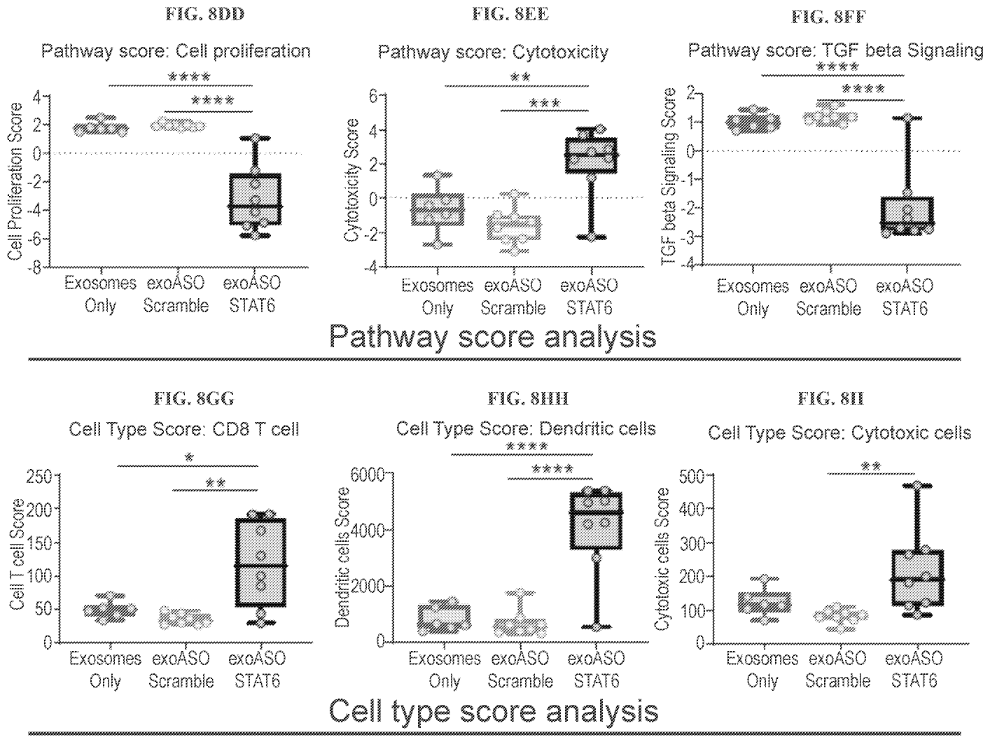

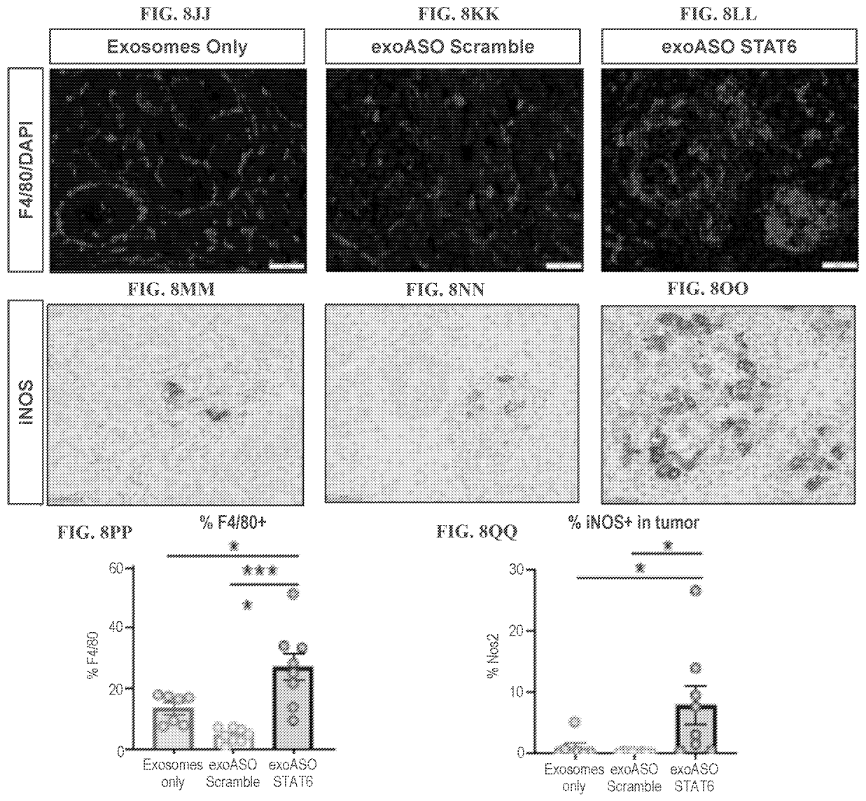

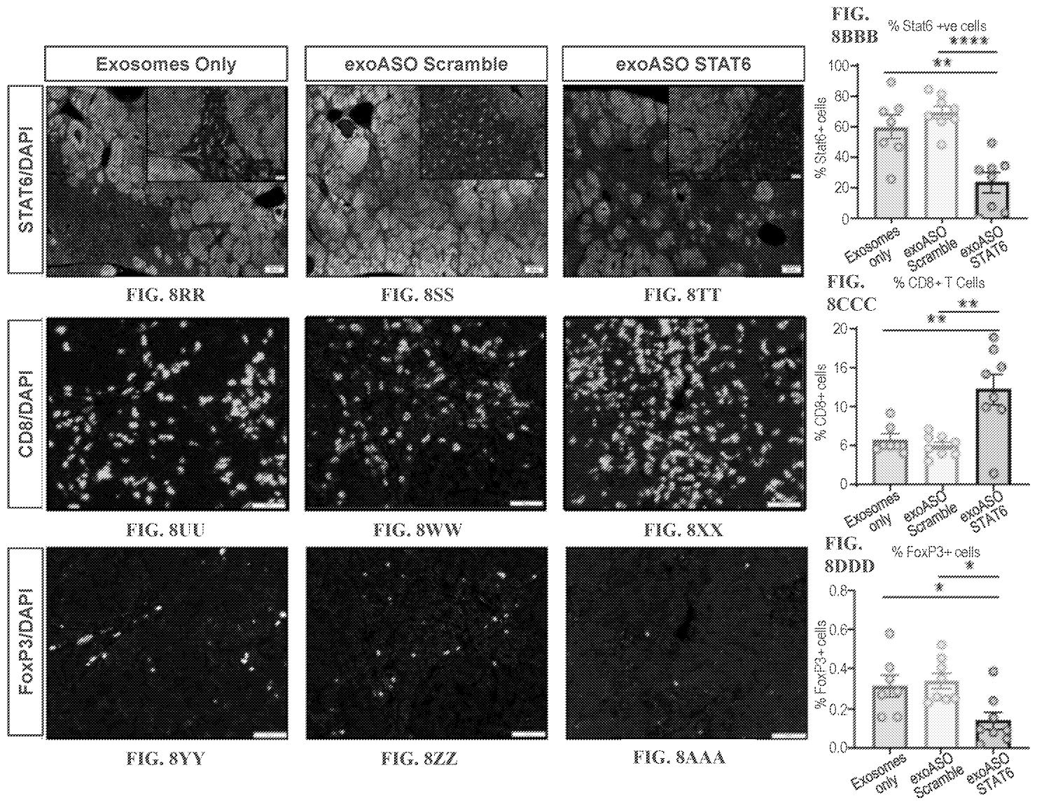

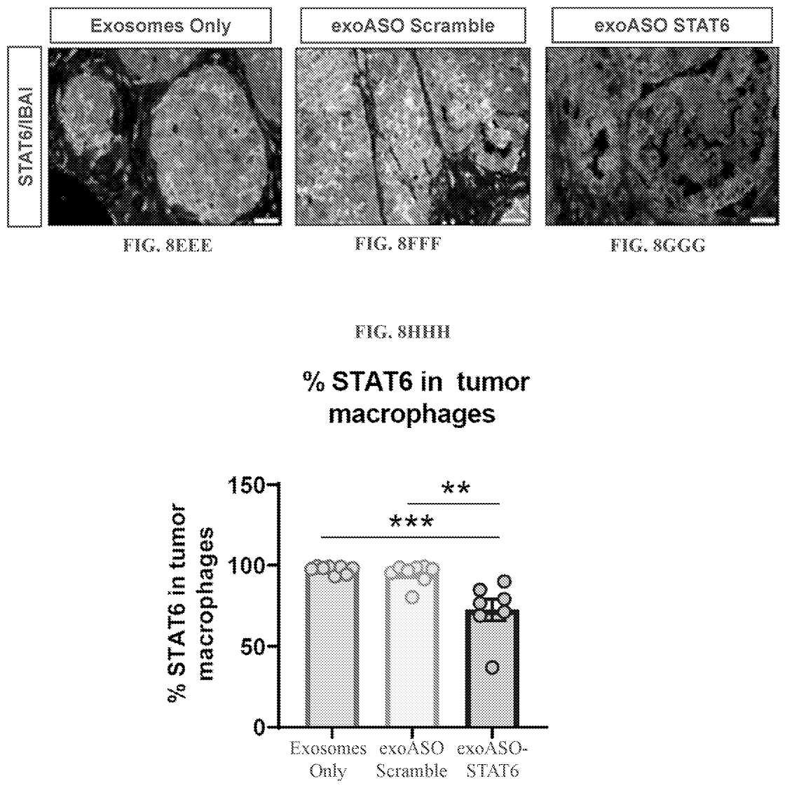

Most HCC patients do not show benefit from anti-PD-1 therapy. These tumors are also highly enriched in TAMs. The biodistribution of exosomes to the liver and macrophages in the tumor make this an ideal cancer type for exoASO-STAT6. In addition, exoASO-STAT6 induced potent Stat6 KD in the liver of naïve mice when dosed iv (69% KD compared to PBS control). In contrast, free STAT6 ASO resulted in only modest 38% KD, demonstrating that exosome-mediated delivery also enhances ASO potency when dosed systemically and suggesting that tumors located in the liver can be targeted by iv administration of exoASO. To assess the anti-tumoral efficacy of systemically dosed exoASO-STAT6 in HCC, we used the Hepa1-6 orthotopic model. As depicted in FIG. 8A, Hepa1-6 cells were injected intra-splenically, followed by immediate splenectomy. The treatment was initiated 3 days post tumor inoculation. This is a very aggressive tumor model which shows minimal response to anti-PD-1 therapy and is non-responsive to other macrophage depleting therapies such as anti-CSF1R (FIGS. 8B-8C).

Hepa1-6 liver orthotopic tumor bearing mice were injected with exoASO-STAT6 or control exoASO or empty exosomes from day 3 as indicated. The study was terminated on day 16 and the liver weight and body weight were measured. Tumor burden was plotted as a ratio of liver to body weight. ExoASO-STAT6 treatment resulted in a 62% reduction in tumor burden. In contrast, the control exoASO treated animals did not show any measurable decrease in tumor burden (FIGS. 8D-8H). Quantification of tumor cells in histological sections also showed a significant decrease (71%) in tumor burden in the exoASO-STAT6 treatment group compared to the exosomes only control (FIG. 8I). Close examination of H&E-stained liver sections showed markedly smaller tumor islands in the exoASO-STAT6 treated mice, and conspicuous immune infiltrate colocalizing with tumor areas (FIGS. 8F-8L). Interestingly, heavy immune cell infiltration was also observed in exoASO-STAT6 treated mice that did not show reduction in tumor burden. Combination therapy with an anti-PD1 antibody resulted in synergistic effects (FIGS. 8M-8U). Moreover, histopathological analysis revealed higher levels of inflammation in the combination group compared to the exoASO-STAT6 monotherapy group (FIG. 8V).

The effect of exoASO-STAT6 treatment on Stat6 expression was evaluated in whole liver tissue by real time RT-qPCR. Results showed that exoASO-STAT6 reduced Stat6 mRNA levels by 34% compared to the exosome-only treated group, demonstrating effective target gene KD (FIG. 8W). Immunohistochemistry assays on liver sections showed that exoASO-STAT6 also induced 61% KD of STAT6 protein compared to baseline expression in the exosome-only group (FIG. 8W). To investigate the mechanism of action underlying the significant antitumoral activity of exoASO-STAT6 in this model, gene expression analysis was performed on whole liver tissue at the end of the study. The expression of a pan-cancer gene panel was assessed by Nanostring. Treatment with exoASO-STAT6 induced significant changes in gene expression compared to exosomes-only and control exoASO (FIGS. 8X-8CC). Notably, several genes associated with an anti-tumoral T cell response were exclusively upregulated in the exoASO-STAT6 group (e.g., CD3e, CD8, ICOS, CD80, CD86) (FIGS. 8X-8CC). In contrast, this group showed decreased expression of M2 genes such as Cd276, Myc and Ccl17. Pathway and cell-type analysis not only confirmed the induction of a potent anti-tumoral immune response but also a decrease in tumor cell proliferation markers (FIGS. 8DD-8II). Compared to both control groups, exoASO-STAT6 group displayed significantly increased scores for cytotoxic cells, CD8 T cells, dendritic cells, and interferon signaling and significantly decreased scores for TGF-β signaling, cell proliferation and angiogenesis. The changes in the TME were confirmed by immunohistochemistry on liver sections. Results showed an increase in macrophage infiltration and a concomitant increase in expression of the M1 macrophage marker iNOS in the exoASO-STAT6 group (2- and 8.5-fold compared to exosomes-only group, respectively) (FIGS. 8JJ-8QQ), a strong CD8 T cell infiltration (2.1-fold increase vs exosome only group) and a significant reduction in Treg infiltration (2.3-fold decrease vs exosome only group) (FIGS. 8RR-8UU and 8WW-8DDD). In addition, immunohistochemistry assays on liver sections showed that exoASO-STAT6 also induced significant knockdown in Iba1+ macrophages within the tumor areas as compared to control groups (FIGS. 8EEE-8HHH). Moreover, the ASO co-localized preferentially with the iNOS negative macrophages, confirming that exoASO-STAT6 also shows selective tropism to M2 macrophages compared to M1 (data not shown).

Given the strong immune response elicited at the tumor site by IV dosing of exoASO-STAT6, we assessed if systemic inflammation was also induced by exoASO treatment by measuring cytokines in the serum of treated animals at the end of the study. Using multiplex flow-based assay, the concentration of 13 cytokines was quantified. No significant changes were observed between exoASO-STAT6 and control exoASO treatments or sham animals that did not have implanted tumors. In addition, no signs of hepatotoxicity were observed in any of the treatment groups as observed in the serum levels of aspartate aminotransferase (AST) and alanine aminotransferase (ALT).

These data demonstrate significant anti-tumor activity with exoASO-STAT6 therapy in the Hepa1-6 orthotopic tumor model and a lack of systemic inflammation by iv dosing of exoASO-STAT6.

### Example 5: STAT6 Macrophage Signature Correlates with Poor Disease Prognosis in HCC

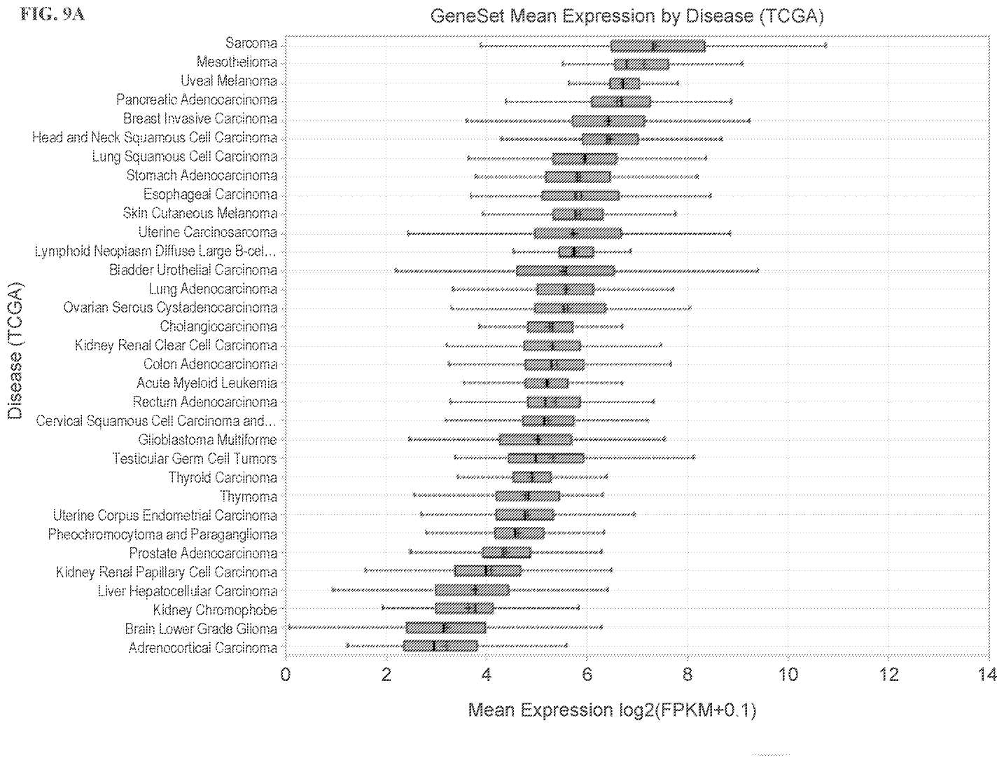

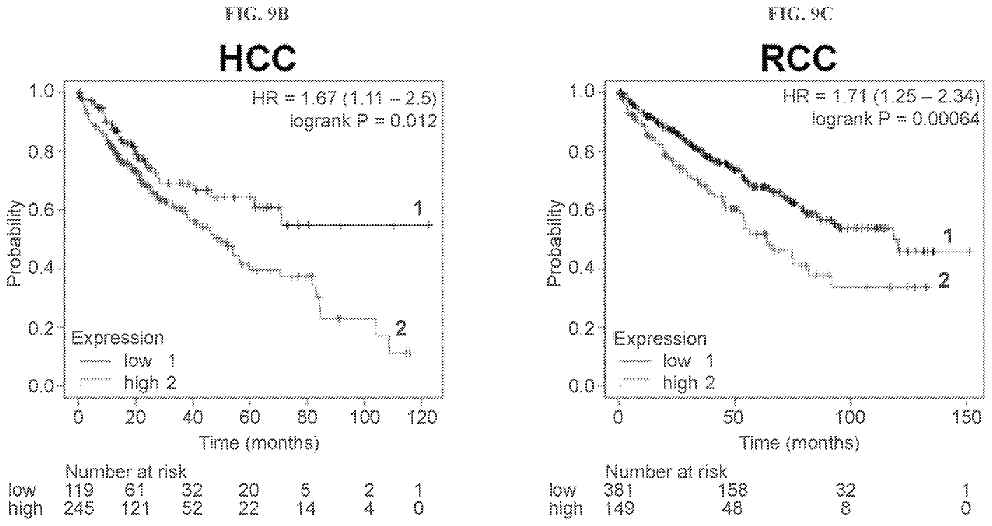

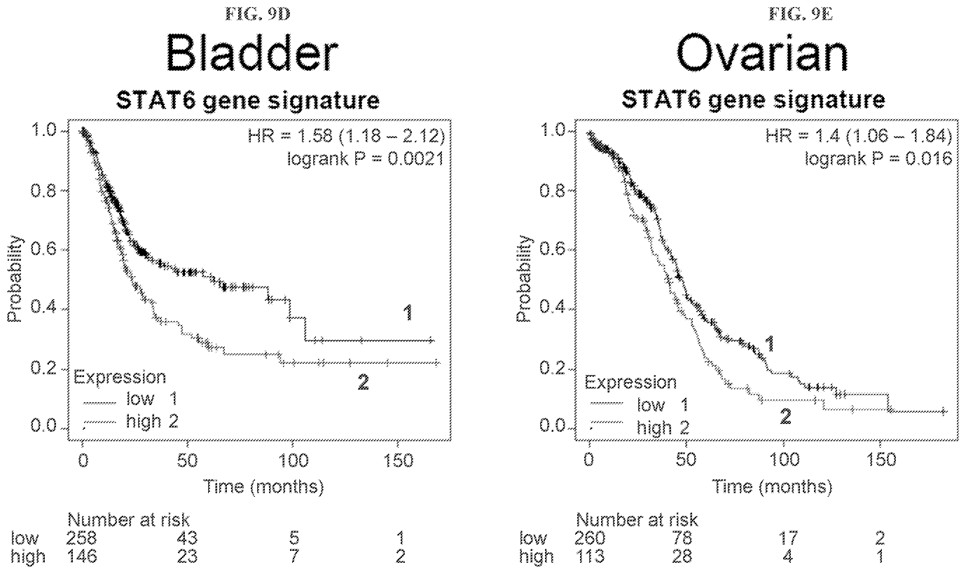

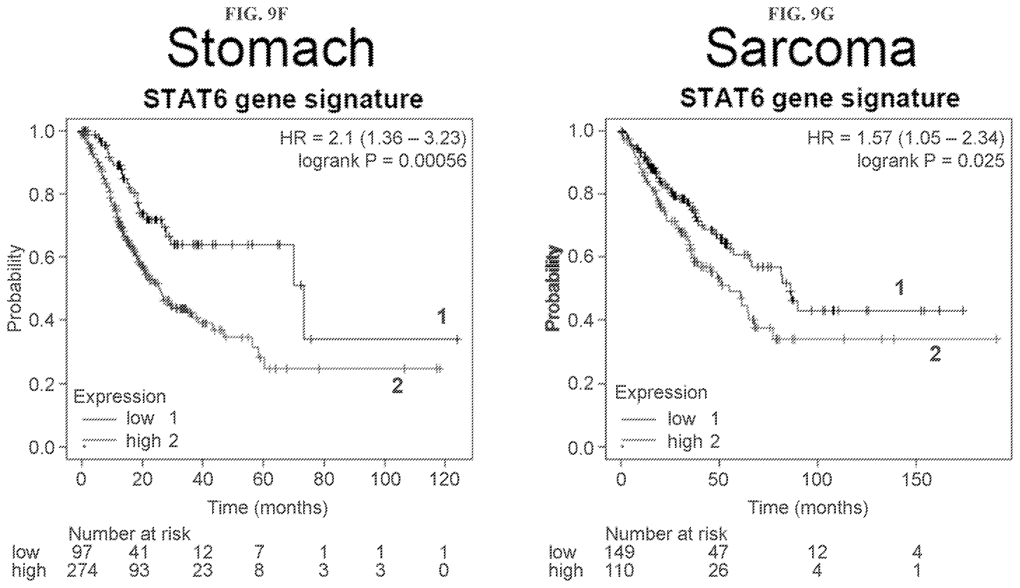

To investigate the translational significance of our preclinical findings, we generated a STAT6 signature based on the gene expression changes induced by exoASO-STAT6 in human M2 macrophages (Table 5). We identified top 40 genes that were modulated by exoASO-STAT6 treatment and analyzed the expression of this set genes in HCC tumors from the TCGA database. Correlation analysis identified a subset of 10 genes from the 40-gene list that are coherently expressed across the HCC tumors that was independent from the previously published IFNγ related tumor inflammation signature (TIS) associated with benefit from anti-PD-1 treatment. This signature is composed of genes such as IL4R, CCL2 which are associated with STAT6 signaling and immune-suppressive cytokines such as TGFβ. It should be noted that immune inflammatory genes such as IL1B are also part of the signature suggesting that the macrophages in the TME may be composed of both inflammatory and immune-suppressive phenotype. The expression of this list of 10-gene STAT6 genes was compared with other cell type specific immune cell markers (B-cells, Tregs, Macrophage and NK cells) and TIS signature in the HCC TCGA cohort. Unsupervised clustering identified 3 molecular subsets in this cohort: immune cell infiltrated (inflamed), immune cell poor/non-inflamed, and macrophage-rich/CD8 T-cell excluded subsets. Based on the gene expression pattern, STAT6 signature enriched tumors were present in both the immune cell rich tumors as well as macrophage-rich/CD8 T-cell poor tumors. Among the immune cell infiltrated tumors, STAT6 signature was enriched in tumors with high macrophage, B-cells and Treg cell markers, and a subset of cases in tumors with a high TIS. Interestingly, the STAT6 gene list is also expressed in a subset of tumors within the CD8 excluded, coinciding with higher levels of macrophage genes. A similar gene-expression pattern was also observed in an independent tumor type, renal cell carcinoma (RCC) patients. We further demonstrated that the STAT6 signature is expressed across other TCGA indications at moderate to high levels (FIG. 9A). Next, we evaluated the impact STAT6 signature on disease progression in primarily HCC as well as RCC and other indications. High expression (quartile cutoff) of the STAT6 signature correlated with poorer survival in HCC and RCC (FIGS. 9A-9B; P=0.012 and P=0.00064 for HCC and RCC, respectively) among other indications (FIGS. 9D-9G). When disease progression was analyzed in both the CD8-enriched and CD8-low HCC tumors, the STAT6 signature negatively affected survival in both cases. Although IL-4 expression was generally low, it was also associated with poor prognosis in CD8 low patients. Altogether, these results suggest that there is a clinical opportunity to target the STAT6 pathway in macrophages in HCC and that both patients with CD8-enriched or CD8-decreased tumors could benefit from treatment with exoASO-STAT6.

TABLE 5
Forty gene list for STAT6 signature

| No. | Gene |
|---|---|
| 1 | CD163 |
| 2 | IL4R |
| 3 | TGFb1 |
| 4 | MRC1 |
| 5 | AIF1 |
| 6 | CD40 |
| 7 | CD276 |
| 8 | MIF |
| 9 | CCL26 |
| 10 | COL1A2 |
| 11 | ALOX15 |
| 12 | SELE |
| 13 | CEBPB |
| 14 | MYC |
| 15 | CD44 |
| 16 | SIGLEC 1 |
| 17 | CD274 |
| 18 | CCL13 |
| 19 | CCL5 |
| 20 | CXCL9 |
| 21 | CTSS |
| 22 | CSFIR |
| 23 | CIQA |
| 24 | C1QB |
| 25 | C1QC |
| 26 | CD68 |
| 27 | FN1 |
| 28 | CD14 |
| 29 | CCL2 |
| 30 | CCR3 |
| 31 | IL1B |
| 32 | CD1A |
| 33 | EDN1 |
| 34 | IL15RA |
| 35 | IRF5 |
| 36 | IL12B |
| 37 | CD86 |
| 38 | CCR7 |
| 39 | PTGS1 |
| 40 | CCL17 |

Based on the results obtained from the CT26 and Hepa1-6 tumor models, herein we propose a model to describe the anti-tumor activity mediated by genetic reprogramming of TAM by exoASO-STAT6. STAT6-expressing TAMs are a determining factor in the generation of a protumoral, immunosuppressive TME by promoting recruitment of Tregs and inhibition of CD8 cytotoxic T cells, among other mechanisms. The ability of exoASO to knockdown STAT6 in immunosuppressive TAMs results in effective reprogramming to an M1 phenotype that promotes the induction of a cytotoxic immune response and an antitumoral TME. Thus, exoASO-STAT6 treatment differs from other macrophage targeting therapies in that it induces the replacement of dysfunctional TAMs by antitumoral TAMs, instead of causing a detrimental depletion of the entire macrophage population.

### Example 6: Filtration of Compositions Comprising Exosomes Loaded with ASOs

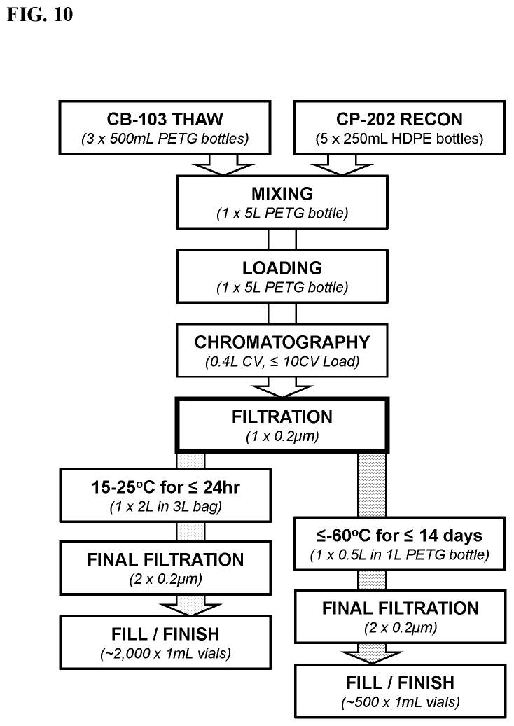

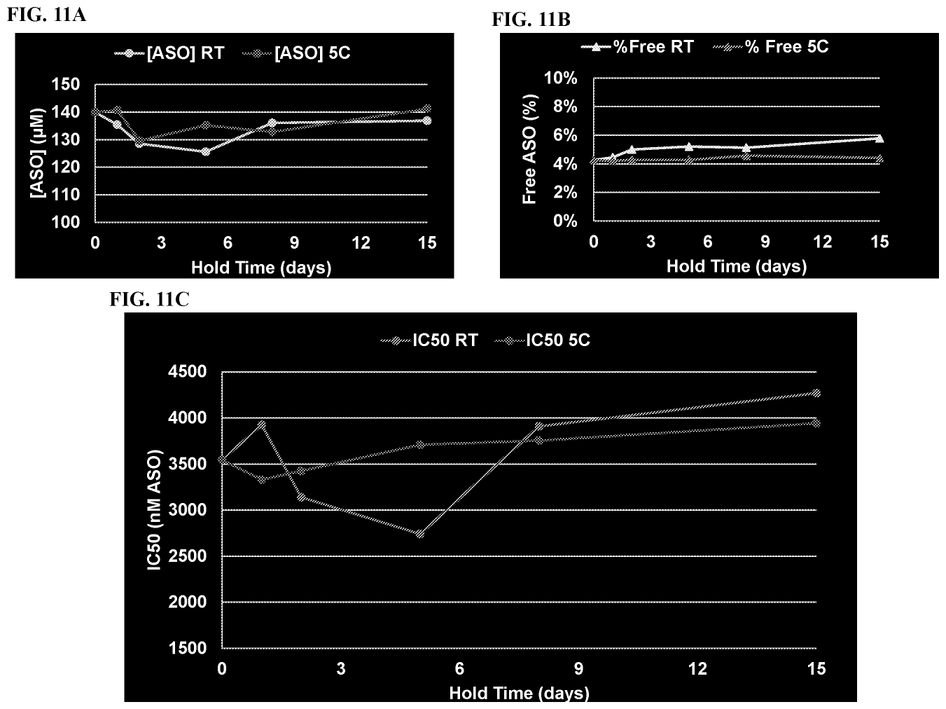

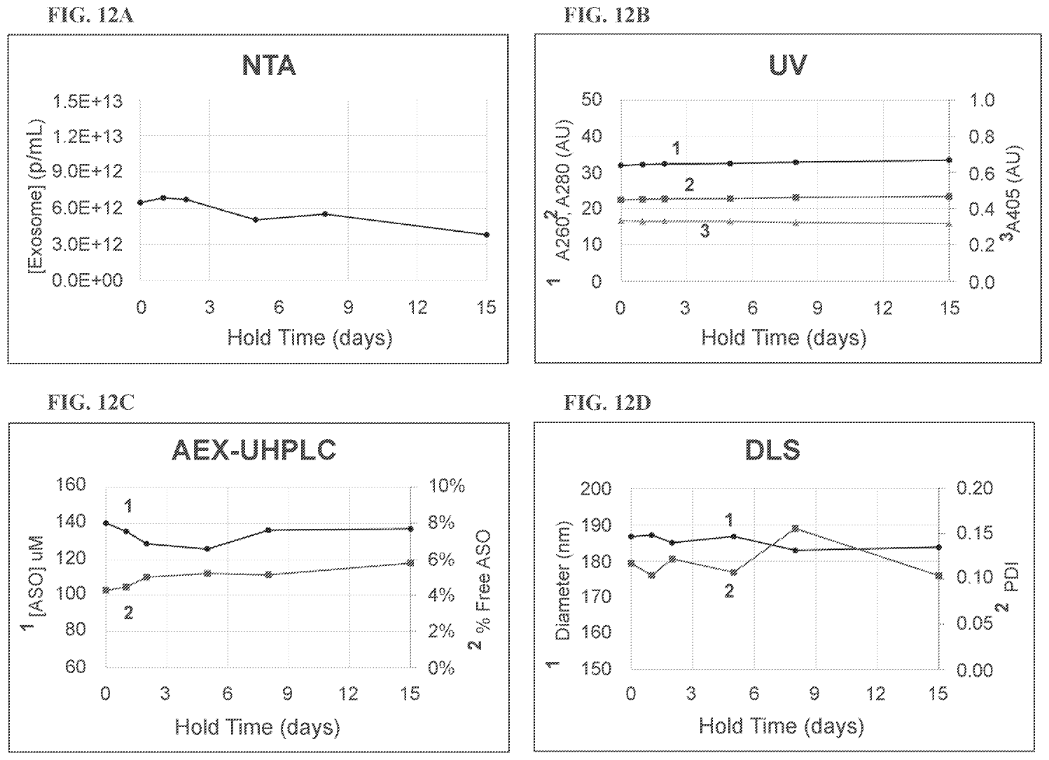

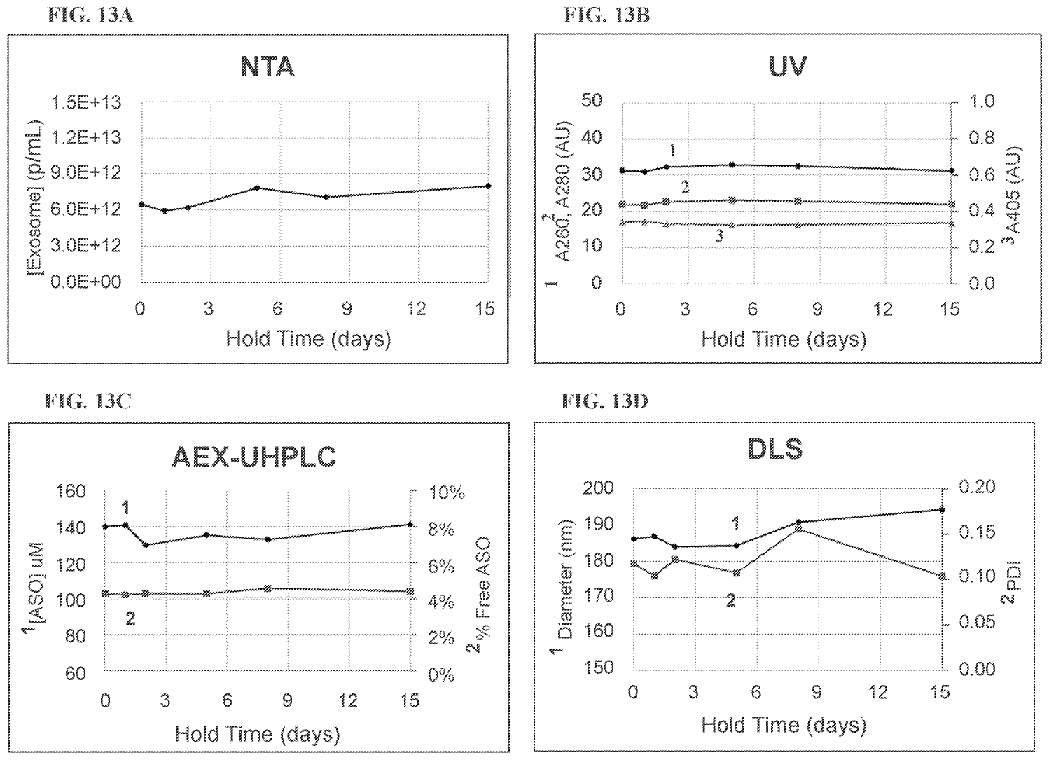

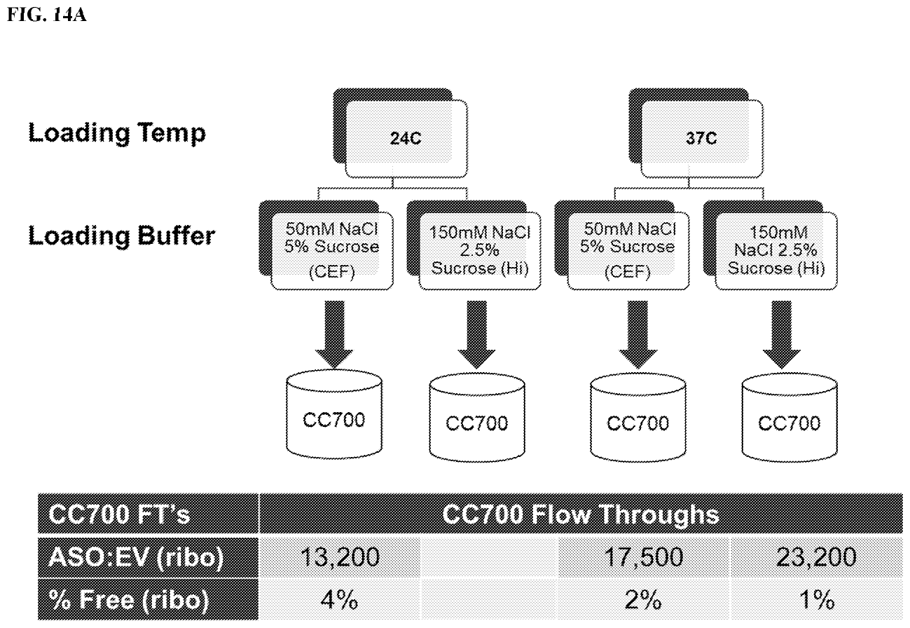

Exosomes loaded with ASOs targeting STAT6, as disclosed herein, were purified according to the protocol shown in FIG. 10. Following the filtration step, samples were held at room temperature or 5° C. for 15 days, and ASO concentration (FIG. 11A), free ASO percent (FIG. 11B), and IC50 (FIG. 11C) were analyzed. Material was held at 20-22° C. (room temperature) and 5° C. for 15 days and tested throughout hold time for ASO content, both total and free, A260, A280, A405, in vitro potency with the STAT6 mRNA knock down cell reporter assay (FIGS. 12B and 13B), NTA for particle concentration (FIGS. 12A and 13A), and DLS (FIGS. 12D and 13D) to measure mean particle diameter and polydispersity index. Total ASO content measured by AEX-UPLC (FIG. 12C) indicates stability across a 15 day (about 2 weeks) hold at both hold temperatures. Percent free of ASO increases slightly as a function of hold time, while 5° C. hold seems to reduce percent free ASO. In vitro potency data shows IC50 values comparable across 8-day hold for RT. 5° C. trends towards less potent with longer hold, however changes are slight.

Following the room temperature hold (20-22° C.), NTA shows stability through 8 day hold. There were no changes observed in A260, A280, and A405, which indicates no loss of product through precipitation or adsorption to hold container, nor increase in turbidity. DLS shows so change in mean particle diameter indicating no observed aggregations across the hold.

Following the 5° C. hold, NTA shows stability through 15 day hold, with no changes observed in A260, A280, and A405, which indicates no loss of product through precipitation or adsorption to hold container, nor increase in turbidity. DLS shows no change in mean particle diameter to 8-day hold, indicating no observed aggregations across the hold. Day-15 hold shows a slight increase in diameter.

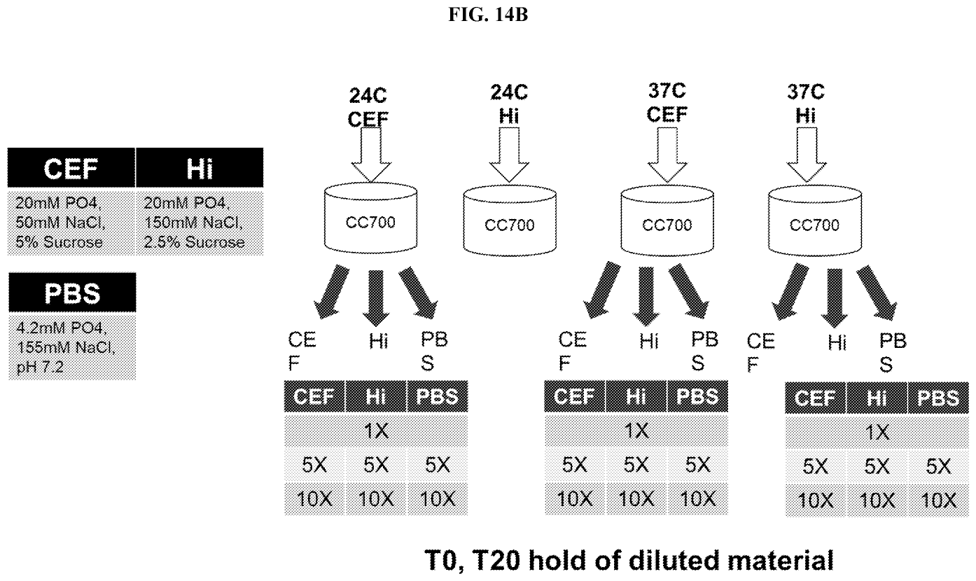

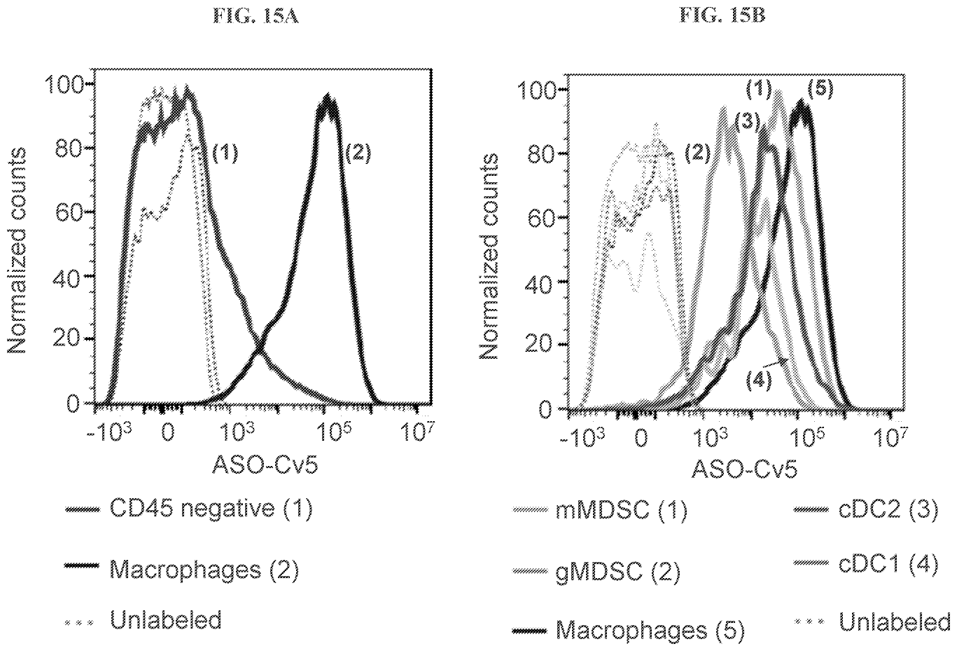

FIG. 14A provides a schematic diagram for a protocol that demonstrates differences in ASO/EV of loaded and cleaned up material when loading at room temperature as compared to loading at 37° C. and in higher salt (150 mM NaCl) as compared to in lower salt (50 mM NaCl) conditions. The highest salt condition when loading at 37° C. yields the highest ASO/EV in the cleaned up material. This ratio decreases when loading at 37° C., but at lower salt concentrations. An additional decrease in the ASO/EV is observed when reducing the loading temperature to 24° C. (or room temp, which ranges 20-25° C.). The percent free ASO shows that capto core is able to clean up the free ASO independent of the loading temperature and salt concentration, indicating that this method has robust cleanup capabilities.

FIG. 14B provides a schematic diagram for a protocol that highlights clean up capabilities of Capto Core for the three conditions run, above, and the effect of dilution factor, diluent (CEF, HI, and PBS), and hold time of diluted material on ASO's that dissociate from exosomes after they have been cleaned up from Capto Core. PBS has a similar salt concentration to a common and potential diluent (0.9% saline) that can be used in the clinic to dilute the exoASO prior to injection. The percent free ASO indicates that when diluting loaded and cleaned up material in a buffer containing a lower salt concentration, more ASO's dissociate from the EV's (Table 6). The dissociation becomes more drastic as the dilution factor increases. However, when diluting material in the higher salt containing buffers (150 mM NaCl), there is no dissociation of ASO's observed. When the diluted cleaned up material is held for 20 hours at room temperature, additional dissociation is observed only when the diluent contains lower salt concentrations (50 mM). For the higher salt containing diluents, no dissociation is observed when the diluted material is held for 20 hours at room temperature. These data indicate that the stability of the loaded exosomes can be optimized by maintaining a salt concentration.

TABLE 6
Capto Core Analysis

| | % Free in Sup t0 | | | | | | | | | | | |
|---|---|---|---|---|---|---|---|---|---|---|---|---|
| Loading Buffer | CEF | | | | | | Hi | | | | | |
| Loading Temperature | 22C | | | 37C | | | RT | | | 37C | | |
| Diluent | CEF | Hi | PBS | CEF | Hi | PBS | CEF | Hi | PBS | CEF | H | PBS |
| 1× | 4% | | | | | | 2% | | | 1% | | |
| 5× | 14% | 2% | 5% | | | | 11% | 3% | 2% | 6% | 1% | 1% |
| 10× | 14% | 2% | 2% | | | | 15% | 2% | 3% | 10% | 1% | 1% |
| | % Free Sup t20 | | | | | | | | | | | |
| | CEF | | | | | | Hi | | | | | |
| | 22C | | | 37C | | | RT | | | 37C | | |
| | CEF | Hi | PBS | CEF | Hi | PBS | CEF | Hi | PBS | CEF | Hi | PBS |
| | 7% | | | | | | 2% | | | 1% | | |
| | 17% | 2% | 2% | | | | 14% | 2% | 2% | 7% | 1% | 1% |
| | 29% | 1% | 1% | | | | 23% | 2% | 3% | 15% | 1% | 1% |

### Example 7: In Vivo Analysis of Exosomes Loaded with ASOs

To facilitate clinical translation, PD studies were conducted in cynomolgus monkeys, which demonstrated a dose dependent and durable silencing of STAT6 (50% and 31% at 1- and 3-weeks post-dose, respectively). STAT6 knockdown correlated with a reduction in STAT6 target genes, IL4R and EGR2, suggesting modulation of the STAT6 pathway.

### Example 8: In Vivo Localization of Exosomes Loaded with ASOs

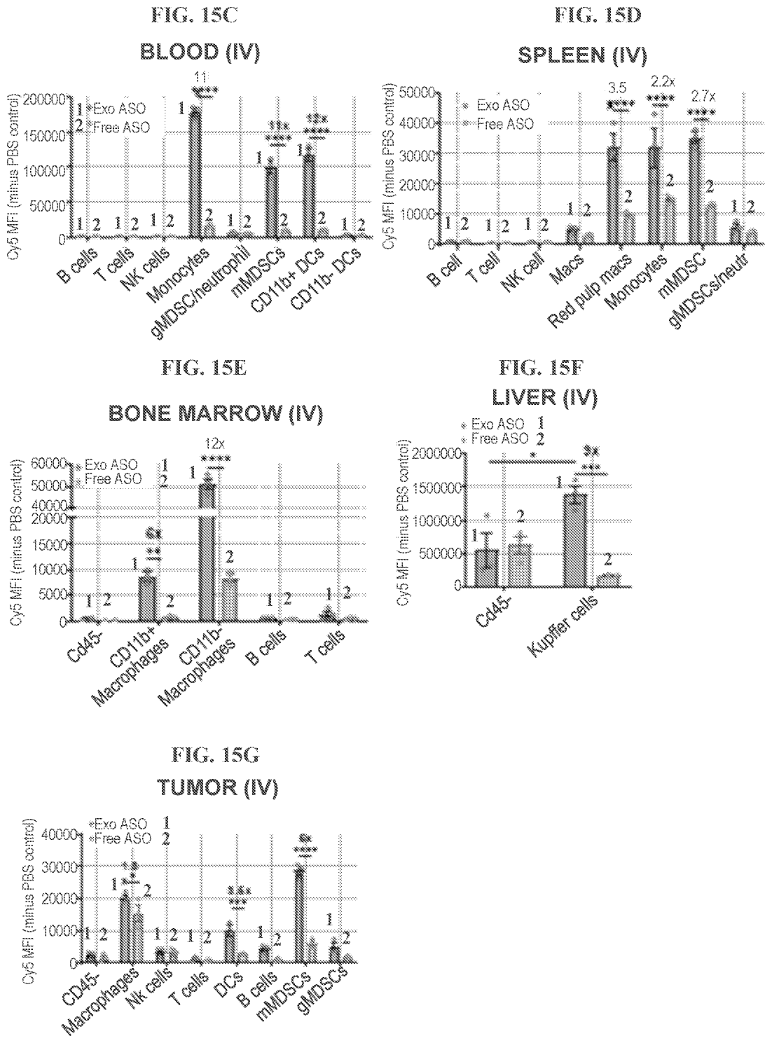

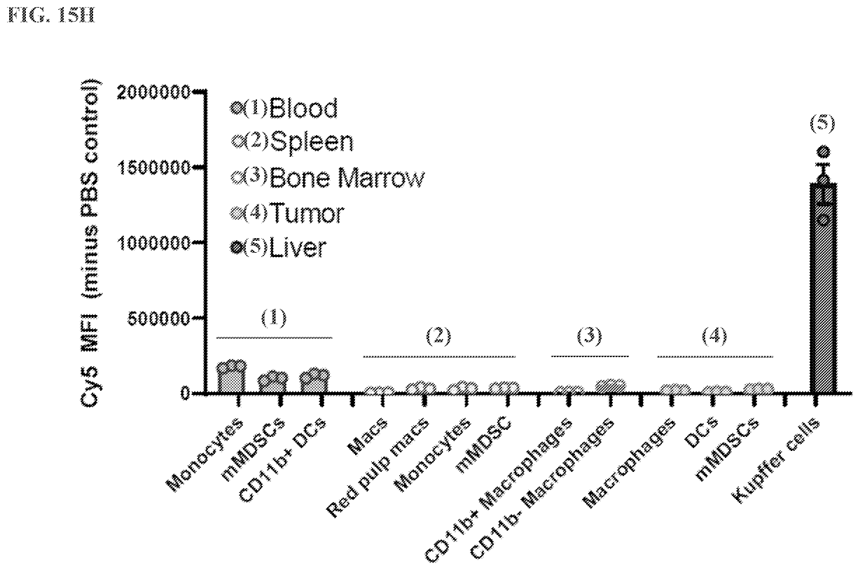

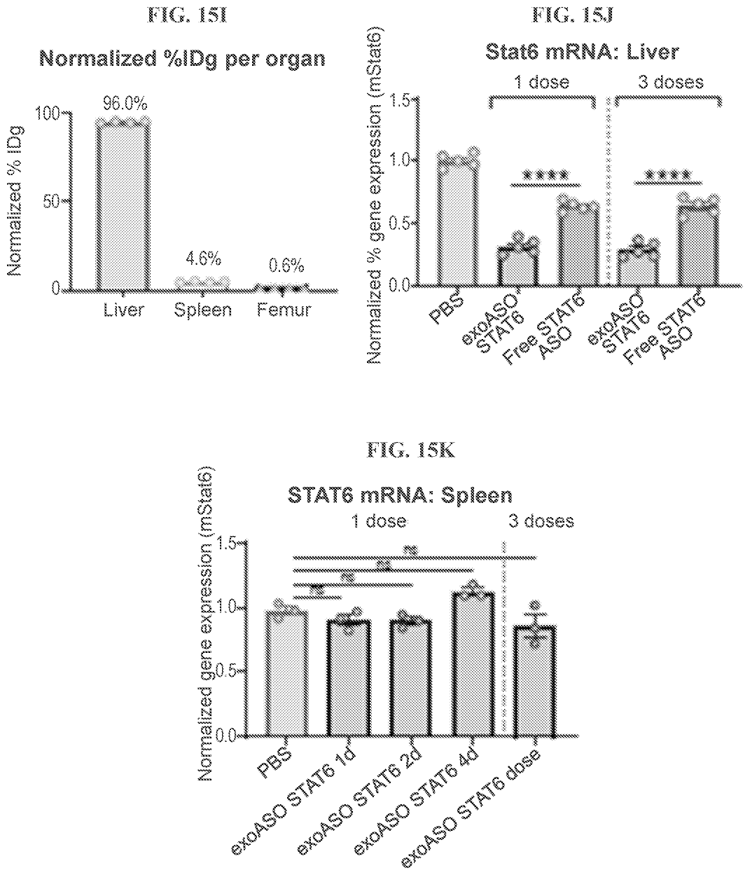

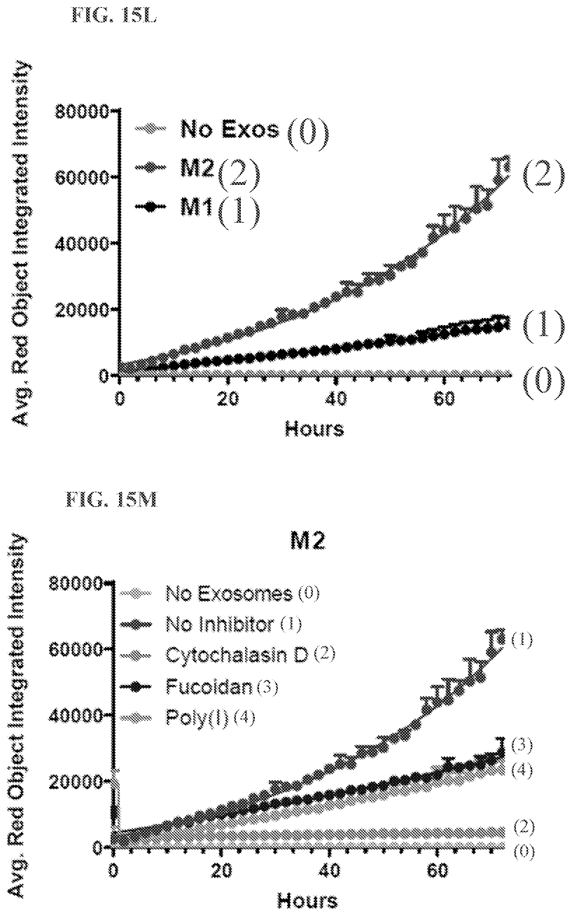

To determine the cellular tropism of exoASO in vivo, the biodistribution of Cy5-labelled exoASO-STAT6 or free ASO was evaluated in CT26 colon cancer tumor-bearing mice. After intratumoral administration, minimal ASO-Cy5 signal was observed in the non-immune compartment (CD45 negative cells; FIG. 15A) while TAMs showed the highest mean fluorescence intensity (FIG. 15B). Other cells of myeloid origin (dendritic cells (DCs), MDSCs) showed intermediate levels of ASO signal. Similarly, 1 hour post IV administration, myeloid cells of the monocytic lineage (monocytes, macrophages, liver Kupffer cells, MDSCs, myeloid dendritic cells) showed the highest ASO-Cy5 signal. In contrast, B cells, T cells, NK cells and granulocytic myeloid cells showed minimal association with exoASO. The exoASO treated group consistently demonstrated significantly higher Cy5 signal compared to the free ASO group in all tissues analyzed, including tumors (FIGS. 15C-15G). In blood monocytes, exosomes enhanced ASO delivery by 11-fold, compared to free ASO. A similar trend was observed in tissue macrophages (3.5-fold, 9-fold and 12-fold increase vs free ASO for spleen red pulp macrophages, liver Kupffer cells and bone marrow macrophages, respectively). Exosomes were also more effective in delivering ASO to myeloid cells in the CT26 subcutaneous tumors (1.3-fold, 3.5-fold and 5-fold increase vs free ASO for TAMs, DCs and MDSCs, respectively). These data demonstrate that exosomes potentiate the delivery of ASO to macrophages, monocytes, and MDSCs in the liver, peripheral blood, bone marrow and tumor microenvironment.

A comparison of the fluorescence intensity among the major cell populations associated with ASO in each tissue clearly demonstrated that liver Kupffer cells display significantly higher ASO signal than any of the other subsets (8-fold, 27-fold, 43-fold and 69-fold higher than the signal in blood, bone marrow, spleen and subcutaneous tumor macrophages/monocytes, respectively) (FIG. 15H). This observation is in line with the result of a tissue/organ biodistribution study using Zr89 labeled PTGFRN exosomes which showed the 95% of the injected IV dose localized in the liver, while only 4.6% and 0.6% accumulated in the spleen and femur, respectively (FIG. 15I). In agreement with its tissue biodistribution pattern, exoASO-STAT6 induced potent Stat6 knockdown in the liver of naïve mice when dosed IV (69% reduction in Stat6 mRNA levels compared to PBS control; FIG. 15J), while no changes in Stat6 expression were observed in the spleen (FIG. 15K). Free STAT6 ASO resulted in only modest 38% knockdown in the liver, demonstrating that exosome-mediated delivery enhances ASO potency when dosed systemically.

To investigate cellular uptake and intracellular trafficking of exoASO-STAT6, exoASO uptake was evaluated in M1 and M2 polarized human monocyte-derived macrophages (MDMs). To monitor phagocytosis and endocytosis, exoASO-STAT6 was labelled with pHrodo dye, a pH sensitive dye that fluoresces in acidic environments such as the endolysosome compartment, and treated macrophages were followed for 72 hr by live cell imaging. M2 macrophages showed higher fluorescence accumulation as compared with M1 macrophages (4-fold, FIG. 15L), demonstrating preferential uptake of exoASO by M2 polarized macrophages. exoASO uptake by M2 macrophages was evaluated in the presence of the phagocytosis inhibitor cytochalasin D, the class A scavenger receptor inhibitor Fucoidan and the non-selective scavenger receptor inhibitor poly(I). Cytochalasin D treatment resulted in maximal inhibition (93%) of uptake, whereas treatment with Fucoidan and poly (I) blocked ˜50% uptake (FIG. 15G). Thus, exoASO uptake is superior in M2 macrophages and is mediated by phagocytosis and scavenger receptor mediated mechanisms.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific aspects have been illustrated and described, the above specification is not restrictive. It will be appreciated that various changes can be made without departing from the spirit and scope of the invention(s). Many variations will become apparent to those skilled in the art upon review of this specification.

What is claimed:

1. A method of preventing or treating a disease or condition in a subject in need thereof, comprising administering to the subject (i) an extracellular vesicle comprising an antisense oligonucleotide (ASO) and (ii) a programmed death-1 (PD-1) antagonist; wherein the ASO comprises a contiguous nucleotide sequence of 10 to 30 nucleotides in length that is complementary to a nucleic acid sequence within a target transcript, wherein the target transcript is a STAT6 transcript (SEQ ID NO: 1 or SEQ ID NO: 3).

2. A method of increasing or enhancing an immune response in a subject in need thereof, comprising administering to the subject (i) an extracellular vesicle comprising an antisense oligonucleotide (ASO) and (ii) a programmed death-1 (PD-1) antagonist; wherein the ASO comprises a contiguous nucleotide sequence of 10 to 30 nucleotides in length that is complementary to a nucleic acid sequence within a target transcript, wherein the target transcript is a STAT6 transcript (SEQ ID NO: 1 or SEQ ID NO: 3).

3. The method of claim 1 or 2, wherein the contiguous nucleotide sequence is complementary to a nucleic acid sequence within nucleotides 1 to 2056 of a STAT6 transcript corresponding to a nucleotide sequence as set forth in SEQ ID NO: 3 or nucleotides 2059 to 3963 of a STAT6 transcript corresponding to a nucleotide sequence as set forth in SEQ ID NO: 3.

4. The method of any one of claims 1 to 3, wherein the ASO is a gapmer, a mixmer, or a totalmer.

5. The method of any one of claims 1 to 4, wherein the ASO comprises one or more nucleoside analogs.

6. The method of claim 5, wherein one or more of the nucleoside analogs comprises a 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-ANA; or bicyclic nucleoside analog.

7. The method of claim 5 or 6, wherein one or more of the nucleoside analogs is a sugar modified nucleoside.

8. The method of claim 7, wherein the sugar modified nucleoside is an affinity enhancing 2′ sugar modified nucleoside.

9. The method of any one of claims 5 to 8, wherein one or more of the nucleoside analogs comprises a nucleoside comprising a bicyclic sugar.

10. The method of any one of claims 5 to 8, wherein one or more of the nucleoside analogs comprises an LNA.

11. The method of any one of claims 5 to 10, wherein one or more of the nucleotide analogs is selected from the group consisting of constrained ethyl nucleoside (cEt), 2′,4′-constrained 2′-O-methoxyethyl (cMOE), α-L-LNA, β-D-LNA, 2′-O,4′-C-ethylene-bridged nucleic acids (ENA), amino-LNA, oxy-LNA, thio-LNA, and any combination thereof.

12. The method of any one of claims 1 to 11, wherein the ASO comprises one or more 5′-methyl-cytosine nucleobases.

13. The method of any one of claims 1 to 12, wherein the contiguous nucleotide sequence is complementary to a nucleic acid sequence within (i) a 5′ untranslated region (UTR); (ii) a coding region; or (iii) a 3′ UTR of the target transcript.

14. The method of any one of claims 1 to 13, wherein the contiguous nucleotide sequence is complementary to a nucleic acid sequence comprising (i) nucleotides 1-700 of SEQ ID NO: 3; (ii) nucleotides 1000-1500 of SEQ ID NO: 3; (iii) nucleotides 1500-2000 of SEQ ID NO: 3; (iv) nucleotides 2000-2500 of SEQ ID NO: 3; (v) 2500-3000 of SEQ ID NO: 3; (vi) 3000-3700 of SEQ ID NO: 3, (vii) nucleotides 413-803 of SEQ ID NO: 3; (viii) nucleotides 952-1688 of SEQ ID NO: 3; (ix) nucleotides 1726-2489 of SEQ ID NO: 3; (x) nucleotides 2682-2912 of SEQ ID NO: 3; (xi) 2970-3203 of SEQ ID NO: 3; (xii) 3331-3561 of SEQ ID NO: 3; (xiii) nucleotides 463-753 of SEQ ID NO: 3; (xiv) nucleotides 1002-1638 of SEQ ID NO: 3; (xv) nucleotides 1776-2439 of SEQ ID NO: 3; (xvi) nucleotides 2682-2862 of SEQ ID NO: 3; (xvii) 3020-3153 of SEQ ID NO: 3; (xviii) 3381-3511 of SEQ ID NO: 3; (xix) nucleotides 503-713 of SEQ ID NO: 3; (xx) nucleotides 1042-1598 of SEQ ID NO: 3; (xxi) nucleotides 1816-2399 of SEQ ID NO: 3; (xxii) nucleotides 2722-2822 of SEQ ID NO: 3; (xxiii) 3060-3113 of SEQ ID NO: 3; or (xxiv) 3421-3471 of SEQ ID NO: 3.

15. The method of any one of claims 1 to 14, wherein the contiguous nucleotide sequence is complementary to a nucleic acid sequence within (i) nucleotides 513-703 of SEQ ID NO: 3; (ii) nucleotides 1052-1588 of SEQ ID NO: 3; (iii) nucleotides 1826-2389 of SEQ ID NO: 3; (iv) nucleotides 2732-2812 of SEQ ID NO: 3; (v) 3070-3103 of SEQ ID NO: 3; or (vi) 3431-3461 of SEQ ID NO: 3.

16. The method of any one of claims 1 to 15, wherein the ASO comprises a nucleic acid sequence selected from GAAAGGTTCCGTCGGGC (SEQ ID NO: 144), CTGAGTCGCTGAAGCGG (SEQ ID NO: 145), GCCCTTGTACTTTTGCATAG (SEQ ID NO: 193), GCAAGATCCCGGATTCGGTC (SEQ ID NO: 185), and any combination thereof.

17. The method of any one of claims 1 to 16, wherein the contiguous nucleotide sequence comprises a nucleotide sequence complementary to a sequence selected from the sequences in FIG. 1.

18. The method of any one of claims 1 to 17, wherein the continuous nucleotide sequence is fully complementary to a nucleotide sequence within the target transcript.

19. The method of any one of claims 1 to 18, wherein the ASO comprises a nucleotide sequence selected from SEQ ID NOs: 91-193, with one or two mismatches.

20. The method of any one of claims 1 to 19, wherein the ASO has a design selected from the group consisting of the designs in FIG. 1, wherein the upper letter is a sugar modified nucleoside and the lower case letter is DNA.

21. The method of any one of claims 1 to 20, wherein the ASO is from 14 to 20 nucleotides in length.

22. The method of any one of claims 1 to 21, wherein the contiguous nucleotide sequence comprises one or more modified internucleoside linkages.

23. The method of claim 22, wherein the one or more modified internucleoside linkages is a phosphorothioate linkage.

24. The method of claim 22 or 23, wherein at least 75%, at least 80%, at least 85%, at least 90%, at least 95%, or 100% of internucleoside linkages are modified.

25. The method of claim 24, wherein each of the internucleoside linkages in the ASO is a phosphorothioate linkage.

26. The method of any one of claims 1 to 25, wherein the ASO is linked to an anchoring moiety.

27. The method of claim 26, wherein the anchoring moiety comprises a sterol, GM1, a lipid, a vitamin, a small molecule, a peptide, or a combination thereof.

28. The method of claim 26 or 27, wherein the anchoring moiety comprises cholesterol.

29. The method of any one of claims 25 to 27, wherein the anchoring moiety comprises a phospholipid, a lysophospholipid, a fatty acid, a vitamin (e.g., vitamin D and/or vitamin E), or any combination thereof.

30. The method of any one of claims 26 to 29, wherein the anchoring moiety is associated with the extracellular vesicle.

31. The method of any one of claims 26 to 30, wherein the extracellular vesicle comprises a lipid bilayer, wherein the anchoring moiety is associated with the lipid bilayer of the extracellular vesicle.

32. The method of any one of claims 26 to 31, wherein the ASO is linked to the anchoring moiety on the exterior surface of the EV.

33. The method of any one of claims 26 to 31, wherein the ASO is linked to the anchoring moiety on the luminal surface of the EV.

34. The method of any one of claims 26 to 31, wherein the ASO is linked to the anchoring moiety.

35. The method of any one of claims 26 to 31, wherein the anchoring moiety comprises a scaffold moiety.

36. The method of any one of claims 1 to 35, wherein the ASO is linked to the EV by a linker.

37. The method of claim 35 or 36, wherein the linker is a polypeptide.

38. The method of claim 35 or 36, wherein the linker is a non-polypeptide moiety.

39. The method of claim 35 or 36, wherein the linker comprise ethylene glycol.

40. The method of claim 39, wherein the linker comprises HEG, TEG, PEG, or any combination thereof.

41. The method of claim 35 or 36, wherein the linker comprises acrylic phosphoramidite (e.g., ACRYDITE™), adenylation, azide (NHS Ester), digoxigenin (NHS Ester), cholesterol-TEG, I-LINKER™, an amino modifier (e.g., amino modifier C6, amino modifier C12, amino modifier C6 dT, or Uni-Link™ amino modifier), alkyne, 5′ Hexynyl, 5-Octadiynyl dU, biotinylation (e.g., biotin, biotin (Azide), biotin dT, biotin-TEG, dual biotin, PC biotin, or desthiobiotin), thiol modification (thiol modifier C3 S—S, dithiol or thiol modifier C6 S—S), or any combination thereof.

42. The method of any one of claims 35 to 41, wherein the linker is a cleavable linker.

43. The method of claim 42, wherein the linker comprises valine-alanine-p-aminobenzylcarbamate or valine-citrulline-p-aminobenzylcarbamate.

44. The method of any one of claims 35 to 43, wherein the linker comprises (i) a maleimide moiety and (ii) valine-alanine-p-aminobenzylcarbamate or valine-citrulline-p-aminobenzylcarbamate.

45. The method of any one of claims 1 to 44, wherein the extracellular vesicle further comprises an exogenous targeting moiety.

46. The method of claim 45, wherein the exogenous targeting moiety comprises a peptide, an antibody or an antigen-binding fragment thereof, a chemical compound, an RNA aptamer, or any combination thereof.

47. The method of claim 45 or 46, wherein the exogenous targeting moiety comprises a peptide.

48. The method of any one of claims 45 to 37, wherein the exogenous targeting moiety comprises a microprotein, a designed ankyrin repeat protein (darpin), an anticalin, an adnectin, an aptamer, a peptide mimetic molecule, a natural ligand for a receptor, a camelid nanobody, or any combination thereof.

49. The method of any one of claims 45 to 48, wherein the exogenous targeting moiety comprises a full-length antibody, a single domain antibody, a heavy chain only antibody (VHH), a single chain antibody, a shark heavy chain only antibody (VNAR), an scFv, a Fv, a Fab, a Fab′, a F(ab′)2, or any combination thereof.

50. The method of claim 52, wherein the antibody is a single chain antibody.

51. The method of any one of claims 45 to 50, wherein the exogenous targeting moiety targets the exosome to the liver, heart, lungs, brain, kidneys, central nervous system, peripheral nervous system, muscle, bone, joint, skin, intestine, bladder, pancreas, lymph nodes, spleen, blood, bone marrow, or any combination thereof.

52. The method of any one of claims 45 to 51, wherein the exogenous targeting moiety targets the extracellular vesicle to a tumor cell, dendritic cell, T cell, B cell, macrophage, neuron, hepatocyte, Kupffer cell, myeloid-lineage cell (e.g., a neutrophils, monocytes, macrophages, hematopoietic stem cell, an MDSC (e.g., a monocytic MDSC or a granulocytic MDSC)), or any combination thereof.

53. The method of any one of claims 45 to 51, wherein the extracellular vesicle comprises a scaffold moiety linking the exogenous targeting moiety to the extracellular vesicle.

54. The method of claim 35 or 53, wherein the scaffold moiety is a Scaffold X.

55. The method of claim 35 or 54, wherein the scaffold moiety is a Scaffold Y.

56. The method of any one of claims 1 to 55, wherein the extracellular vesicle is an exosome.

57. The method of any one of claims 1 to 56, wherein the PD-1 antagonist comprises an antibody or an antigen-binding portion thereof that specifically bind to human PD-1 and blocks or inhibits the interaction between PD-1 and PD-L1 (“an anti-PD-1 antibody”).

58. The method of claim 57, wherein the anti-PD-1 antibody is selected from the group consisting of nivolumab, pembrolizumab, PDR001, MEDI-0680, cemiplimab, JS001, BGB-A317, INCSHR1210, TSR-042, GLS-010, AM-0001, STI-1110, AGEN2034, MGA012, IBI308, and any combination thereof.

59. The method of any one of claims 1 to 56, wherein the PD-1 antagonist comprises an antibody or an antigen-binding portion thereof that specifically bind to human PD-L1 and blocks or inhibits the interaction between PD-1 and PD-L1 (“an anti-PD-L1 antibody”).

60. The method of claim 59, wherein the anti-PD-L1 antibody is selected from the group consisting of atezolizumab, durvalumab, avelumab, STI-1014, CX-072, KN035, LY3300054, CK-301, BMS-936559, and any combination thereof.

61. The method of any one of claims 1 to 60, wherein the extracellular vesicle and the PD-1 antagonist are administered concurrently.

62. The method of any one of claims 1 to 61, wherein the extracellular vesicle and the PD-1 antagonist are administered sequentially.

63. The method of claim 62, wherein the extracellular vesicle and the PD-1 antagonist are administered on different days.

64. The method of any one of claims 1 to 63, wherein the PD-1 antagonist is linked to or associated with the extracellular vesicle.

65. The method of any one of claims 1 to 64, wherein the extracellular vesicle, the PD-1 antagonist, or both are administered by a route selected from the groups consisting of parenteral administration, topical administration, intravenous administration, oral administration, subcutaneous administration, intra-arterial administration, intradermal administration, transdermal administration, rectal administration, intracranial administration, intraperitoneal administration, intrathecal administration, intranasal administration, intratumoral administration, intramuscular administration, inhalation, and any combination thereof.

66. The method of any one of claims 1 to 65, wherein the subject is afflicted with a cancer.

67. The method of claim 66, wherein the cancer is selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell cancer, squamous cell cancer of the head and neck cancer, colorectal cancer, lymphoma, leukemia, liver cancer, glioblastoma, melanoma, myeloma basal cell cancer, adenocarcinoma, sweat gland cancer, sebaceous gland cancer, papillary cancer, papillary adenocarcinomas, cystadenocarcinoma, medullary cancer, bronchogenic cancer, renal cell cancer, hepatoma, bile duct cancer, choriocarcinoma, seminoma, nonseminoma, embryonal cancer, Wilms' tumor, cervical cancer, testicular cancer, lung cancer, small cell lung cancer, bladder cancer, epithelial cancer, glioma, glioblastoma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, follicular lymphoma, Hodgkin's lymphoma, B cell lymphoma, and any combination thereof.
